(19) United States
(12) Patent Application Publication
(10) Pub. No.: US 2014/0088124 A1
(43) Pub. Date: Mar. 27, 2014

(54) IMIDAZOLE DERIVATIVES

(71) Applicants: Robert J. DeVita, Westfield, NJ (US); QingMei Hong, Scotch Plains, NJ (US); Zhong Lai, Rahway, NJ (US); Kevin D. Dykstra, West Milford, NJ (US); Yang Yu, Clark, NJ (US); Jian Liu, Edison, NJ (US); Donald M. Sperbeck, East Hanover, NJ (US); Tianying Jian, Westfield, NJ (US); Deodial Guiadeen, Chesterfield, NJ (US); Ginger Xu-qiang Yang, Edison, NJ (US); Zhicai Wu, Montvale, NJ (US); Shuwen He, Edison, NJ (US); Pauline C. Ting, New Providence, NJ (US); Robert Aslanian, Rockaway, NJ (US); Jeffrey T. Kuethe, Somerset, NJ (US); James Balkovec, Martinsville, NJ (US); Rongze Kuang, Green Brook, NJ (US); Gang Zhou, Bridgewater, NJ (US); Heping Wu, Edison, NJ (US)

(72) Inventors: Robert J. DeVita, Westfield, NJ (US); QingMei Hong, Scotch Plains, NJ (US); Zhong Lai, Rahway, NJ (US); Kevin D. Dykstra, West Milford, NJ (US); Yang Yu, Clark, NJ (US); Jian Liu, Edison, NJ (US); Donald M. Sperbeck, East Hanover, NJ (US); Tianying Jian, Westfield, NJ (US); Deodial Guiadeen, Chesterfield, NJ (US); Ginger Xu-qiang Yang, Edison, NJ (US); Zhicai Wu, Montvale, NJ (US); Shuwen He, Edison, NJ (US); Pauline C. Ting, New Providence, NJ (US); Robert Aslanian, Rockaway, NJ (US); Jeffrey T. Kuethe, Somerset, NJ (US); James Balkovec, Martinsville, NJ (US); Rongze Kuang, Green Brook, NJ (US); Gang Zhou, Bridgewater, NJ (US); Heping Wu, Edison, NJ (US)

(21) Appl. No.: 14/123,246

(22) PCT Filed: Jun. 1, 2012
(86) PCT No.: PCT/EP2012/060381

§ 371 Date: Dec. 2, 2013

Related U.S. Application Data
(60) Provisional application No. 61/492,428, filed on Jun. 2, 2011.

(51) Int. Cl. C07D 401/14; C07D 417/14; C07D 493/04; C07D 471/04

(52) U.S. Cl. CPC C07D 401/14; C07D 471/04; C07D 417/14; C07D 493/04
USPC 514/269; 546/194; 514/318; 546/118; 514/303; 544/298

(57) ABSTRACT

Described herein are compounds of formula (I), The compounds of formula I act as DGAT1 inhibitors and can be useful in preventing, treating or acting as a remedial agent for hyperlipidemia, diabetes mellitus and obesity.

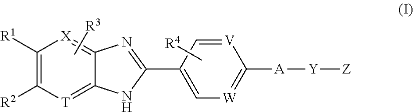

### CROSS-REFERENCE TO RELATED APPLICATIONS

This application claims the benefit of U.S. Provisional Application Ser. No. 61/492,428, filed Jun. 2, 2011, the contents of which are herein incorporated by reference in their entirety.

### TECHNICAL FIELD

The present invention is directed to novel imidazole derivative compounds. Specifically, the compounds act as diacylglycerol O-acyltransferase type 1 inhibitors (hereinafter also referred to as “DGAT1”), and can be useful in preventing, treating or acting as a remedial agent for hyperlipidemia, diabetes mellitus and obesity.

### BACKGROUND

Obesity is a medical condition in which excess body fat has accumulated to the extent that it may have an adverse effect on health, leading to reduced life expectancy and increased health problems. As such, obesity is recognized as an upstream risk factor for many conditions such as diabetes mellitus, lipidosis and hypertension (Journal of Japan Society for the Study of Obesity, Vol. 12, Extra Edition, 2006). Although the need to treat obesity is recognized to be important, there are extremely limited drug therapies for obesity that are currently available, and thus, the advent of novel anti-obesity drugs having more definite action and few side-effects is desired.

In general, obesity is caused by the accumulation of triacylglycerol (TG) in adipose tissue which is a result of lack of exercise, intake of excessive calories and aging. In the body there are two TG synthesis pathways, a glycerol phosphate pathway, which is present in most organs and causes de novo TG synthesis, and a monoacylglycerol pathway, which is involved principally in absorption of aliphatic acid from the small intestine. Diacylglycerol acyltransferases (DGATs, EC 2.3.1.20), which are membrane-bound enzymes present in the endoplasmic reticulum, catalyze the final step of the TG synthesis common to the two TG synthesis pathways. The final reaction consists of transferring an acyl group from acyl-coenzyme A to the 3-position of 1,2-diacylglycerol to generate TG (Prog. Lipid Res., 43, 134-176, 2004 and Ann. Med., 36, 252-261, 2004). There are two subtypes of DGATs, DGAT-1 and DGAT-2. There is no significant homology at the generic or amino acid level between the DGAT-1 and DGAT-2, which are encoded by different genes (Proc. Natl. Acad. Sci. USA., 95, 13018-13023, 1998 and JBC, 276, 38870-38876, 2001). DGAT-1 is present in the small intestine, adipose tissue and liver and is believed to be involved in lipid absorption in the small intestine; lipid accumulation in the fat cell; and VLDL secretion and lipid accumulation in the liver (Ann. Med., 36, 252-261, 2004 and JBC, 280, 21506-21514, 2005). In consideration of these functions, a DGAT-1 inhibitor is expected to be an effective obesity treatment through inhibition of lipid absorption in the small intestine, lipid accumulation in the adipose tissue and the liver, and lipid secretion from the liver.

In order to carry out in vivo examination of the physiological function(s) of DGAT-1 and inhibitory activity against DGAT-1, DGAT-1-knockout mice deficient in DGAT-1 at the genetic level was produced and analyzed. As a result, the DGAT-1-knockout mice have been found to have smaller fat masses than those of wild-type mice and became resistant to obesity, abnormal glucose tolerance, insulin resistance and fatty liver due when fed a high-fat diet (Nature Genetics, 25, 87-90, 2000 and JCI, 109, 1049-1055, 2002). In addition, energy expense has been reported to be accelerated in the DGAT-1-knockout mice; and transplantation of the adipose tissues of DGAT-1-knockout mice into wild-type mice has been reported to make the wild-type mice resistant to obesity and abnormal glucose tolerance, induced by a high-fat diet (JCI, 111, 1715-1722, 2003 and Diabetes, 53, 1445-1451, 2004). In contrast, obesity and diabetes mellitus due to a high-fat diet have been reported to worsen in mice with overexpression of DGAT-1 in adipose tissue (Diabetes, 51, 3189-3195, 2002 and Diabetes, 54, 3379-3386, 2005).

From the results, DGAT-1 inhibitors are likely to be therapeutic drugs with efficacy for obesity, type 2 diabetes mellitus, lipidosis, hypertension, fatty liver, arteriosclerosis, cerebrovascular disorder, coronary artery disease and metabolic syndrome.

### SUMMARY OF THE INVENTION

The compounds described herein are DGAT-1 inhibitors, which are useful in the treatment of obesity, type 2 diabetes mellitus, lipidosis, hypertension, fatty liver, arteriosclerosis, cerebrovascular disorder, coronary artery disease and metabolic syndrome, particularly, obesity and diabetes.

Described herein are compounds of formula I

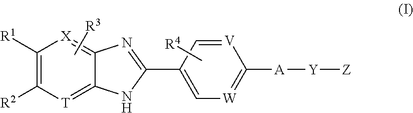

wherein A, T, V, W, X, Y, Z, R¹, R², R³, R⁴, R⁵ and R⁶ are further described below.

### DETAILED DESCRIPTION OF THE INVENTION

### Compounds

Described herein are compounds of formula (I):

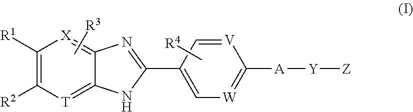

or pharmaceutically acceptable salts thereof, wherein A is a non-aromatic, nitrogen-containing ring selected from the group consisting of:

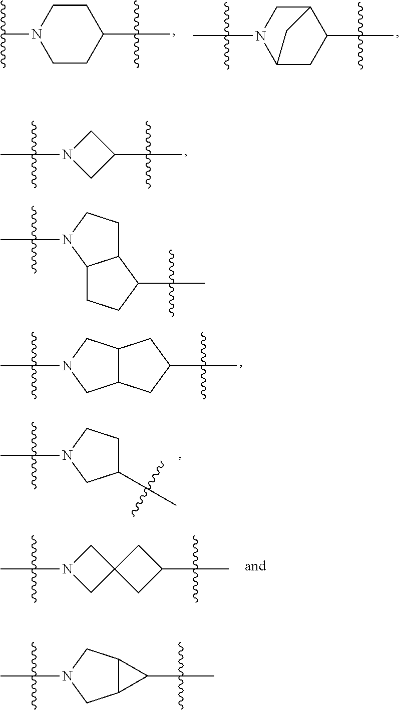

wherein A is unsubstituted or substituted with one or more substituents selected from R⁵;
wherein each occurrence of T, X, V and W are independently selected from the group consisting of —CH— and —N—;
wherein Y is —(CH₂)m-O—(CH₂)m-;
Z is selected from the group consisting of C₁-C₆alkyl, aryl, C₃-C₈cycloalkyl and heterocycle, wherein the C₁-C₆alkyl, aryl, cycloalkyl and heterocycle can be unsubstituted or substituted with 1-3 substituents selected from R⁶;
R¹, R², R³, Wand R⁵ are independently selected from the group consisting of hydrogen, halogen, C₁-C₆alkyl, halogen-substitutedC₁-C₆alkyl, —OH, C₁-C₆alkylOH, —OC₁-C₆alkyl, —Ohalogen-substitutedC₁-C₆alkyl, —SO²C₁-C₆alkyl and —CN or when taken together R¹ and R² form pyrazol;
R⁶ is selected from the group consisting of halogen, C₁-C₆alkyl, halogen-substitutedC₁-C₆alkyl, COC₁-C₆alkyl, COhalogen-substitutedC₁-C₆alkyl, —OH, C₁-C₆alkylOH, —COOH, —COCOOH, —COOC₁-C₆alkyl, —C₁-C₆alkylCOOC₁-C₆alkyl, —C₁-C₆alkylCOOH, —OC₁-C₆alkylCOOH, —CN, C₁-C₆alkylCN, heterocycle, CONHSO₂C₁-C₆alkyl, CONHSO₂halogen-substitutedC₁-C₆alkyl, CONHSO₂C₃-C₆cycloalkyl, CONHSO₂C₃-C₆cycloalkylC₁-C₆alkyl, CONHSO₂heteroaryl, CONHSO₂aryl, CONHSO₂halogen-substitutedaryl and CONHSO₂ arylhalogen-substitutedC₁-C₆alkyl; and
m and n are independently selected from the list consisting of 0, 1 or 2.

Of the compounds described herein A is a non-aromatic, nitrogen-containing ring selected from the group consisting of:

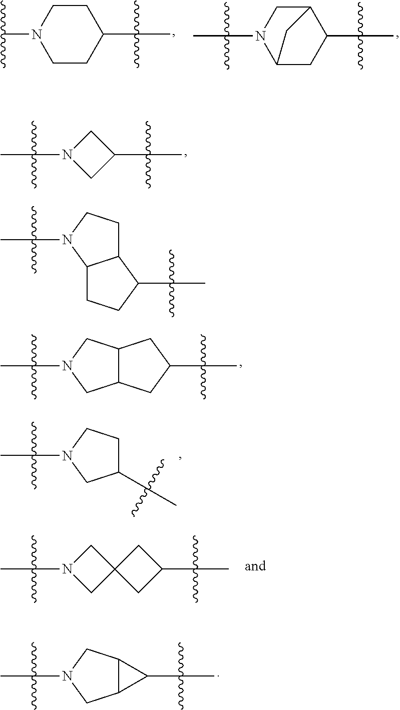

In certain embodiments of the compounds described herein A is

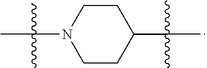 [SMILES: *C1CCN(*)CC1]

In certain embodiments A is

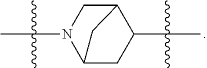 [SMILES: *C1CC2CC1CN2*]

In certain embodiments A is

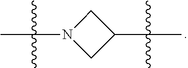 [SMILES: *C1CN(*)C1]

In certain embodiments A is

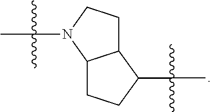 [SMILES: *C1CCC2C1CCN2*]

In certain embodiments A is

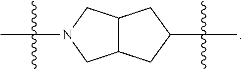 [SMILES: *C1CC2CN(*)CC2C1]

In certain embodiments A is

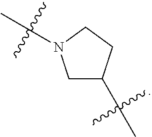 [SMILES: *C1CCN(*)C1]

In certain embodiments A is

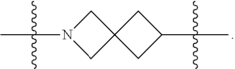 [SMILES: *C1CC2(C1)CN(*)C2]

In certain embodiments A is

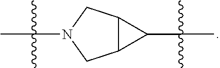 [SMILES: *C1C2CN(*)CC12]

In certain embodiments A is unsubstituted. In other embodiments, A is substituted with one or more substituents selected from R⁵. In some embodiments of the compounds described herein A is substituted with one substituent selected from R⁵. In other embodiments of the compounds described herein A is substituted with two substituents selected from R⁵. In still other embodiments of the compounds described herein A is substituted with three substituents selected from R⁵.

Of the compounds described herein, R⁵ is selected from the group consisting of hydrogen, halogen, C₁-C₆alkyl, halogen-substitutedC₁-C₆alkyl, —OH, C₁-C₆alkylOH, —OC₁-C₆alkyl, —Ohalogen-substitutedC₁-C₆alkyl and —CN. In certain embodiments, R⁴ is halogen. Suitable examples of halogen include, but are not limited to, fluorine.

Of the compounds described herein, each occurrence of T, X, V and W are independently selected from the group consisting of —CH— and —N—. In certain embodiments, T is —CH—. In other embodiments, T is —N—. In certain embodiments, X is —CH—. In other embodiments, X is —N—. It should be noted that when T or X are —CH— the hydrogen can be replaced with R³. In certain embodiments, V is —CH—. In other embodiments, V is —N—. In certain embodiments, W is —CH—. In other embodiments, W is —N—. In some embodiments, T and X are both —CH—. In other embodiments, V is —N— and W is —CH—. In other embodiments, T is —N— and X is —CH—.

Of the compounds described herein, R¹ is selected from the group consisting of hydrogen, halogen, C₁-C₆alkyl, halogen-substitutedC₁-C₆alkyl, —OH, C₁-C₆alkylOH, —OC₁-C₆alkyl, —Ohalogen-substitutedC₁-C₆alkyl, —SO²C₁-C₆alkyl and —CN or when taken together R¹ and R² form pyrazol. In certain embodiments, R¹ is hydrogen. In other embodiments R¹ is selected from the group consisting of halogen, C₁-C₆alkyl, halogen-substitutedC₁-C₆alkyl, —OC₁-C₆alkyl, —CN, —SO²CH₂. In still other embodiments, R¹ is hydrogen or halogen. In yet other embodiments, taken together R¹ and R² form pyrazol

Of the compounds described herein, R² is selected from the group consisting of hydrogen, halogen, C₁-C₆alkyl, halogen-substitutedC₁-C₆alkyl, —OH, C₁-C₆alkylOH, —OC₁-C₆alkyl, —Ohalogen-substitutedC₁-C₆alkyl, —SO²C₁-C₆alkyl and —CN or when taken together R¹ and R² form pyrazol. In certain embodiments, R² is hydrogen. In other embodiments R² is selected from the group consisting of halogen, C₁-C₆alkyl, halogen-substitutedC₁-C₆alkyl, —OC₁-C₆alkyl, —CN, —SO²CH₂. In still other embodiments, R² is hydrogen or halogen. In yet other embodiments, taken together R¹ and R² form pyrazol.

Of the compounds described herein, R³ is selected from the group consisting of hydrogen, halogen, C₁-C₆alkyl, halogen-substitutedC₁-C₆alkyl, —OH, C₁-C₆alkylOH, —OC₁-C₆alkyl, —Ohalogen-substitutedC₁-C₆alkyl, —SO²C₁-C₆alkyl and —CN. In certain embodiments, R³ is hydrogen. In still other embodiments, R³ is hydrogen or halogen.

Of the compounds described herein, R⁴ is selected from the group consisting of hydrogen, halogen, C₁-C₆alkyl, halogen-substitutedC₁-C₆alkyl, —OH, C₁-C₆alkylOH, —OC₁-C₆alkyl, —Ohalogen-substitutedC₁-C₆alkyl, —SO²C₁-C₆alkyl and —CN. In certain embodiments, R⁴ is hydrogen. In still other embodiments, R⁴ is hydrogen or halogen.

Of the compounds described herein, Y is —(CH₂)m-O—(CH₂)n-. In certain embodiments, m is 0. In other embodiments, m is 1. In still other embodiments, m is 2. In certain embodiments, n is 0. In other embodiments, n is 1. In still other embodiments, n is 2. In certain embodiments, m and n are both 0. In other embodiments, m is 1 and n is 0. In still other embodiments, m is 0 and n is 1.

Of the compounds described herein, Z is selected from the group consisting of C₁-C₆alkyl, aryl, C₃-C₈cycloalkyl and heterocycle, wherein the C₁-C₆alkyl, aryl, cycloalkyl and heterocycle can be unsubstituted or substituted with 1-3 substituents selected from R⁶. In certain embodiments, Z is C₁-C₆alkyl. Suitable alkyls include methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl, 1-ethyl-1-methylpropyl. In certain embodiments, Z is aryl. Suitable aryls include, but are not limited to, phenyl. In other embodiments, Z is cycloalkyl. Suitable cycloalkyls include cycloalkyls with three to eight carbons including cyclopropyl, cyclobutyl, cyclopentyl, cyclohexyl, cyclopentyl and cyclooctyl. In still other embodiments, Z is heterocycle. Suitable heterocycles include oxetane, pyridyl, pyran, tetrahydrofuran, tetrahydropyran, pyrimidinyl and oxazole.

In certain embodiments, Z is selected from the group consisting of: C₁-C₆alkyl, phenyl, cyclohexyl, cyclobutyl, cyclopropyl, tetrahydropyran, pyridyl, pyrimidinyl, oxazole,

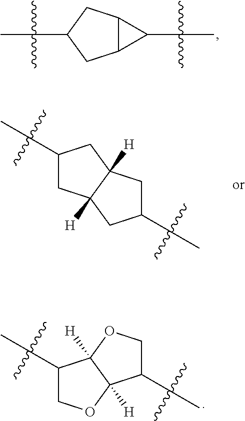 [SMILES: *C1CC2C(*)C2C1 ; *C1C[C@H]2CC(*)C[C@H]2C1 ; *C1CO[C@@H]2C(*)CO[C@H]12]

In certain embodiments, Z is cyclohexyl. In other embodiments, Z is cyclopentyl.

In certain embodiments, Z is unsubstituted. In other embodiments, Z is substituted with one or more substitutents selected from R⁶. In still other embodiments, Z is substituted with 1-3 substitutents selected from R⁶. In still other embodiments, Z is substituted with one substituent selected from R⁶. In still other embodiments, Z is substituted with 2 substituents selected from R⁶. In still other embodiments, Z is substituted with 3 substituents selected from R⁶.

Of the compounds described herein, R⁶ is selected from the group consisting of halogen, C₁-C₆alkyl, halogen-substitutedC₁-C₆alkyl, COC₁-C₆alkyl, COhalogen-substitutedC₁-C₆alkyl, —OH, C₁-C₆alkylOH, —COOH, —COCOOH, —COOC₁-C₆alkyl, —C₁-C₆alkylCOOC₁-C₆alkyl, —C₁-C₆alkylCOOH, —OC₁-C₆alkylCOOH, —CN, C₁-C₆alkylCN, heterocycle, CONHSO₂C₁-C₆alkyl, CONHSO₂halogen-substitutedC₁-C₆alkyl, CONHSO₂C₃-C₆cycloalkyl, CONHSO₂C₃-C₆cycloalkylC₁-C₆alkyl, CONHSO₂heteroaryl, CONHSO₂aryl, CONHSO₂halogen-substitutedaryl and CONHSO₂arylhalogen-substitutedC₁-C₆alkyl. In certain embodiments, R⁶ is selected from the group consisting of —OH, —COOH, —COOC₁-C₆alkyl, —C₁-C₆alkylCOOC₁-C₆alkyl, C₁-C₆alkyl or —C₁-C₆alkylCOOH. In other embodiments, R⁶ is CONHSO₂C₁-C₆alkyl, CONHSO₂halogen-substitutedC₁-C₆alkyl, CONHSO₂C₃-C₆cycloalkyl, CONHSO₂C₃-C₆cycloalkylC₁-C₆alkyl, CONHSO₂heteroaryl, CONHSO₂aryl, CONHSO₂halogen-substitutedaryl and CONHSO₂arylhalogen-substitutedC₁-C₆alkyl. In still other embodiments, R⁶ is —COOH. In yet other embodiments, R⁶ is —C₁-C₆alkylCOOH.

Also described herein are formulas Ia through Il:

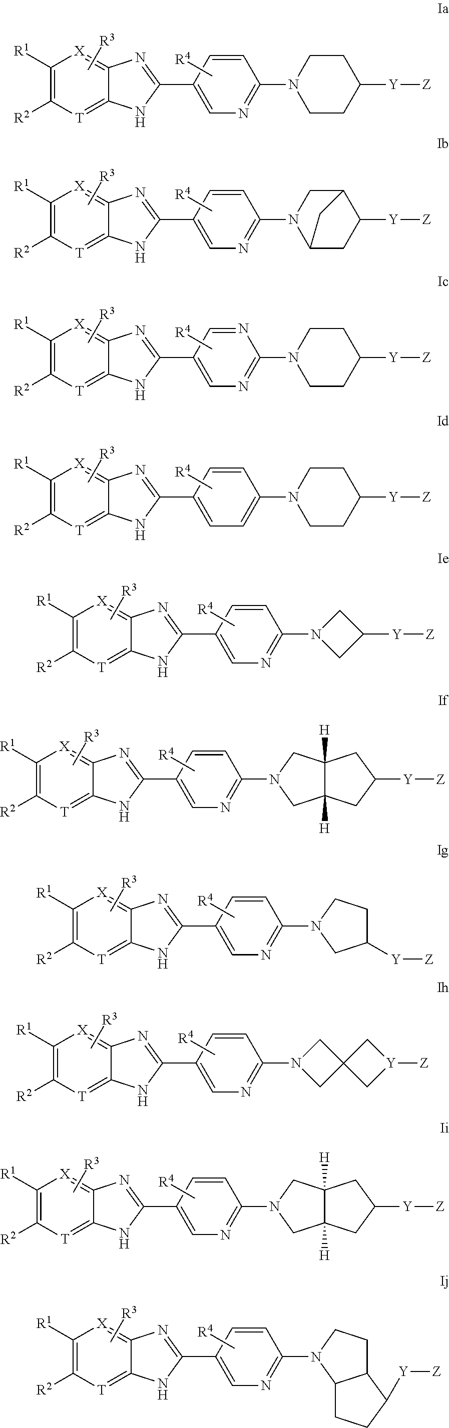

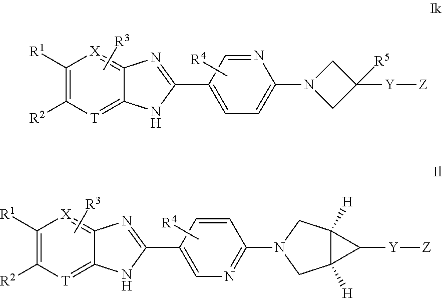

wherein T, X, Y, Z and R¹, R², R³, R⁴, R⁵ and R⁶ are described above.

Also described herein are compounds of formulas Im and In:

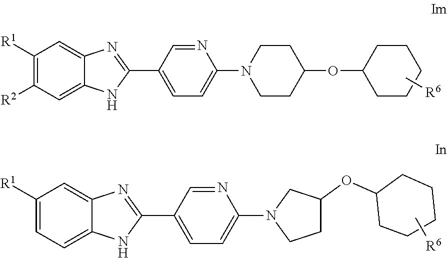

wherein R¹, R² and R⁶ are described above.

Examples of the compounds described herein include, but are not limited to:

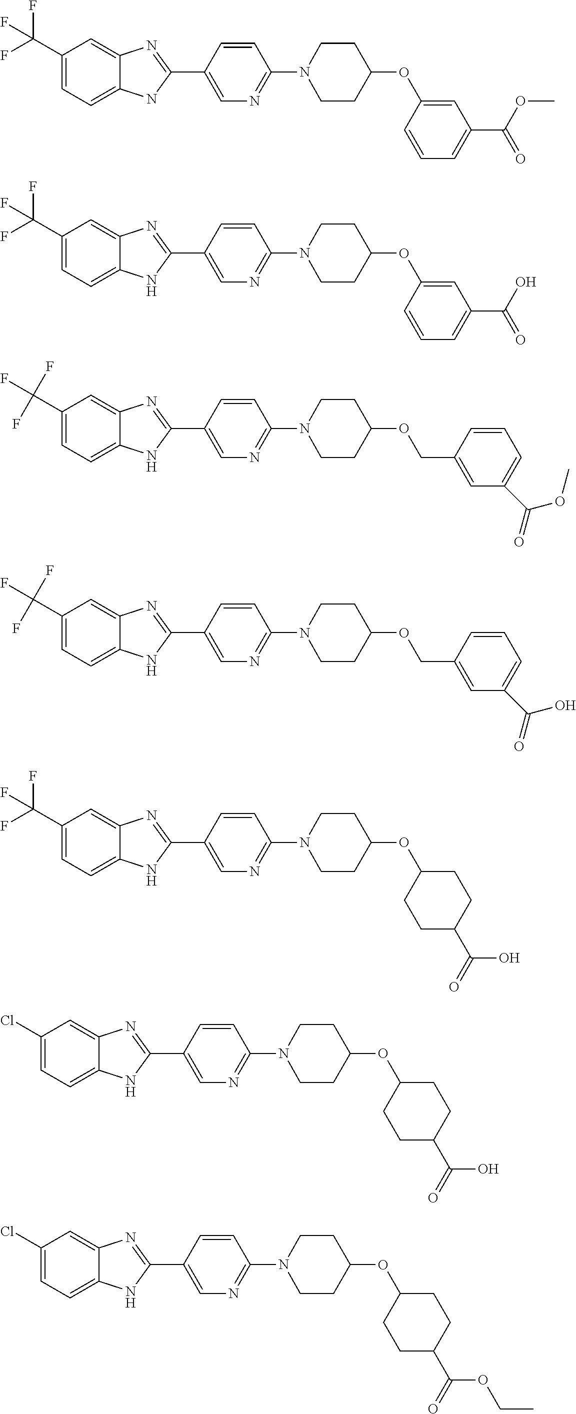

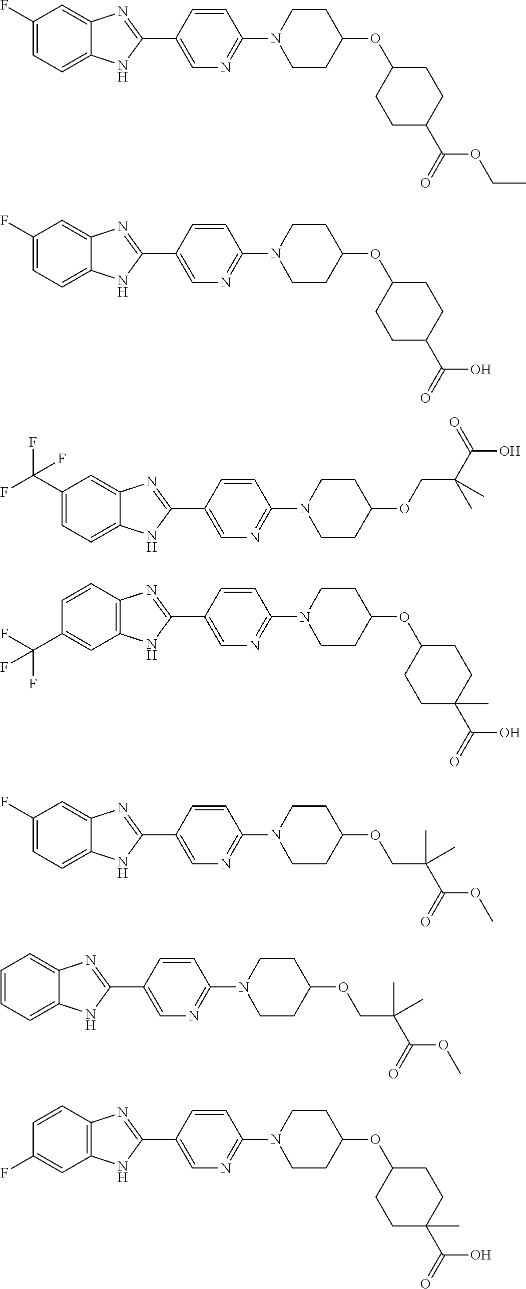

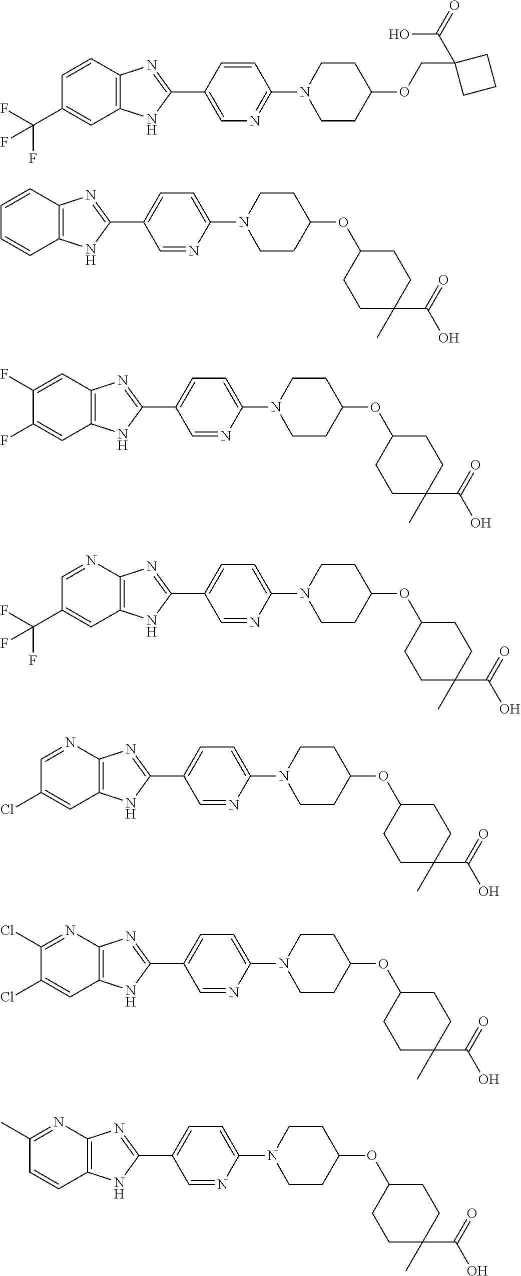

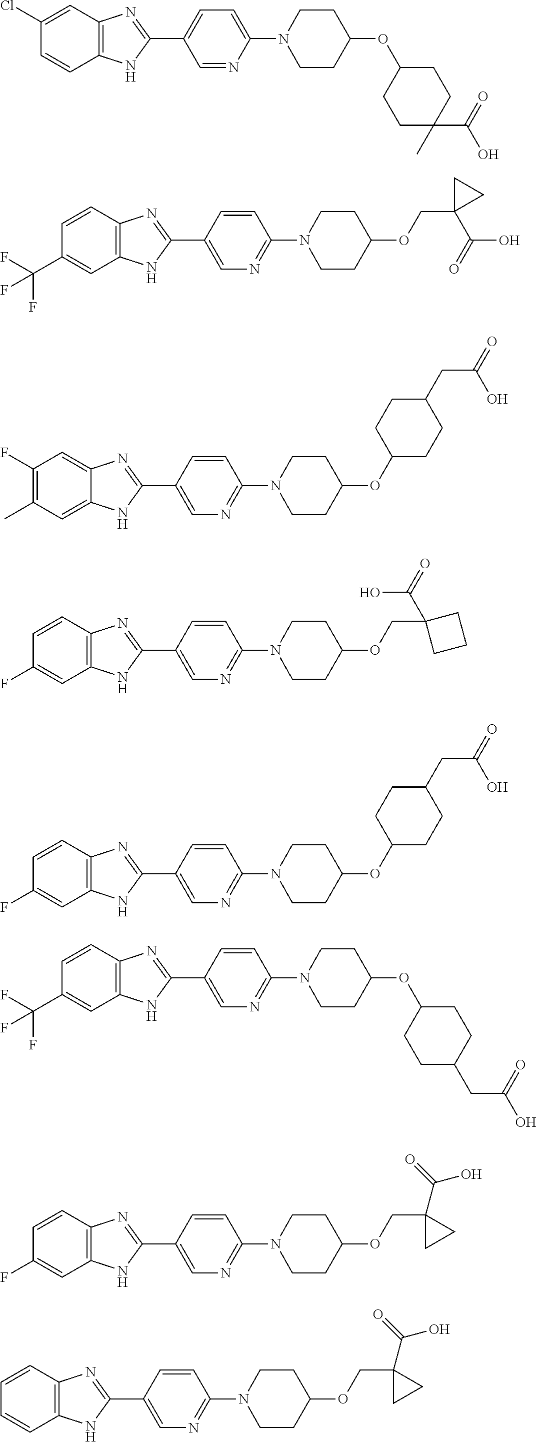

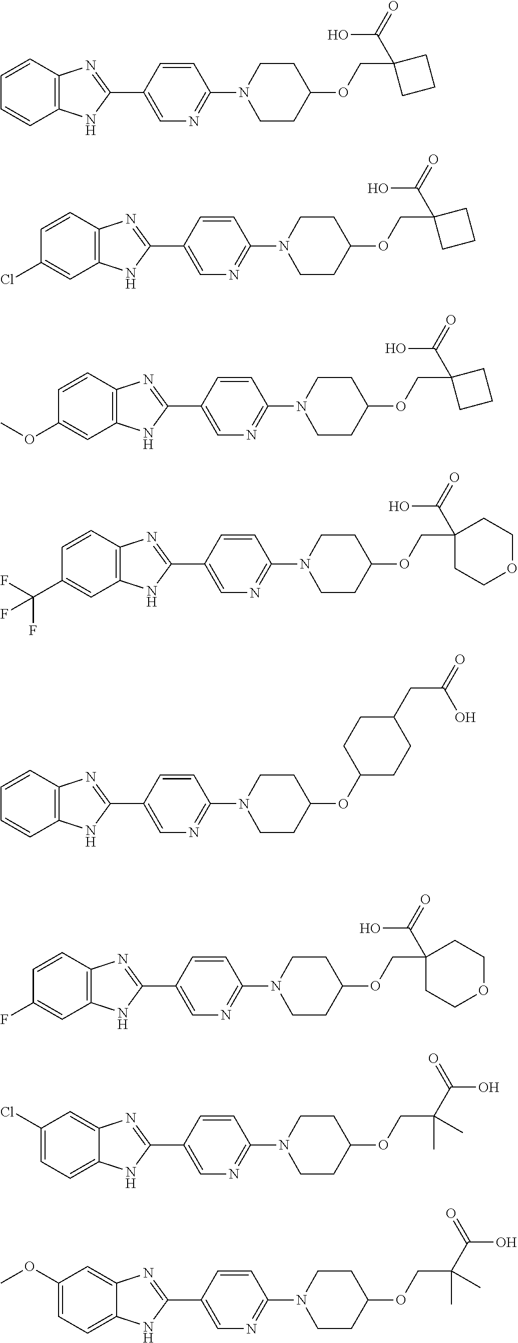

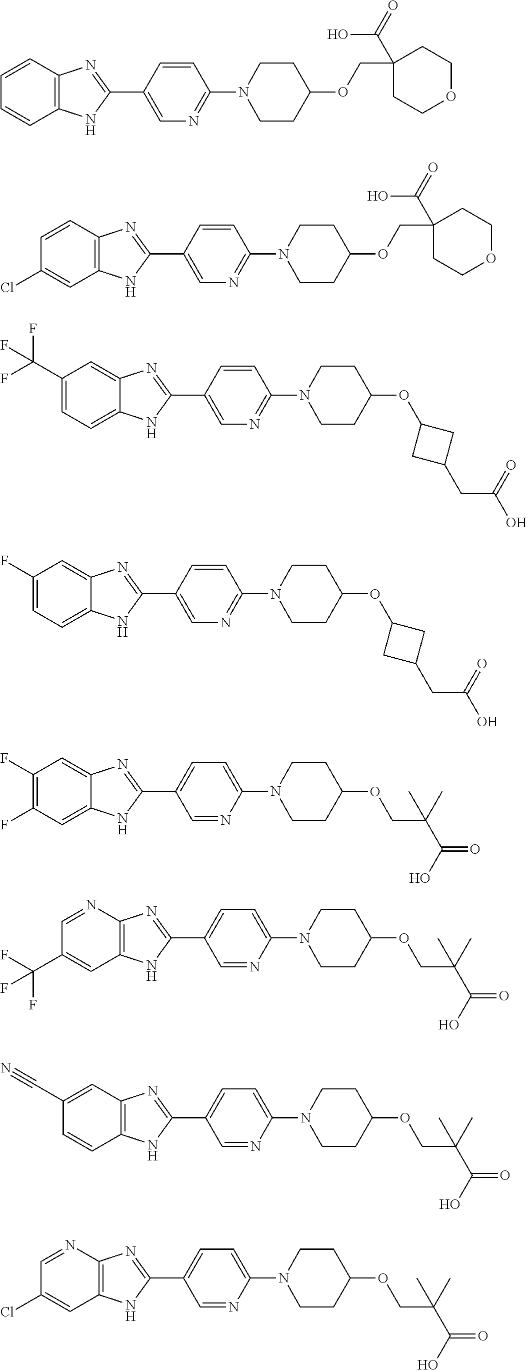

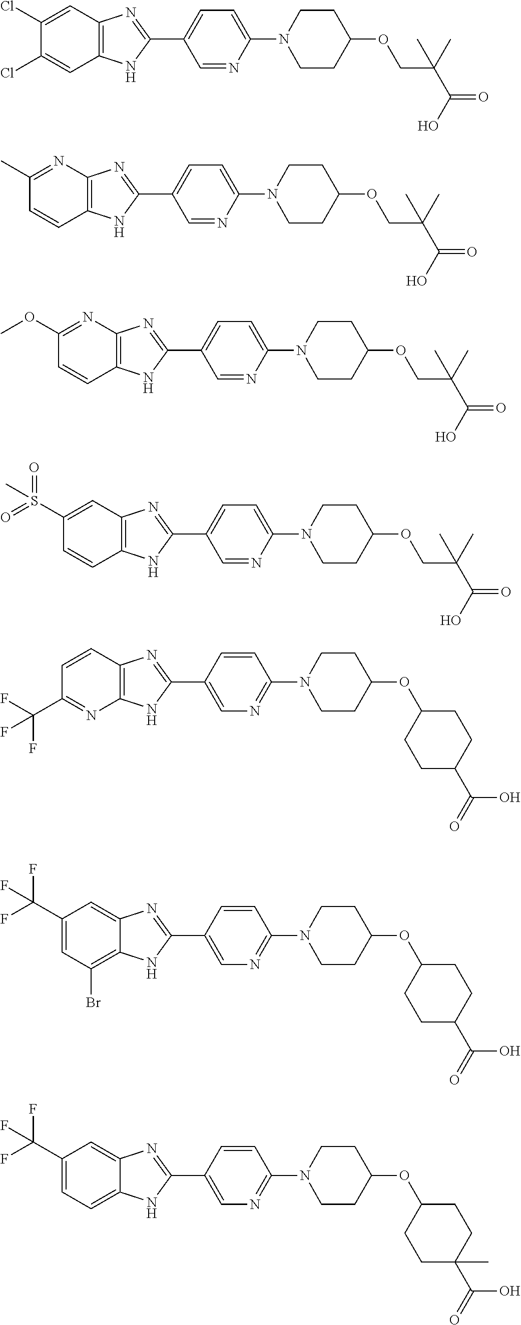

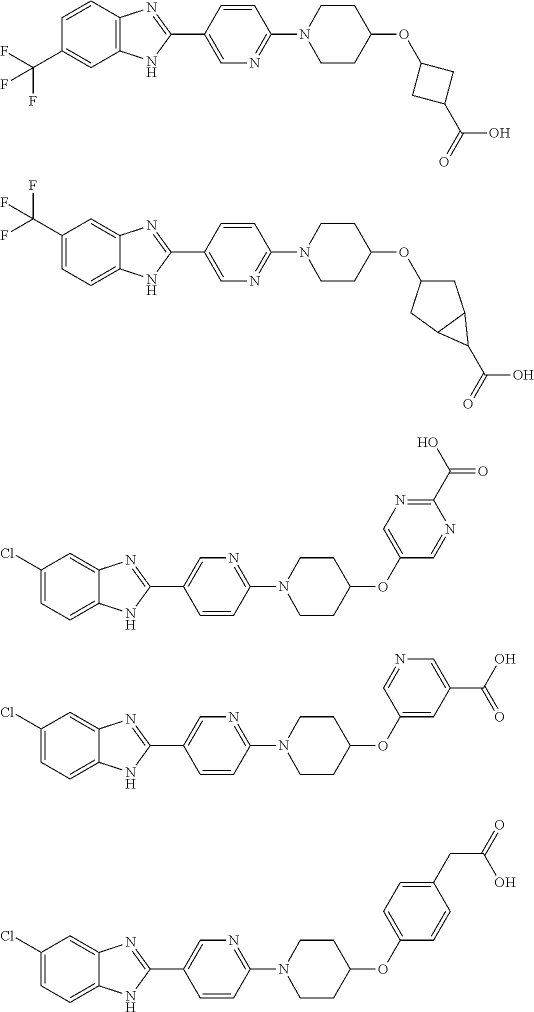 [SMILES: O=C(O)C1CC(OC2CCN(c3ccc(-c4nc5ccc(C(F)(F)F)cc5[nH]4)cn3)CC2)C1 ; O=C(O)C1C2CC(OC3CCN(c4ccc(-c5nc6cc(C(F)(F)F)ccc6[nH]5)cn4)CC3)CC21 ; O=C(O)c1ncc(OC2CCN(c3ccc(-c4nc5cc(Cl)ccc5[nH]4)cn3)CC2)cn1 ; O=C(O)c1cncc(OC2CCN(c3ccc(-c4nc5cc(Cl)ccc5[nH]4)cn3)CC2)c1 ; O=C(O)Cc1ccc(OC2CCN(c3ccc(-c4nc5cc(Cl)ccc5[nH]4)cn3)CC2)cc1]

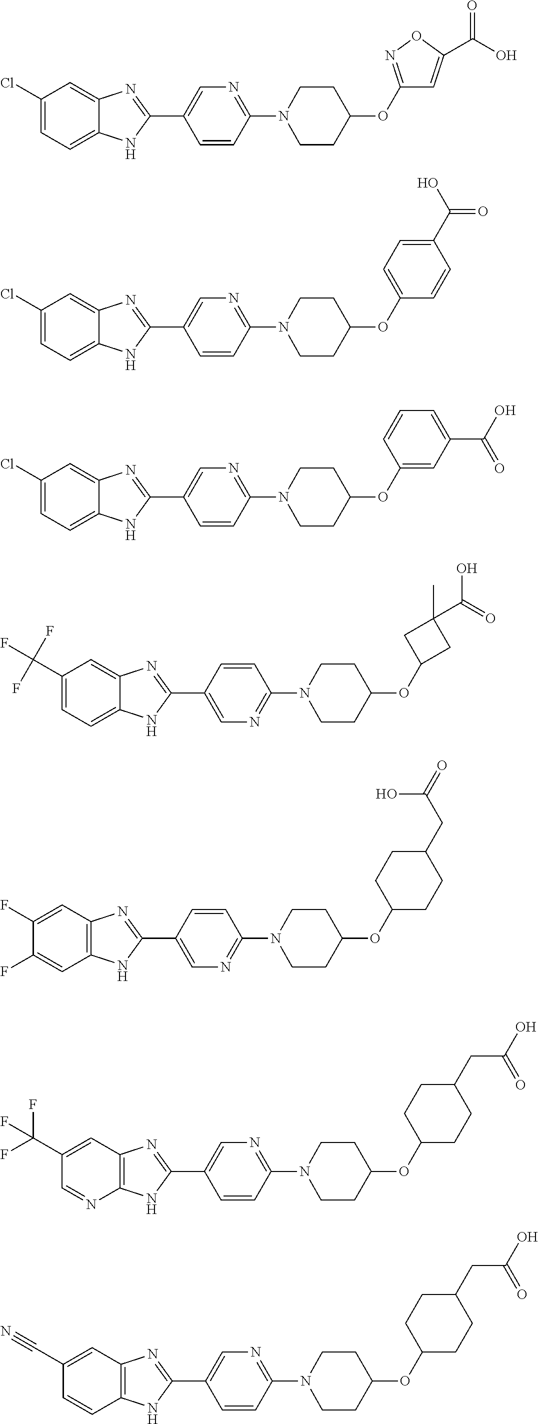

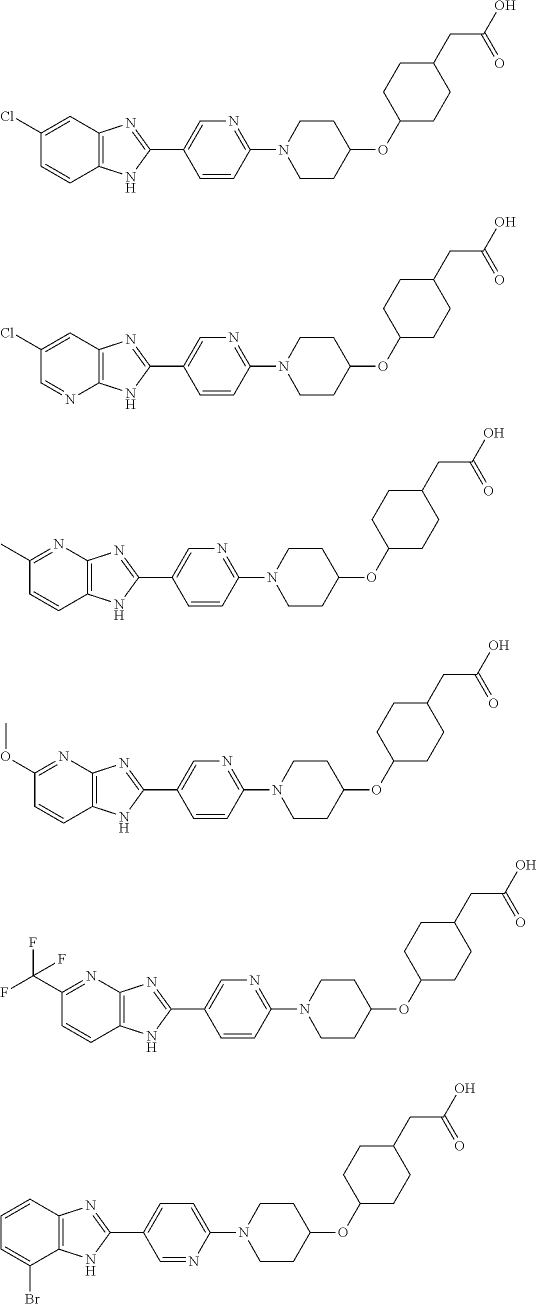 [SMILES: O=C(O)CC1CCC(OC2CCN(c3ccc(-c4nc5cc(Cl)ccc5[nH]4)cn3)CC2)CC1 ; O=C(O)CC1CCC(OC2CCN(c3ccc(-c4nc5cc(Cl)cnc5[nH]4)cn3)CC2)CC1 ; Cc1ccc2[nH]c(-c3ccc(N4CCC(OC5CCC(CC(=O)O)CC5)CC4)nc3)nc2n1 ; COc1ccc2[nH]c(-c3ccc(N4CCC(OC5CCC(CC(=O)O)CC5)CC4)nc3)nc2n1 ; O=C(O)CC1CCC(OC2CCN(c3ccc(-c4nc5nc(C(F)(F)F)ccc5[nH]4)cn3)CC2)CC1 ; O=C(O)CC1CCC(OC2CCN(c3ccc(-c4nc5cccc(Br)c5[nH]4)cn3)CC2)CC1]

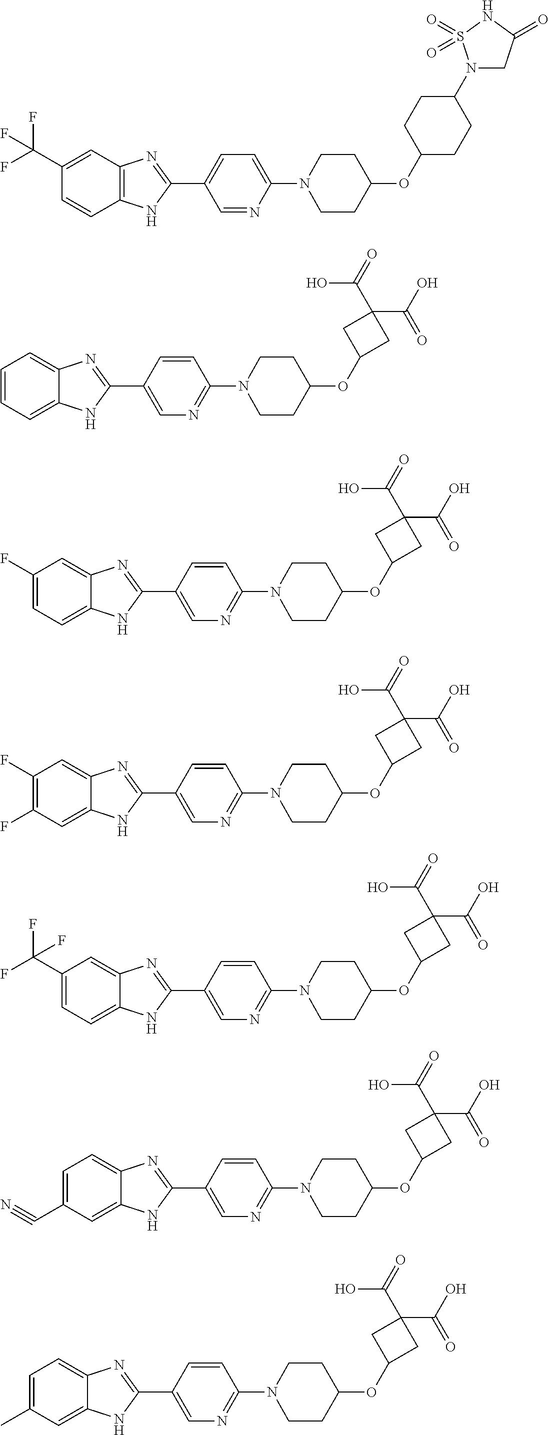

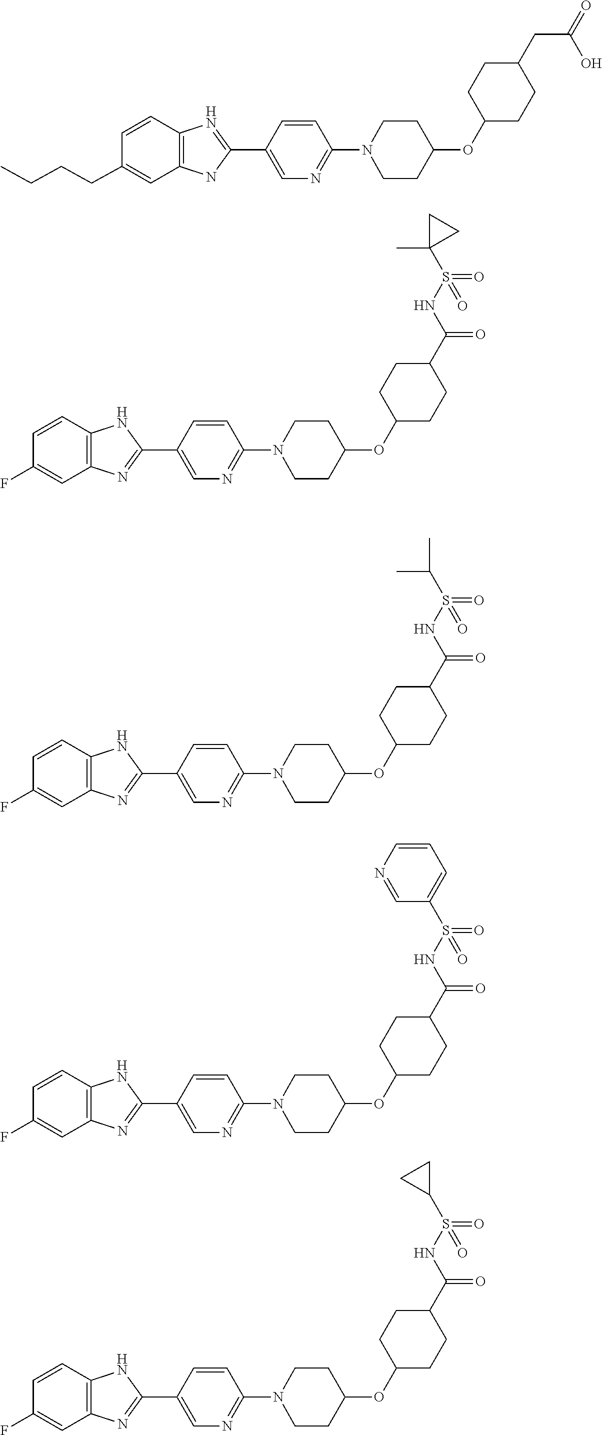

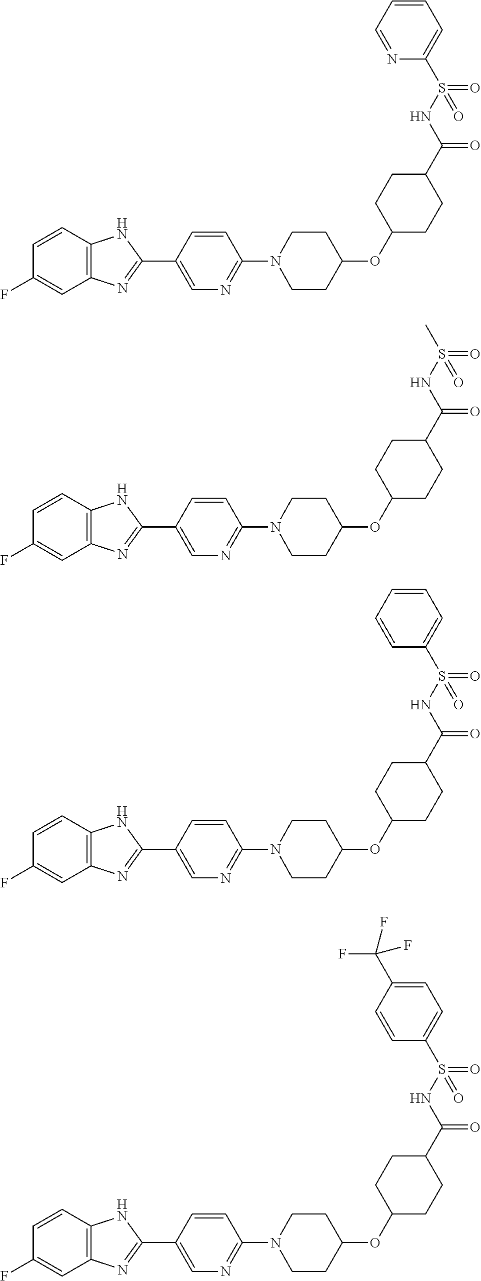 [SMILES: O=C(NS(=O)(=O)c1ccccn1)C1CCC(OC2CCN(c3ccc(-c4nc5cc(F)ccc5[nH]4)cn3)CC2)CC1 ; CS(=O)(=O)NC(=O)C1CCC(OC2CCN(c3ccc(-c4nc5cc(F)ccc5[nH]4)cn3)CC2)CC1 ; O=C(NS(=O)(=O)c1ccccc1)C1CCC(OC2CCN(c3ccc(-c4nc5cc(F)ccc5[nH]4)cn3)CC2)CC1 ; O=C(NS(=O)(=O)c1ccc(C(F)(F)F)cc1)C1CCC(OC2CCN(c3ccc(-c4nc5cc(F)ccc5[nH]4)cn3)CC2)CC1]

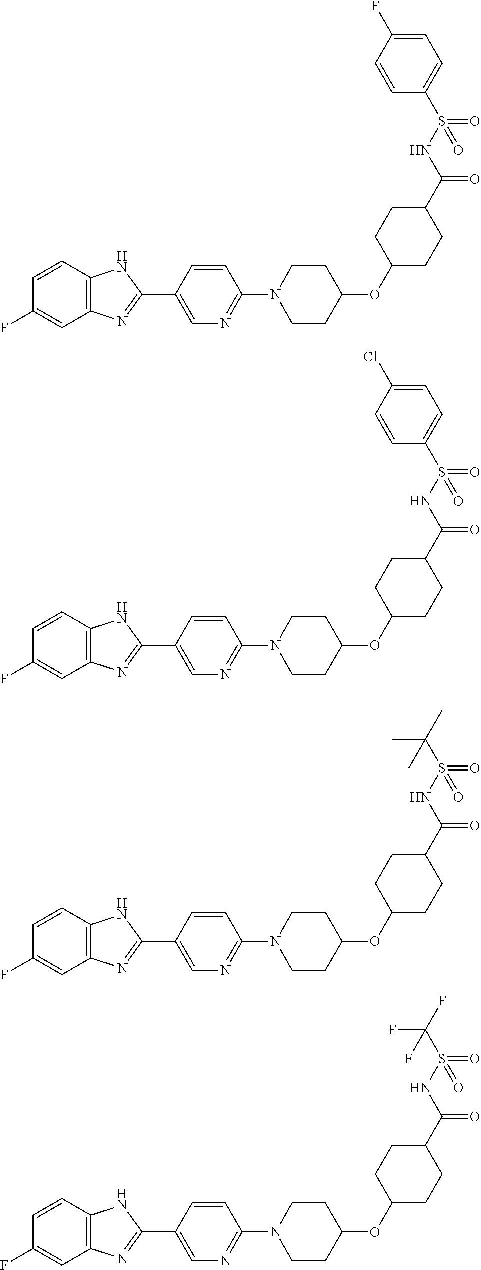 [SMILES: O=C(NS(=O)(=O)c1ccc(F)cc1)C1CCC(OC2CCN(c3ccc(-c4nc5cc(F)ccc5[nH]4)cn3)CC2)CC1 ; O=C(NS(=O)(=O)c1ccc(Cl)cc1)C1CCC(OC2CCN(c3ccc(-c4nc5cc(F)ccc5[nH]4)cn3)CC2)CC1 ; CC(C)(C)S(=O)(=O)NC(=O)C1CCC(OC2CCN(c3ccc(-c4nc5cc(F)ccc5[nH]4)cn3)CC2)CC1 ; O=C(NS(=O)(=O)C(F)(F)F)C1CCC(OC2CCN(c3ccc(-c4nc5cc(F)ccc5[nH]4)cn3)CC2)CC1]

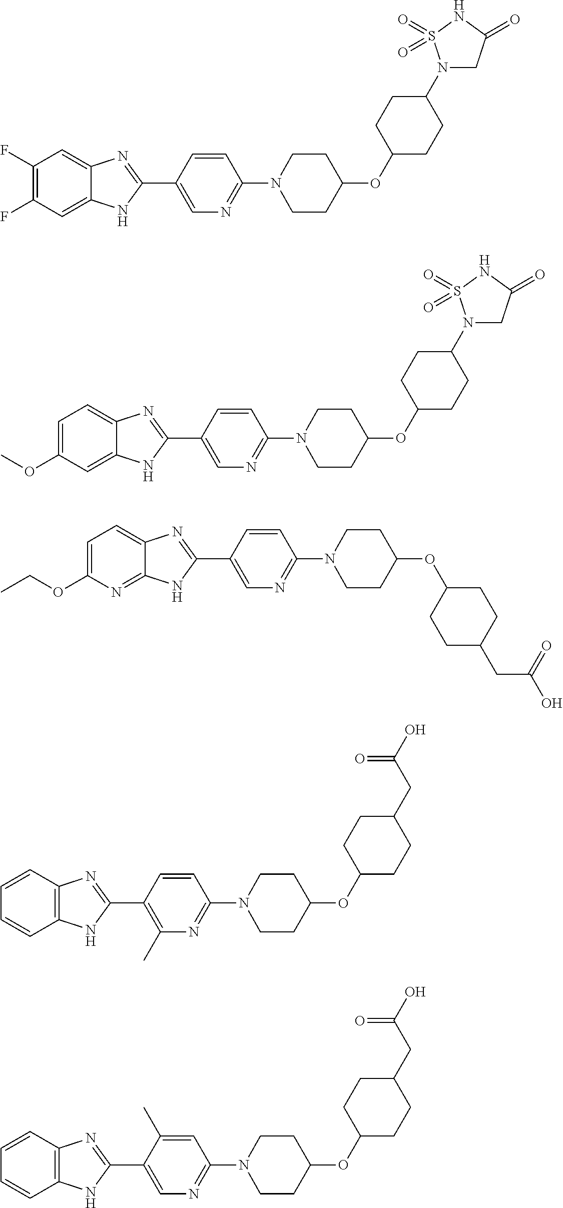 [SMILES: O=C1CN(C2CCC(OC3CCN(c4ccc(-c5nc6cc(F)c(F)cc6[nH]5)cn4)CC3)CC2)S(=O)(=O)N1 ; COc1ccc2nc(-c3ccc(N4CCC(OC5CCC(N6CC(=O)NS6(=O)=O)CC5)CC4)nc3)[nH]c2c1 ; CCOc1ccc2nc(-c3ccc(N4CCC(OC5CCC(CC(=O)O)CC5)CC4)nc3)[nH]c2n1 ; Cc1nc(N2CCC(OC3CCC(CC(=O)O)CC3)CC2)ccc1-c1nc2ccccc2[nH]1 ; Cc1cc(N2CCC(OC3CCC(CC(=O)O)CC3)CC2)ncc1-c1nc2ccccc2[nH]1]

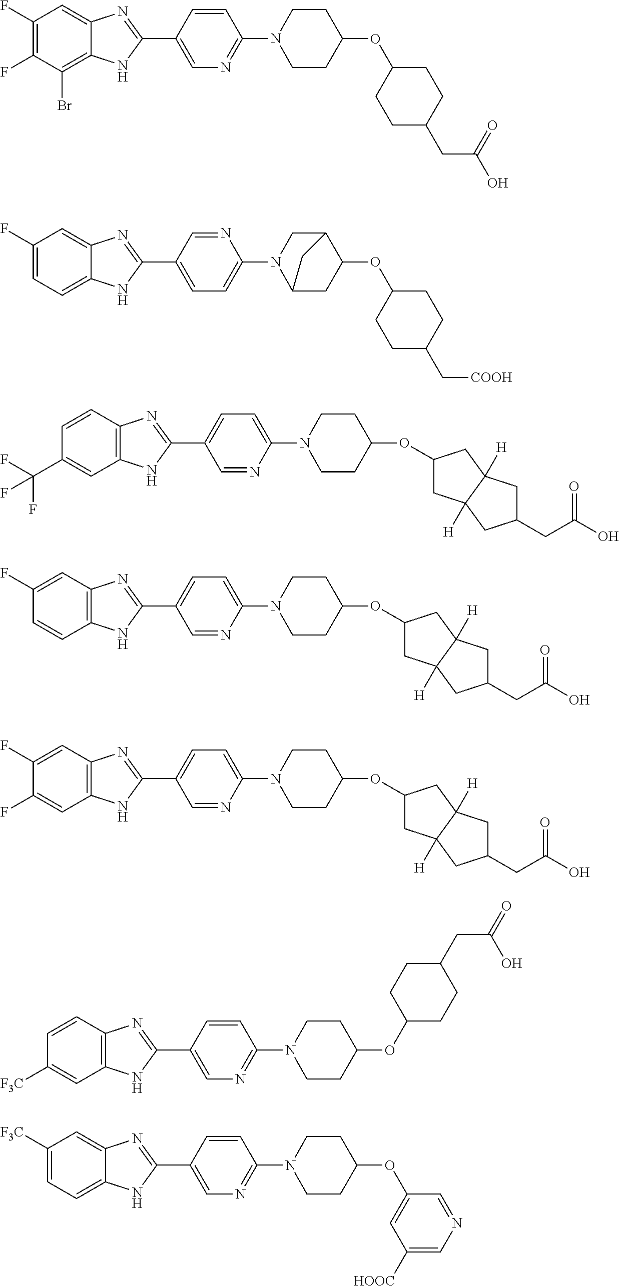

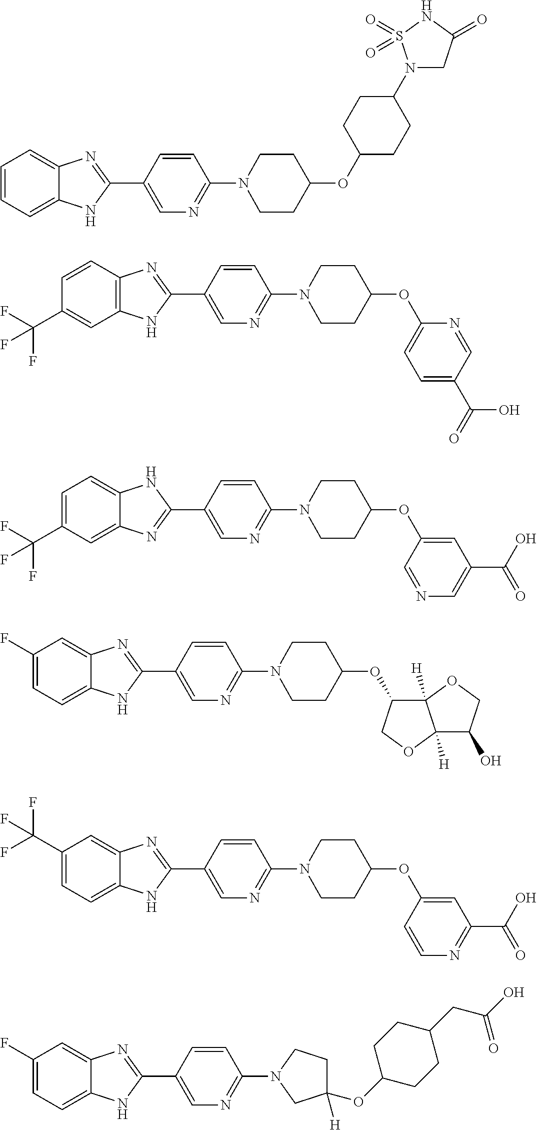

In certain embodiments, the compounds described herein, include:

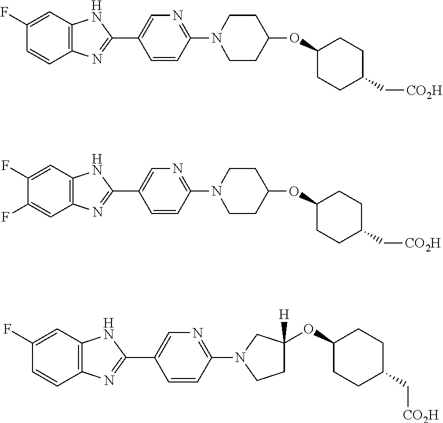 [SMILES: O=C(O)C[C@H]1CC[C@H](OC2CCN(c3ccc(-c4nc5ccc(F)cc5[nH]4)cn3)CC2)CC1 ; O=C(O)C[C@H]1CC[C@H](OC2CCN(c3ccc(-c4nc5cc(F)c(F)cc5[nH]4)cn3)CC2)CC1 ; O=C(O)C[C@H]1CC[C@H](O[C@@H]2CCN(c3ccc(-c4nc5ccc(F)cc5[nH]4)cn3)C2)CC1]

### DEFINITIONS

Examples of “halogen” include a fluorine atom, a chlorine atom, a bromine atom, and an iodine atom.

The term “C₁-C₆alkyl” encompasses straight alkyl having a carbon number of 1 to 6 and branched alkyl having a carbon number of 3 to 6. Specific examples thereof include methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl, 1-ethyl-1-methylpropyl, and the like.

The term “—OC₁-C₆alkyl” refers to an alkyl group having 1 to 6 carbons linked to oxygen, also known as an alkoxy group. Examples include methoxy, ethoxy, butoxy and propoxy.

The term “—OC₁-C₆alkylCOOH” refers to an alkoxy group having 1 to 6 carbons substituted with a carboxylic acid (—COOH) group.

The term “halogen-substitutedC₁-C₆ alkyl” encompasses C₁-C₆ alkyl with the hydrogen atoms thereof being partially or completely substituted with halogen, examples thereof including fluoromethyl, difluoromethyl, trifluoromethyl, 2-fluoroethyl, 1,2-difluoroethyl, 2,2-difluoroethyl and the like.

The term “—Ohalogen-substitutedC₁-C₆alkyl” means a —OC₁-C₆alkyl as defined above, which is substituted with 1-3 halogen atoms which are identical or different, and specifically includes, for example, a trifluoromethoxy group.

The term “—COC₁-C₆alkyl” means groups having C₁-C₆alkyl bonded to carbonyl, and encompasses alkylcarbonyl having a carbon number of 1 to 6. Specific examples thereof include acetyl, propionyl, butyryl, isobutyryl, valeryl, isovaleryl, pivaloyl, and the like.

The term “—COhalogen-substitutedC₁-C₆alkyl” means a —COC₁-C₆alkyl as defined above, which is substituted with 1-3 halogen atoms which are identical or different.

The term “C₁-C₆alkylOH” means a C₁-C₆alkyl substituted with an alcohol (—OH). Examples include methanol, propanol, butanol and t-butanol.

The term “C₁-C₆alkylCN” means a C₁-C₆alkyl substituted with an cyano group (—CN).

The term “halogen-substituted C₁-C₆alkylOH” means a halogen-substituedC1-C6alkyl substituted with an alcohol (—OH).

The term “COOC₁-C₆alkyl” means a —COOH group wherein the —OH is replaced with an alkoxy group as defined above. Examples include methoxycarbonyl, ethoxycarbonyl and butoxycarbonyl.

The term “SO₂C₁-C₆alkyl” means a group having C₁-C₆alkyl bonded to sulfonyl (—SO₂—). Specific examples thereof include methanesulfonyl, ethanesulfonyl, n-propanesulfonyl, isopropanesulfonyl, n-butanesulfonyl, sec-butanesulfonyl, tert-butanesulfonyl, and the like.

The term “C₃-C₈cycloalkyl” encompasses cycloalkyls having 3 to 8 carbons, forming one or more carbocyclic rings that are fused. “Cycloalkyl” also includes monocyclic rings fused to an aryl group in which the point of attachment is on the non-aromatic portion. Examples of cycloalkyl include cyclopropyl, cyclobutyl, cyclopentyl, cyclohexyl, cycloheptyl, tetrahydronaphthyl, decahydronaphthyl, indanyl and the like.

Examples of “aryl” include phenyl, naphthyl, tolyl, and the like.

The term “heterocycle” means mono- or bicyclic or bridged unsaturated, partially unsaturated and saturated rings containing at least one heteroatom selected from N, S and O, each of said ring having from 3 to 10 atoms in which the point of attachment may be carbon or nitrogen. Examples thereof include pyrrolyl, furyl, thienyl, imidazolyl, pyrazolyl, thiazolyl, isothiazolyl, oxazolyl, isoxazolyl, triazolyl, tetrazolyl, oxadiazolyl, 1,2,3-thiadiazolyl, 1,2,4-thiadiazolyl, 1,3,4-thiadiazolyl, pyridyl, pyrazinyl, pyrimidinyl, pyridazinyl, 1,2,4-triazinyl, 1,3,5-triazinyl, indolyl, benzofuranyl, benzothienyl, benzimidazolyl, benzopyrazolyl, benzoxazolyl, benzisoxazolyl, benzothiazolyl, benzisothiazolyl, indazolyl, purinyl, quinolyl, isoquinolyl, phthalazinyl, naphthyridinyl, quinoxalinyl, quinazolinyl, cinnolinyl, pteridinyl, pyrido[3,2-b]pyridyl, and the like. Examples also include tetrahydropyranyl, tetrahydrofuranyl, pyrrolidinyl, piperidinyl, piperazinyl, dioxanyl, imidazolidinyl, 2,3-dihydrofuro(2,3-b)pyridyl, benzoxazinyl, benzoxazolinyl, 2-H-phthalazinyl, isoindolinyl, benzoxazepinyl, 5,6-dihydroimidazo[2,1-b]thiazolyl, tetrahydroquinolinyl, morpholinyl, tetrahydroisoquinolinyl, dihydroindolyl, tetrahydropyran, and the like. The term also includes partially unsaturated monocyclic rings that are not aromatic, such as 2- or 4-pyridones attached through the nitrogen or N-substituted-(1H, 3H)-pyrimidine-2,4-diones (N-substituted uracils). The term also includes bridged rings such as 5-azabicyclo[2.2.1]heptyl, 2,5-diazabicyclo[2.2.1]heptyl, 2-azabicyclo[2.2.1]heptyl, 7-azabicyclo[2.2.1]heptyl, 2,5-diazabicyclo[2.2.2]octyl, 2-azabicyclo[2.2.2]octyl, and 3-azabicyclo[3.2.2]nonyl, and azabicyclo[2.2.1]heptanyl.

The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

The compounds of the present invention contain one or more asymmetric centers and can thus occur as racemates, racemic mixtures, single enantiomers, diastereomeric mixtures, and individual diastereomers. The present invention is meant to comprehend all such isomeric forms of these compounds.

Some of the compounds described herein contain olefinic double bonds, and unless specified otherwise, are meant to include both E and Z geometric isomers.

The independent syntheses of these diastereomers or their chromatographic separations may be achieved as known in the art by appropriate modification of the methodology disclosed herein. Their absolute stereochemistry may be determined by the X-ray crystallography of crystalline products or crystalline Intermediates which are derivatized, if necessary, with a reagent containing an asymmetric center of known absolute configuration.
If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

It will be understood that, as used herein, references to the compounds of the structural formulas described herein are meant to also include the pharmaceutically acceptable salts, and also salts that are not pharmaceutically acceptable when they are used as precursors to the free compounds or their pharmaceutically acceptable salts or in other synthetic manipulations.

Solvates, and in particular, the hydrates of the compounds of the structural formulas described herein are included in the present invention as well. Some of the compounds described herein may exist as tautomers, which have different points of attachment of hydrogen accompanied by one or more double bond shifts. For example, a ketone and its enol form are keto-enol tautomers. The individual tautomers as well as mixtures thereof are encompassed with compounds of the present invention.

In the compounds of the formulas described herein, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of the formulas described herein. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic formula can be prepared without undue experimentation by conventional techniques well known to those skilled in the art or by processes analogous to those described in the Schemes and Examples herein using appropriate isotopically-enriched reagents and/or Intermediates.

### Methods of Treatment

Also encompassed by the present invention are methods of treating DGAT1-related diseases. The compounds described herein are effective in preventing or treating various DGAT1-related diseases, such as metabolic diseases such as obesity, diabetes, hormone secretion disorder, hyperlipemia, gout, fatty liver, and the like; circulatory diseases such as angina pectoris, acute/congestive cardiac insufficiency, myocardial infarction, coronary arteriosclerosis, hypertension, nephropathy, electrolyte abnormality, and the like; central and peripheral nervous system diseases such as bulimia, affective disorder, depression, anxiety, epilepsy, delirium, dementia, schizophrenia, attention deficit/hyperactivity disorder, dysmnesia, somnipathy, cognitive impairment, dyskinesia, dysesthesia, dysosmia, morphine resistance, drug dependence, alcohol dependence, and the like; reproductive system diseases such as infertility, premature delivery, sexual dysfunction, and the like; and other conditions including digestive diseases, respiratory diseases, cancer, and chromatosis. The compound of the invention is especially useful as a preventive or a remedy for obesity, diabetes, fatty liver, bulimia, depression, or anxiety.

Accumulation of triglycerides leads to the obesity and associated with insulin-resistance, so inhibition of triglycerides synthesis represents a potential therapeutic strategy for human obesity and type 2 diabetes. One aspect of the invention described herein provides a method for the treatment and control of obesity or metabolic syndrome, which comprises administering to a patient in need of such treatment a therapeutically effective amount of a compound having the formulas described herein or a pharmaceutically acceptable salt thereof. For example, the compounds described herein are useful for treating or preventing obesity by administering to a subject in need thereof a composition comprising a compound of any of the formulas described herein.

Methods of treating or preventing obesity and conditions associated with obesity refer to the administration of the pharmaceutical formulations described herein to reduce or maintain the body weight of an obese subject or to reduce or maintain the body weight of an individual at risk of becoming obese. One outcome of treatment may be reducing the body weight of an obese subject relative to that subject's body weight immediately before the administration of the compounds or combinations of the present invention. Another outcome of treatment may be preventing body weight, regain of body weight previously lost as a result of diet, exercise, or pharmacotherapy and preventing weight gain from cessation of smoking. Another outcome of treatment may be decreasing the occurrence of and/or the severity of obesity-related diseases. Yet another outcome of treatment may be decreasing the risk of developing diabetes in an overweight or obese subject. The treatment may suitably result in a reduction in food or calorie intake by the subject, including a reduction in total food intake, or a reduction of intake of specific components of the diet such as carbohydrates or fats; and/or the inhibition of nutrient absorption; and/or the inhibition of the reduction of metabolic rate; and in weight reduction in patients in need thereof. The treatment may also result in an alteration of metabolic rate, such as an increase in metabolic rate, rather than or in addition to an inhibition of the reduction of metabolic rate; and/or in minimization of the metabolic resistance that normally results from weight loss.

Prevention of obesity and obesity-related disorders refers to the administration of the pharmaceutical formulations described herein to reduce or maintain the body weight of a subject at risk of obesity. One outcome of prevention may be reducing the body weight of a subject at risk of obesity relative to that subject's body weight immediately before the administration of the compounds or combinations of the present invention. Another outcome of prevention may be preventing body weight regain of body weight previously lost as a result of diet, exercise, or pharmacotherapy. Another outcome of prevention may be preventing obesity from occurring if the treatment is administered prior to the onset of obesity in a subject at risk of obesity. Another outcome of prevention may be decreasing the occurrence and/or severity of obesity-related disorders if the treatment is administered prior to the onset of obesity in a subject at risk of obesity. Moreover, if treatment is commenced in already obese subjects, such treatment may prevent the occurrence, progression or severity of obesity-related disorders, such as, but not limited to, arteriosclerosis, type 2 diabetes, polycystic ovary disease, cardiovascular diseases, osteoarthritis, dermatological disorders, hypertension, insulin resistance, hypercholesterolemia, hypertriglyceridemia, and cholelithiasis.

The following diseases, disorders and conditions are related to Type 2 diabetes, and therefore may be treated, controlled or in some cases prevented, by treatment with the compounds described herein: (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) irritable bowel syndrome, (15) inflammatory bowel disease, including Crohn's disease and ulcerative colitis, (16) other inflammatory conditions, (17) pancreatitis, (18) abdominal obesity, (19) neurodegenerative disease, (20) retinopathy, (21) nephropathy, (22) neuropathy, (23) Syndrome X, (24) ovarian hyperandrogenism (polycystic ovarian syndrome), and other disorders where insulin resistance is a component. In Syndrome X, also known as Metabolic Syndrome, obesity is thought to promote insulin resistance, diabetes, dyslipidemia, hypertension, and increased cardiovascular risk. Therefore, DPP-4 inhibitors may also be useful to treat hypertension associated with this condition.

Another aspect of the invention that is of interest relates to a method of treating hyperglycemia, hypertriglyceridemia, diabetes or insulin resistance in a mammalian patient in need of such treatment which comprises administering to said patient a compound in accordance with the formulas described herein or a pharmaceutically acceptable salt thereof in an amount that is effective to treat hyperglycemia, diabetes or insulin resistance.

More particularly, another aspect of the invention that is of interest relates to a method of treating type 2 diabetes in a mammalian patient in need of such treatment comprising administering to the patient a compound in accordance with the formulas described herein or a pharmaceutically acceptable salt thereof in an amount that is effective to treat type 2 diabetes.

Yet another aspect of the invention that is of interest relates to a method of treating non-insulin dependent diabetes mellitus in a mammalian patient in need of such treatment comprising administering to the patient a compound in accordance with the formulas described herein or a pharmaceutically acceptable salt thereof in an amount that is effective to treat non-insulin dependent diabetes mellitus.

The present invention is also directed to the use of a compound of any of the formulas described herein in the manufacture of a medicament for use in treating various DGAT1-related diseases, such as metabolic diseases such as obesity, diabetes, hormone secretion disorder, hyperlipemia, gout, fatty liver, and the like; circulatory diseases such as angina pectoris, acute/congestive cardiac insufficiency, myocardial infarction, coronary arteriosclerosis, hypertension, nephropathy, electrolyte abnormality, and the like; central and peripheral nervous system diseases such as bulimia, affective disorder, depression, anxiety, epilepsy, delirium, dementia, schizophrenia, attention deficit/hyperactivity disorder, dysmnesia, somnipathy, cognitive impairment, dyskinesia, dysesthesia, dysosmia, morphine resistance, drug dependence, alcohol dependence, and the like; reproductive system diseases such as infertility, premature delivery, sexual dysfunction, and the like; and other conditions including digestive diseases, respiratory diseases, cancer, and chromatosis. The compounds described herein are especially useful as a preventive or a remedy for obesity, diabetes, fatty liver, bulimia, depression, or anxiety.

For example, the present invention is directed to the use of a compound of any of the formulas described herein in the manufacture of a medicament for use in treating obesity, diabetes, hormone secretion disorder, hyperlipemia, gout and fatty liver.

Additionally, the present invention is directed to the use of a compound of any of the formulas described herein in the manufacture of a medicament for use in treating obesity.

### Pharmaceutical Compositions

Compounds of the invention may be administered orally or parenterally. As formulated into a dosage form suitable for the administration route, the compound of the invention can be used as a pharmaceutical composition for the prevention, treatment, or remedy of the above diseases.

In clinical use of the compound of the invention, usually, the compound is formulated into various preparations together with pharmaceutically acceptable additives according to the dosage form, and may then be administered. By “pharmaceutically acceptable” it is meant the additive, carrier, diluent or excipient must be compatible with the other ingredients of the formulation and not deleterious to the recipient thereof. As such additives, various additives ordinarily used in the field of pharmaceutical preparations are usable. Specific examples thereof include gelatin, lactose, sucrose, titanium oxide, starch, crystalline cellulose, hydroxypropyl methylcellulose, carboxymethylcellulose, corn starch, microcrystalline wax, white petrolatum, magnesium metasilicate aluminate, anhydrous calcium phosphate, citric acid, trisodium citrate, hydroxypropylcellulose, sorbitol, sorbitan fatty acid ester, polysorbate, sucrose fatty acid ester, polyoxyethylene, hardened castor oil, polyvinylpyrrolidone, magnesium stearate, light silicic acid anhydride, talc, vegetable oil, benzyl alcohol, gum arabic, propylene glycol, polyalkylene glycol, cyclodextrin, hydroxypropyl cyclodextrin, and the like.

Preparations to be formed with those additives include, for example, solid preparations such as tablets, capsules, granules, powders, suppositories; and liquid preparations such as syrups, elixirs, injections. These may be formulated according to conventional methods known in the field of pharmaceutical preparations. The liquid preparations may also be in such a form that may be dissolved or suspended in water or in any other suitable medium in their use. Especially for injections, if desired, the preparations may be dissolved or suspended in physiological saline or glucose liquid, and a buffer or a preservative may be optionally added thereto.

The pharmaceutical compositions may contain the compound of the invention in an amount of from 1 to 99.9% by weight, preferably from 1 to 60% by weight of the composition. The compositions may further contain any other therapeutically-effective compounds.

In case where the compounds of the invention are used for prevention or treatment for the above-mentioned diseases, the dose and the dosing frequency may be varied, depending on the sex, the age, the body weight and the disease condition of the patient and on the type and the range of the intended remedial effect. In general, when orally administered, the dose may be from 0.001 to 50 mg/kg of body weight/day, and it may be administered at a time or in several times. The dose is preferably from about 0.01 to about 25 mg/kg/day, more preferably from about 0.05 to about 10 mg/kg/day. For oral administration, the compositions are preferably provided in the form of tablets or capsules containing from 0.01 mg to 1,000 mg, preferably 0.01, 0.05, 0.1, 0.2, 0.5, 1.0, 2.5, 5, 10, 15, 20, 25, 30, 40, 50, 75, 100, 125, 150, 175, 200, 225, 250, 500, 750, 850 and 1,000 milligrams of a compound described herein. This dosage regimen may be adjusted to provide the optimal therapeutic response.

### Combination Therapy

The compounds of the present invention are further useful in methods for the prevention or treatment of the aforementioned diseases, disorders and conditions in combination with other therapeutic agents.

The compounds of the present invention may be used in combination with one or more other drugs in the treatment, prevention, suppression or amelioration of diseases or conditions for which compounds of any of the formulas described herein or the other drugs may have utility, where the combination of the drugs together are safer or more effective than either drug alone. Such other drug(s) may be administered, by a route and in an amount commonly used therefore, contemporaneously or sequentially with a compound of any of the formulas described herein. When a compound of any of the formulas described herein is used contemporaneously with one or more other drugs, a pharmaceutical composition in unit dosage form containing such other drugs and the compound of any of the formulas described herein is preferred. However, the combination therapy may also include therapies in which the compound of any of the formulas described herein and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of any of the formulas described herein.

Examples of other active ingredients that may be administered in combination with a compound of any of the formulas described herein, and either administered separately or in the same pharmaceutical composition, include, but are not limited to:

(1) dipeptidyl peptidase-IV (DPP-4) inhibitors;

(2) insulin sensitizers, including (i) PPARγ agonists, such as the glitazones (e.g. pioglitazone, rosiglitazone, netoglitazone, rivoglitazone, and balaglitazone) and other PPAR ligands, including (1) PPARα/γ □dual agonists, such as muraglitazar, aleglitazar, sodelglitazar, and naveglitazar, (2) PPARα agonists, such as fenofibric acid derivatives (gemfibrozil, clofibrate, ciprofibrate, fenofibrate and bezafibrate), (3) selective PPARγ modulators (SPPARγM's), such as those disclosed in WO 02/060388 WO 02/08188, WO 2004/019869, WO 2004/020409, WO 2004/020408, and WO 2004/066963, and (4) PPARγ □partial agonists; (ii) biguanides, such as metformin and its pharmaceutically acceptable salts, in particular, metformin hydrochloride, and extended-release formulations thereof, such as Glumetza®, Fortamet®, and GlucophageXR®; (iii) protein tyrosine phosphatase-1B (PTP-1B) inhibitors;

(3) insulin or insulin analogs, such as insulin lispro, insulin detemir, insulin glargine, insulin glulisine, and inhalable formulations of each thereof;

(4) leptin and leptin derivatives and agonists;

(5) amylin and amylin analogs, such as pramlintide;

(6) sulfonylurea and non-sulfonylurea insulin secretagogues, such as tolbutamide, glyburide, glipizide, glimepiride, mitiglinide, and meglitinides, such as nateglinide and repaglinide;
(7) α-glucosidase inhibitors (such as acarbose, voglibose and miglitol);

(8) glucagon receptor antagonists, such as those disclosed in WO 98/04528, WO 99/01423, WO 00/39088, and WO 00/69810;

(9) incretin mimetics, such as GLP-1, GLP-1 analogs, derivatives, and mimetics; and GLP-1 receptor agonists, such as exenatide, liraglutide, taspoglutide, AVE0010, CJC-1131, and BIM-51077, including intranasal, transdermal, and once-weekly formulations thereof;

(10) LDL cholesterol lowering agents such as (i) HMG-CoA reductase inhibitors (lovastatin, simvastatin, pravastatin, cerivastatin, fluvastatin, atorvastatin, pitavastatin, and rosuvastatin), (ii) bile acid sequestering agents (such as cholestyramine, colestimide, colesevelam hydrochloride, colestipol, and dialkylaminoalkyl derivatives of a cross-linked dextran, (iii) inhibitors of cholesterol absorption, such as ezetimibe, and (iv) acyl CoA:cholesterol acyltransferase inhibitors, such as avasimibe;

(11) HDL-raising drugs, such as niacin or a salt thereof and extended-release versions thereof; MK-524A, which is a combination of niacin extended-release and the DP-1 antagonist MK-524; and nicotinic acid receptor agonists;

(12) antiobesity compounds;

(13) agents intended for use in inflammatory conditions, such as aspirin, non-steroidal anti-inflammatory drugs (NSAIDs), glucocorticoids, and selective cyclooxygenase-2 (COX-2) inhibitors;

(14) antihypertensive agents, such as ACE inhibitors (such as enalapril, lisinopril, ramipril, captopril, quinapril, and tandolapril), A-II receptor blockers (such as losartan, candesartan, irbesartan, olmesartan medoxomil, valsartan, telmisartan, and eprosartan), renin inhibitors (such as aliskiren), beta blockers (such as and calcium channel blockers (such as;

(15) glucokinase activators (GKAs), such as LY2599506;

(16) inhibitors of 11β-hydroxysteroid dehydrogenase type 1, such as those disclosed in U.S. Pat. No. 6,730,690; WO 03/104207; and WO 04/058741;

(17) inhibitors of cholesteryl ester transfer protein (CETP), such as torcetrapib and MK-0859;

(18) inhibitors of fructose 1,6-bisphosphatase, such as those disclosed in U.S. Pat. Nos. 6,054,587; 6,110,903; 6,284,748; 6,399,782; and 6,489,476;

(19) inhibitors of acetyl CoA carboxylase-1 or 2 (ACC1 or ACC2);

(20) AMP-activated Protein Kinase (AMPK) activators;

(21) agonists of the G-protein-coupled receptors: GPR-109, GPR-119, and GPR-40;

(22) SSTR3 antagonists, such as those disclosed in WO 2009/011836;

(23) neuromedin U receptor agonists, such as those disclosed in WO2009/042053, including, but not limited to, neuromedin S (NMS);

(24) inhibitors of stearoyl-coenzyme A delta-9 desaturase (SCD);

(25) GPR-105 antagonists, such as those disclosed in WO 2009/000087;

(26) inhibitors of glucose uptake, such as sodium-glucose transporter (SGLT) inhibitors and its various isoforms, such as SGLT-1; SGLT-2, such as PF-04971729, dapagliflozin and remogliflozin; and SGLT-3;

(27) inhibitors of acyl coenzyme A:diacylglycerol acyltransferase 1 and 2 (DGAT-1 and DGAT-2);

(28) inhibitors of fatty acid synthase;

(29) inhibitors of acetyl-CoA carboxylase-1 and 2 (ACC-1 and ACC-2);

(30) inhibitors of acyl coenzyme A:monoacylglycerol acyltransferase 1 and 2 (MGAT-1 and MGAT-2);

(31) agonists of the TGR5 receptor (also known as GPBAR1, BG37, GPCR19, GPR131, and M BAR); and

(32) bromocriptine mesylate and rapid-release formulations thereof.

Dipeptidyl peptidase-IV (DPP-4) inhibitors that can be used in combination with compounds of any of the formulas described herein include, but are not limited to, sitagliptin (disclosed in U.S. Pat. No. 6,699,871), vildagliptin, saxagliptin, alogliptin, denagliptin, carmegliptin, dutogliptin, melogliptin, linagliptin, and pharmaceutically acceptable salts thereof, and fixed-dose combinations of these compounds with metformin hydrochloride, pioglitazone, rosiglitazone, simvastatin, atorvastatin, or a sulfonylurea.

Other dipeptidyl peptidase-IV (DPP-4) inhibitors that can be used in combination with compounds of any of the formulas described herein include, but are not limited to:
(2R,3S,5R)-5-(1-methyl-4,6-dihydropyrrolo[3,4-c]pyrazol-5(1H)-yl)-2-(2,4,5-trifluorophenyl)tetrahydro-2H-pyran-3-amine;
(2R,3S,5R)-5-(1-methyl-4,6-dihydropyrrolo[3,4-c]pyrazol-5(1H)-yl)-2-(2,4,5-trifluorophenyl)tetrahydro-2H-pyran-3-amine;
(2R,3S,5R)-2-(2,5-difluorophenyl)tetrahydro)-5-(4,6-dihydropyrrolo[3,4-c]pyrazol-5(1H)-yl)tetrahydro-2H-pyran-3-amine;
(3R)-4-[(3R)-3-amino-4-(2,4,5-trifluorophenyl)butanoyl]-hexahydro-3-methyl-2H-1,4-diazepin-2-one;
4-[(3R)-3-amino-4-(2,5-difluorophenyl)butanoyl]hexahydro-1-methyl-2H-1,4-diazepin-2-one hydrochloride; and
(3R)-4-[(3R)-3-amino-4-(2,4,5-trifluorophenyl)butanoyl]-hexahydro-3-(2,2,2-trifluoroethyl)-2H-1,4-diazepin-2-one; and
   pharmaceutically acceptable salts thereof.

Antiobesity compounds that can be combined with compounds of any of the formulas described herein include topiramate; zonisamide; naltrexone; phentermine; bupropion; the combination of bupropion and naltrexone; the combination of bupropion and zonisamide; the combination of topiramat and phentermine; fenfluramine; dexfenfluramine; sibutramine; lipase inhibitors, such as orlistat and cetilistat; melanocortin receptor agonists, in particular, melanocortin-4 receptor agonists; CCK-1 agonists; melanin-concentrating hormone (MCH) receptor antagonists; neuropeptide Y₁ or Y₅ antagonists (such as MK-0557); CB1 receptor inverse agonists and antagonists (such as rimonabant and taranabant); β₃ adrenergic receptor agonists; ghrelin antagonists; bombesin receptor agonists (such as bombesin receptor subtype-3 agonists); and 5-hydroxytryptamine-2c (5-HT2c) agonists, such as lorcaserin. For a review of anti-obesity compounds that can be combined with compounds of the preset invention, see S. Chaki et al., “Recent advances in feeding suppressing agents: potential therapeutic strategy for the treatment of obesity,” Expert Opin. Ther. Patents, 11: 1677-1692 (2001); D. Spanswick and K. Lee, “Emerging antiobesity drugs,” Expert Opin. Emerging Drugs, 8: 217-237 (2003); J. A. Fernandez-Lopez, et al., “Pharmacological Approaches for the Treatment of Obesity,” Drugs, 62: 915-944 (2002); and K. M. Gadde, et al., “Combination pharmaceutical therapies for obesity,” Exp. Opin. Pharmacother., 10: 921-925 (2009).

Glucagon receptor antagonists that can be used in combination with the compounds of any of the formulas described herein include, but are not limited to:
N-[4-((1S)-1-{3-(3,5-dichlorophenyl)-5-[6-(trifluoromethoxy)-2-naphthyl]-1H-pyrazol-1-yl}ethyl)benzoyl]-β-alanine;
N-[4-((1R)-1-{3-(3,5-dichlorophenyl)-5-[6-(trifluoromethoxy)-2-naphthyl]-1H-pyrazol-1-yl}ethyl)benzoyl]-β-alanine;
N-(4-{1-[3-(2,5-dichlorophenyl)-5-(6-methoxy-2-naphthyl)-1H-pyrazol-1-yl]ethyl}benzoyl)-β-alanine;
N-(4-{(1S)-1-[3-(3,5-dichlorophenyl)-5-(6-methoxy-2-naphthyl)-1H-pyrazol-1-yl]ethyl}benzoyl)-β-alanine;
N-(4-{(1S)-1-[(R)-(4-chlorophenyl)(7-fluoro-5-methyl-1H-indol-3-yl)methyl]butyl}benzoyl)-β-alanine; and
N-(4-{(1S)-1-[(4-chlorophenyl)(6-chloro-8-methylquinolin-4-yl)methyl]butyl}benzoyl)-β-alanine; and
   pharmaceutically acceptable salts thereof.

Inhibitors of stearoyl-coenzyme A delta-9 desaturase (SCD) that can be used in combination with the compounds of any of the formulas described herein include, but are not limited to:
[5-(5-{4-[2-(trifluoromethyl)phenoxy]piperidin-1-yl}-1,3,4-thiadiazol-2-yl)-2H-tetrazol-2-yl]acetic acid;
(2′-{4-[2-(trifluoromethyl)phenoxy]piperidin-1-yl}-2,5′-bi-1,3-thiazol-4-yl)acetic acid;
(5-{3-[4-(2-bromo-5-fluorophenoxy)piperidin-1-yl]isoxazol-5-yl}-2H-tetrazol-2-yl)acetic acid;
(3-{3-[4-(2-bromo-5-fluorophenoxy)piperidin-1-yl]-1,2,4-oxadiazol-5-yl}-1H-pyrrol-1-yl)acetic acid;
(5-{5-[4-(2-bromo-5-fluorophenoxy)piperidin-1-yl]pyrazin-2-yl}-2H-tetrazol-2-yl)acetic acid; and
(5-{2-[4-(5-bromo-2-chlorophenoxy)piperidin-1-yl]pyrimidin-5-yl}-2H-tetrazol-2-yl)acetic acid;
   and pharmaceutically acceptable salts thereof.

Glucokinase activators that can be used in combination with the compounds of any of the formulas described herein include, but are not limited to:
3-(6-ethanesulfonylpyridin-3-yloxy)-5-(2-hydroxy-1-methyl-ethoxy)-N-(1-methyl-1H-pyrazol-3-yl)benzamide;
5-(2-hydroxy-1-methyl-ethoxy)-3-(6-methanesulfonylpyridin-3-yloxy)-N-(1-methyl-1H-pyrazol-3-yl)benzamide;
5-(1-hydroxymethyl-propoxy)-3-(6-methanesulfonylpyridin-3-yloxy)-N-(1-methyl-1H-pyrazol-3-yl)benzamide;
3-(6-methanesulfonylpyridin-3-yloxy)-5-(1-methoxymethyl-propoxy)-N-(1-methyl-1H-pyrazol-3-yl)benzamide;
5-isopropoxy-3-(6-methanesulfonylpyridin-3-yloxy)-N-(1-methyl-1H-pyrazol-3-yl)benzamide;
5-(2-fluoro-1-fluoromethyl-ethoxy)-3-(6-methanesulfonylpyridin-3-yloxy)-N-(1-methyl-1H-pyrazol-3-yl)benzamide;
3-({4-[2-(dimethylamino)ethoxy]phenyl}thio)-N-(3-methyl-1,2,4-thiadiazol-5-yl)-6-[(4-methyl-4H-1,2,4-triazol-3-yl)thio]pyridine-2-carboxamide;
3-({4-[(1-methylazetidin-3-yl)oxy]phenyl}thio)-N-(3-methyl-1,2,4-thiadiazol-5-yl)-6-[(4-methyl-4H-1,2,4-triazol-3-yl)thio]pyridine-2-carboxamide;
N-(3-methyl-1,2,4-thiadiazol-5-yl)-6-[(4-methyl-4H-1,2,4-triazol-3-yl)thio]-3-{[4-(2-pyrrolidin-1-ylethoxy)phenyl]thio}pyridine-2-carboxamide; and
3-[(4-{2-[(2R)-2-methylpyrrolidin-1-yl]ethoxy}phenyl)thio-N-(3-methyl-1,2,4-thiadiazol-5-yl)-6-[(4-methyl-4H-1,2,4-triazol-3-yl)thio]pyridine-2-carboxamide; and pharmaceutically acceptable salts thereof.

Agonists of the GPR-119 receptor that can be used in combination with the compounds of any the formulas described herein include, but are not limited to:
rac-cis 5-chloro-2-{4-[2-(2-{[5-methylsulfonyl)pyridin-2-yl]oxy}ethyl)cyclopropyl]piperidin-1-yl}pyrimidine;
5-chloro-2-{4-[(1R,2S)-2-(2-{[5-(methylsulfonyl)pyridin-2-yl]oxy}ethyl)cyclopropyl]piperidin-1-yl}pyrimidine;
rac cis-5-chloro-2-[4-(2-{2-[4-(methylsulfonyl)phenoxy]ethyl}cyclopropyl)piperidin-1-yl]pyrimidine;
5-chloro-2-[4-((1S,2R)-2-{2-[4-(methylsulfonyl)phenoxy]ethyl}cyclopropyl) piperidin-1-yl]pyrimidine;
5-chloro-2-[4-((1R,2S)-2-{2-[4-(methylsulfonyl)phenoxy]ethyl}cyclopropyl)piperidin-1-yl]pyrimidine;
rac cis-5-chloro-2-[4-(2-{2-[3-(methylsulfonyl)phenoxy]ethyl}cyclopropyl)piperidin-1-yl]pyrimidine; and
rac cis-5-chloro-2-[4-(2-{2-[3-(5-methyl-1,3,4-oxadiazol-2-yl)phenoxy]ethyl}cyclopropyl) piperidin-1-yl]pyrimidine; and pharmaceutically acceptable salts thereof.

Selective PPARγ modulators (SPPARγM's) that can be used in combination with the compound of any of the formulas described herein include, but are not limited to:
(2S)-2-({6-chloro-3-[6-(4-chlorophenoxy)-2-propylpyridin-3-yl]-1,2-benzisoxazol-5-yl}oxy)propanoic acid;
(2S)-2-({6-chloro-3-[6-(4-fluorophenoxy)-2-propylpyridin-3-yl]-1,2-benzisoxazol-5-yl}oxy)propanoic acid;
(2S)-2-{[6-chloro-3-(6-phenoxy-2-propylpyridin-3-yl)-1,2-benzisoxazol-5-yl]oxy}propanoic acid;
(2R)-2-({6-chloro-3-[6-(4-chlorophenoxy)-2-propylpyridin-3-yl]-1,2-benzisoxazol-5-yl}oxy)propanoic acid;
(2R)-2-{3-[3-(4-methoxy)benzoyl-2-methyl-6-(trifluoromethoxy)-1H-indol-1-yl]phenoxy}butanoic acid;
(2S)-2-{3-[3-(4-methoxy)benzoyl-2-methyl-6-(trifluoromethoxy)-1H-indol-1-yl]phenoxy}butanoic acid;
2-{3-[3-(4-methoxy)benzoyl-2-methyl-6-(trifluoromethoxy)-1H-indol-1-yl]phenoxy}-2-methylpropanoic acid; and
(2R)-2-{3-[3-(4-chloro)benzoyl-2-methyl-6-(trifluoromethoxy)-1H-indol-1-yl]phenoxy}propanoic acid; and pharmaceutically acceptable salts thereof.

Inhibitors of 11β-hydroxysteroid dehydrogenase type 1 that can be used in combination with the compounds of any of the formulas described herein include, but are not limited to:
3-[1-(4-chlorophenyl)-trans-3-fluorocyclobutyl]-4,5-dicyclopropyl-r-4H-1,2,4-triazole; 3-[1-(4-chlorophenyl)-trans-3-fluorocyclobutyl]-4-cyclopropyl-5-(1-methylcyclopropyl)-r-4H-1,2,4-triazole;
3-[1-(4-chlorophenyl)-trans-3-fluorocyclobutyl]-4-methyl-5-[2-(trifluoromethoxy)phenyl]-r-4H-1,2,4-triazole;
3-[1-(4-chlorophenyl)cyclobutyl]-4-methyl-5-[2-(trifluoromethyl)phenyl]-4H-1,2,4-triazole;
3-{4-[3-(ethylsulfonyl)propyl]bicyclo[2.2.2]oct-1-yl}-4-methyl-5-[2-(trifluoromethyl)phenyl]-4H-1,2,4-triazole;
4-methyl-3-{4-[4-(methylsulfonyl)phenyl]bicyclo[2.2.2]oct-1-yl}-5-[2-(trifluoromethyl)phenyl]-4H-1,2,4-triazole;
3-(4-{4-methyl-5-[2-(trifluoromethyl)phenyl]-4H-1,2,4-triazol-3-yl}bicyclo[2.2.2]oct-1-yl)-5-(3,3,3-trifluoropropyl)-1,2,4-oxadiazole;
3-(4-{4-methyl-5-[2-(trifluoromethyl)phenyl]-4H-1,2,4-triazol-3-yl}bicyclo[2.2.2]oct-1-yl)-5-(3,3,3-trifluoroethyl)-1,2,4-oxadiazole;
5-(3,3-difluorocyclobutyl)-3-(4-{4-methyl-5-[2-(trifluoromethyl)phenyl]-4H-1,2,4-triazol-3-yl}bicyclo[2.2.2]oct-1-yl)-1,2,4-oxadiazole;
5-(1-fluoro-1-methylethyl)-3-(4-{4-methyl-5-[2-(trifluoromethyl)phenyl]-4H-1,2,4-triazol-3-yl}bicyclo[2.2.2]oct-1-yl)-1,2,4-oxadiazole;
2-(1,1-difluoroethyl)-5-(4-{4-methyl-5-[2-(trifluoromethyl)phenyl]-4H-1,2,4-triazol-3-yl}bicyclo[2.2.2]oct-1-yl)-1,3,4-oxadiazole;
2-(3,3-difluorocyclobutyl)-5-(4-{4-methyl-5-[2-(trifluoromethyl)phenyl]-4H-1,2,4-triazol-3-yl}bicyclo[2.2.2]oct-1-yl)-1,3,4-oxadiazole; and
5-(1,1-difluoroethyl)-3-(4-{4-methyl-5-[2-(trifluoromethyl)phenyl]-4H-1,2,4-triazol-3-yl}bicyclo[2.2.2]oct-1-yl)-1,2,4-oxadiazole; and pharmaceutically acceptable salts thereof.

Somatostatin subtype receptor 3 (SSTR3) antagonists that can be used in combination with the compounds of any of the formulas described herein include, but are not limited to:

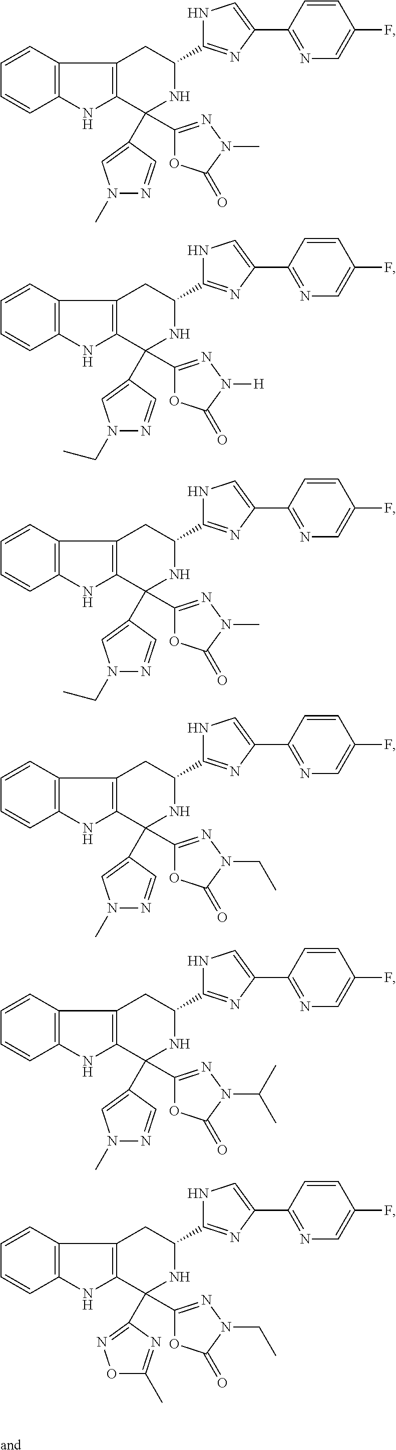

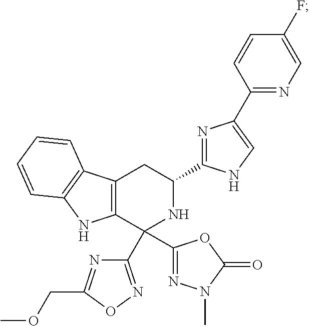 [SMILES: COCc1nc(C2(c3nn(C)c(=O)o3)N[C@@H](c3nc(-c4ccc(F)cn4)c[nH]3)Cc3c2[nH]c2ccccc32)no1]

and pharmaceutically acceptable salts thereof.

AMP-activated Protein Kinase (AMPK) activators that can be used in combination with the compounds of any of the formulas described herein include, but are not limited to:

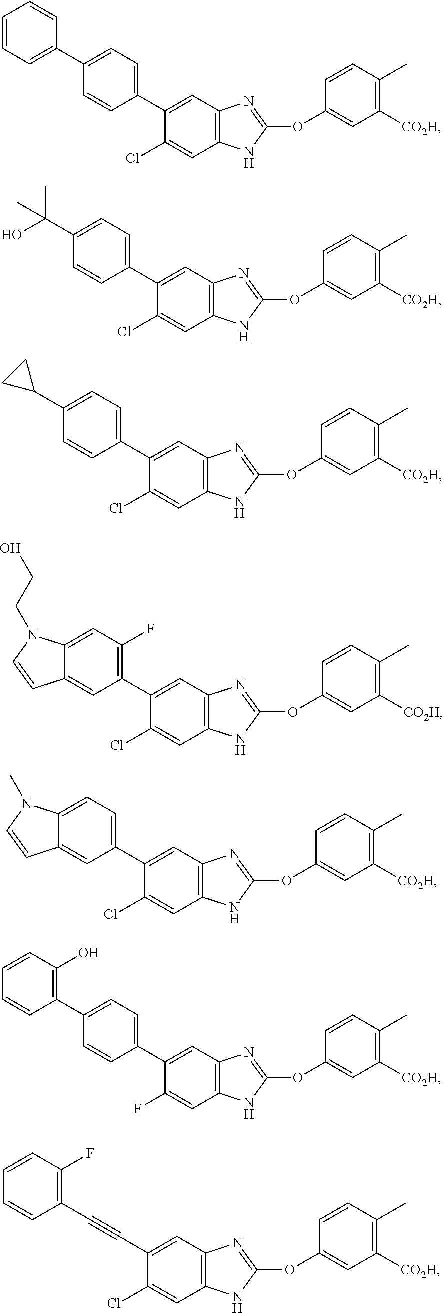

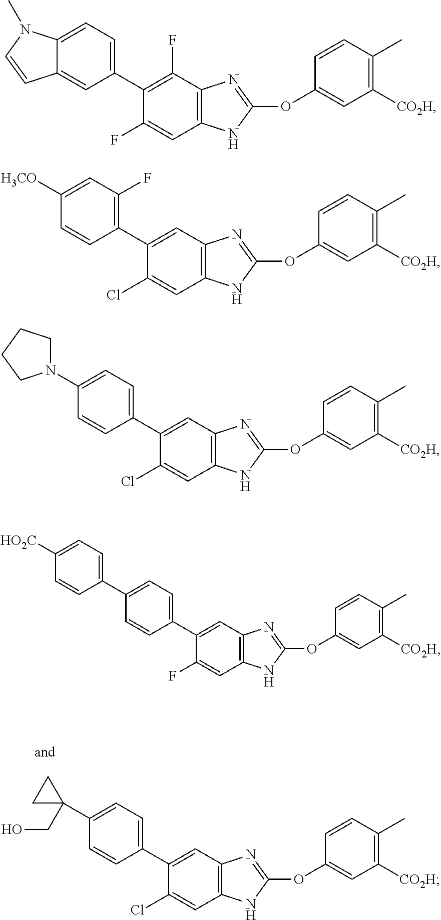 [SMILES: Cc1ccc(Oc2nc3c(F)c(-c4ccc5c(ccn5C)c4)c(F)cc3[nH]2)cc1C(=O)O ; COc1ccc(-c2cc3nc(Oc4ccc(C)c(C(=O)O)c4)[nH]c3cc2Cl)c(F)c1 ; Cc1ccc(Oc2nc3cc(-c4ccc(N5CCCC5)cc4)c(Cl)cc3[nH]2)cc1C(=O)O ; Cc1ccc(Oc2nc3cc(-c4ccc(-c5ccc(C(=O)O)cc5)cc4)c(F)cc3[nH]2)cc1C(=O)O ; Cc1ccc(Oc2nc3cc(-c4ccc(C5(CO)CC5)cc4)c(Cl)cc3[nH]2)cc1C(=O)O]

and pharmaceutically acceptable salts thereof.

Inhibitors of acetyl-CoA carboxylase-1 and 2 (ACC-1 and ACC-2) that can be used in combination with the compounds of any of the formulas described herein include, but are not limited to
3-{1′-[(1-cyclopropyl-4-methoxy-1H-indol-6-yl)carbonyl]-4-oxospiro[chroman-2,4′-piperidin]-6-yl}benzoic acid;
5-{1′-[(1-cyclopropyl-4-methoxy-1H-indol-6-yl)carbonyl]-4-oxospiro[chroman-2,4′-piperidin]-6-yl}nicotinic acid;
1′-[(1-cyclopropyl-4-methoxy-1H-indol-6-yl)carbonyl]-6-(1H-tetrazol-5-yl)spiro[chroman-2,4′-piperidin]-4-one;
1′-[(1-cyclopropyl-4-ethoxy-3-methyl-1H-indol-6-yl)carbonyl]-6-(1H-tetrazol-5-yl)spiro[chroman-2,4′-piperidin]-4-one; and
5-{1′-[(1-cyclopropyl-4-methoxy-3-methyl-1H-indol-6-yl)carbonyl]-4-oxo-spiro[chroman-2,4′-piperidin]-6-yl}nicotinic acid; and
   pharmaceutically acceptable salts thereof.

In another aspect of the invention, a pharmaceutical composition is disclosed which comprises one or more of the following agents:

(a) a compound of any of the formulas described herein;

(b) one or more compounds selected from the group consisting of:
(1) dipeptidyl peptidase-IV (DPP-4) inhibitors;
(2) insulin sensitizers, including (i) PPARγ agonists, such as the glitazones (e.g. pioglitazone, rosiglitazone, netoglitazone, rivoglitazone, and balaglitazone) and other PPAR ligands, including (1) PPARα/γ □dual agonists, such as muraglitazar, aleglitazar, sodelglitazar, and naveglitazar;
(2) PPARα agonists, such as fenofibric acid derivatives (gemfibrozil, clofibrate, ciprofibrate, fenofibrate and bezafibrate), (3) selective PPARγ modulators (SPPARγM's), and (4) PPARγ partial agonists; (ii) biguanides, such as metformin and its pharmaceutically acceptable salts, in particular, metformin hydrochloride, and extended-release formulations thereof, such as Glumetza®, Fortamet®, and GlucophageXR®; (iii) protein tyrosine phosphatase-1B (PTP-1B) inhibitors;
(3) sulfonylurea and non-sulfonylurea insulin secretagogues, such as tolbutamide, glyburide, glipizide, glimepiride, mitiglinide, and meglitinides, such as nateglinide and repaglinide;
(4) α-glucosidase inhibitors (such as acarbose, voglibose and miglitol);
(5) glucagon receptor antagonists;
(6) LDL cholesterol lowering agents such as (i) HMG-CoA reductase inhibitors (lovastatin, simvastatin, pravastatin, cerivastatin, fluvastatin, atorvastatin, pitavastatin, and rosuvastatin (ii) bile acid sequestering agents (such as cholestyramine, colestimide, colesevelam hydrochloride, colestipol, and dialkylaminoalkyl derivatives of a cross-linked dextran, (iii) inhibitors of cholesterol absorption, such as ezetimibe, and (iv) acyl CoA:cholesterol acyltransferase inhibitors, such as avasimibe;
(7) HDL-raising drugs, such as niacin or a salt thereof and extended-release versions thereof; MK-524A, which is a combination of niacin extended-release and the DP-1 antagonist MK-524 and nicotinic acid receptor agonists;
(8) antiobesity compounds;
(9) agents intended for use in inflammatory conditions, such as aspirin, non-steroidal ant inflammatory drugs (NSAIDs), glucocorticoids, and selective cyclooxygenase-2 (COX-2) inhibitors;
(10) antihypertensive agents, such as ACE inhibitors (such as enalapril, lisinopril, ramipril, captopril, quinapril, and tandolapril), A-II receptor blockers (such as losartan, candesartan, irbesartan, olmesartan medoxomil, valsartan, telmisartan, and eprosartan), renin inhibitors (such as aliskiren), beta blockers (such as and calcium channel blockers (such as;
(11) glucokinase activators (GKAs), such as LY2599506;

(12) inhibitors of 11β-hydroxysteroid dehydrogenase type 1;
(13) inhibitors of cholesteryl ester transfer protein (CETP), such as torcetrapib and MK-0859;
(14) inhibitors of fructose 1,6-bisphosphatase;
(15) inhibitors of acetyl CoA carboxylase-1 or 2 (ACCT or ACC2);
(16) AMP-activated Protein Kinase (AMPK) activators;
(17) agonists of the G-protein-coupled receptors: GPR-109, GPR-119, and GPR-40;
(18) SSTR3 antagonists;
(19) neuromedin U receptor agonists, including, but not limited to, neuromedin S (NMS)
(20) inhibitors of stearoyl-coenzyme A delta-9 desaturase (SCD);
(21) GPR-105 antagonists;
(22) inhibitors of glucose uptake, such as sodium-glucose transporter (SGLT) inhibitors and its various isoforms, such as SGLT-1; SGLT-2, such as dapagliflozin and remogliflozin; and SGLT 3;
(23) inhibitors of acyl coenzyme A:diacylglycerol acyltransferase 1 and 2 (DGAT-1 and DGAT-2);
(24) inhibitors of fatty acid synthase;
(25) inhibitors of acetyl-CoA carboxylase-1 and 2 (ACC-1 and ACC-2);
(26) inhibitors of acyl coenzyme A:monoacylglycerol acyltransferase 1 and 2 (MGAT-1 and MGAT-2);
(27) agonists of the TGR5 receptor (also known as GPBAR1, BG37, GPCR19, GPR131 and M-BAR); and
(28) bromocriptine mesylate and rapid-release formulations thereof; and

(c) a pharmaceutically acceptable carrier.

In certain embodiments, the compounds described herein can be combined with a DPP-IV inhibitor, such as sitagliptin. DPP 4 is responsible on the inactivation of incretin hormones GLP-1(glucagon-like peptide-1) and GIP (glucose-dependent insulinotropic polypeptide). Thus sitagliptin would inhitbit the inactivation of incretin hormones while DGAT-1 would inhibit tryglicride synthesis.

When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention.

The weight ratio of the compound of the present invention to the second active ingredient may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used. In such combinations the compound of the present invention and other active agents may be administered separately or in conjunction. In addition, the administration of one element may be prior to, concurrent to, or subsequent to the administration of other agent(s).

### EXAMPLES

### General Method

### A (Examplified by):

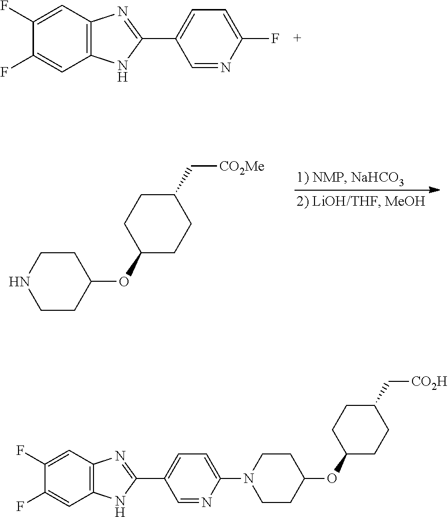

### B (Examplified by):

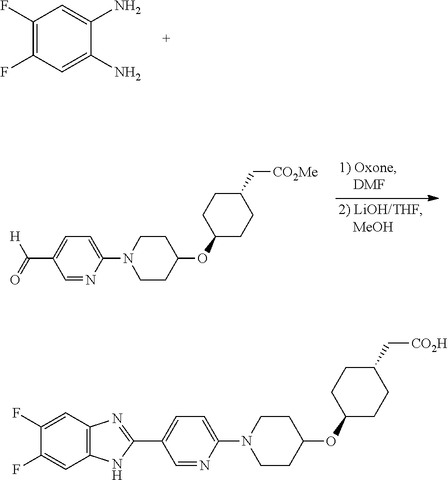 [SMILES: Nc1cc(F)c(F)cc1N ; "+" ; COC(=O)C[C@H]1CC[C@H](OC2CCN(c3ccc(C=O)cn3)CC2)CC1 ; O=C(O)C[C@H]1CC[C@H](OC2CCN(c3ccc(-c4nc5cc(F)c(F)cc5[nH]4)cn3)CC2)CC1]

### C (Examplified by):

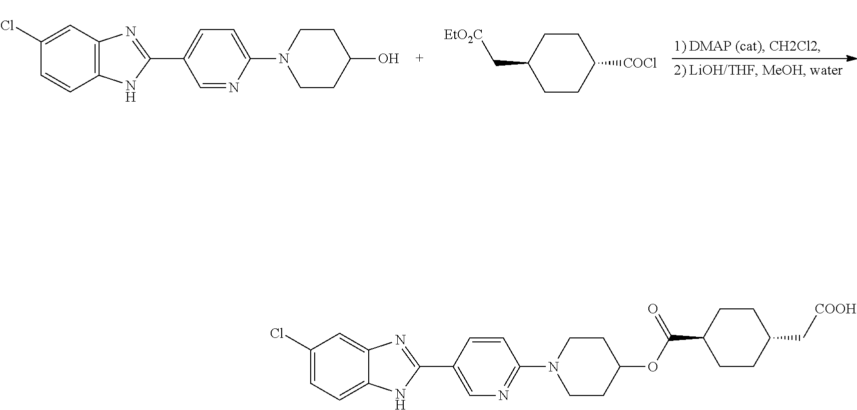

### D (Examplified by):

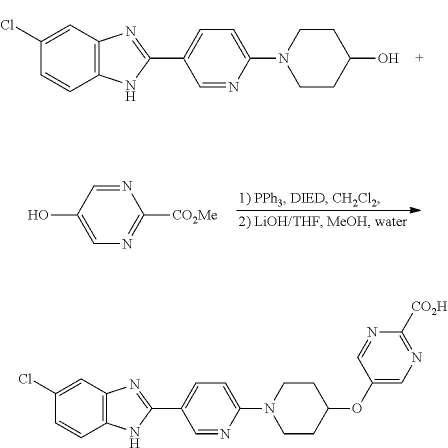

### E (Examplified by):

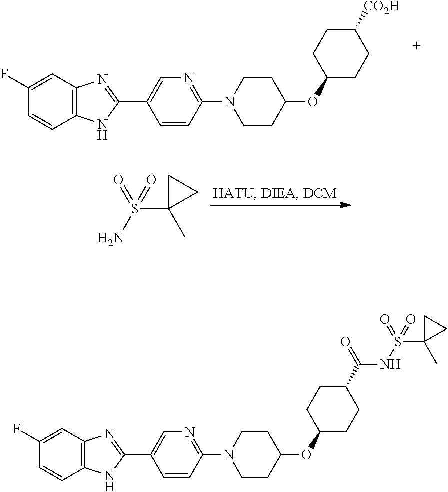

### F (Exemplified by):

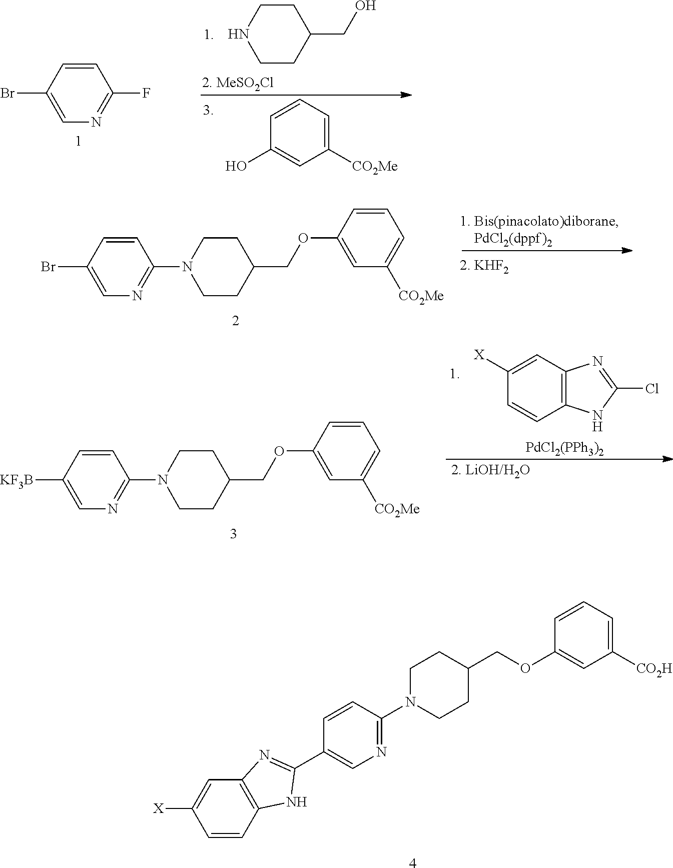

### G (Exemplified by)

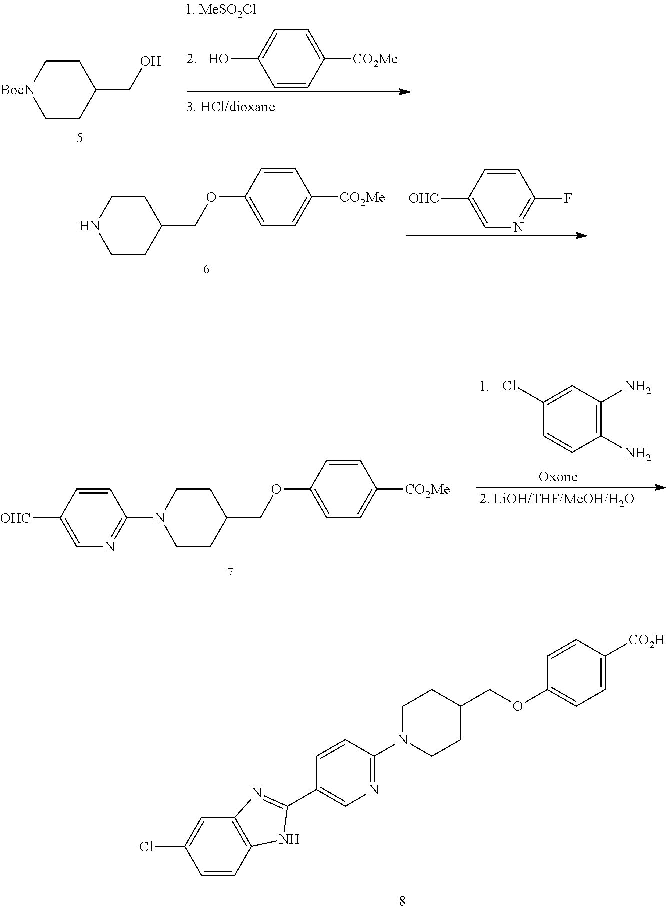

### Intermediate 1: 6-fluoro-2-(6-fluoropyridin-3-yl)-1H-benzimidazole

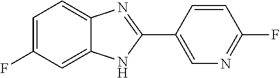 [SMILES: Fc1ccc2nc(-c3ccc(F)nc3)[nH]c2c1]

In a 2 L round-bottom flask equipped with magnetic stirring and nitrogen inlet, 6-fluoropyridine-3-carbaldehyde (25 g, 196 mmol) was dissolved in DMA (400 ml) and the solution was cooled to 0° C. 4-Fluorobenzene-1,2-diamine (25.5 g, 196 mmol) was added (exotherm). Water (360 ml) was added followed by slow addition of potassium peroxymonosulfate (78 g, 127 mmol). The dark brown slurry was allowed to age at room temperature. After 3 h, the reaction mixture was diluted with water (2 L) and the remaining slurry was allowed to age overnight at room temperature. The reaction mixture was filtered (slow filtration) and the wet cake was washed with additional water. The wet cake was dried over nitrogen sweep and vacuum. The filter cake was later transferred to a round-bottom flask and triturated with MeCN. The mixture was filtered and the solid was dried over nitrogen sweep and under vacuum to afford solid product 6-fluoro-2-(6-fluoropyridin-3-yl)-1H-benzimidazole. LC-MS (ES, m/z) C₁₂H₇F₂N₃: 231; Found: 232 [M+H]⁺.

### Intermediate 2: 2-(6-fluoropyridin-3-yl)-1H-benzimidazole

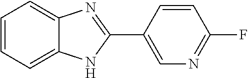 [SMILES: Fc1ccc(-c2nc3ccccc3[nH]2)cn1]

Performed the same as the synthesis of Intermediate 1 except that benzene-1,2-diamine was used as the starting material and the solid product was isolated by aqueous extraction with ethyl acetate followed by trituration with MTBE/heptane. LC-MS (ES, m/z): C₁₂H₈FN₃: 213; Found: 214 [M+H]⁺.

### Intermediate 3: 5,6-difluoro-2-(6-fluoropyridin-3-yl)-1H-benzimidazole

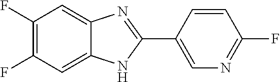 [SMILES: Fc1ccc(-c2nc3cc(F)c(F)cc3[nH]2)cn1]

Performed the same as the synthesis of Intermediate 1 except that 4,5-difluorobenzene-1,2-diamine was used as the starting material and the solid product was isolated by aqueous extraction with ethyl acetate followed by precipitation from dichloromethane/heptanes. LC-MS (ES, m/z): C₁₂H₆F₃N₃: 249; Found: 250 [M+H]⁺.

### Intermediate 4: 2-(6-fluoropyridin-3-yl)-5-(trifluoromethyl)-1H-benzimidazole

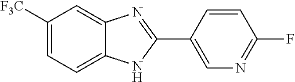 [SMILES: Fc1ccc(-c2nc3cc(C(F)(F)F)ccc3[nH]2)cn1]

Performed as same as the synthesis of Intermediate 1 except that 4-(trifluoromethyl)benzene-1,2-diamine was used as the starting material. LC-MS (ES, m/z): C₁₃H₇F₄N₃: 281; Found: 282 [M+H]⁺.

### Intermediate 5: 5-chloro-2-(6-fluoropyridin-3-yl)-1H-benzimidazole

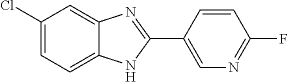 [SMILES: Fc1ccc(-c2nc3cc(Cl)ccc3[nH]2)cn1]

Performed the same as the synthesis of Intermediate 1 except that 4-chlorobenzene-1,2-diamine was used as the starting material. LC-MS (ES, m/z): C₁₂H₇ClFN₃: 247; Found: 248 [M+H]⁺.

### Intermediate 6: 5-ethoxy-2-(6-fluoropyridin-3-yl)-1H-imidazo[4,5-b]pyridine

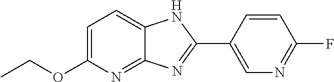 [SMILES: CCOc1ccc2[nH]c(-c3ccc(F)nc3)nc2n1]

To a mixture of 6-ethoxypyridine-2,3-diamine (2.91 g, 18.98 mmol) and 6-fluoropyridine-3-carbaldehyde (2.5 g, 19.98 mmol) in DMF (40 ml) and water (4 ml) at room temperature was added potassium peroxymonosulfate (7.99 g, 12.99 mmol) in portions over 1 hour. The reaction mixture was stirred at room temperature over night, then poured into 50 ml water, extracted with 3×50 ml ethyl acetate. The organic layers were combined, washed with 2×5 mL of saturated brine, dried over anhydrous sodium sulfate and concentrated under vacuum. The crude material was purified by a silica gel column eluted with acetone/dichloromethane (0-50%). The material was further triturated with acetone/dichloromethane. This resulted in 5-ethoxy-2-(6-fluoropyridin-3-yl)-1H-imidazo[4,5-b]pyridine as a brown solid. LC-MS (ES, m/z) C₁₃H₁₁FN₄O: 258; Found: 259 [M+H]⁺.

### Intermediate 7: 3-bromo-4,5-difluorobenzene-1,2-diamine

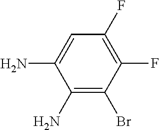 [SMILES: Nc1cc(F)c(F)c(Br)c1N]

### Step 1

4,5-difluoro-2-nitroaniline (4.26 g, 24.47 mmol) was dissolved in acetic acid (40.8 ml). Bromine (1.336 ml, 25.9 mmol) was added drop wise at room temperature. The reaction mixture was stirred for 2 hours then poured into ice water (200 ml). The mixture was allowed to stand overnight. The mixture was filtered to afford a yellow solid, which was purified by a silica gel column eluted with ethyl acetate/hexane 0-40%. This resulted in 2-bromo-3,4-difluoro-6-nitroaniline as yellow solid. LC-MS (ES, m/z) C₆H₃BrF₂N₂O₂: 253; Found: 254 [M+H]⁺.

### Step 2

To a solution of 2-bromo-3,4-difluoro-6-nitroaniline (1.62 g, 6.40 mmol) in ethanol (9.85 ml) and conc. HCl (2.5 ml) was added tin(II) chloride dihydrate (7.22 g, 32.0 mmol). The mixture was stirred at 60° C. under N₂ for 2 hours. The reaction mixture was cooled to room temperature then poured into 2N NaOH (30 ml) with ice, extracted with 3×50 ml DCM. The organic layers were combined, dried over anhydrous sodium sulfate and concentrated under vacuum to afford 3-bromo-4,5-difluorobenzene-1,2-diamine as greenish brown solid. LC-MS (ES, m/z) C₆H₅BrF₂N₂: 224; Found: 225 [M+H]⁺.

### Intermediate 8: 7-bromo-2-(6-fluoropyridin-3-yl)-5-(trifluoromethyl)-1H-benzimidazole

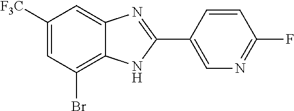 [SMILES: Fc1ccc(-c2nc3cc(C(F)(F)F)cc(Br)c3[nH]2)cn1]

To a mixture of 6-fluoronicotinaldehyde (2.5 g, 19.98 mmol), 3-bromo-5-(trifluoromethyl)benzene-1,2-diamine (4.84 g, 19.98 mmol) in DMF (40 ml) and water (4 ml) added potassium peroxymonosulfate (7.99 g, 12.99 mmol) in portions over 1 hour. The reaction mixture was stirred overnight under N₂ then pour into water (50 ml), extract with 3×80 ml ethyl acetate. The organic layers were combined, washed with 2×25 ml of saturated brine, dried over anhydrous sodium sulfate and concentrated under vacuum. Part of the product was crystallized from dichloromethane. The mother liquor was purified by reverse phase HPLC to afford 7-bromo-2-(6-fluoropyridin-3-yl)-5-(trifluoromethyl)-1H-benzimidazole as brown solid. LC-MS (ES, m/z) C₁₃H₆BrF₄N₃: 361; Found: 362 [M+H]⁺.

### Intermediate 9: 1-[5-(5-chloro-1H-benzimidazol-2-yl)pyridin-2-yl]piperidin-4-ol

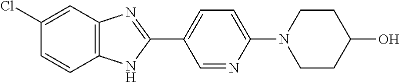 [SMILES: OC1CCN(c2ccc(-c3nc4cc(Cl)ccc4[nH]3)cn2)CC1]

4-Hydroxypiperidine (1.225 g, 12.11 mmol) and 5-chloro-2-(6-fluoropyridin-3-yl)-1H-benzimidazole (3 g, 12.11 mmol) were combined in anhydrous DMF along with sodium bicarbonate (5.09 g, 60.6 mmol) and heated at 110° C. for 18 h. The reaction mixture was cooled to room temperature. Water was added and the mixture was lyophilized to give 8.27 g crude material, which was purified by silica gel column eluted with 40-100% acetone in dichloromethane to afford 1.5 gram of 1-[5-(5-chloro-1H-benzimidazol-2-yl)pyridin-2-yl]piperidin-4-ol as a solid. LC-MS (ES, m/z) C₁₇H₁₇ClN₄O: 328; Found: 329 [M+H]⁺.

### Intermediate 10: methyl (trans/cis-4-hydroxycyclohexyl)acetate

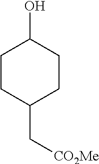 [SMILES: COC(=O)CC1CCC(O)CC1]

Methyl (trans/cis-4-hydroxycyclohexyl)acetate was prepared from methyl 2-(4-hydroxyphenyl) acetate according to a known procedure (Birch, Alan Martin et. al. PCT Int. Appl., 2009024821, 26 Feb. 2009). LC-MS (ES, m/z): C₉H₁₆O₃: 172; Found: 173 [M+H]⁺.

### Intermediate 11: methyl (trans-4-hydroxycyclohexyl)acetate and Intermediate 12: methyl (cis-4-hydroxycyclohexyl)acetate

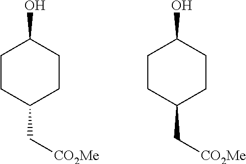 [SMILES: COC(=O)C[C@H]1CC[C@H](O)CC1 ; COC(=O)C[C@H]1CC[C@@H](O)CC1]

Methyl (trans & cis-4-hydroxycyclohexyl)acetate were separated by SFC (ChiralPak IC-5μ. 250×50 mmI.D, Mobile phase: A for CO₂ and B for ethanol. Gradient: B 15%) to afford methyl (trans-4-hydroxycyclohexyl)acetate, LC-MS (ES, m/z): C₉H₁₆O₃: 172; Found: 156 [M−16]⁺ and methyl (cis-4-hydroxycyclohexyl)acetate), LC-MS (ES, m/z): C₉H₁₆O₃: 172; Found: 173 [M+H]⁺.

### Intermediate 13: methyl [trans/cis 4-(pyridin-4-yloxy)cyclohexyl]acetate

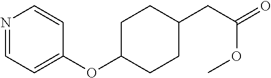 [SMILES: COC(=O)CC1CCC(Oc2ccncc2)CC1]

To a mixture of 4-hydroxypyridine (2.76 g, 29 mmol), methyl (trans & cis-4-hydroxycyclohexyl)acetate (5 g, 29 mmol) and triphenylphosphine (9.52 g, 36.3 mmol) in THF (100 ml) was added diisopropylazodicarboxylate (7.34 g, 36.3 mmol) drop wise. The reaction mixture was heated at 55° C. in an oil bath for 2 days under N₂. The reaction mixture was cooled to room temperature, concentrated under vacuum then purified by SFC (ChiralPak IA 250×30 mmI.D. Mobile phase: A for CO₂ and B for MeOH:MeCN (2:1), Gradient: B 30%). This resulted in methyl [trans & cis 4-(pyridin-4-yloxy)cyclohexyl]acetate as a colorless oil. LC-MS (ES, m/z) C₁₄H₁₉NO₃: 249; Found: 250 [M+H]⁺.

### Intermediate 14: methyl [trans & cis 4-(piperidin-4-yloxy)cyclohexyl]acetate

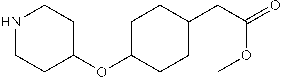 [SMILES: COC(=O)CC1CCC(OC2CCNCC2)CC1]

Methyl 2-(trans/cis-4-(pyridin-4-yloxy)cyclohexyl)acetate (1.2 g, 4.81 mmol) was dissolved in acetic acid (80 ml). The solution was passed through Rh/Al₂O₃ cartridge on H-Cube at 80° C. under 80 bars. The reaction mixture was concentrated under vacuum to result in 1.13 g (92%) of methyl [trans/cis 4-(piperidin-4-yloxy)cyclohexyl]acetate as a colorless oil. LC-MS (ES, m/z) C₁₄H₂₅NO₃: 255; Found: 256 [M+H]⁺.

### Intermediate 15: benzyl 4-(cis-4-(2-methoxy-2-oxoethyl)cyclohexyloxy)piperidine-1-carboxylate and Intermediate 16: benzyl 4-(trans-4-(2-methoxy-2-oxoethyl)cyclohexyloxy)piperidine-1-carboxylate

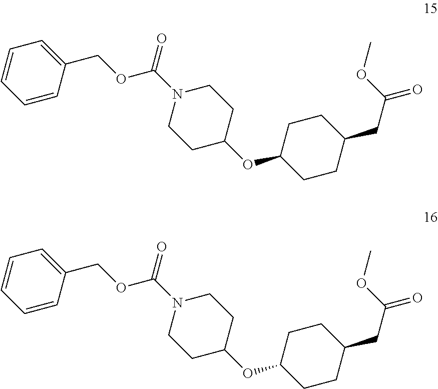 [SMILES: COC(=O)C[C@H]1CC[C@@H](OC2CCN(C(=O)OCc3ccccc3)CC2)CC1 ; COC(=O)C[C@H]1CC[C@H](OC2CCN(C(=O)OCc3ccccc3)CC2)CC1]

Methyl 2-(trans/cis-4-hydroxycyclohexyl)acetate (15 g, 87 mmol) was dissolved in anhydrous THF (150 ml) at 0° C., TEA (13.35 ml, 96 mmol) added, followed by drop wise addition of TMS-Cl (11.69 ml, 91 mmol). The reaction mixture was aged for 30 min then diluted with hexane (100 ml) and filtered through a small pad of celite eluting with hexane and concentrated. The crude product and benzyl 4-oxopiperidine-1-carboxylate (10.40 g, 44.6 mmol) were dissolved in dichloromethane (150 ml) at −60-65° C., triethylsilane (13.91 ml, 87 mmol) added, followed by drop wise addition of TMS-OTf (7.87 ml, 43.5 mmol) and reaction was allowed warm to 0° C. and aged for 30 min. The reaction mixture was diluted with EtOAc (100 ml), 1 M H₃PO₄ (30 ml) added, the organic layer was washed with brine (2×20 ml) and dried over anhydrous sodium sulfate and concentrated under vacuum. This trans/cis mixture was separated by SFC (ChiralPak AD—10 μm, 300×50 mmI.D. Mobile phase: A for SF CO₂ and B for ethanol. Gradient: B 40%.) to give benzyl 4-(cis-4-(2-methoxy-2-oxoethyl)cyclohexyloxy)piperidine-1-carboxylate, LC-MS (ES, m/z): C₂₂H₃₁NO₅: 389; Found: 390 [M+H]⁺ and benzyl 4-(trans-4-(2-methoxy-2-oxoethyl)cyclohexyloxy)piperidine-1-carboxylate, LC-MS (ES, m/z): C₂₂H₃₁NO₅: 389; Found: 390 [M+H]⁺.

Alternatively, benzyl 4-(trans-4-(2-methoxy-2-oxoethyl)cyclohexyloxy) piperidine-1-carboxylate was synthesized from methyl 2-(trans-4-hydroxycyclohexyl)acetate, while benzyl 4-(cis-4-(2-methoxy-2-oxoethyl)cyclohexyloxy)piperidine-1-carboxylate was synthesized from methyl 2-(cis-4-hydroxycyclohexyl)acetate.

### Intermediate 17: methyl [trans-4-(piperidin-4-yloxy)cyclohexyl]acetate

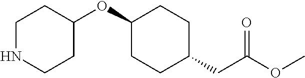 [SMILES: COC(=O)C[C@H]1CC[C@H](OC2CCNCC2)CC1]

Benzyl 4-(trans-4-(2-methoxy-2-oxoethyl)cyclohexyloxy)piperidine-1-carboxylate (3.12 g, 8.01 mmol) was dissolved in methanol (10 ml), 5% palladium on carbon (0.043 g, 0.4 mmol) was added. The reaction mixture was stirred at 1 atm H₂ over night. The reaction mixture was concentrated under vacuum to result in methyl [trans-4-(piperidin-4-yloxy)cyclohexyl]acetate as a colorless oil. LC-MS (ES, m/z) C₁₄H₂₅NO₃: 255; Found: 256 [M+H]⁺.

### Intermediate 18: methyl [cis-4-(piperidin-4-yloxy)cyclohexyl]acetate

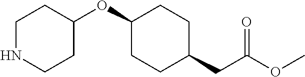 [SMILES: COC(=O)C[C@H]1CC[C@@H](OC2CCNCC2)CC1]

Benzyl 4-(cis-4-(2-methoxy-2-oxoethyl)cyclohexyloxy)piperidine-1-carboxylate (9 g, 23.11 mmol) was dissolved in methanol (40 ml), 5% palladium on carbon (0.123 g, 1.155 mmol) was added. The reaction mixture was stirred at 1 atm H₂ for 2 days. The reaction mixture was concentrated under vacuum to result in methyl [cis-4-(piperidin-4-yloxy)cyclohexyl]acetate as a colorless oil. LC-MS (ES, m/z) C₁₄H₂₅NO₃: 255; Found: 256 [M+H]⁺.

### Intermediate 19: [cis-4-(piperidin-4-yloxy)cyclohexyl]acetic acid

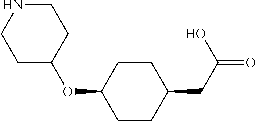 [SMILES: O=C(O)C[C@H]1CC[C@@H](OC2CCNCC2)CC1]

A mixture of methyl [cis-4-(piperidin-4-yloxy)cyclohexyl]acetate (2.23 g, 8.73 mmol) and lithium hydroxide (627 mg, 26.2 mmol) in THF (4 ml), MeOH (6 ml) and water (3 ml). The reaction mixture stirred at room temperature over night then concentrated under vacuum to result in [cis-4-(piperidin-4-yloxy)cyclohexyl]acetic acid as a colorless oil and used as crude. LC-MS (ES, m/z) C₁₃H₂₃NO₃: 241; Found: 242 [M+H]⁺.

### Intermediate 20: [trans-4-(piperidin-4-yloxy)cyclohexyl]acetic acid

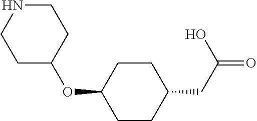 [SMILES: O=C(O)C[C@H]1CC[C@H](OC2CCNCC2)CC1]

Performed the same as described above starting from methyl [trans-4-(piperidin-4-yloxy)cyclohexyl]acetate to result in [trans-4-(piperidin-4-yloxy)cyclohexyl]acetic acid as a colorless oil and used as crude. LC-MS (ES, m/z) C₁₃H₂₃NO₃: 241; Found: 242 [M+H]⁺.

### Intermediate 21: methyl [cis-4-({1-[5-(6-fluoro-1H-benzimidazol-2-yl)pyridin-2-yl]piperidin-4-yl}oxy)cyclohexyl]acetate and Intermediate 22: methyl [trans-4-({1-[5-(6-fluoro-1H-benzimidazol-2-yl)pyridin-2-yl]piperidin-4-yl}oxy)cyclohexyl]acetate

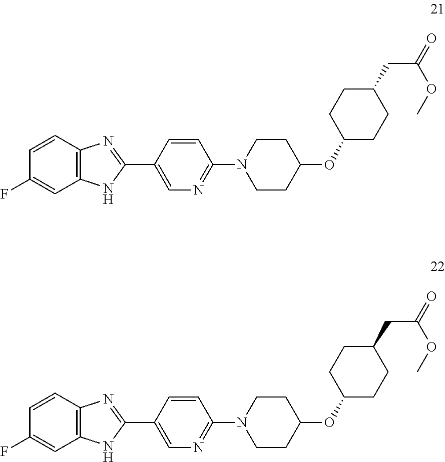 [SMILES: COC(=O)C[C@H]1CC[C@@H](OC2CCN(c3ccc(-c4nc5ccc(F)cc5[nH]4)cn3)CC2)CC1 ; COC(=O)C[C@H]1CC[C@H](OC2CCN(c3ccc(-c4nc5ccc(F)cc5[nH]4)cn3)CC2)CC1]

A mixture of methyl [trans/cis 4-(piperidin-4-yloxy)cyclohexyl]acetate (0.403 g, 1.58 mmol), 6-fluoro-2-(6-fluoropyridin-3-yl)-1H-benzimidazole (0.365 g, 1.58 mmol) and sodium bicarbonate (1.33 g, 15.8 mmol) in NMP (6 ml) was heated at 140° C. in an oil bath over night under N₂. The reaction mixture was cooled to room temperature, water (20 ml) added, extracted with 3×20 mL ethyl acetate. The organic layers were combined, washed with 2×10 ml of saturated brine, dried over anhydrous sodium sulfate and concentrated under vacuum. The residual was applied onto a silica gel column and eluted with ethyl acetate/hexane 0-90%. This resulted in methyl [cis/trans-4-({1-[5-(6-fluoro-1H-benzimidazol-2-yl)pyridin-2-yl]piperidin-4-yl}oxy)cyclohexyl]acetate as a white solid. This trans/cis mixture was separated by SFC, IA column (30×250 mmI.D). Mobile phase: A for SF CO₂ and B for 2:1 MeOH/MeCN. Gradient: B 50%. This resulted in methyl [cis-4-({1-[5-(6-fluoro-1H-benzimidazol-2-yl)pyridin-2-yl]piperidin-4-yl}oxy)cyclohexyl]acetate as a white solid (LC-MS (ES, m/z): C₂₆H₃₁FN₄O₃: 466; Found: 467 [M+H]⁺) and methyl [trans-4-({1-[5-(6-fluoro-1H-benzimidazol-2-yl)pyridin-2-yl]piperidin-4-yl}oxy)cyclohexyl]acetate as a white solid (LC-MS (ES, m/z): C₂₆H₃₁FN₄O₃: 466; Found: 467 [M+H]⁺).

Alternatively, methyl [trans-4-({1-[5-(6-fluoro-1H-benzimidazol-2-yl)pyridin-2-yl]piperidin-4-yl}oxy)cyclohexyl]acetate was prepared from methyl [trans-4-(piperidin-4-yloxy)cyclohexyl]acetate using the method described above.

### Intermediate 23: methyl (trans-4-{[1-(5-formylpyridin-2-yl)piperidin-4-yl]oxy}cyclohexyl)acetate

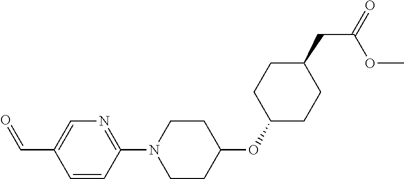 [SMILES: COC(=O)C[C@H]1CC[C@H](OC2CCN(c3ccc(C=O)cn3)CC2)CC1]

A mixture of methyl [trans-4-(piperidin-4-yloxy)cyclohexyl]acetate (1 g, 3.92 mmol), 2-fluoro-5-formylpyridine (0.49 g, 3.92 mmol) and sodium bicarbonate (1.97 g, 23.5 mmol) in DMSO (15 ml) was heated at 110° C. in an oil bath over night under N₂. The reaction mixture was cooled to room temperature, and concentrated under vacuum. The residual was applied onto a silica gel column and eluted with ethyl acetate/hexane 5-100%. This resulted in 1 g (70.8%) of methyl (trans-4-{[1-(5-formylpyridin-2-yl)piperidin-4-yl]oxy}cyclohexyl)acetate as a white solid. LC-MS (ES, m/z) C₂₀H₂₈N₂O₄: 360; Found: 361 [M+H]⁺.

### Intermediate 24: (trans-4-{[1-(5-formylpyridin-2-yl)piperidin-4-yl]oxy}cyclohexyl)acetic acid

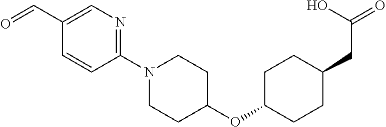 [SMILES: O=Cc1ccc(N2CCC(O[C@H]3CC[C@H](CC(=O)O)CC3)CC2)nc1]

A mixture of [trans-4-(piperidin-4-yloxy)cyclohexyl]acetic acid (0.8 g, 3.32 mmol), 3-fluoro-5-formylpyridine (0.415 g, 3.32 mmol) and sodium bicarbonate (1.67 g, 19.89 mmol) in NMP (6 ml) was heated at 110° C. in an oil bath over night under N₂. The reaction mixture was cooled to room temperature then concentrated under vacuum. The residual was applied onto a silica gel column and eluted with acetone/dichloromethane 0-100%. This resulted in 0.28 g (24.4%) of: (trans-4-{[1-(5-formylpyridin-2-yl)piperidin-4-yl]oxy}cyclohexyl)acetic acid as a white solid. LC-MS (ES, m/z): C₁₉H₂₆N₂O₄: 346; Found: 347 [M+H]⁺.

### Intermediates 25-29

Performed the same as described for methyl (trans-4-{[1-(5-formylpyridin-2-yl)piperidin-4-yl]oxy}cyclohexyl)acetate using appropriate starting materials.

| Intermediate | Structure | [MH]⁺ m/z found |
|---|---|---|
| 25 | 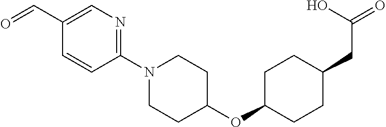 [SMILES: O=Cc1ccc(N2CCC(O[C@H]3CC[C@@H](CC(=O)O)CC3)CC2)nc1] | Found: 347 [M + H]⁺ |
| 26 | 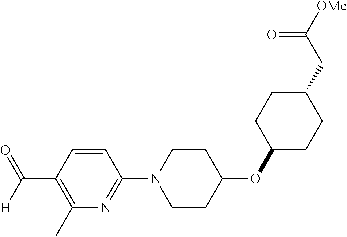 | Found: 375 [M + H]⁺ |
| 27 | 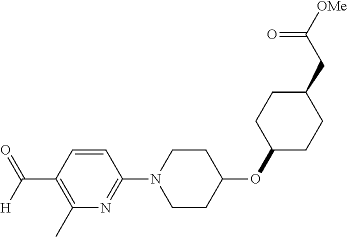 | Found: 375 [M + H]⁺ |
| 28 | 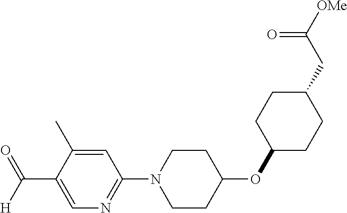 | Found: 375 [M + H]⁺ |
| 29 | 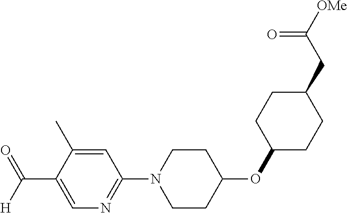 | Found: 375 [M + H]⁺ |

### Intermediate 30: methyl 4-(hydroxymethyl)tetrahydro-2H-pyran-4-carboxylate

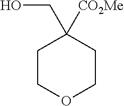 [SMILES: COC(=O)C1(CO)CCOCC1]

### Step 1

To the solution of tetrahydropyran-4-4-dicarboxylic acid dimethyl ester (10 g, 49.5 mmol) in CH₂Cl₂ (150 mL) at −78° C. was added diisobutylaluminum hydride (1.0 M in hexane, 99 ml, 99 mmol). After being stirred at −78° C. for 3 h., the reaction was quenched with NH₄Cl (sat., 8 ml) followed by 1N HCl (15 ml) at −78° C. The reaction mixture was then warmed to room temperature and white solid was filtered and rinsed with CH₂Cl₂ (100 ml). The organic filtrate was washed with water, dried over MgSO₄, filtered and concentrated to give methyl 4-formyltetrahydro-2H-pyran-4-carboxylate (6.8 g) as colorless oil. ¹H-NMR showed an aldehyde H peak at 9.559 ppm in CDCl₃. The sample was used for the further reaction without purification.

### Step 2

To the solution of crude methyl 4-formyltetrahydro-2H-pyran-4-carboxylate (6.7 g, 38.9 mmol) in MeOH (20 mL) at 0° C. was added NaBH₄ (0.294 g, 7.78 mmol) in two portions. After being stirred at 0° C. for one hour, the reaction mixture was concentrated. The residue was purified by MPLC (10%-100% EtOAc in hexane) to give methyl 4-(hydroxymethyl)tetrahydro-2H-pyran-4-carboxylate as oil (4.5 g). LC-MS (ES, m/z): C₈H₁₄O₄: 174; Found: 175 [M+H]⁺.

### Intermediate 31: ethyl 3-hydroxycyclobutanecarboxylate

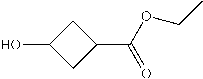

A solution of 1.0 g ketoreductase MIF20 (CODEXIS) and 0.5 g NADP in 450 ml pH 7.0, 50 mM phosphate buffer was charged to a flask. To the enzyme solution, a mixture of 10 g ethyl 3-oxocyclobutanecarboxylate with 50 ml iPrOH was added over 1 h. The reaction solution was agitated for 18 h at 20-23° C. to complete the reduction. MTBE (100 ml) and 100 ml of brine were added to extract the alcohol. The MTBE extraction was repeated twice. Solka Floc (5 g) was added to the organic solution. After mixing for 10 min, the solution was filtered to remove the insoluble. The solvent was removed by evaporation to obtain ethyl 3-hydroxycyclobutanecarboxylate. GC analysis showed that the ratio of cis-alcohol versus trans-alcohol was 1.5:1 (60% vs 40%). LC-MS (ES, m/z) C₇H₁₂O₃: 144; Found: 145 [M+H]⁺.

### Intermediate 32: Ethyl 2-(3-hydroxycyclobutyl)acetate

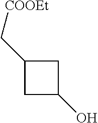 [SMILES: CCOC(=O)CC1CC(O)C1]

### Step 1

To a suspension of NaH (60% in oil, 4.09 g, 102 mmol) in THF (100 mL) was added triethyl phosphonoacetate (25.6 ml, 128 mmol) drop wise at 0° C. The mixture was stirred at 0° C. for 1 h. 3-(benzyloxy)cyclobutanone (15 g, 85 mmol) was added dropwise at 0° C. The reaction mixture was allowed to warm to room temperature and stirred overnight. The reaction mixture was cooled to −78° C., quenched with sat NaHCO₃ (sat.), and then reaction mixture was warmed to room temperature, diluted with water, extracted with EtOAc, dried over MgSO₄ filtered and concentrated. Purified by MG-III (OJ-H, 50 mm×250 mm; 10% MeOH/CO₂, 220 mL/min; 100 bar, 35° C., 220 nm; inject volume: 0.30 ml; feed concentration: 100.00 mg/mL in 1:1 DCM/MeOH; Dissolved in MeOH/DCM 1:1, 245.00 ml) to give Ethyl 2-(3-(benzyloxy)cyclobutylidene)acetate as brown liquid (24.5 g). LC-MS (ES, m/z) C₁₅H₁₈O₃: 246; Found: 247 [M+H]⁺.

### Step 2

To the solution of ethyl 2-(3-(benzyloxy)cyclobutylidene)acetate (5.4 g, 21.92 mmol) in MeOH (100 ml) was added Pd(OH)₂/C (Pearlman's catalyst, 1.08 g) and then hydrogenated under 45 psi. for 18 hr. Catalyst was filtered through celite, washed with MeOH, filtrate was concentrated, and residue was separated by column (10-100% EtOAc in hexane) to give ethyl 2-(3-hydroxycyclobutyl)acetate (3.0 g) as colorless liquid. LC-MS (ES, m/z) C₈H₁₄O₃: 158; Found: 159 [M+H]⁺.

### Intermediate 33: (3a′R,6a′S)-tetrahydro-1′H-spiro[1,3-dioxolane-2,2′-pentalen]-5′(3′H)-one

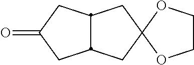 [SMILES: O=C1CC2CC3(CC2C1)OCCO3]

Tertrahydropentalene-2,5-dione (3.25 g, 23.52 mmol) in toluene (100 ml) was added p-toluenesulfonic acid (0.447 g, 2.35 mmol), and ethylene glycol (1.049 ml, 18.82 mmol). The mixture was heated to 110° C. for 2 hours. The reaction was cooled to room temperature, removed the solvent by rotary evaporation. The residue was dissolved in ethyl acetate (200 ml), washed with water, and brine, then the organic was dried over MgSO₄, filtered and concentrated in vacuo to afforded an oil. Chromatography (0-20% ethyl acetate:hexane) to afford the title compound as a colorless oil. ¹HNMR (500 MHz, CDCl₃) δ: 3.55 (4H, s,), 2.86 (2H, s), 2.48 (2H, m), 2.19 (4H, m), 1.75 (2H, m) ppm.

### Intermediate 34: methyl (2E)-(3a′R,6a′S)-tetrahydro-1′H-spiro[1,3-dioxolane-2,2′-pentalen]-5′(3′H)-ylideneethanoate

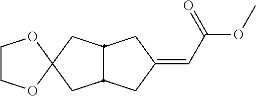 [SMILES: COC(=O)C=C1CC2CC3(CC2C1)OCCO3]

A suspension of sodium hydride (258 mg, 6.46 mmol) in THF (20 ml) cooled to 0° C. and treated with trimethyl phosphonoacetate (0.76 ml, 4.74 mmol). The mixture was stirred at ° C. for 20 min, then (3a′R,6a′S)-tetrahydro-1′H-spiro[1,3-dioxolane-2,2′-pentalen]-5′(3′H)-one in THF (10 ml) was added to the mixture at ° C. Allowed to warm to room temperature and stirred at room temperature for 16 hours. Quenched with water, extracted with ethyl acetate (100 ml), washed the organic with brine, then the organic was dried over MgSO₄, filtered and concentrated in vacuo to afford an oil. Chromatography (0-20% ethyl acetate:hexane) to afford the title compound as a colorless oil. ¹HNMR (500 MHz, CDCl₃) δ: 5.78 (1H, s), 3.85 (4H, s,), 3.68 (3H, s), 3.01 (1H, m), 2.81 (1H, dd, 5 Hz), 2.71 (2H, m), 2.60 (1H, s), 2.41 (1H, dd, 5 Hz), 2.08 (2H, m), 1.65 (2H, m) ppm.

### Intermediate 35: methyl (3a′R,6a′S)-hexahydro-1′H-spiro[1,3-dioxolane-2,2′-pentalen]-5′-ylacetate

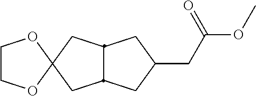 [SMILES: COC(=O)CC1CC2CC3(CC2C1)OCCO3]

Methyl (2E)-(3a′R,6a′S)-tetrahydro-1′H-spiro[1,3-dioxolane-2,2′-pentalen]-5′(3′H)-ylideneethanoate (380 mg, 1.59 mmol) in ethanol (4 ml) was added Pd—C (94 mg, 0.08 mmol). The mixture was degassed and refilled H₂ several times. The mixture was stirred under H₂ for 16 hour. Filtered through a pad of celite, and washed with ethanol. Concentrated to afford an oil. ¹HNMR (500 MHz, CDCl₃) δ: 3.92 (4H, m), 3.71 (3H, s,), 2.52 (2H, m), 2.39 (2H, d, 7.5), 2.20 (1H, m), 2.11 (2H, m), 1.98 (2H, m), 1.61 (2H, dd, 5 Hz), 1.15 (2H, m) ppm.

### Intermediate 36: methyl [(3aR,6aS)-5-oxooctahydropentalen-2-yl]acetate

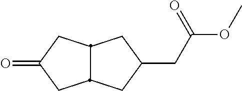

Methyl (3a′R,6a′S)-hexahydro-1′H-spiro[1,3-dioxolane-2,2′-pentalen]-5′-ylacetate (873 mg, 3.63 mmol) in THF (4 ml)/2N HCl (1 ml) was stirred for overnight. Concentrated in vacuo, then chromatography (0-20% ethyl acetate:hexane) to afford the title compound as a colorless oil. ¹HNMR (500 MHz, CDCl₃) δ: 3.65 (3H, s), 2.70 (2H, m,), 2.52 (2H, dd, 9 Hz), 2.38 (2H, s), 2.24 (2H, m), 2.05 (2H, dd, 5 Hz), 1.03 (2H, m), 0.85 (1H, m) ppm.

### Intermediate 37: methyl [(3aR,6aS)-5-hydroxyoctahyropentalen-2-yl]acetate

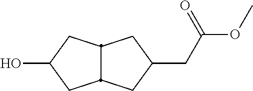

Methyl [(3aR,6aS)-5-oxooctahydropentalen-2-yl]acetate (1.63 g, 8.31 mmol) in MeOH (20 ml) at 0° C. was slowly added sodium hydride (0.361 g, 9.55 mmol). The mixture was stirred at 0° C. 3 hours. Then the solvent was concentrated in vacuo. Chromatography (0-40% ethyl acetate:hexane) to afford the title compound as a colorless oil. ¹HNMR (500 MHz, CDCl₃) δ: 3.65 (3H, s), 3.35 (2H, d,), 3.25 (2H, m), 3.15 (2H, d, 10 Hz), 2.88 (2H, m), 2.23 (2H, m), 2.15 (1H, m), 1.08 (2H, m) ppm.

### Intermediate 38: Methyl 2,2-dimethyl-3-(pyridin-4-yloxy)propanoate

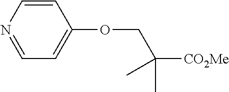

To a stirred solution of 4-hydroxypyridine (10 g, 105 mmol) in anhydrous THF (200 ml) at room temperature was added hydroxypivalic acid methyl ester (16.77 ml, 131 mmol). Triphenylphosphine (34.5 g, 131 mmol) was then added followed by drop wise addition of diisopropyl azodicarboxylate (25.9 ml, 131 mmol) at 0° C. The reaction was then heated to 55° C. and allowed to stir at this temperature over night. The reaction mixture was concentrated. The residue was treated with EtOAc (100 ml) and then Hexane (100 ml), the solid was filtered off. The filtrate was concentrated, separated by Thar 200 preparative SFC (column: ChiralPak AD-H, 250×50 mmI.D.; Mobile phase: A for SF CO₂ and B for Ethanol; Gradient: B 30%; Flow rate: 150 ml/min; Sample preparation: dissolved in ethanol, 200 mg/ml; Injection: 4.5 ml per injection). After separation, the desired fractions were dried off via rotary evaporator at bath temperature 40° C. to give Methyl 2,2-dimethyl-3-(pyridin-4-yloxy)propanoate (26.2 g, containing some solvent). LC-MS (ES, m/z) C₁₁H₁₅NO₃: 209; Found: 210 [M+H]⁺.

### Intermediate 39: Methyl 2,2-dimethyl-3-(piperidin-4-yloxy)propanoate

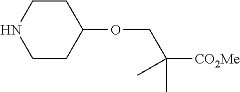

### Method A:

To a solution of methyl 2,2-dimethyl-3-(pyridin-4-yloxy)propanoate (11.25 g, 53.8 mmol) in acetic acid (100 ml) was added Rh/C (5%, 2.25 g), then the reaction mixture was hydrogenated under 40 psi at 80° C. for 18 hrs. The catalyst was filtered through celite, washed with MeOH and filtrate was concentrated to give Methyl 2,2-dimethyl-3-(piperidin-4-yloxy)propanoate. LC-MS (ES, m/z) C₁₁H₂₁NO₃: 215; Found: 216 [M+H]⁺.

### Method B:

Methyl 2,2-dimethyl-3-(pyridin-4-yloxy)propanoate (1 g, 4.78 mmol) was dissolved in acetic acid (70 ml). The solution passed through Rh/C cartridge on H-Cube at 80° C. under 80 bars. The reaction mixture concentrated under vacuum to afford methyl 2,2-dimethyl-3-(piperidin-4-yloxy)propanoate as a colorless oil. LC-MS (ES, m/z) 215; Found: 216 [M+H]⁺.

### Intermediates 40-45

Synthesized following the procedure described for methyl 2,2-dimethyl-3-(pyridin-4-yloxy)propanoate starting from the appropriate hydroxy ester.

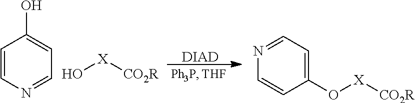

| | | [MH]⁺ |
|---|---|---|
| Inter- | | m/z |
| mediate | Structure | found |
| 40 | 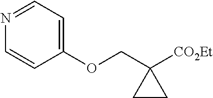 [SMILES: CCOC(=O)C1(COc2ccncc2)CC1] | 222 |
| 41 | 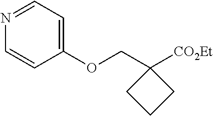 [SMILES: CCOC(=O)C1(COc2ccncc2)CCC1] | 236 |
| 42 | 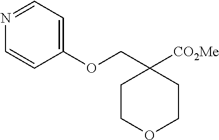 [SMILES: COC(=O)C1(COc2ccncc2)CCOCC1] | 252 |
| 43 | 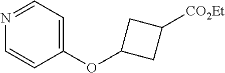 [SMILES: CCOC(=O)C1CC(Oc2ccncc2)C1] | 222 |
| 44 | 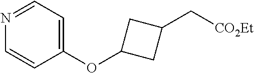 [SMILES: CCOC(=O)CC1CC(Oc2ccncc2)C1] | 236 |
| 45 | 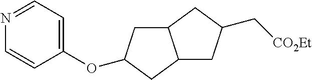 [SMILES: CCOC(=O)CC1CC2CC(Oc3ccncc3)CC2C1] | 276 |

### Intermediates 46-51

Performed following the procedure described for methyl 2,2-dimethyl-3-(piperidin-4-yloxy)propanoate starting from corresponding pyridine intermediate prepared above.

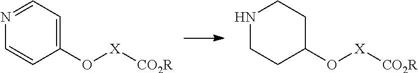

| | | [MH]⁺ |
|---|---|---|
| Inter- | | m/z |
| mediate | Structure | found |
| 46 | 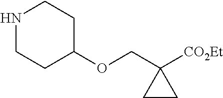 [SMILES: CCOC(=O)C1(COC2CCNCC2)CC1] | 228 |
| 47 | 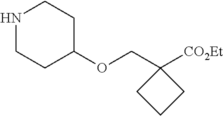 [SMILES: CCOC(=O)C1(COC2CCNCC2)CCC1] | 242 |
| 48 | 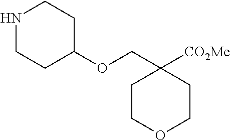 [SMILES: COC(=O)C1(COC2CCNCC2)CCOCC1] | 258 |
| 49 | 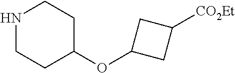 [SMILES: CCOC(=O)C1CC(OC2CCNCC2)C1] | 228 |
| 50 | 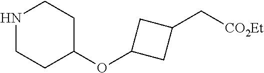 [SMILES: CCOC(=O)CC1CC(OC2CCNCC2)C1] | 242 |
| 51 | 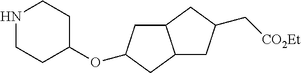 [SMILES: CCOC(=O)CC1CC2CC(OC3CCNCC3)CC2C1] | 282 |

### Intermediate 52: ethyl cis-3-[(1-{5-[6-(trifluoromethyl)-1H-benzimidazol-2-yl],pyridin-2-yl}piperidin-4-yl)oxy]cyclobutanecarboxylate and Intermediate 53: ethyl trans-3-[(1-{5-[6-(trifluoromethyl)-1H-benzimidazol-2-yl]pyridin-2-yl}piperidin-4-yl)oxy]cyclobutanecarboxylate

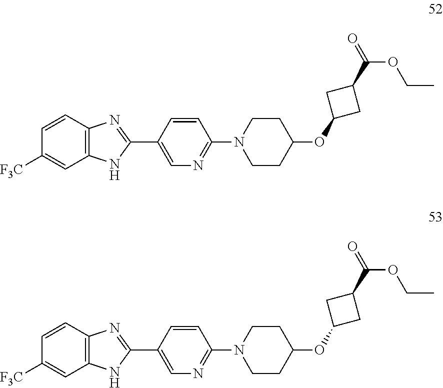 [SMILES: CCOC(=O)[C@H]1C[C@@H](OC2CCN(c3ccc(-c4nc5ccc(C(F)(F)F)cc5[nH]4)cn3)CC2)C1 ; CCOC(=O)[C@H]1C[C@H](OC2CCN(c3ccc(-c4nc5ccc(C(F)(F)F)cc5[nH]4)cn3)CC2)C1]

Ethyl 3-(piperidin-4-yloxy)cyclobutanecarboxylate (0.299 g, 1.316 mmol), 2-(6-fluoropyridin-3-yl)-5-(trifluoromethyl)-1H-benzimidazole (0.37 g, 1.316 mmol) and sodium bicarbonate (1.1 g, 13.16 mmol) in NMP (3.5 ml) was heated at 110° C. in an oil bath over night under N₂. The reaction mixture was cooled to room temperature, water (20 ml) added, extracted with 3×20 mL ethyl acetate. The organic layers were combined, washed with 2×10 ml of saturated brine, dried over anhydrous sodium sulfate and concentrated under vacuum. The residue was applied onto a silica gel column and eluted with ethyl acetate/hexane 0-90%. This resulted in ethyl cis&trans-3-[(1-{5-[6-(trifluoromethyl)-1H-benzimidazol-2-yl]pyridin-2-yl}piperidin-4-yl)oxy]cyclobutanecarboxylate as brown oil, which was separated by SFC, chiralcel OJ (20 nm, 300×50 mmI.D). Mobile phase: A for SF CO₂ and B for ethanol (0.2% DEA). Gradient: B 30%. This resulted in ethyl cis-3-[(1-{5-[6-(trifluoromethyl)-1H-benzimidazol-2-yl]pyridin-2-yl}piperidin-4-yl)oxy]cyclobutanecarboxylate as white solid. LC-MS (ES, m/z) C₂₅H₂₇F₃N₄O₃: 488; Found: 489[M+H]⁺ and ethyl trans-3-[(1-{5-[6-(trifluoromethyl)-1H-benzimidazol-2-yl]pyridin-2-yl}piperidin-4-yl)oxy]cyclobutanecarboxylate as white solid. LC-MS (ES, m/z) C₂₅H₂₇F₃N₄O₃: 488; Found: 489[M+H]⁺.

### Intermediate 54: methyl 3-(1-(5-formylpyridin-2-yl)piperidin-4-yloxy)-2,2-dimethylpropanoate

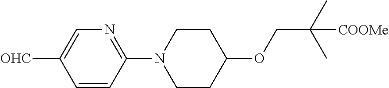 [SMILES: COC(=O)C(C)(C)COC1CCN(c2ccc(C=O)cn2)CC1]

A mixture of methyl 2,2-dimethyl-3-(piperidin-4-yloxy)propanoate (3.72 g, 8.63 mmol), 2-fluoro-5-formylpyridine (1.2 g, 9.59 mmol) and sodium bicarbonate (16.12 g, 192 mmol) in NMP (19 ml) was heated at 110° C. in an oil bath over night under N₂. The reaction mixture was stirred at room temperature over night then poured into 50 ml water, extracted with 3×50 ml ethyl acetate. The organic layers were combined, washed with 2×10 ml of saturated brine, dried over anhydrous sodium sulfate and concentrated under vacuum. The crude material was applied onto a silica gel column and eluted with ethyl acetate/hexane 10-100%. This resulted in methyl 3-(1-(5-formylpyridin-2-yl)piperidin-4-yloxy)-2,2-dimethylpropanoate as a brown solid. LC-MS (ES, m/z) C₁₇H₂₄N₂O₄: 320; Found: 321 [M+H]⁺.

### Intermediates 55 and 56: ethyl cis-4-(pyridin-4-yloxy)cyclohexanecarboxylate and ethyl trans-4-(pyridin-4-yloxy)cyclohexanecarboxylate

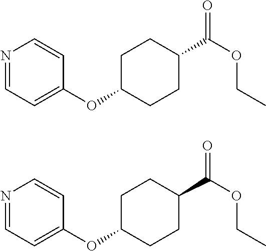 [SMILES: CCOC(=O)[C@H]1CC[C@@H](Oc2ccncc2)CC1 ; CCOC(=O)[C@H]1CC[C@H](Oc2ccncc2)CC1]

To a stirred solution of pyridin-4-ol (15 g, 158 mmol) in anhydrous THF (300 ml) at RT was added ethyl 4-hydroxycyclohexanecarboxylate (31.8 ml, 197 mmol), triphenylphosphine (51.7 g, 197 mmol), and then followed by drop wise addition of diisopropyl azodicarboxylate (25.9 ml, 131 mmol) at 0° C. The reaction was then heated to 55° C. and allowed to stir at this temperature under nitrogen for 48 hrs. The reaction mixture was concentrated. The residue was treated with EtOAc (25 ml) and then Hexane (25 ml) and stirred over night. The solid was removed by filtration. The filtrate was concentrated. The crude product was separated by Thar 200 preparative SFC (column: ChiralPak AD-H, 250×50 mmI.D.; Mobile phase: A for SF CO₂ and B for Ethanol; Gradient: A:B: 60:40%; Flow rate: 130 ml/min; Sample preparation: dissolved in ethanol, 200 mg/ml; Injection: 4.5 ml per injection). After separation, the fractions were dried off via rotary evaporator at bath temperature 40° C. to give the mixture of two isomers which was underwent the second separation by SFC (column: ChiralPak AD-H, 250×50 mmI.D.; Mobile phase: A for SF CO₂ and B for isopropanol; Gradient: A:B: 75:25%; Flow rate: 160 ml/min; Sample preparation: dissolved in ethanol, 25 mg/ml; Injection: 4 ml per injection). After separation, the fractions were dried off via rotary evaporator at bath temperature 40° C. to provide ethyl cis-4-(pyridin-4-yloxy)cyclohexanecarboxylate (slower eluting isomer, 4.25 g) LC-MS (ES, m/z) C₁₄H₁₉NO₃: 249; Found: 250 [M+H]⁺ and ethyl trans-4-(pyridin-4-yloxy)cyclohexanecarboxylate (faster eluting isomer, 9.5 g) LC-MS (ES, m/z) C₁₄H₁₉NO₃: 249; Found: 250 [M+H]⁺.

### Intermediate 57: ethyl cis-4-(piperidin-4-yloxy)cyclohexanecarboxylate

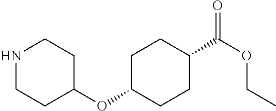 [SMILES: CCOC(=O)[C@H]1CC[C@@H](OC2CCNCC2)CC1]

To a solution of ethyl cis-4-(pyridin-4-yloxy)cyclohexanecarboxylate (0.5 g, 2.01 mmol) in acetic acid (15 ml) was added platinum (IV) oxide (0.125 g, 0.550 mmole). The reaction mixture was degassed and purged nitrogen for 3 times, then vacuumed and hydrogenated under hydrogen balloon overnight. The catalyst was filtered through celite, washed with MeOH and filtrate was concentrated and lyophilized to give ethyl cis-4-(piperidin-4-yloxy)cyclohexanecarboxylate (0.63 g). LC-MS (ES, m/z) C₁₄H₂₅NO₃: 255; Found: 256 [M+H]⁺.

### Intermediate 58: ethyl trans-4-(piperidin-4-yloxy)cyclohexanecarboxylate

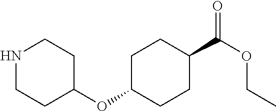 [SMILES: CCOC(=O)[C@H]1CC[C@H](OC2CCNCC2)CC1]

To a solution of ethyl trans-4-(pyridin-4-yloxy)cyclohexanecarboxylate (0.66 g, 2.65 mmol) in acetic acid (15 ml) was added platinum (IV) oxide (0.165 g, 0.727 mmole). The reaction mixture was degassed and purged nitrogen for 3 times, then vacuumed again and hydrogenated under hydrogen balloon weekend. The catalyst was filtered through celite, washed with MeOH and filtrate was concentrated and lyophilized to give ethyl trans-4-(piperidin-4-yloxy)cyclohexanecarboxylate (0.79 g). LC-MS (ES, m/z) C₁₄H₂₅NO₃: 255; Found: 256 [M+H]⁺.

Alternatively, ethyl trans-4-(piperidin-4-yloxy)cyclohexanecarboxylate was prepared by the following method:

To a solution of ethyl trans-4-(pyridin-4-yloxy)cyclohexanecarboxylate (3.15 g, 12.64 mmol) in acetic acid (30 ml) was added Rh/C (5%, 0.63 g), then the reaction mixture was hydrogenated under 400 psi at 100° C. for 18 hrs. The catalyst was filtered through celite, washed with MeOH and filtrate was concentrated to give ethyl trans-4-(piperidin-4-yloxy)cyclohexanecarboxylate. LC-MS (ES, m/z) C₁₄H₂₅NO₃: 255; Found: 256 [M+H]⁺.

Alternatively, ethyl trans-4-(piperidin-4-yloxy)cyclohexanecarboxylate was prepared by the following method:

### Step 1

To a solution of 1.427 L of water was added 9.7 g of mono potassium phosphate and 12.4 grams of dipotassium phosphate. To this was added 5.71 g of MIF-20 and 1.43 g of NAPD to give a pH of 7. To the mixture was added 256.78 g (1.509 mol) of ethyl 4-oxocyclohexanecarboxylate in 1.427 L of 2-propanol. The pH of the mixture was controlled at 7 by the addition of 1 M HCl. The mixture was stirred at 30° C. for 20 h. The reaction mixture was then extracted with 1.5 L of MTBE. The aqueous layer was back extracted with a 3:1 mixture of MTBE/2-propanol (2×600 mL). The organic layer was then concentrated under reduced pressure and re-dissolved in 1.5 L of MTBE. The organic layer was washed with brine (2×300 mL), dried over sodium sulfate, concentrated and flushed with 1 L of MTBE to give ethyl trans-4-hydroxycyclohexanecarboxylate as colorless oil and >99:1 trans/cis selectivity.

### Step 2

To a solution of 10 g (58.1 mmol) of ethyl trans-4-hydroxycyclohexanecarboxylate in 150 mL of anhydrous THF at 0° C. was added 8.9 mL (63.9 mmol) of triethyl amine followed by the drop wise addition of 7.79 mL (61.0 mmol) of TMSCl. The resulting slurry was stirred at 0° C. for 30 min and then diluted with 150 mL of hexane. The slurry was filtered and the filter cake was washed with additional hexane. The filtrate was concentrated under reduced pressure and then flushed with 100 mL of CH₂Cl₂. The crude oil was re-dissolved in 150 mL of CH₂Cl₂ and cooled to −60-65° C. To this was added 13.0 g (55.7 mmol) of (4-oxopiperidin-1-yl)methyl benzoate, 10.2 mL (63.9 mmol) of triethylsilane, and 5.25 mL (29.0 mmol) of TMSOTf. The mixture was allowed to slowly warm to 0° C. and aged for 30 min. The reaction mixture was diluted with EtOAc and 1M H₃PO₄. The layers were separated and the organic layer was washed with brine, dried over MgSO₄, filtered and concentrated. The crude residue was purified by silica gel chromatography (0-100% EtOAc/hexane) to give benzyl 4-{[trans-4-(ethoxycarbonyl)cyclohexyl]oxy}piperidine-1-carboxylate.

### Step 3

Benzyl 4-{[trans-4-(ethoxycarbonyl)cyclohexyl]oxy}piperidine-1-carboxylate (18.2 g, 46.7 mmol) was dissolved in 180 mL of a 1:1 mixture of EtOH/EtOAc and catalytic Pd/C was added. The mixture was hydrogenated under a balloon pressure of H₂ for 6 h. The catalyst was filtered through celite eluting with 1:1 EtOH/CHCl₃. The solvent was removed under reduced pressure and the resulting solid was slurried in 150 mL of hexane and filtered. The wet cake was dried under vacuum/N₂ sweep overnight to give (trans)-ethyl 4-(piperidin-4-yloxy)cyclohexanecarboxylate as a colorless solid. LC-MS (ES, m/z) C₁₄H₂₅NO₃: 255; Found: 256 [M+H]

### Intermediates 59 and 60: tert-butyl 4-(cis-4-(ethoxycarbonyl)cyclohexyloxy) piperidine-1-carboxylate and tert-butyl 4-(trans-4-(ethoxycarbonyl)cyclohexyloxy) piperidine-1-carboxylate

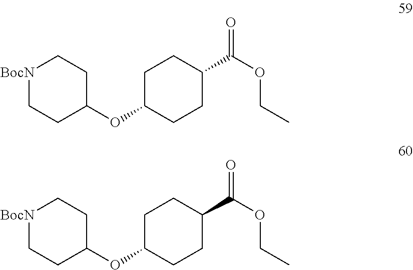 [SMILES: CCOC(=O)[C@H]1CC[C@@H](OC2CCN(C(=O)OC(C)(C)C)CC2)CC1 ; CCOC(=O)[C@H]1CC[C@H](OC2CCN(C(=O)OC(C)(C)C)CC2)CC1]

### Step 1

To a solution of ethyl cis-4-(pyridin-4-yloxy)cyclohexanecarboxylate (2.0 g, 8.02 mmol) in ethanol (20 ml) was added Ts-OH (3.05 g, 16.04 mmol), and reaction mixture was degassed by blowing N₂, and added platinum (IV) oxide hydrate (0.983 g, 4.01 mmole). The reaction mixture was hydrogenated in a Parr apparatus at 45 psi for 4 days. The catalyst was filtered through celite, washed with ethanol and filtrate was concentrated to give crude ethyl cis-4-(piperidin-4-yloxy)cyclohexanecarboxylate.

### Step 2

To a solution of ethyl trans-4-(pyridin-4-yloxy)cyclohexanecarboxylate (2.0 g, 8.02 mmol) in ethanol (20 ml) was added Ts-OH (3.05 g, 16.04 mmol), and reaction mixture was degassed by blowing N₂, and added platinum (IV) oxide hydrate (0.983 g, 4.01 mmole). The reaction mixture was hydrogenated in a Parr apparatus at 45 psi for 4 days. The catalyst was filtered through celite, washed with ethanol and filtrate was concentrated to give crude ethyl trans-4-(piperidin-4-yloxy)cyclohexanecarboxylate.

### Step 3

To the mixture of crude ethyl cis-4-(pyridin-4-yloxy)cyclohexanecarboxylate and crude ethyl trans-4-(pyridin-4-yloxy)cyclohexanecarboxylate in EtOH (50 ml) at 0° C. was added Et₃N (10.92 ml, 78 mmol), Boc₂O (4.27 g, 19.58 mmol) and DMAP (2.392 g, 19.58 mmol). The reaction mixture was warmed to room temperature and stirred overnight. The reaction mixture was concentrated, treated with water, extracted with EtOAc (2×), washed with brine, and dried over Na₂SO₄, filtered and concentrated. The crude product was separated by Thar 200 preparative SFC (column: ChiralPak AD-H, 250×50 mmI.D; Mobile phase: A for SF CO₂ and B for ethanol; Gradient: B: 25%; Flow rate: 150 ml/min; Sample preparation: dissolved in ethanol, 71 mg/ml; Injection: 4 ml per injection). After separation, the fractions were dried off via rotary evaporator at bath temperature 40° C. to give the mixture of two isomers which underwent the second separation by SFC (column: ChiralPak AD-H, 250×50 mmI.D.; Mobile phase: A for SF CO₂ and B for methanol; Gradient: B 20%; Flow rate: 150 ml/min; Sample preparation: dissolved in methanol, 20 mg/ml; Injection: 4 ml per injection). After separation, the fractions were dried off via rotary evaporator at bath temperature 40° C. to provide tert-butyl 4-(cis-4-(ethoxycarbonyl)cyclohexyloxy)piperidine-1-carboxylate and tert-butyl 4-(trans-4-(ethoxycarbonyl)cyclohexyloxy)piperidine-1-carboxylate. LC-MS (ES, m/z) C₁₉H₃₃NO₅: 355; Found: 378 [M+Na]⁺.

### Intermediate 61: ethyl trans-4-{[1-(5-formylpyridin-2-yl)piperidin-4-yl]oxy}cyclohexanecarboxylate

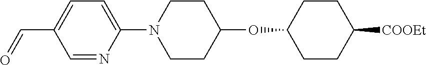 [SMILES: CCOC(=O)[C@H]1CC[C@H](OC2CCN(c3ccc(C=O)cn3)CC2)CC1]

Ethyl trans-4-(piperidin-4-yloxy)cyclohexanecarboxylate (260 mg, 1.02 mmol) in DMSO (3 ml) was added 2-fluoro-5-formypyridine (166 mg, 1.32 mmol), and sodium bicarbonate (855 mg, 10.2 mmol). The mixture was heated at 110° C. under N₂ for 2 hours. The reaction was cooled to RT, quenched with water, and extracted with ethyl acetate (2×40 ml). Dried over MgSO₄, filtered and concentrated. The residue was purified by preparative TLC (40% EtOAc/Hexane) to give the title compound as a white solid. LC-MS (ES, m/z): C₂₀H₂₈N₂O₄: 360; Found: 361 [M+H]⁺.

### Intermediate 62: Diethyl 3-(1-(benzyloxycarbonyl)piperidin-4-yloxy)cyclobutane-1,1-dicarboxylate

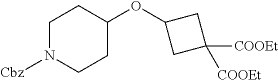 [SMILES: CCOC(=O)C1(C(=O)OCC)CC(OC2CCN(C(=O)OCc3ccccc3)CC2)C1]

Performed following the procedure describe above staring from benzyl 4-oxopiperidine-1-carboxylate and diethyl 3-hydroxycyclobutane-1,1-dicarboxylate (synthesized by a known procedure Avram et al. Chemische Berichte, 1957, vol. 90, p. 1424, 1427)

### Intermediate 63: diethyl 3-(piperidin-4-yloxy)cyclobutane-1,1-dicarboxylate

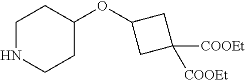 [SMILES: CCOC(=O)C1(C(=O)OCC)CC(OC2CCNCC2)C1]

Diethyl 3-(1-(benzyloxycarbonyl)piperidin-4-yloxy)cyclobutane-1,1-dicarboxylate (0.5 g, 1.153 mmol) was dissolved in ethanol (12 ml), 5% palladium on carbon (0.006 g, 0.058 mmol) added. The reaction mixture stirred at 1 atm H₂ for 2 days. The reaction mixture concentrated under vacuum to result in 0.345 g (100%) of diethyl 3-(piperidin-4-yloxy)cyclobutane-1,1-dicarboxylate as a colorless oil. LC-MS (ES, m/z) C₁₅H₂₅NO₅: 299; Found: 300 [M+H]⁺.

### Intermediate 64: diethyl 3-(1-(5-formylpyridin-2-yl)piperidin-4-yloxy)cyclobutane-1,1-dicarboxylate

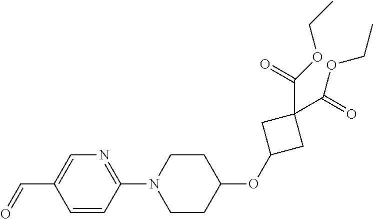 [SMILES: CCOC(=O)C1(C(=O)OCC)CC(OC2CCN(c3ccc(C=O)cn3)CC2)C1]

A mixture of diethyl 3-(piperidin-4-yloxy)cyclobutane-1,1-dicarboxylate (0.345 g, 1.152 mmol), 2-fluoro-5-formylpyridine (0.144 g, 1.152 mmol) and sodium bicarbonate (0.581 g, 6.91 mmol) in DMSO (6 ml) was heated at 110° C. in an oil bath over night under N₂. The reaction mixture was cooled to room temperature, water (10 ml) added, extracted with 3×15 ml ethyl acetate. The organic layers were combined, washed with 2×10 ml of saturated brine, dried over anhydrous sodium sulfate and concentrated under vacuum. Then applied onto a silica gel column and eluted with ethyl acetate/hexane 10-90%. This resulted in 0.24 g (51.5%) of diethyl 3-(1-(5-formylpyridin-2-yl)piperidin-4-yloxy)cyclobutane-1,1-dicarboxylate as a white solid. LC-MS (ES, m/z) C₂₁H₂₈N₂O₆: 404; Found: 405 [M+H]⁺.

### Intermediate 65: ethyl 8-methyl-1,4-dioxaspiro[4.5]decane-8-carboxylate

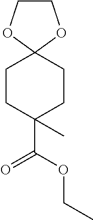 [SMILES: CCOC(=O)C1(C)CCC2(CC1)OCCO2]

A solution of lithium diisopropylamide (31.1 ml, 46.7 mmol) in THF (100 ml) was cooled to −78° C. A solution of ethyl 1,4-dioxaspiro[4.5]decane-8-carboxylate (5 g, 23.34 mmol) in THF (100 ml) was added slowly and the mixture was stirred for 30 min. iodomethane (3.65 ml, 58.3 mmol) was added, and the mixture was continued to stirred for 2 hr at −78° C. The reaction mixture was quenched with water (100 ml), separated two layers, the aqueous layer was extracted with Et₂O (2×150 ml), dried over Na₂SO₄, concentrated and separated by MPLC (0-50% EtOAc in Hexane) to give ethyl 8-methyl-1,4-dioxaspiro[4.5]decane-8-carboxylate (4.4 g) as yellow oil. LC-MS (ES, m/z) C₁₂H₂₀O₄: 228; Found: 229 [M+H]⁺.

### Intermediate 66: ethyl 1-methyl-4-oxocyclohexanecarboxylate

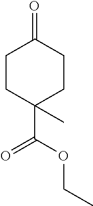

To a solution of ethyl 8-methyl-1,4-dioxaspiro[4.5]decane-8-carboxylate (2.0 g, 8.76 mmol) in aceton (60 ml) was added HCl (2.5 M, 60 ml, 150 mmol) at room temperature. After stirring at room temperature over 48 hours, the reaction mixture was poured into DCM, the organic layer was then separated and the aqueous was extracted with DCM, washed with brine, dried over Na₂SO₄, filtered and concentrated, and purified by MPLC (5-60% EtOAc in hexane) to provide ethyl 1-methyl-4-oxocyclohexanecarboxylate as colorless liquid (1.12 g). LC-MS (ES, m/z) C₁₀H₁₆O₃: 184; Found: 185 [M+H]⁺.

### Intermediate 67: ethyl 4-hydroxy-1-methylcyclohexanecarboxylate

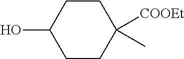

To a solution of ethyl 1-methyl-4-oxocyclohexanecarboxylate (7.02 g, 38.1 mmol) in methanol (15 ml) at 0° C. added sodium borohydride (0.721 g, 190.5 mmol) in small portions over 30 min. The reaction mixture aged for 1 hour. Then concentrated under vacuum and applied onto a silica gel column and eluted with ethyl acetate/hexane 10-100%. This resulted in 5.58 g (79%) of ethyl 4-hydroxy-1-methylcyclohexanecarboxylate (cis&trans mixture) as colorless oil. LC-MS (ES, m/z): C₁₀H₁₈O₃: 186; Found: 187 [M+H]⁺.

### Intermediate 68: ethyl trans-1-methyl-4-(pyridin-4-yloxy)cyclohexanecarboxylate and Intermediate 69: ethyl cis-1-methyl-4-(pyridin-4-yloxy)cyclohexanecarboxylate

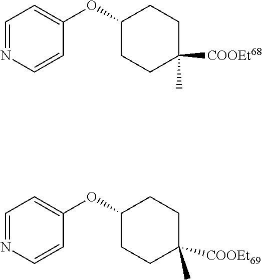

To a mixture of 4-hydroxypyridine (1.2 g, 12.62 mmol), ethyl 4-hydroxy-1-methylcyclohexanecarboxylate (2.82 g, 15.14 mmol) and triphenylphosphine (3.97 g, 15.14 mmol) in THF (25 ml) was added diisopropylazodicarboxylate (3.06 g, 15.14 mmol) drop wise. The reaction mixture was heated at 55° C. in an oil bath for 2 days under N₂. The reaction mixture was cooled to room temperature, concentrated under vacuum then separated by SFC (ChiraPak AY-H column (150×4.6 mm I.D). Mobile phase: A for SF CO₂ and B for isopropanol (0.05% DEA). Gradient: B 15-40%). This resulted in ethyl trans-1-methyl-4-(pyridin-4-yloxy)cyclohexanecarboxylate as a white solid. LC-MS (ES, m/z): C₁₅H₂₁NO₃: 263; Found: 264 [M+H]⁺, and ethyl cis-1-methyl-4-(pyridin-4-yloxy)cyclohexanecarboxylate as a white solid. LC-MS (ES, m/z): C₁₅H₂₁NO₃: 263; Found: 264 [M+H]⁺.

Alternatively, ethyl cis-1-methyl-4-(pyridin-4-yloxy)cyclohexanecarboxylate was prepared from ethyl trans-4-(pyridin-4-yloxy)cyclohexanecarboxylate:

To a solution of diisopropylamine (3.11 ml, 21.84 mmol) in THF (50 ml) at −78° C. under N₂ added 2.5 M n-butyllithium (7.94 ml, 19.68 mmol) drop wise. The reaction mixture was aged for 30 min, then ethyl trans-4-(pyridin-4-yloxy)cyclohexanecarboxylate (4.5 g, 18.05 mmol) in THF (50 ml) was added slowly to the reaction mixture. After 30 min, iodomethane (1.467 mL, 23.47 mmol) was added. The reaction mixture was allowed to warm to room temperature over two hours. Water (10 ml) added, extracted with 3×25 ml ethyl acetate. The organic layers were combined, washed with 2×10 mL of saturated brine, dried over anhydrous sodium sulfate and concentrated under vacuum. Then applied onto a IA column (30×250 mm I.D) and eluted with 25% 2:1 MeOH:MeCN/CO₂. This resulted in 1.5 g (31.6%) of ethyl cis-1-methyl-4-(pyridin-4-yloxy)cyclohexanecarboxylate as a white solid. LC-MS (ES, m/z) C₁₅H₂₁NO₃: 263; Found: 264 [M+H]⁺.

### Intermediate 70: ethyl cis-1-methyl-4-(piperidin-4-yloxy)cyclohexanecarboxylate

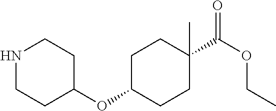 [SMILES: CCOC(=O)[C@]1(C)CC[C@H](OC2CCNCC2)CC1]

Ethyl cis-1-methyl-4-(pyridin-4-yloxy)cyclohexanecarboxylate (1 g, 3.8 mmol) was dissolved in acetic acid (61 ml). The solution was passed through Rh/C cartridge on H-Cube at 80° C. under 80 bars. The reaction mixture was concentrated under vacuum to result in 1.02 g (100%) of ethyl cis-1-methyl-4-(piperidin-4-yloxy)cyclohexanecarboxylate as a colorless oil. LC-MS (ES, m/z) C₁₅H₂₇NO₃: 269; Found: 270 [M+H]⁺.

### Intermediate 71: ethyl trans-1-methyl-4-(piperidin-4-yloxy)cyclohexanecarboxylate

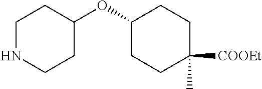 [SMILES: CCOC(=O)[C@]1(C)CC[C@@H](OC2CCNCC2)CC1]

Ethyl trans-1-methyl-4-(pyridin-4-yloxy)cyclohexanecarboxylate (0.93 g, 3.53 mmol) was dissolved in acetic acid (60 ml). The solution was passed through Rh/Al₂O₃ cartridge on H-Cube at 80° C. under 80 bars. The reaction mixture was concentrated under vacuum to result in 0.95 g (100%) of trans-ethyl 1-methyl-4-(piperidin-4-yloxy)cyclohexanecarboxylate as a colorless oil. LC-MS (ES, m/z) C₁₅H₂₇NO₃: 269; Found: 270 [M+H]⁺.

### Intermediate 72: ethyl 1-methyl-3-oxocyclobutanecarboxylate

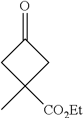

### Step 1

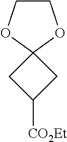 [SMILES: CCOC(=O)C1CC2(C1)OCCO2]

To a solution of 5 g (35.2 mmol) of ethyl 3-oxocyclobutane carboxylate in 50 mL of toluene was added 3.27 g (52.8 mmol) of ethylene glycol followed by 0.1 mL of conc. H₂SO₄. The mixture was heated at reflux overnight employing a Dean-Stark trap to remove the liberated water. The solvent was removed under reduced pressure and the residue dissolved in MTBE and washed with sat. NaHCO₃, dried over MgSO₄ and concentrated. The crude product (ethyl 5,8-dioxaspiro[3.4]octane-2-carboxylate) was used in the next reaction without further purification.

### Step 2

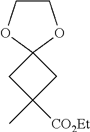

To a solution of 2.93 g (29.0 mmol) of diisopropylamine in anhydrous THF at −20° C. was added drop wise 11.6 mL (29.0 mmol) of a 2.5 M solution of BuLi while maintaining the internal temperature <at −10° C. The LDA solution was then cooled to −78° C. and 3.60 g (19.3 mmol) of ethyl 5,8-dioxaspiro[3.4]octane-2-carboxylate was added drop wise. The resulting mixture was stirred for 30 min at which point 8.23 g (58.0 mmol) of MeI was added. The reaction mixture was allowed to warm to room temperature. The reaction was quenched with sat. NH₄Cl and extracted with MTBE, dried over MgSO₄, concentrated under reduced pressure and purified by silica gel chromatography to afford ethyl 2-methyl-5,8-dioxaspiro[3.4]octane-2-carboxylate.

### Step 3

To a solution of 1.60 g (7.99 mmol) of ethyl 2-methyl-5,8-dioxaspiro[3.4]octane-2-carboxylate in acetone was added 7.99 mL of a 1 M solution of HCl and the mixture was stirred at room temperature for 4 days. The acetone was removed under reduced pressure and the aqueous layer extracted with MTBE, dried over MgSO₄ and concentrated. The crude carboxylic acid was re-dissolved in CH₂Cl₂ and 1.01 g (7.99 mmol) of oxalyl chloride was added followed by 1 drop of DMF. The mixture was stirred at rt for 2 h, concentrated under reduced pressure and re-dissolved in CH₂Cl₂ and added drop wise to a solution of EtOH in CH₂Cl₂. After 1 h, the mixture was washed with sat NaHCO₃, dried over MgSO₄ and concentrated under reduced pressure and used in the next step without further purification to afford ethyl 1-methyl-3-oxocyclobutanecarboxylate.

### Intermediate 73: ethyl cis-3-hydroxy-1-methylcyclobutanecarboxylate

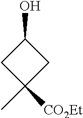

Dissolved into 180 mL of 0.1M pH 7 phosphate buffer was 3.66 g of P1B2 and 1.83 g

NADP then 3.6 g ethyl 1-methyl-3-oxocyclobutanecarboxylate dissolved into 180 mL IPA was slowly added. The pH was adjusted to 7 and then (capped vial) shaken over night. The reaction was transferred to a 1 L sep funnel and 180 mL MTBE was added. The layers were separated and then back extracted aq. first with 2×150 mL of 2/1 MTBE/IPA. The combined organics were then concentrated to dryness and then dissolved into 100 mL MTBE. This MTBE solution was then washed with 2×100 mL water then 100 mL brine (back extracted these combined aq. washes with MTBE). MTBE layers were then dried over Na₂SO₄, filtered and concentrated to dryness. Ethyl cis-3-hydroxy-1-methylcyclobutanecarboxylate was isolated as a colorless oil.

### Intermediate 74: ethyl trans-3-hydroxy-1-methylcyclobutanecarboxylate

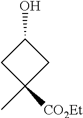

3.66 g of MIF20 and 1.8 g NADP were dissolved into 180 mL of 0.1M pH 7 phosphate buffer then 3.66 g ethyl 1-methyl-3-oxocyclobutanecarboxylate dissolved into 180 mL IPA was slowly added. The pH was adjusted to 7 and then (capped vial) shaken over night. The reaction was transferred to a 1 L sep funnel and 180 mL MTBE was added. The layers were separated and then back extracted aq. first with 2×150 mL of 2/1 MTBE/IPA. The combined organics were then concentrated to dryness and then dissolved into 100 mL MTBE. This MTBE solution was then washed with 2×100 mL water then 100 mL brine (back extracted these combined aq. washes with MTBE). MTBE layers were then dried over Na₂SO₄, filtered and concentrated to dryness. Ethyl trans-3-hydroxy-1-methylcyclobutanecarboxylate were isolated as a colorless oil.

### Intermediate 75: benzyl 4-(trans-3-(ethoxycarbonyl)-3-methylcyclobutoxy)piperidine-1-carboxylate and Intermediate 76: benzyl 4-((cis-3-(ethoxycarbonyl)-3-methylcyclobutoxy)piperidine-1-carboxylate

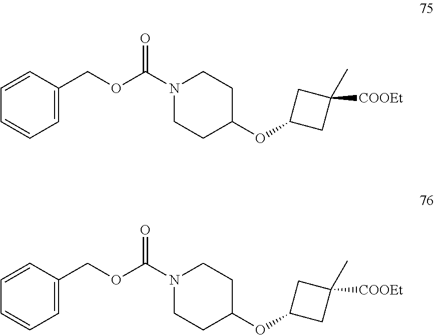 [SMILES: CCOC(=O)[C@]1(C)C[C@@H](OC2CCN(C(=O)OCc3ccccc3)CC2)C1 ; CCOC(=O)[C@]1(C)C[C@H](OC2CCN(C(=O)OCc3ccccc3)CC2)C1]

Ethyl 3-hydroxy-1-methylcyclobutanecarboxylate (1.06 g, 6.7 mmol) (˜1:1 mixture of cis:trans) was dissolved in anhydrous THF (70 ml) at 0° C., TEA (1.027 ml, 7.37 mmol) was added, followed by drop wise addition of TMS-Cl (0.899 ml, 7.03 mmol). The reaction mixture aged for 30 min then diluted with hexane (70 ml) and filtered through a small pad of celite eluted with hexane and concentrated. The crude product and benzyl 4-oxopiperidine-1-carboxylate (1.5 g, 6.43 mmol) was dissolved in dichloride methane (70 ml) at −60-65° C., triethylsilane (1.18 ml, 7.37 mmol) was added, followed by drop wise addition of TMS-OTf (0.605 ml, 3.35 mmol) and the mixture was allowed to warm to 0° C. and aged for 30 min. The reaction mixture was diluted with EtOAc (50 ml), 1 M H₃PO₄ (10 ml) was added, the organic layer washed with brine (2×20 ml) and dried over anhydrous sodium sulfate and concentrated under vacuum. This trans/cis mixture was separated by SFC on a Chiralpak AD-H column, 250×50 mm. Mobile phase: A for SF CO₂ and B for ethanol. Gradient: B 20%. This resulted in benzyl 4-(trans-3-(ethoxycarbonyl)-3-methylcyclobutoxy)piperidine-1-carboxylate. LC-MS (ES, m/z) C₂₁H₂₉NO₅: 375; Found: 376 [M+H]⁺ and benzyl 4-(cis-3-(ethoxycarbonyl)-3-methylcyclobutoxy)piperidine-1-carboxylate. LC-MS (ES, m/z) C₂₁H₂₉NO₅: 375; Found: 376 [M+H]⁺.

### Intermediate 77: ethyl trans-1-methyl-3-(piperidin-4-yloxy)cyclobutanecarboxylate

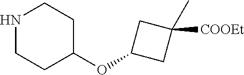 [SMILES: CCOC(=O)[C@]1(C)C[C@@H](OC2CCNCC2)C1]

Benzyl 4-(trans-3-(ethoxycarbonyl)-3-methylcyclobutoxy)piperidine-1-carboxylate (0.067 g, 0.178 mmol) was dissolved in ethanol (10 ml), 5% palladium on carbon (0.001 g, 0.009 mmol) was added. The reaction mixture stirred at 1 atm H₂ for 2 days. The reaction mixture was concentrated under vacuum to result in 0.043 g (100%) of ethyl trans-1-methyl-3-(piperidin-4-yloxy)cyclobutanecarboxylate as a colorless oil. LC-MS (ES, m/z) C₁₃H₂₃NO₃: 241; Found: 242 [M+H]⁺.

### Intermediate 78: ethyl cis-1-methyl-3-(piperidin-4-yloxy)cyclobutanecarboxylate

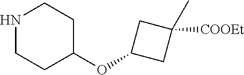 [SMILES: CCOC(=O)[C@]1(C)C[C@H](OC2CCNCC2)C1]

Benzyl 4-(cis-3-(ethoxycarbonyl)-3-methylcyclobutoxy)piperidine-1-carboxylate (0.104 g, 0.277 mmol) was dissolved in ethanol (10 ml), 5% palladium on carbon (0.002 g, 0.014 mmol) was added. The reaction mixture stirred at 1 atm H₂ for 2 days. The reaction mixture was concentrated under vacuum to result in 0.067 g (100%) of ethyl cis-1-methyl-3-(piperidin-4-yloxy)cyclobutanecarboxylate as a colorless oil. LC-MS (ES, m/z) C₁₃H₂₃NO₃: 241; Found: 242 [M+H]⁺.

### Intermediate 79: ethyl cis-4-{[1-(5-formylpyridin-2-yl)piperidin-4-yl]oxy}-1-methylcyclohexanecarboxylate

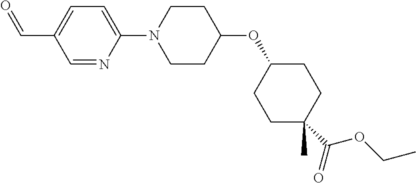 [SMILES: CCOC(=O)[C@]1(C)CC[C@H](OC2CCN(c3ccc(C=O)cn3)CC2)CC1]

A mixture of ethyl cis-1-methyl-4-(piperidin-4-yloxy)cyclohexanecarboxylate (1 g, 3.71 mmol), 2-fluoro-5-formylpyridine (0.464 g, 3.71 mmol) and sodium bicarbonate (3.12 g, 37.1 mmol) in DMSO (8 ml) was heated at 110° C. in an oil bath for 4 hours under N₂. The reaction mixture was cooled to room temperature, water (10 ml) was added, extracted with 3×15 ml ethyl acetate. The organic layers were combined, washed with 2×10 ml of saturated brine, dried over anhydrous sodium sulfate and concentrated under vacuum. The residue was applied onto a silica gel column and eluted with ethyl acetate/hexane 0-100%. This resulted in 0.7 g (50.4%) of ethyl cis-4-{[1-(5-formylpyridin-2-yl)piperidin-4-yl]oxy}-1-methylcyclohexanecarboxylate as a white solid. LC-MS (ES, m/z) C₂₁H₃₀N₂O₄: 374; Found: 375 [M+H]⁺.

### Intermediate 80: methyl 5-(1-(tert-butoxycarbonyl)piperidin-4-yloxy)nicotinate

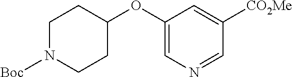 [SMILES: COC(=O)c1cncc(OC2CCN(C(=O)OC(C)(C)C)CC2)c1]

Methyl 5-hydroxynicotinate (3 g, 19.59 mmol), tert-butyl 4-hydroxypiperidine-1-carboxylate (4.93 g, 24.49 mmol), and triphenylphosphine (6.42 g, 24.49 mmol) in THF (106 ml) at 0° C. was added diisopropyl azodicarboxylate (4.85 ml, 24.49 mmol) drop wise over 10 min. The reaction was removed from the ice bath and stirred at RT for 10 min, then heated to 55° C. and stirred under nitrogen for 40 hours. The reaction mixture was concentrated, and residue was treated with EtOAc (45 ml) followed by Hexanes (45 ml). The mixture was stirred at RT overnight. The white precipitate was filtered off with a glass funnel, rinsed with EtOAc/Hexanes (1:1) and discarded. The filtrate was concentrated and purified by MPLC (330 g column, 0-100% EtOAc in Hexanes to yield Methyl 5-(1-(tert-butoxycarbonyl)piperidin-4-yloxy)nicotinate (5.98 g) LC-MS (ES, m/z) C₁₇H₂₄N₂O₅: 336; Found: 337 [M+H]⁺.

### Intermediate 81: methyl 4-(1-(tert-butoxycarbonyl)piperidin-4-yloxy)picolinate

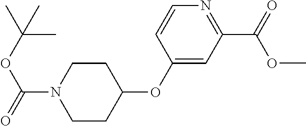 [SMILES: COC(=O)c1cc(OC2CCN(C(=O)OC(C)(C)C)CC2)ccn1]

To a mixture of methyl 4-hydroxypicolinate (5 g, 32.7 mmol), tert-butyl 4-hydroxypiperidine-1-carboxylate (6.57 g, 32.7 mmol) and triphenylphosphine (10.7 g, 40.8 mmol) in THF (200 ml) added diisopropylazodicarboxylate (8.25 g, 40.8 mmol) drop wise. The reaction mixture was heated at 55° C. in an oil bath for 2 days under N₂. The reaction mixture was cooled to room temperature, concentrated under vacuum then purified by SFC, chiralpak AS (20 μm, 300×50 mm I.D). Mobile phase: A for CO₂ and B for ethanol, Gradient: B 20%. This resulted in 8.8 g (80%) of methyl 4-(1-(tert-butoxycarbonyl)piperidin-4-yloxy)picolinate as a white solid. LC-MS (ES, m/z) C₁₇H₂₄N₂O₅: 336; Found: 337 [M+H]⁺.

### Intermediate 82: 6-(piperidin-4-yloxy)pyridine-3-carboxylic acid

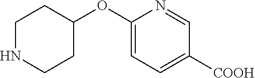 [SMILES: O=C(O)c1ccc(OC2CCNCC2)nc1]

A mixture of tert-butyl 4-[(5-cyanopyridin-2-yl)oxy]piperidine-1-carboxylate (1 g, 3.3 mmol) in HCl (conc. aq., 10 mL, 122 mmol) was heated to reflux overnight, The mixture was cooled to room temperature and concentrated in vacuo to afford HCl salt of 6-(piperidin-4-yloxy)pyridine-3-carboxylic acid. LC-MS (ES, m/z) C₁₁H₁₄N₂O₃: 222; Found: 223 [M+H]⁺.

### Intermediate 83: Sodium 3-oxo-5-[trans-4-(piperidin-4-yloxy)cyclohexyl]-1,2,5-thiadiazolidin-2-ide 1,1-dioxide

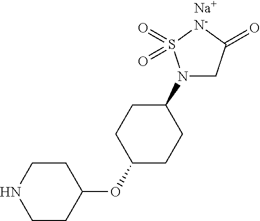 [SMILES: [Na+] ; O=C1CN([C@H]2CC[C@H](OC3CCNCC3)CC2)S(=O)(=O)[N-]1]

### Step 1

To a suspension of trans-4-{[tert-butyl(dimethyl)silyl]oxy}cyclohexanamine (1.54 g, 6.71 mmol) in acetonitrile (20 mL) was added triethylamine (2.82 mL, 20.2 mmol). Ethyl chloroacetate (0.72 mL, 6.71 mmol) was added and the mixture was heated to reflux for 1 hour. The solvent was evaporated to dryness and the residue was partitioned between ethyl acetate and water. The organic layer was dried over sodium sulfate and concentrated to give ethyl N-(trans-4-{[tert-butyl(dimethyl)silyl]oxy}cyclohexyl) glycinate which was used without further purification. LC-MS (ES, m/z) C₁₆H₃₃NO₃Si: 315: Found: 316[M+H]⁺.

### Step 2

To a solution of chlorosulfonyl isocyanate (0.33 mL, 3.80 mmol) in CH₂Cl₂ (1 mL) cooled to 0° C. was added benzyl alcohol (0.40 mL, 3.80 mmol). The mixture was stirred under nitrogen for 30 min. and a mixture of ethyl N-(trans-4-{[tert-butyl(dimethyl)silyl]oxy}cyclohexyl) glycinate (1.2 g, 3.80 mmol) and triethylamine (1.59 mL, 11.41 mmol) in CH₂Cl₂ (5 mL) was added. The cooling bath was removed and the mixture was stirred at room temperature for 1 hour. The mixture was washed with water and the organic layer was dried over sodium sulfate. The solvent was evaporated and the residue was chromatographed on silica gel using 1-5% methanol/CH₂Cl₂ as gradient. The fractions were evaporated to give ethyl N-{[(benzyloxy)carbonyl]sulfamoyl}-N-(trans-4-{[tert-butyl(dimethyl)silyl]oxy}cyclohexyl)glycinate. LC-MS (ES, m/z) C₂₄H₄₀N₂O₇SSi: 528: Found: 529[M+H]⁺.

### Step 3

A solution of ethyl N-{[(benzyloxy)carbonyl]sulfamoyl}-N-(trans-4-{[tert-butyl(dimethyl)silyl]oxy}cyclohexyl)glycinate (103 mg, 0.19 mmol) and benzyl 4-oxopiperidine-1-carboxylate (45 mg, 0.19 mmol) dissolved in CH₂Cl₂ (5 mL) was cooled to −70° C. under nitrogen. Triethylsilane (0.068 mL, 0.43 mmol) was added, followed by trimethylsilyl trifluoromethanesulfonate (0.07 mL, 0.30 mmol). The mixture was stirred at −70° C. for 10 min. and warmed to 0° C. The mixture was partitioned between ethyl acetate and 1N HCl. The organic layer was dried over sodium sulfate and concentrated. The residue was purified by chromatography on silica gel using 30-100% ethyl acetate/hexanes as gradient to give benzyl 4-({trans-4-[{[(benzyloxy)carbonyl]sulfamoyl}(2-ethoxy-2-oxoethyl)amino]cyclohexyl}oxy)piperidine-1-carboxylate. LC-MS (ES, m/z) C₃₁H₄₁N₃O₉S: 631: Found: 632 [M+H]

### Step 4

A solution of benzyl 4-({trans-4-[{[(benzyloxy)carbonyl]sulfamoyl}(2-ethoxy-2-oxoethyl)amino]cyclohexyl}oxy)piperidine-1-carboxylate (50 mg, 0.079 mmol) in ethanol (5 mL) was hydrogenated at 30 bar and 30° C. on an H-Cube™ apparatus using 10% Pd on carbon as catalyst for 15 min. The solvent was evaporated to give ethyl N-[trans-4-(piperidin-4-yloxy)cyclohexyl]-N-sulfamoylglycinate as a clear oil. LC-MS (ES, m/z) C₁₅H₂₉N₃O₅S: 363: Found: 364 [M+H]⁺.

### Step 5

A solution of ethyl N-[trans-4-(piperidin-4-yloxy)cyclohexyl]-N-sulfamoylglycinate (20 mg, 0.055 mmol) in methanol (1 mL) was treated with sodium methoxide 0.5M (0.011 mL, 0.055 mmol) at room temperature for 4 hours. The solvent was evaporated to dryness to afford sodium 3-oxo-5-[trans-4-(piperidin-4-yloxy)cyclohexyl]-1,2,5-thiadiazolidin-2-ide 1,1-dioxide as a tan solid. LC-MS (ES, m/z) C₁₃H₂₃N₃NaO4S: 317: Found: 318 [M+H]⁺.

### Intermediate 84: (3R,3aR,6S,6aR)-6-(piperidin-4-yloxy)hexahydrofuro[3,2-b]furan-3-ol

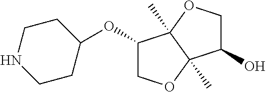 [SMILES: C[C@]12OC[C@H](OC3CCNCC3)[C@@]1(C)OC[C@H]2O]

### Step 1

In a 100 ml round-bottom flask equipped with magnetic stirring and nitrogen inlet was charged with (3R,3aR,6R,6aR)-hexahydrofuro[3,2-b]furan-3,6-diol (isomannide, 3.30 g, 22.6 mmol), pyridin-4-ol (0.716 g, 7.53 mmol), triphenylphosphane (2.47 g, 9.41 mmol), THF (36 ml) followed by dipropan-2-yl (E)-diazene-1,2-dicarboxylate (1.90 g, 9.41 mmol) drop wise at room temperature. The resultant mixture was stirred overnight at 55° c. Saturated solution of ammonium chloride was added to the reaction solution and the reaction mixture was stirred at RT for 30 min. The product mixtures were partitioned between EtOAc and water, separated the organic phase and the aqueous phase was extracted with EtOAc (2×50 ml). The combined organic phase was washed with sodium bicarbonate, brine, dried over Na2SO4, filtered and concentrated under reduced pressure on a rotary evaporator. The residue was purified by flash chromatography on an ISCO CombiFlash using a 80 g ISCO silica gel cartridge eluting with linear gradient of 0-6% over 5 CV, isocratic 6% over 5 CV, linear gradient of 6-15% over 3 CV. The product containing fractions were collected and concentrated under reduced pressure to afford (3R,3aR,6S,6aR)-6-(pyridin-4-yloxy)hexahydrofuro[3,2-b]furan-3-ol s off-white solids 437 mg (23%). LC-MS (ES, m/z) C₁₁H₁₃NO₄: 223; Found: 224 [M+H]⁺.

### Step 2

A solution of (3R,3aR,6S,6aR)-6-(pyridin-4-yloxy)hexahydrofuro[3,2-b]furan-3-ol (437 mg, 1.96 mmol) in acetic acid/MeOH (12 ml/4 ml) was treated with 5% Rh/c (33 mg) under hydrogen (400 psi) at 80 c for 15 hrs. The product ((3R,3aR,6S,6aR)-6-(piperidin-4-yloxy)hexahydrofuro[3,2-b]furan-3-ol) was isolated by filtration to remove catalyst, concentration under reduced pressure and used in the next step without further purification. LC-MS (ES, m/z) C₁₁H₁₉NO₄: 229; Found: 230 [M+H]⁺.

### Intermediate 85: benzyl 4-(cyclopent-3-en-1-yloxy)piperidine-1-carboxylate

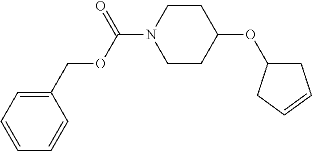 [SMILES: O=C(OCc1ccccc1)N1CCC(OC2CC=CC2)CC1]

Cyclopent-3-enol (5 g, 59.4 mmol) was dissolved in anhydrous THF (150 ml) at 0° C., TEA (9.11 ml, 65.4 mmol) added, followed by drop wise addition of TMS-Cl (7.98 ml, 62.4 mmol). The reaction mixture was aged for 30 min then diluted with hexane (150 ml) and filtered through a small pad of celite eluting with hexane and concentrated. The crude product and benzyl 4-oxopiperidine-1-carboxylate (13.31 g, 57.1 mmol) were dissolved in dichloride methane (150 ml) at −60-65° C., triethylsilane (10.44 ml, 65.4 mmol) added, followed by drop wise addition of TMS-OTf (5.37 ml, 29.7 mmol). The mixture was allowed to warm to 0° C. and aged for 30 min. The reaction mixture was diluted with EtOAc (100 ml), 1 M H₃PO₄ (30 ml) added, the organic layer washed with brine (2×20 ml) and dried over anhydrous sodium sulfate and concentrated under vacuum. The residual was purified by silica gel chromatography (eluted with ethyl acetate/hexane 0-50%) to afford 12 g (67%) of benzyl 4-(cyclopent-3-en-1-yloxy) piperidine-1-carboxylate as colorless oil. LC-MS (ES, m/z) C₁₈H₂₃NO₃: 301; Found: 302 [M+H]⁺.

### Intermediate 86: benzyl 4-{[(1R,3R,5S,6r)-6-(ethoxycarbonyl)bicyclo[3.1.0]hex-3-yl]oxy}piperidine-1-carboxylate

### Intermediate 87: benzyl 4-{[(1R,3S,5S,6r)-6-(ethoxycarbonyl)bicyclo[3.1.0]hex-3-yl]oxy}piperidine-1-carboxylate

### Intermediate 88: benzyl 4-{[(1R,3r,5S,6s)-6-(ethoxycarbonyl)bicyclo[3.1.0]hex-3-yl]oxy}piperidine-1-carboxylate and

### Intermediate 89: benzyl 4-{[(1R,3s,5S,6s)-6-(ethoxycarbonyl)bicyclo[3.1.0]hex-3-yl]oxy}piperidine-1-carboxylate

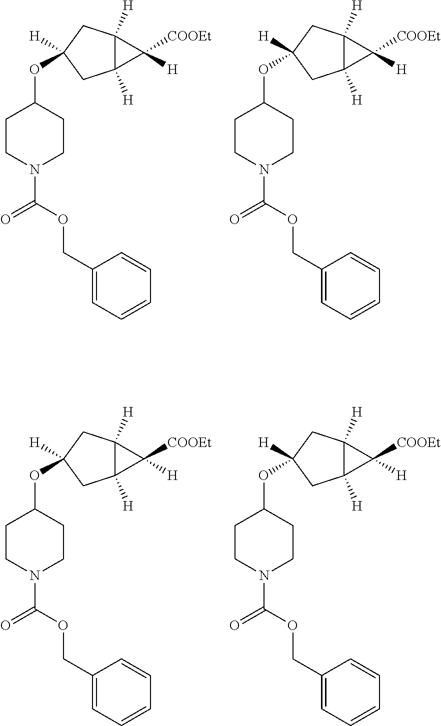 [SMILES: CCOC(=O)[C@H]1[C@@H]2C[C@H](OC3CCN(C(=O)OCc4ccccc4)CC3)C[C@@H]21 ; CCOC(=O)[C@H]1[C@@H]2C[C@@H](OC3CCN(C(=O)OCc4ccccc4)CC3)C[C@@H]21 ; CCOC(=O)[C@@H]1[C@@H]2C[C@H](OC3CCN(C(=O)OCc4ccccc4)CC3)C[C@@H]21 ; CCOC(=O)[C@@H]1[C@@H]2C[C@@H](OC3CCN(C(=O)OCc4ccccc4)CC3)C[C@@H]21]

To a solution of benzyl 4-(cyclopent-3-en-1-yloxy) piperidine-1-carboxylate (4 g, 13.27 mmol) and rhodium(II) acetate dimmer (0.117 g, 0.265 mmol) in dichloromethane (250 ml) was added ethyl diazoacetate (1.514 ml, 14.6 mmol) in dichloromethane (40 ml) via syringe pump for 5 hours at room temperature. The reaction mixture aged for one hour then was filtered through a pad of silica gel, the silica pad was washed with 3×20 ml EtOAc. The organic layers were combined, washed with 2×10 ml of saturated brine, dried over anhydrous sodium sulfate and concentrated under vacuum. This mixture was purified first by SFC on a chiralpak AD-H column (300×50 mm ID) (Mobile phase: A for SF CO₂ and B for methanol. Gradient: B 40%). The material was then separated by chiralpak AD-10 μm column (300×50 mm ID) (Mobile phase: A for SF CO₂ and B for ethanol. Gradient: B 25%). This resulted in benzyl 4-{[(1R,3R,5S,6r)-6-(ethoxycarbonyl)bicyclo[3.1.0]hex-3-yl]oxy}piperidine-1-carboxylate as white solid. LC-MS (ES, m/z) C₂₂H₂₉NO₅: 387; Found: 388 [M+H]⁺, benzyl 4-{[(1R,3S,5S,6r)-6-(ethoxycarbonyl)bicyclo[3.1.0]hex-3-yl]oxy}piperidine-1-carboxylate as white solid. LC-MS (ES, m/z) C₂₂H₂₉NO₅: 387; Found: 388 [M+H]⁺, benzyl 4-{[(1R,3r,5S,6s)-6-(ethoxycarbonyl)bicyclo[3.1.0]hex-3-yl]oxy}piperidine-1-carboxylate as white solid. LC-MS (ES, m/z) C₂₂H₂₉NO₅: 387; Found: 388 [M+H]⁺, and benzyl 4-{[(1R,3s,5S,6s)-6-(ethoxycarbonyl)bicyclo[3.1.0]hex-3-yl]oxy}piperidine-1-carboxylate as white solid. LC-MS (ES, m/z) C₂₂H₂₉NO₅: 387; Found: 388 [M+H]⁺.

### Intermediates 90-93: benzyl 3-{[4-(2-methoxy-2 oxoethyl)cyclohexyl]oxy}pyrrolidine-1-carboxylate

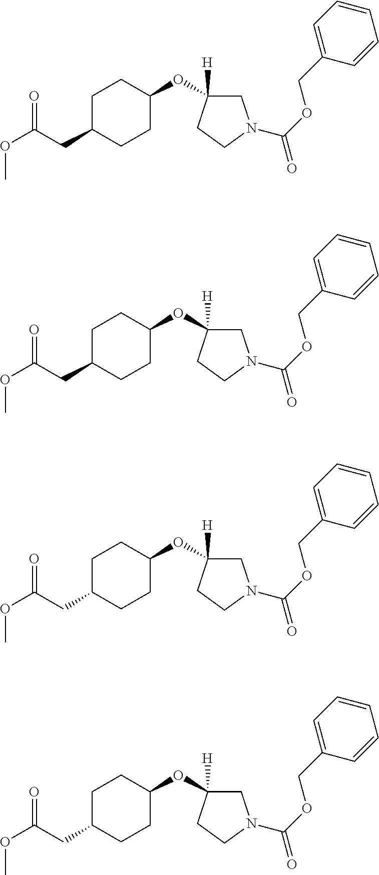 [SMILES: COC(=O)C[C@H]1CC[C@@H](O[C@H]2CCN(C(=O)OCc3ccccc3)C2)CC1 ; COC(=O)C[C@H]1CC[C@@H](O[C@@H]2CCN(C(=O)OCc3ccccc3)C2)CC1 ; COC(=O)C[C@H]1CC[C@H](O[C@H]2CCN(C(=O)OCc3ccccc3)C2)CC1 ; COC(=O)C[C@H]1CC[C@H](O[C@@H]2CCN(C(=O)OCc3ccccc3)C2)CC1]

Racemic benzyl 3-hydroxypyrrolidine-1-carboxylate (5 g, 22.6 mmol) was dissolved in anhydrous THF (150 ml) at 0° C., TEA (3.46 ml, 24.86 mmol) added, followed by drop wise addition of TMS-Cl (3.03 ml, 23.73 mmol). The reaction mixture aged for 30 min then diluted with hexane (150 ml) and filtered through a small pad of celite eluting with hexane and concentrated. The crude product and cis/trans methyl 2-(4-oxocyclohexyl)acetate (3.69 g, 21.69 mmol) were dissolved in dichloromethane (150 ml) at −60° C., triethylsilane (3.97 ml, 24.86 mmol) was added, followed by drop wise addition of TMS-OTf (2.04 ml, 11.3 mmol). The mixture was allowed to warm to 0° C. and age for 30 min. The reaction mixture was diluted with EtOAc (100 ml), 1 M H₃PO₄ (30 ml) was added, the organic layer was washed with brine (2×20 ml) and dried over anhydrous sodium sulfate and concentrated under vacuum. This mixture was separated by SFC (ChiralPak AD-H, (250×50 mmI.D). Mobile phase: A for SF CO₂ and B for methanol. Gradient: B 40%) followed by a second SFC (ChiralCel OJ-H, (250×50 mmI.D). Mobile phase: A for SF CO₂ and B for ethanol. Gradient: B 25%). This resulted in benzyl 3-(4-(2-methoxy-2-oxoethyl)cyclohexyloxy)pyrrolidine-1-carboxylate (isomer A, intermediates 85) C₂₁H₂₉NO₅: 375; Found: 376 [M+H]⁺, benzyl 3-(4-(2-methoxy-2-oxoethyl)cyclohexyloxy)pyrrolidine-1-carboxylate (isomer B, intermediates 86) C₂₁H₂₉NO₅: 375; Found: 376 [M+H]⁺, benzyl 3-(4-(2-methoxy-2-oxoethyl)cyclohexyloxy)pyrrolidine-1-carboxylate (isomer C, intermediates 87) C₂₁H₂₉NO₅: 375; Found: 376 [M+H]⁺., and benzyl 3-(4-(2-methoxy-2-oxoethyl)cyclohexyloxy)pyrrolidine-1-carboxylate (isomer D, intermediates 88) C₂₁H₂₉NO₅: 375; Found: 376 [M+H]⁺.

### Intermediate 94: methyl 2-(4-(pyrrolidin-3-yloxy)cyclohexyl)acetate

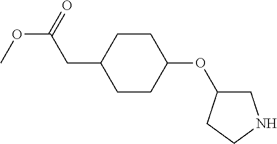 [SMILES: COC(=O)CC1CCC(OC2CCNC2)CC1]

Benzyl 3-(4-(2-methoxy-2-oxoethyl)cyclohexyloxy)pyrrolidine-1-carboxylate (a mixture of isomer A and isomer C, 0.56 g, 1.492 mmol) was dissolved in ethanol (5 ml), 5% palladium on carbon (0.008 g, 0.075 mmol) added. The reaction mixture stirred at 1 atm H₂ for 2 days. The reaction mixture concentrated under vacuum to result in methyl 2-(4-(pyrrolidin-3-yloxy)cyclohexyl)acetate as a colorless oil. LC-MS (ES, m/z) C₁₃H₂₃NO₃: 241; Found: 242 [M+H]⁺.

### Intermediates 95: methyl 2-(4-(pyrrolidin-3-yloxy)cyclohexyl)acetate

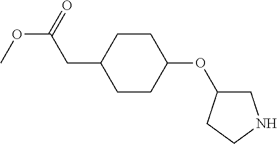 [SMILES: COC(=O)CC1CCC(OC2CCNC2)CC1]

Benzyl 3-(4-(2-methoxy-2-oxoethyl)cyclohexyloxy)pyrrolidine-1-carboxylate (a mixture of isomer B and isomer D, 0.52 g, 1.385 mmol) was dissolved in ethanol (5 ml), 5% palladium on carbon (0.007 g, 0.07 mmol) added. The reaction mixture stirred at 1 atm H₂ for 2 days. The reaction mixture was concentrated under vacuum to afford of methyl 244-(pyrrolidin-3-yloxy)cyclohexyl)acetate as a colorless oil. LC-MS (ES, m/z) C₁₃H₂₃NO₃: 241; Found: 242 [M+H]⁺.

### Intermediate 96: methyl [trans-4-({(3R)-1-[5-(5-fluoro-1H-benzimidazol-2-yl)pyridin-2-yl]pyrrolidin-3-yl}oxy)cyclohexyl]acetate and

### Intermediate 97: methyl [cis-4-({1-[5-(5-fluoro-1H-benzimidazol-2-yl)pyridin-2-yl]pyrrolidin-3-yl}oxy)cyclohexyl]acetate

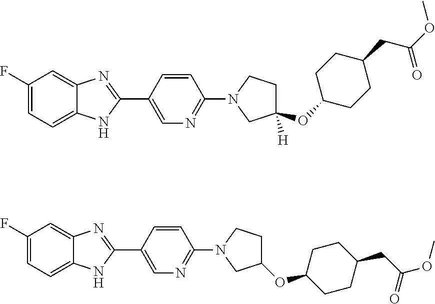 [SMILES: COC(=O)C[C@H]1CC[C@H](O[C@@H]2CCN(c3ccc(-c4nc5cc(F)ccc5[nH]4)cn3)C2)CC1 ; COC(=O)C[C@H]1CC[C@@H](OC2CCN(c3ccc(-c4nc5cc(F)ccc5[nH]4)cn3)C2)CC1]

A mixture of methyl 2-(4-(pyrrolidin-3-yloxy)cyclohexyl)acetate (Intermediate 94, 360 mg, 1.492 mmol), 6-fluoro-2-(6-fluoropyridin-3-yl)-1H-benzimidazole (345 mg, 1.492 mmol) and sodium bicarbonate (1.25 g, 14.92 mmol) in NMP (4 ml) was heated at 110° C. in an oil bath over night under N₂. Then 20 ml water added to the reaction mixture, extract with 3×15 ml ethyl acetate. The organic layers were combined, washed with 2×5 mL of saturated brine, dried over anhydrous sodium sulfate and concentrated under vacuum. Then separated by SFC, chiralCel OD column (10u, 300×50 mm I.D). Mobile phase: A for SF CO₂ and B for methanol (0.2% DEA). Gradient: B 40%. This resulted in 0.188 g (11.75%) of methyl [trans-4-({(3R)-1-[5-(5-fluoro-1H-benzimidazol-2-yl)pyridin-2-yl]pyrrolidin-3-yl}oxy)cyclohexyl]acetate as a white solid. LC-MS (ES, m/z) C₂₅H₂₉FN₄O₃: 452; Found: 453 [M+H]+, 0.135 g (8.44%) of methyl [cis-4-({1-[5-(5-fluoro-1H-benzimidazol-2-yl)pyridin-2-yl]pyrrolidin-3-yl}oxy)cyclohexyl]acetate (single isomer, absolute configuration not determined) as a white solid. LC-MS (ES, m/z) C₂₅H₂₉FN₄O₃: 452; Found: 453 [M+H]⁺.

Alternatively, methyl [trans-4-({(3R)-1-[5-(5-fluoro-1H-benzimidazol-2-yl)pyridin-2-yl]pyrrolidin-3-yl}oxy)cyclohexyl]acetate was prepared as the following:

### Step 1

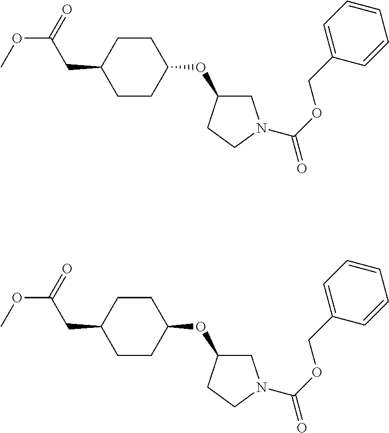 [SMILES: COC(=O)C[C@H]1CC[C@H](O[C@@H]2CCN(C(=O)OCc3ccccc3)C2)CC1 ; COC(=O)C[C@H]1CC[C@@H](O[C@@H]2CCN(C(=O)OCc3ccccc3)C2)CC1]

(R)-benzyl 3-hydroxypyrrolidine-1-carboxylate (7 g, 31.6 mmol) was dissolved in anhydrous THF (100 ml) at 0° C., TEA (4.85 ml, 34.8 mmol) added, followed by drop wise addition of TMS-Cl (4.25 ml, 33.2 mmol). The reaction mixture aged for 30 min then diluted with hexane (150 ml) and filtered through a small pad of celite eluting with hexane and concentrated. The crude product and methyl 2-(4-oxocyclohexyl)acetate (5.17 g, 30.4 mmol) dissolved in dichloride methane (150 ml) at −60-65° C., triethylsilane (5.56 ml, 34.8 mmol) added, followed by drop wise addition of TMS-OTf (2.86 ml, 15.82 mmol) and allow to warm to 0° C. and aged for 30 min. The reaction mixture diluted with EtOAc (100 ml), 1 M H₃PO₄ (30 ml) added, the organic layer washed with brine (2×20 ml) and dried over anhydrous sodium sulfate and concentrated under vacuum. Then applied onto a silica gel column and eluted with ethyl acetate/hexane 0-80%. This resulted in 4 g (33.7%) of trans/cis mixture. This trans/cis mixture separated by SFC on a chiralpak AD, 250×50 mm, 20% 2:1 MeOH/MeCN. This resulted in benzyl (3R)-3-{[trans-4-(2-methoxy-2-oxoethyl)cyclohexyl]oxy}pyrrolidine-1-carboxylate as colorless oil 1.1 g (18.5%), LC-MS (ES, m/z) C₂₁H₂₉NO₅: 375; Found: 376 [M+H]⁺ and benzyl (3R)-3-{[cis-4-(2-methoxy-2-oxoethyl)cyclohexyl]oxy}pyrrolidine-1-carboxylate as colorless oil 1.1 g (18.5%), LC-MS (ES, m/z) C₂₁H₂₉NO₅: 375; Found: 376 [M+H]⁺.

Alternatively benzyl (3R)-3-{[trans-4-(2-methoxy-2-oxoethyl)cyclohexyl]oxy}pyrrolidine-1-carboxylate and its enantiomers were obtained as following:

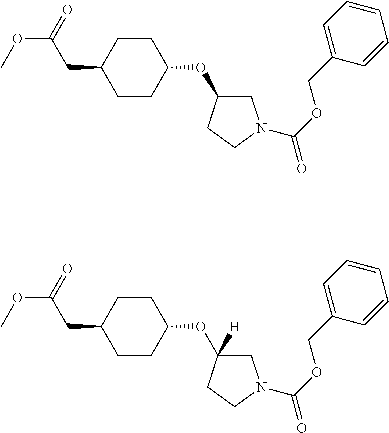 [SMILES: COC(=O)C[C@H]1CC[C@H](O[C@@H]2CCN(C(=O)OCc3ccccc3)C2)CC1 ; COC(=O)C[C@H]1CC[C@H](O[C@H]2CCN(C(=O)OCc3ccccc3)C2)CC1]

Methyl 2-(trans-4-hydroxycyclohexyl)acetate (3.84 g, 22.3 mmol) was dissolved in anhydrous THF (120 ml) at 0° C., DIEA (4.28 ml, 24.53 mmol) added, followed by drop wise addition of TMS-Cl (2.99 ml, 23.41 mmol). The reaction mixture was aged for 2 hours then diluted with hexane (50 ml) and filtered through a small pad of celite eluted with hexane and concentrated. The crude product and benzyl 3-oxopyrrolidine-1-carboxylate (4.64 g, 21.18 mmol) were dissolved in dichloromethane (80 ml) at −60-65° C. Triethylsilane (7.12 ml, 44.6 mmol) was added, followed by drop wise addition of TMS-OTf (2.02 ml, 11.15 mmol). The reaction mixture was allowed to warm to 0° C. and aged for 2 days. The reaction mixture was diluted with EtOAc (100 ml), 1 M H₃PO₄ (30 ml) was added, the organic layer was washed with brine (2×20 ml) and dried over anhydrous sodium sulfate and concentrated under vacuum. The residue was purified by a silica gel column eluted with ethyl acetate/hexane 0-80%. This resulted in racemic mixture, which was resolved by SFC (Chiral AD column, 250×30 mm, 30% 2:1 MeOH:MeCN/CO₂) to afford benzyl (3R)-3-{[trans-4-(2-methoxy-2-oxoethyl)cyclohexyl]oxy}pyrrolidine-1-carboxylate as colorless oil, LC-MS (ES, m/z) C₂₁H₂₉NO₅: 375; Found: 376 [M+H]⁺ and benzyl (3S)-3-{[trans-4-(2-methoxy-2-oxoethyl)cyclohexyl]oxy}pyrrolidine-1-carboxylate as colorless oil, LC-MS (ES, m/z) C₂₁H₂₉NO₅: 375; Found: 376 [M+H]⁺.

### Step 2

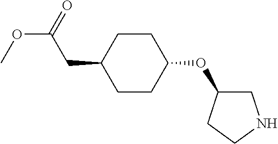 [SMILES: COC(=O)C[C@H]1CC[C@H](O[C@@H]2CCNC2)CC1]

Benzyl (3R)-3-{[trans-4-(2-methoxy-2-oxoethyl)cyclohexyl]oxy}pyrrolidine-1-carboxylate (1 g, 2.66 mmol) was dissolved in methanol (20 ml), 5% palladium on carbon (0.014 g, 0.133 mmol) added. The reaction mixture was stirred at 1 atm H₂ over night. The reaction mixture concentrated under vacuum to result in 0.643 g (100%) of methyl {trans-4-[(3R)-pyrrolidin-3-yloxy]cyclohexyl}acetate as a colorless oil. LC-MS (ES, m/z) C₁₃H₂₃NO₃: 241; Found: 242 [M+H]⁺.

### Step 3

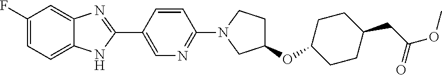 [SMILES: COC(=O)C[C@H]1CC[C@H](O[C@@H]2CCN(c3ccc(-c4nc5cc(F)ccc5[nH]4)cn3)C2)CC1]

A mixture of methyl {trans-4-[(3R)-pyrrolidin-3-yloxy]cyclohexyl}acetate (0.64 g, 2.85 mmol), 6-fluoro-2-(6-fluoropyridin-3-yl)-1H-benzimidazole (0.613 g, 2.65 mmol) and sodium bicarbonate (2.23 g, 26.5 mmol) in NMP (8 ml) was heated at 110° C. in an oil bath over night under N₂. The reaction mixture was cooled to room temperature, water (20 ml) added, extracted with 3×30 mL ethyl acetate. The organic layers were combined, washed with 2×10 mL of saturated brine, dried over anhydrous sodium sulfate and concentrated under vacuum. Then applied onto a silica gel column and eluted with ethyl acetate/hexane 20-100%. This resulted in 0.18 g (15%) of methyl [trans-4-({(3R)-1-[5-(5-fluoro-1H-benzimidazol-2-yl)pyridin-2-yl]pyrrolidin-3-yl}oxy)cyclohexyl]acetate as a white solid. LC-MS (ES, m/z) C₂₅H₂₉FN₄O₃: 452; Found: 453 [M+H]⁺.

### Intermediate 98: methyl [trans-4-({(3S)-1-[5-(5-fluoro-1H-benzimidazol-2-yl)pyridin-2-yl]pyrrolidin-3-yl}oxy)cyclohexyl]acetate and

### Intermediate 99: methyl [cis-4-({1-[5-(5-fluoro-1H-benzimidazol-2-yl)pyridin-2-yl]pyrrolidin-3-yl}oxy)cyclohexyl]acetate

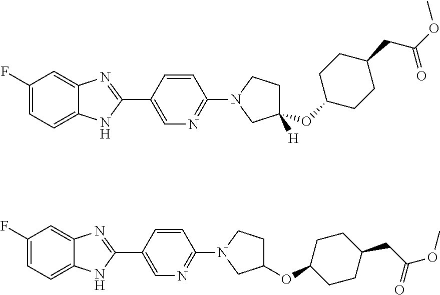 [SMILES: COC(=O)C[C@H]1CC[C@H](O[C@H]2CCN(c3ccc(-c4nc5cc(F)ccc5[nH]4)cn3)C2)CC1 ; COC(=O)C[C@H]1CC[C@@H](OC2CCN(c3ccc(-c4nc5cc(F)ccc5[nH]4)cn3)C2)CC1]

A mixture of methyl 2-(4-(pyrrolidin-3-yloxy)cyclohexyl)acetate (Intermediates 95, 334 mg, 1.384 mmol), 6-fluoro-2-(6-fluoropyridin-3-yl)-1H-benzimidazole (320 mg, 1.384 mmol) and sodium bicarbonate (1.16 g, 13.84 mmol) in NMP (4 ml) was heated at 110° C. in an oil bath over night under N₂. Then 20 ml water added to the reaction mixture, extract with 3×15 ml ethyl acetate. The organic layers were combined, washed with 2×5 mL of saturated brine, dried over anhydrous sodium sulfate and concentrated under vacuum. Then separated by SFC, chiralCel OD column (10u, 300×50 mm I.D). Mobile phase: A for SF CO₂ and B for methanol (0.2% DEA). Gradient: B 40%. This resulted in 0.191 g (11.94%) of methyl [trans-4-({(3S)-1-[5-(5-fluoro-1H-benzimidazol-2-yl)pyridin-2-yl]pyrrolidin-3-yl}oxy)cyclohexyl]acetate as a white solid. LC-MS (ES, m/z) C₂₅H₂₉FN₄O₃: 452; Found: 453 [M+H]⁺, and 0.136 g (8.5%) of methyl [cis-4-({1-[5-(5-fluoro-1H-benzimidazol-2-yl)pyridin-2-yl]pyrrolidin-3-yl}oxy)cyclohexyl]acetate (single isomer, absolute configuration not determined) as a white solid. LC-MS (ES, m/z) C₂₅H₂₉FN₄O₃: 452; Found: 453 [M+H]⁺.

### Intermediate 100

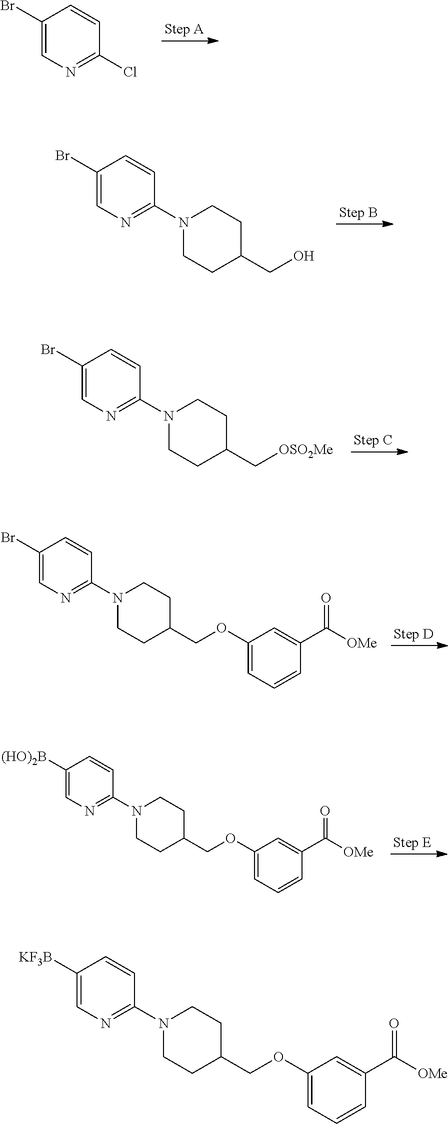

### Step A: [1-(5-bromopyridin-2-yl)piperidin-4-yl]methanol

A mixture of 5-bromo-2-chloropyridine (3.52 g, 0.020 mol) and piperidin-4-ylmethanol (2.30 g, 0.020 mol) and N,N-diisopropylethylamine (3.10 g, 4.2 mL, 0.024 mol) in THF (12 mL) was heated at 120° C. for 2 h in a microwave reactor then cooled to RT and concentrated. To the residue was added EtOAc (100 mL), washed with sat. NaHCO₃ aqueous and brine. The organic phase was dried (Na₂SO₄), filtered, and concentrated. Purification by silica gel column chromatography (eluant: 0-60% EtOAc/hexane) to yield (1-(5-bromopyridin-2-yl)piperidin-4-yl)methanol as a white solid. LC/MS=272 [M+1].

### Step B: [1-(5-bromopyridin-2-yl)piperidin-4-yl]methyl methanesulfonate

To a solution of (1-(5-bromopyridin-2-yl)piperidin-4-yl)methanol (2.60 g, 9.60 mmol) in CH₂Cl₂ (30 mL) cooled to 0° C. was added triethylamine (1.26 g, 1.7 mL, 12.5 mol) and mesyl chloride (1.21 g, 0.82 mL, 10.6 mmol). The reaction mixture was stirred at 0° C. for 15 mins then at room temperature for 60 mins. Water (100 mL) was added, and the aqueous solution was extracted with EtOAc. The combined extracts were dried (MgSO₄), filtered, and concentrated to yield (1-(5-bromopyridin-2-yl)piperidin-4-yl)methyl methanesulfonate as a yellow solid. LC/MS=350 [M+1].

### Step C: methyl 3-[[1-(5-bromopyridin-2-yl)piperidin-4-yl]methoxy]benzoate

To a solution of methyl 3-hydroxybenzoate (2.19 g, 14.39 mmol) in dry DMF (50 mL) under nitrogen was added sodium hydride (0.58 g of 60 wt % in oil, 14.39 mmol). The mixture was stirred at RT for 15 mins then added (1-(5-bromopyridin-2-yl)piperidin-4-yl)methyl methanesulfonate (3.35 g, 9.59 mmol) in dry DMF (10 mL). The resulting mixture was heated at 50° C. for 18 h then cooled and diluted with EtOAc/hexane (2:1, 100 mL). Water (150 mL) was added, and organic phase was separated. The aqueous solution was extracted with EtOAc/hexane (2:1, 100 mL). The combined extracts were dried (MgSO₄), filtered, and concentrated. Purification by silica gel column chromatography (eluant: 0-60% EtOAc/hexane) to yield methyl 3-((1-(5-bromopyridin-2-yl)piperidin-4-yl)methoxy)benzoate as a white solid. LC/MS=406 [M+1].

### Step D: 6-[4-[[3-(methoxycarbonyl)phenoxy]methyl]piperidin-1-yl]pyridin-3-ylboronic acid

A flask was charged with methyl 3-[[1-(5-bromopyridin-2-yl)piperidin-4-yl]methoxy]benzoate (1.01 g, 2.50 mmol), bis(pinacolato)diboron (0.76 g, 3.0 mmol), 1,1′-bis(diphenylphosphino)ferrocenepalladium(II)dichloride dichloromethane (0.11 g, 0.13 mmol), potassium acetate (0.74 g, 7.50 mmol) and dioxane (10 mL), and the air was exchanged with nitrogen by pulling a vacuum then refilling with nitrogen for two cycles. The mixture was heated at 80° C. for 10 h then cooled, filtered, concentrated. Purification by Gilson HPLC to yield 6-[4-[[3-(methoxycarbonyl)phenoxy]methyl]piperidin-1-yl]pyridine-3-ylboronic acid as a white solid. LC/MS=371 [M+1].

### Step E: potassium 6-[4-[[3-(methoxycarbonyl)phenoxy]methyl]piperidin-1-yl]pyridin-3-yltrifluoroborate

To a mixture of 6-[4-[[3-(methoxycarbonyl)phenoxy]methyl]piperidin-1-yl]pyridin-3-ylboronic acid (0.54 g, 1.46 mmol) in MeOH/H₂O (1:2, 2.1 mL) in a plastic vial was added potassium hydrogen fluoride then stirred vigorously at room temperature for 2 h. The mixture was cooled in an ice/water bath for 1 h, filtered, washed with cooled MeOH/H₂O (3:1, 5.0 mL) and dried under vacuum to yield intermediate 1 as a white solid. LC/MS=432 [M+39].

### Intermediate 101

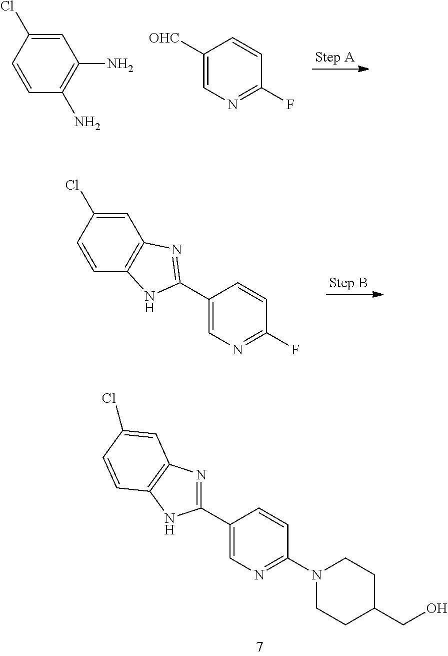 [SMILES: Nc1ccc(Cl)cc1N ; O=Cc1ccc(F)nc1 ; Fc1ccc(-c2nc3cc(Cl)ccc3[nH]2)cn1 ; OCC1CCN(c2ccc(-c3nc4cc(Cl)ccc4[nH]3)cn2)CC1]

### Step A: 5-chloro-2-(6-fluoropyridin-3-yl)-1H-benzo[d]imidazole

To a reaction flask with p-chloro-o-phenylenediamine (5.0 g, 0.04 mol) in N,N-dimethylformamide (30 mL) and water (1 mL) was added 6-fluoronicotinaldehyde (4.4 g, 0.035 mol) slowly. Oxone (14.0 g, 0.023 mol) was then added in one portion. The reaction was stirred at RT for 30 mins then poured onto water. Potassium carbonate (1 M in water, 30 mL) was added slowly. The reaction was filtered, and the solid was washed with water. The solid was dried under vacuum to give 5-chloro-2-(6-fluoropyridin-3-yl)-1H-benzo[d]imidazole as a brown solid. LC/MS=248 [M+1].

### Step B: (1-(5-(5-chloro-1H-benzo[d]imidazol-2-yl)pyridin-2-yl)piperidin-4-yl)methanol

To 4-(hydroxymethyl)piperidine (0.46 g, 4.0 mmol) in DMF (7 mL) was added 5-chloro-2-(6-fluoropyridin-3-yl)-1H-benzo[d]imidazole (0.98 g, 4.0 mmol) and N,N-diisopropylethylamine (1.26 mL, 7.2 mmol). The reaction mixture was heated at 100° C. for 5 h using an oil bath then cooled to RT and concentrated. Water (200 mL) was added, and the aqueous solution was extracted with CH₂Cl₂ (3×50 mL). The combined organic extract was dried (MgSO₄), filtered, and concentrated. The residue was purified by silica gel chromatography (eluant: 3:1 CH₂Cl₂:methanol) to obtain the product (1-(5-(5-chloro-1H-benzo[d]imidazol-2-yl)pyridin-2-yl)piperidin-4-yl)methanol as a brown solid. LC/MS=343 [M+1].

### Intermediate 102

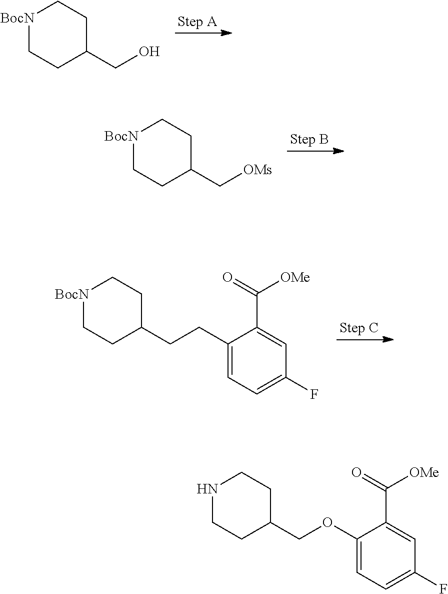 [SMILES: CC(C)(C)OC(=O)N1CCC(CO)CC1 ; CC(C)(C)OC(=O)N1CCC(COS(C)(=O)=O)CC1 ; COC(=O)c1cc(F)ccc1CCC1CCN(C(=O)OC(C)(C)C)CC1 ; COC(=O)c1cc(F)ccc1OCC1CCNCC1]

### Step A: N-Boc-4-methanesulfonyloxymethylpiperidine

To N-Boc-4-piperidinemethanol (10.8 g, 50 mmol) dissolved in CH₂Cl₂ (150 mL) and cooled to 0° C. was added diisopropylethylamine (10.7 mL, 60 mmol) and mesyl chloride (4.6 mL, 60 mmol). The reaction mixture was stirred at 0° C. for 15 mins then at RT overnight. Water (150 mL) was added, and the aqueous solution was extracted with CH₂Cl₂. The combined extracts were dried (MgSO₄), filtered, and concentrated. Purification by silica gel column chromatography to give N-Boc-4-methanesulfonyloxymethylpiperidine as a white solid.

### Step B: N-Boc-4-[[4-fluoro-2-(methoxycarbonyl)phenoxy]methyl]-piperidine

To a solution of methyl 5-fluoro-2-hydroxybenzoate (0.51 g, 3.0 mmol) in dry DMF (6 mL) under nitrogen was added sodium hydride (0.18 g of 60 wt % in mineral oil, 4.5 mmol). The mixture was stirred at RT for 15 mins then N-Boc-4-methanesulfonyloxymethylpiperidine (0.59 g, 2 mmol) was added. The resulting mixture was heated at 100° C. overnight then cooled and poured into 100 mL of water. The product was extracted with EtOAc (2×100 mL). The combined extracts were dried (MgSO₄), filtered, and concentrated. Purification by silica gel column chromatography (eluant: 0-40% EtOAc/hexane) to yield N-Boc-4-[[4-fluoro-2-(methoxycarbonyl)phenoxy]methyl]-piperidine as a white solid.

### Step C: 4-[[4-fluoro-2-(methoxycarbonyl)phenoxy]methyl]-piperidine

N-Boc-4-[[4-fluoro-2-(methoxycarbonyl)phenoxy]methyl]-piperidine (0.43 g) was treated with 10 mL of 4 N HCl in dioxane at RT for 4 h. The mixture was concentrated to give 4-[[4-fluoro-2-(methoxycarbonyl)phenoxy]methyl]-piperidine as the HCl salt.

### Intermediate 103

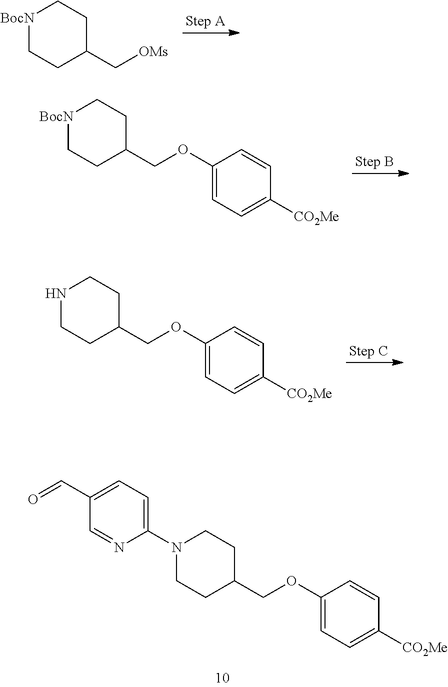

### Step A: N-Boc-4-[[4-(methoxycarbonyl)phenoxy]methyl]-piperidine

To a solution of methyl 4-hydroxybenzoate (0.46 g, 3.0 mmol) in dry DMF (6 mL) under nitrogen was added sodium hydride (0.18 g of 60 wt % in mineral oil, 4.5 mmol). The mixture was stirred at RT for 15 mins then N-Boc-4-methanesulfonyloxymethylpiperidine (0.59 g, 2 mmol) was added. The resulting mixture was heated at 100° C. overnight then cooled and poured into 100 mL of water. Product was extracted with EtOAc (2×100 mL). The combined extracts were dried (MgSO₄), filtered, and concentrated. Purification by silica gel column chromatography (eluant: 0-60% EtOAc/hexane) to yield N-Boc-4-[[4-(methoxycarbonyl)phenoxy]methyl]-piperidine as a white solid.

### Step B: 4-[[4-(methoxycarbonyl)phenoxy]methyl]-piperidine

N-Boc-4-[[4-(methoxycarbonyl)phenoxy]methyl]-piperidine (0.45 g) was treated with 10 mL of 4 N HCl in dioxane at RT for 4 h. The mixture was concentrated to give 4-[[2-(methoxycarbonyl)phenoxy]methyl]-piperidine as the HCl salt (100%).

### Step C: methyl 4-[[1-[5-formyl-2-pyridinyl]-piperidin-4-yl]methoxy]-benzoate

4-[[4-(methoxycarbonyl)phenoxy]methyl]-piperidine (HCl salt, 120 mg. 0.4 mmol) was mixed with 2-fluoro-5-formylpyridine (52 mg, 0.42 mmol) and diisopropylethylamine (0.15 mL, 0.84 mmol) in 2 mL of DMF. Mixture was heated to 150° C. for 30 mins by a microwave reactor. The mixture was used in the next step without further purification.

The following Intermediates were prepared by using method described for Intermediate 103.

| Intermediate | Structure | LC-MS |
|---|---|---|
| 104 | 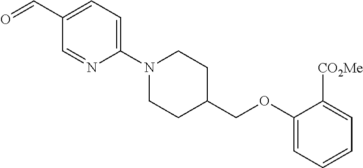 [SMILES: COC(=O)c1ccccc1OCC1CCN(c2ccc(C=O)cn2)CC1] | 348 [M + 1]. |
| 105 | 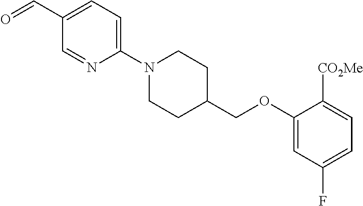 [SMILES: COC(=O)c1ccc(F)cc1OCC1CCN(c2ccc(C=O)cn2)CC1] | 373 [M + 1]. |
| 106 | 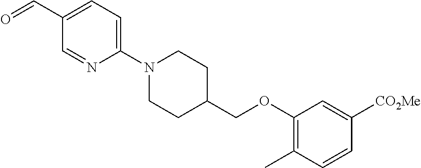 [SMILES: COC(=O)c1ccc(C)c(OCC2CCN(c3ccc(C=O)cn3)CC2)c1] | 369 [M + 1]. |
| 107 | 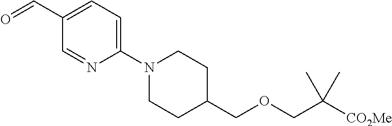 [SMILES: COC(=O)C(C)(C)COCC1CCN(c2ccc(C=O)cn2)CC1] | 335 [M + 1]. |
| 108 | 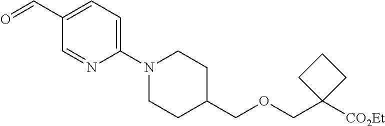 [SMILES: CCOC(=O)C1(COCC2CCN(c3ccc(C=O)cn3)CC2)CCC1] | 361 [M + 1]. |

### Intermediate 109

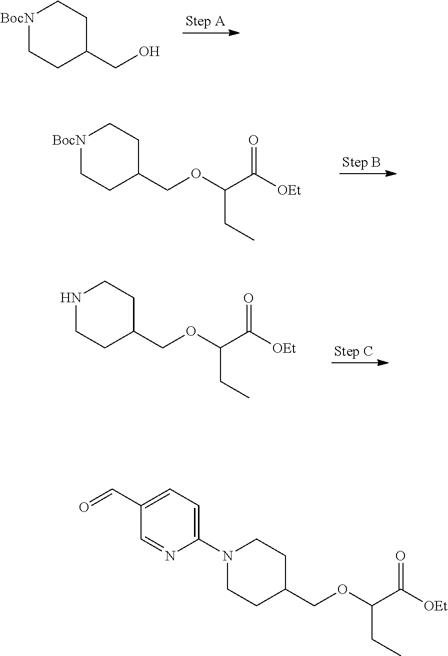 [SMILES: CC(C)(C)OC(=O)N1CCC(CO)CC1 ; CCOC(=O)C(CC)OCC1CCN(C(=O)OC(C)(C)C)CC1 ; CCOC(=O)C(CC)OCC1CCNCC1 ; CCOC(=O)C(CC)OCC1CCN(c2ccc(C=O)cn2)CC1]

### Step A: 2-[(N-Boc-piperidin-4-yl)methoxy]-butyric acid ethyl ester

To N-Boc-4-piperidinemethanol (0.43 g, 2 mmol) and 2-bromobutyric acid ethyl ester (0.3 mL, 2.1 mmol) in 5 mL of dry DMF was added NaH (88 mg of 60% in oil, 2.2 mmol). The mixture was heated to 100° C. overnight. After cooling to RT, the mixture was poured into 150 mL of water, and the product was extracted with ethyl acetate. The combined organic extracts were washed with brine, dried (Na₂SO₄), and concentrated. Purification by silica gel column chromatography to give 2-[(N-Boc-piperidin-4-yl)methoxy]-butyric acid ethyl ester as an oil (0.18 g).

### Step B: 2-[(piperidin-4-yl)methoxy]-butyric acid ethyl ester

2-[(N-Boc-piperidin-4-yl)methoxy]-butyric acid ethyl ester (0.15 g) was treated with 8 mL of 4 N HCl in dioxane at RT for 4 h. The mixture was concentrated to give 2-[(piperidin-4-yl)methoxy]-butyric acid ethyl ester as the HCl salt.

### Step C: ethyl 2-[[1-[5-formyl-2-pyridinyl]-piperidin-4-yl]methoxy]-butyrate

2-[(Piperidin-4-yl)methoxy]-butyric acid ethyl ester (HCl salt, 110 mg. 0.4 mmol) was mixed with 2-fluoro-5-formylpyridine (52 mg, 0.42 mmol) and diisopropylethylamine (0.15 mL, 0.84 mmol) in 2 mL of DMF. The mixture was heated to 70° C. overnight and used in the next step without further purification.

### Example 1

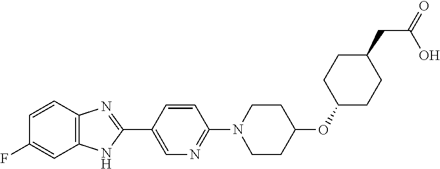 [SMILES: O=C(O)C[C@H]1CC[C@H](OC2CCN(c3ccc(-c4nc5ccc(F)cc5[nH]4)cn3)CC2)CC1]

### [trans-4-({1-[5-(6-fluoro-1H-benzimidazol-2-yl)pyridin-2-yl]piperidin-4-yl}oxy)cyclohexyl]acetic acid

Methyl [trans-4-({1-[5-(6-fluoro-1H-benzimidazol-2-yl)pyridin-2-yl]piperidin-4-yl}oxy)cyclohexyl]acetate (100 mg, 0.2146 mmol) was treated with 18.5% hydrochloric acid (2.7 ml, 16.5 mmol). The reaction mixture was heated at 95° C. in an oil bath for 30 min, and then concentrated under vacuum. The residue was purified by Gilson (acetonitrile (0.05% TFA)/water (0.05% TFA) 20-100%). This resulted in 35 mg (58%) of the TFA salt of [trans-4-({1-[5-(6-fluoro-1H-benzimidazol-2-yl)pyridin-2-yl]piperidin-4-yl}oxy)cyclohexyl]acetic acid as a white solid. LC-MS (ES, m/z) C₂₅H₂₉FN₄O₃: 452; Found: 453 [M+H]⁺.

Alternatively, methyl [trans-4-({1-[5-(6-fluoro-1H-benzimidazol-2-yl)pyridin-2-yl]piperidin-4-yl}oxy)cyclohexyl]acetate (5.45 g, 11.68 mmol) was slurred in 2:1 MeOH/THF (150 ml) and 2 M NaOH (aq.) (29.2 ml, 58.4 mmol) was added. The reaction mixture was heated at 65° C. for 1 hour, then concentrated under vacuum and re-dissolved in water (100 ml). The mixture was neutralized to pH=6.5-7 with 6 N HCl (8.76 ml, 52.6 mmol), filtered and washed with 2×20 ml water and the solid was dried under vacuum with N₂ sweep for 2 days. This resulted in [trans-4-({1-[5-(6-fluoro-1H-benzimidazol-2-yl)pyridin-2-yl]piperidin-4-yl}oxy)cyclohexyl]acetic acid as white solid. LC-MS (ES, m/z) C₂₅H₂₉FN₄O₃: 452; Found: 453 [M+H]⁺.

### Example 2

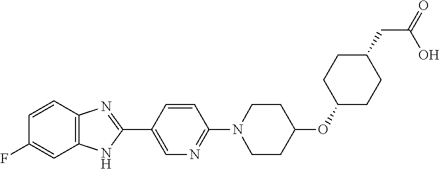 [SMILES: O=C(O)C[C@H]1CC[C@@H](OC2CCN(c3ccc(-c4nc5ccc(F)cc5[nH]4)cn3)CC2)CC1]

### [cis-4-({1-[5-(6-fluoro-1H-benzimidazol-2-yl)pyridin-2-yl]piperidin-4-yl}oxy)cyclohexyl]acetic acid

A mixture of methyl [cis-4-({1-[5-(6-fluoro-1H-benzimidazol-2-yl)pyridin-2-yl]piperidin-4-yl}oxy)cyclohexyl]acetate (80 mg, 0.171 mmol) and lithium hydroxide (28.7 mg, 1.2 mmol) in THF (2 ml) and water (1 ml) was stirred at room temperature over night then concentrated under vacuum. The crude material was purified by Gilson on reverse HPLC (acetonitrile (0.05% TFA)/water (0.05% TFA) 20-100%). This resulted in 48 mg (49.4%) of the TFA salt of [cis-4-({1-[5-(6-fluoro-1H-benzimidazol-2-yl)pyridin-2-yl]piperidin-4-yl}oxy)cyclohexyl]acetic acid as a white solid. LC-MS (ES, m/z) C₂₅H₂₉FN₄O₃: 452; Found: 453 [M+H]⁺.

### Example 3

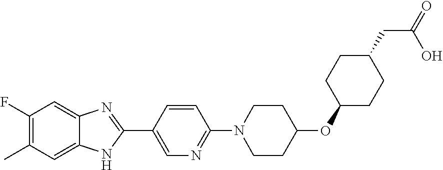 [SMILES: Cc1cc2[nH]c(-c3ccc(N4CCC(O[C@H]5CC[C@H](CC(=O)O)CC5)CC4)nc3)nc2cc1F]

### [trans-4-({1-[5-(5-fluoro-6-methyl-1H-benzimidazol-2-yl)pyridin-2-yl]piperidin-4-yl}oxy)cyclohexyl]acetic acid

A mixture of 4-fluoro-5-methylbenzene-1,2-diamine (40 mg, 0.285 mmol), (trans-4-{[1-(5-formylpyridin-2-yl)piperidin-4-yl]oxy}cyclohexyl)acetic acid (99 mg, 0.285 mmol) and potassium peroxymonosulfate (114 mg, 0.186 mmol) in DMF (2 ml) and water (0.2 ml) was stirred for 40 mins at room temperature. Then poured into 1M K₂CO₃ (5 ml), extracted with 3×10 ml ethyl acetate. The organic layers were combined, washed with 2×5 mL of saturated brine, dried over anhydrous sodium sulfate and concentrated under vacuum. Purified by Gilson, acetonitrile (0.1% TFA)/water (0.1% TFA) 25-55%. This resulted in the TFA salt of [trans-4-({1-[5-(5-fluoro-6-methyl-1H-benzimidazol-2-yl)pyridin-2-yl]piperidin-4-yl}oxy)cyclohexyl]acetic acid as a white solid. LC-MS (ES, m/z) C₂₆H₃₁FN₄O₃: 466; Found: 467 [M+H]⁺.

### Example 4

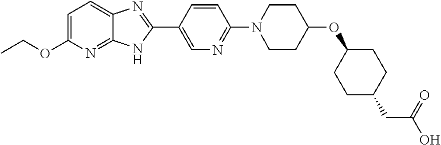 [SMILES: CCOc1ccc2nc(-c3ccc(N4CCC(O[C@H]5CC[C@H](CC(=O)O)CC5)CC4)nc3)[nH]c2n1]

### [trans-4-({1-[5-(5-ethoxy-3H-imidazo[4,5-b]pyridin-2-yl)pyridin-2-yl]piperidin-4-yl}oxy)cyclohexyl]acetic acid

### Step 1

A mixture of 6-ethoxypyridine-2,3-diamine (120 mg, 0.783 mmol), methyl (trans-4-{[1-(5-formylpyridin-2-yl)piperidin-4-yl]oxy}cyclohexyl)acetate (282 mg, 0.783 mmol) and potassium peroxymonosulfate (313 mg, 0.509 mmol) in DMF (2 ml) and water (0.2 ml) was stirred for 40 mins at room temperature. Then poured into 1M K₂CO₃ (5 ml), extracted with 3×10 ml ethyl acetate. The organic layers were combined, washed with 2×5 ml of saturated brine, dried over anhydrous sodium sulfate and concentrated under vacuum. The residue was applied onto a silica gel column and eluted with ethyl acetate/hexane 15-100%. This resulted in methyl [trans-4-({1-[5-(5-ethoxy-3H-imidazo[4,5-b]pyridin-2-yl)pyridin-2-yl]piperidin-4-yl}oxy)cyclohexyl]acetate as a white solid. LC-MS (ES, m/z) C₂₇H₃₅N₅O₄: 493; Found: 494 [M+H]⁺.

### Step 2

A mixture of methyl [trans-4-({1-[5-(5-ethoxy-3H-imidazo[4,5-b]pyridin-2-yl)pyridin-2-yl]piperidin-4-yl}oxy)cyclohexyl]acetate (0.22 g, 0.446 mmol) and lithium hydroxide (0.075 g, 3.12 mmol) in THF (3 ml) and water (1 ml) was stirred at room temperature over night then concentrated under vacuum. The residue was purified by Gilson reverse HPLC (acetonitrile (0.05% TFA)/water (0.05% TFA) 20-100%). This resulted in TFA salt of [trans-4-({1-[5-(5-ethoxy-3H-imidazo[4,5-b]pyridin-2-yl)pyridin-2-yl]piperidin-4-yl}oxy)cyclohexyl]acetic acid as a white solid. LC-MS (ES, m/z) C₂₆H₃₃N₅O₄: 479; Found: 480 [M+H]⁺.

### Examples 5-15

Synthesized following the procedures described above using appropriate starting materials.

| Example | Structure | [MH]⁺ m/z found |
|---|---|---|
| 5 | 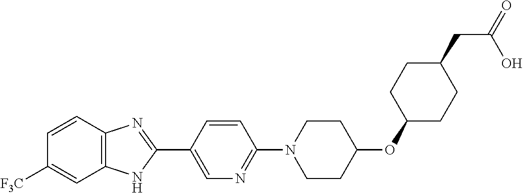 [SMILES: O=C(O)C[C@H]1CC[C@@H](OC2CCN(c3ccc(-c4nc5ccc(C(F)(F)F)cc5[nH]4)cn3)CC2)CC1] | 503 |
| 6 | 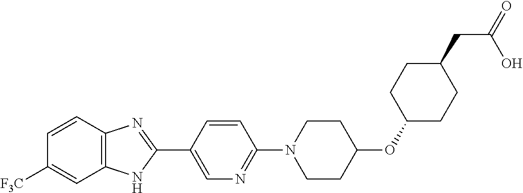 [SMILES: O=C(O)C[C@H]1CC[C@H](OC2CCN(c3ccc(-c4nc5ccc(C(F)(F)F)cc5[nH]4)cn3)CC2)CC1] | 503 |
| 7 | 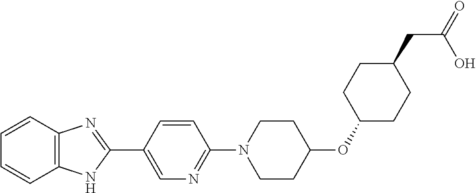 [SMILES: O=C(O)C[C@H]1CC[C@H](OC2CCN(c3ccc(-c4nc5ccccc5[nH]4)cn3)CC2)CC1] | 435 |
| 8 | 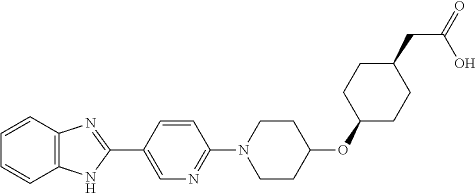 [SMILES: O=C(O)C[C@H]1CC[C@@H](OC2CCN(c3ccc(-c4nc5ccccc5[nH]4)cn3)CC2)CC1] | 435 |
| 9 | 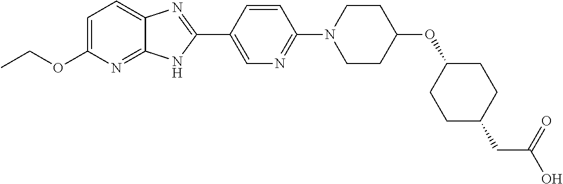 [SMILES: CCOc1ccc2nc(-c3ccc(N4CCC(O[C@H]5CC[C@@H](CC(=O)O)CC5)CC4)nc3)[nH]c2n1] | 480 |
| 10 | 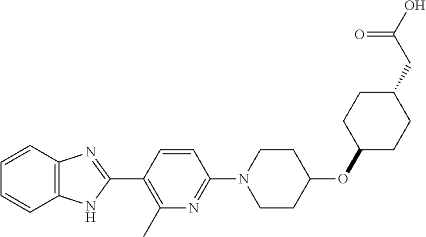 | 449 |
| 11 | 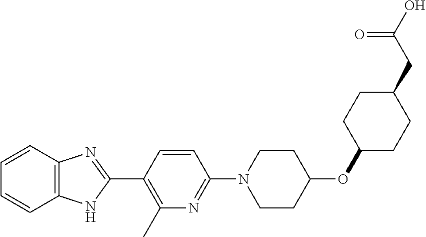 | 449 |
| 12 | 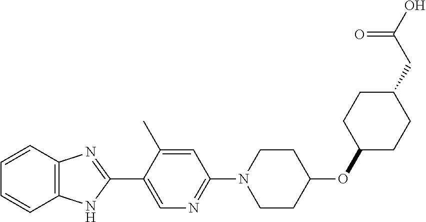 | 449 |
| 13 | 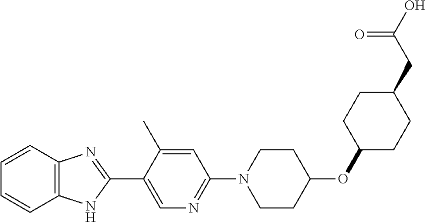 | 449 |
| 14 | 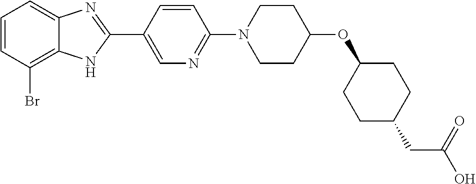 [SMILES: O=C(O)C[C@H]1CC[C@H](OC2CCN(c3ccc(-c4nc5cccc(Br)c5[nH]4)cn3)CC2)CC1] | 515 |
| 15 | 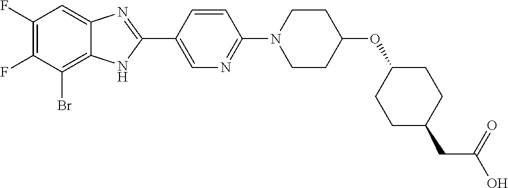 [SMILES: O=C(O)C[C@H]1CC[C@H](OC2CCN(c3ccc(-c4nc5cc(F)c(F)c(Br)c5[nH]4)cn3)CC2)CC1] | 551 |

### Example 16

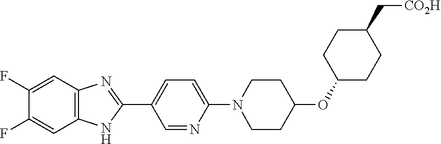 [SMILES: O=C(O)C[C@H]1CC[C@H](OC2CCN(c3ccc(-c4nc5cc(F)c(F)cc5[nH]4)cn3)CC2)CC1]

### [trans-4-({1-[5-(5,6-difluoro-1H-benzimidazol-2-yl)pyridin-2-yl]piperidin-4-yl}oxy)cyclohexyl]acetic acid

### Method A:

### Step 1

To a mixture of methyl (trans-4-{[1-(5-formylpyridin-2-yl)piperidin-4-yl]oxy}cyclohexyl)acetate (35 mg, 0.097 mmol) and 4,5-difluorobenzene-1,2-diamine (28 mg, 0.194 mmol) in 3% HOAc/DMF (1.5 mL) was added Oxone (59.6 mg, 0.097 mmol). The reaction was stirred at 80° C. for 16 hours. LC-MS showed that the reaction was completed. The solution was neutralized with solid K₂CO₃ and was extracted between EtOAc (4 mL×2) and water (2 mL). The organic phase was combined and evaporated.

### Step 2

The residue obtained from Step 1 was dissolved in MeOH/THF (1:1, 2 mL). LiOH/H₂O (2.5 M, 1 mL) was added and the reaction was stirred at ambient temperature for 3 hours. LC-MS showed that the hydrolysis was completed. The solvent was evaporated and 0.1 mL HOAc was added. The residue was extracted between EtOAc (4 mL×2) and water (2 mL). The organic phase was combined and concentrated. The crude product was purified by using reversed-phase HPLC to give [trans-4-({1-[5-(5,6-difluoro-1H-benzimidazol-2-yl)pyridin-2-yl]piperidin-4-yl}oxy)cyclohexyl]acetic acid as a TFA salt. LC-MS (ES, m/z) C₂₅H₂₈F₂N₄O₃: 470; Found: 471 [M+H]⁺.

### Method B:

Alternatively, [trans-4-({1-[5-(5,6-difluoro-1H-benzimidazol-2-yl)pyridin-2-yl]piperidin-4-yl}oxy)cyclohexyl]acetic acid was prepared following the procedure described for [trans-4-({1-[5-(6-fluoro-1H-benzimidazol-2-yl)pyridin-2-yl]piperidin-4-yl}oxy)cyclohexyl]acetic acid except that 5,6-difluoro-2-(6-fluoropyridin-3-yl)-1H-benzimidazole and methyl [trans-4-(piperidin-4-yloxy)cyclohexyl]acetate were used as the starting material.

### Examples 17-23

Synthesized using the aldehyde intermediate methyl (trans-4-{[1-(5-formylpyridin-2-yl)piperidin-4-yl]oxy}cyclohexyl)acetate and appropriate diamines, following Method A described for [trans-4-({1-[5-(5,6-difluoro-1H-benzimidazol-2-yl)pyridin-2-yl]piperidin-4-yl}oxy)cyclohexyl]acetic acid.

| Example | Structure | [MH]⁺ m/z found |
|---|---|---|
| 17 | 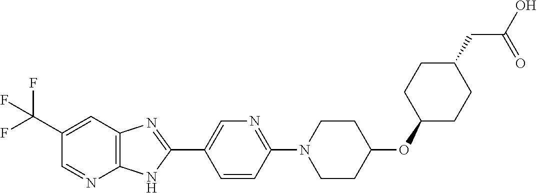 [SMILES: O=C(O)C[C@H]1CC[C@H](OC2CCN(c3ccc(-c4nc5cc(C(F)(F)F)cnc5[nH]4)cn3)CC2)CC1] | Found: 504 [M + H]⁺ |
| 18 | 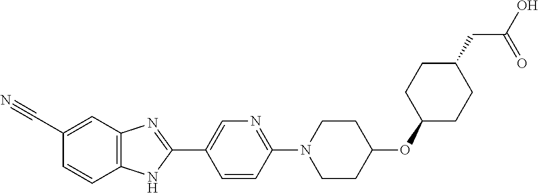 [SMILES: N#Cc1ccc2[nH]c(-c3ccc(N4CCC(O[C@H]5CC[C@H](CC(=O)O)CC5)CC4)nc3)nc2c1] | Found: 460 [M + H]⁺ |
| 19 | 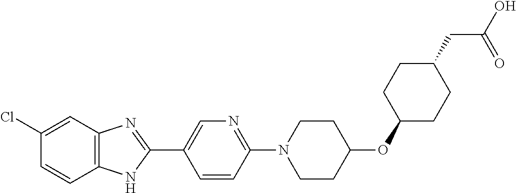 [SMILES: O=C(O)C[C@H]1CC[C@H](OC2CCN(c3ccc(-c4nc5cc(Cl)ccc5[nH]4)cn3)CC2)CC1] | Found: 469 [M + H]⁺ |
| 20 | 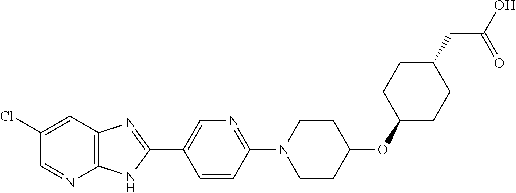 [SMILES: O=C(O)C[C@H]1CC[C@H](OC2CCN(c3ccc(-c4nc5cc(Cl)cnc5[nH]4)cn3)CC2)CC1] | Found: 470 [M + H]⁺ |
| 21 | 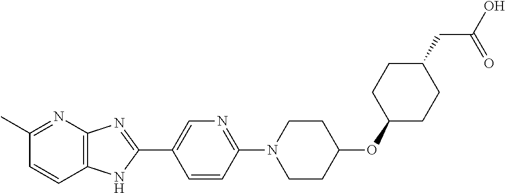 [SMILES: Cc1ccc2[nH]c(-c3ccc(N4CCC(O[C@H]5CC[C@H](CC(=O)O)CC5)CC4)nc3)nc2n1] | Found: 450 [M + H]⁺ |
| 22 | 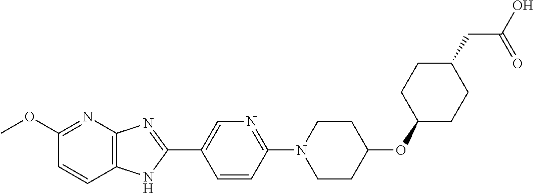 [SMILES: COc1ccc2[nH]c(-c3ccc(N4CCC(O[C@H]5CC[C@H](CC(=O)O)CC5)CC4)nc3)nc2n1] | Found: 466 [M + H]⁺ |
| 23 | 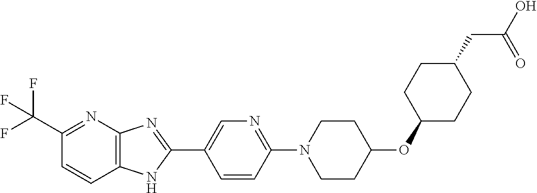 [SMILES: O=C(O)C[C@H]1CC[C@H](OC2CCN(c3ccc(-c4nc5nc(C(F)(F)F)ccc5[nH]4)cn3)CC2)CC1] | Found: 504 [M + H]⁺ |

### Example 24

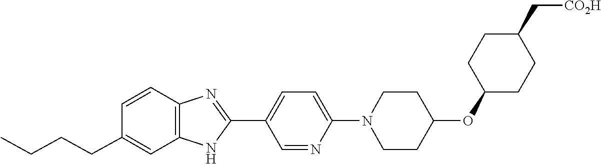 [SMILES: CCCCc1ccc2nc(-c3ccc(N4CCC(O[C@H]5CC[C@@H](CC(=O)O)CC5)CC4)nc3)[nH]c2c1]

### [cis-4-({1-[5-(6-butyl-1H-benzimidazol-2-yl)pyridin-2-yl]piperidin-4-yl}oxy)cyclohexyl]acetic acid

Following Step 1 of Method A described for the preparation of [trans-4-({1-[5-(5,6-difluoro-1H-benzimidazol-2-yl)pyridin-2-yl]piperidin-4-yl}oxy)cyclohexyl]acetic acid, starting from (cis-4-{[1-(5-formylpyridin-2-yl)piperidin-4-yl]oxy}cyclohexyl)acetic acid (35 mg, 0.101 mmol), [cis-4-({1-[5-(6-butyl-1H-benzimidazol-2-yl)pyridin-2-yl]piperidin-4-yl}oxy)cyclohexyl]acetic acid was afforded as the TFA salt after RP HPLC purification. LC-MS (ES, m/z) C₂₉H₃₈N₄O₃: 490; Found: 491 [M+H]⁺.

### Examples 25-27

Synthesized following the same procedure described for [cis-4-({1-[5-(6-butyl-1H-benzimidazol-2-yl)pyridin-2-yl]piperidin-4-yl}oxy)cyclohexyl]acetic acid.

| Example | Structure | [MH]⁺ m/z found |
|---|---|---|
| 25 | 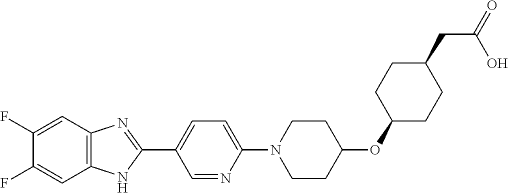 [SMILES: O=C(O)C[C@H]1CC[C@@H](OC2CCN(c3ccc(-c4nc5cc(F)c(F)cc5[nH]4)cn3)CC2)CC1] | Found: 471 [M + H]⁺ |
| 26 | 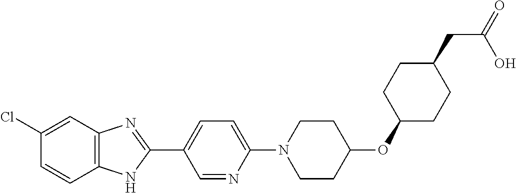 [SMILES: O=C(O)C[C@H]1CC[C@@H](OC2CCN(c3ccc(-c4nc5cc(Cl)ccc5[nH]4)cn3)CC2)CC1] | Found: 469 [M + H]⁺ |
| 27 | 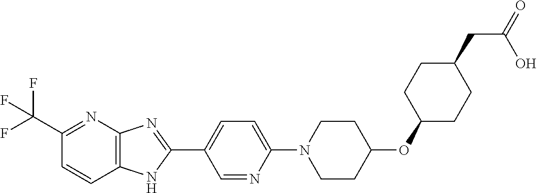 [SMILES: O=C(O)C[C@H]1CC[C@@H](OC2CCN(c3ccc(-c4nc5nc(C(F)(F)F)ccc5[nH]4)cn3)CC2)CC1] | Found: 504 [M + H]⁺ |

### Example 28

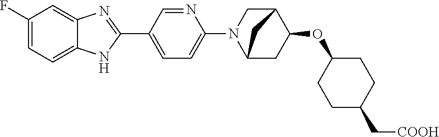 [SMILES: O=C(O)C[C@H]1CC[C@@H](O[C@H]2C[C@H]3C[C@@H]2CN3c2ccc(-c3nc4cc(F)ccc4[nH]3)cn2)CC1]

### [cis-4-({2-[5-(5-fluoro-1-benzimidazol-2-yl)pyridin-2-yl]-2-azabicyclo[2.2.1]hept-5-yl}oxy)cyclohexyl]acetic acid enantiomer 1

### Step 1

To a 100 mL one neck round bottom flask was charged with racemic tert-butyl 5-hydroxy-2-azabicyclo[2.2.1]heptane-2-carboxylate (2 g, 9.38 mmol) along with dioxane (15 mL). The mixture was stirred while a solution of HCl in dioxane (10 mL, 40.0 mmol) was added drop wise. The mixture was then stirred at room temperature for 1 hr. The reaction mixture was concentrated by rotary evaporation. The residue was then dissolved in dichloromethane (50 ml) along with TEA (2.85 g, 28.1 mmol). The mixture was stirred while benzyl carbonochloridate (1.616 g, 9.47 mmol) was added drop wise in 10 min. The resulting reaction mixture was stirred at room temperature for 2 hrs. The mixture was diluted with hexanes (50 mL) and the triethylamine chloride solid was filtered and washed with ethyl acetate/hexanes (1/1 50 mL). The filtrate was concentrated and the crude was purified by MPLC (40 g silica gel, 0 to 50% ethyl acetate in hexanes) to afford white solid product racemic benzyl 5-hydroxy-2-azabicyclo[2.2.1]heptane-2-carboxylate. LC-MS (ES, m/z) C₁₄H₁₇NO₃: 247; Found: 248 [M+H]⁺.

### Step 2

To a 100 mL one neck round bottom flask was charged with racemic benzyl 5-hydroxy-2-azabicyclo[2.2.1]heptane-2-carboxylate (0.42 g, 1.698 mmol) along with THF (15 mL) and TEA (0.189 g, 1.868 mmol). The mixture was cooled to 0° C. and TMSCl (0.194 g, 1.783 mmol) was added drop wise. After 30 min the mixture was diluted with hexanes and filtered through a small pad of celite eluting with hexane and concentrated. The mixture diluted with methylenechloride (30 mL) and concentrated. Then the crude product was dissolved in methylene chloride (20 mL) along with benzyl (4-oxocyclohexyl)acetate (0.402 g, 1.630 mmol), cooled to −65° C. followed by addition of triethylsilane (0.217 g, 1.868 mmol) and TMSOTf (0.189 g, 0.849 mmol). The mixture was stirred for 2 hrs and allowed to warm up to 0° C. in half hour. LC-MS showed complete reaction. The reaction was diluted by ethyl acetate and washed with aqueous ammonium chloride (sat, 30 mL). The organic layer was separated and the combined organic phases were washed with brined, dried over MgSO₄, filtered and concentrated. The product was separated by MPLC (80 g silica gel, 5 to 30% ethyl acetate in hexanes, 20 column volume) to afford two products racemic benzyl 5-({trans-4-[2-(benzyloxy)-2-oxoethyl]cyclohexyl}oxy)-2-azabicyclo[2.2.1]heptane-2-carboxylate and racemic benzyl 5-({cis-4-[2-(benzyloxy)-2-oxoethyl]cyclohexyl}oxy)-2-azabicyclo[2.2.1]heptane-2-carboxylate. LC-MS (ES, m/z) C₂₉H₃₅NO₅: 477; Found: 478 [M+H]⁺.

### Step 3

The racemic benzyl 5-({cis-4-[2-(benzyloxy)-2-oxoethyl]cyclohexyl}oxy)-2-azabicyclo[2.2.1]heptane-2-carboxylate was submitted to Chiral Resolution using the AD column, 4.6×250 mm, 40% MeOH/CO₂, 2.4 ml/min, 100 barr, 40° C. to afford two enantiomers benzyl 5-({cis-4-[2-(benzyloxy)-2-oxoethyl]cyclohexyl}oxy)-2-azabicyclo [2.2.1]heptane-2-carboxylate enantiomer 1 (RT=2.73 min) and benzyl 5-({cis-4-[2-(benzyloxy)-2-oxoethyl]cyclohexyl}oxy)-2-azabicyclo[2.2.1]heptane-2-carboxylate enantiomer 2 (RT=3.11 min). LC-MS (ES, m/z) C₂₉H₃₅NO₅: 477; Found: 478 [M+H]⁺.

### Step 4

To a 25 mL one neck round bottom flask was charged with benzyl 5-({cis-4-[2-(benzyloxy)-2-oxoethyl]cyclohexyl}oxy)-2-azabicyclo[2.2.1]heptane-2-carboxylate enantiomer 1 (68 mg, 0.142 mmol) along with palladium on carbon (58 mg, 0.055 mmol) and solvent Ethanol (3 ml). Water (0.3 ml). The flask was then connected to a hydrogen balloon through a 3-way joint. The system was then vacuumed and refilled with hydrogen three times and the reaction mixture was stirred under hydrogen atmosphere at room temperature for 1 hr. LC-MS showed complete hydrolysis of Cbz protection and benzyl ester. The catalyst was filtered and washed by ethanol (3×1 mL). The filtrate was concentrated to afford product {cis-4-[2-azabicyclo[2.2.1]hept-5-yloxy]cyclohexyl}acetic acid enantiomer 1. LC-MS (ES, m/z) C₁₄H₂₃NO₃: 253; Found: 254 [M+H]⁺.

### Step 5

To a 20 mL sample vial was charged with Intermediate 51 (35.1 mg, 0.152 mmol), {cis-4-[2-azabicyclo[2.2.1]hept-5-yloxy]cyclohexyl}acetic acid enantiomer 1 (35 mg, 0.138 mmol), sodium bicarbonate (58.0 mg, 0.691 mmol) and NMP (2 ml). The resulting reaction mixture was then stirred at 110° C. for 18 hrs overnight. LC-MS showed complete consumption of starting material and formation of desired product. The mixture was cooled and filtered through a syringe filter, washed with ethyl acetate (3×1 ml). The filtrate was then concentrated by rotary evaporation to remove all solvent. The residue was dissolved in DMSO/CH₃CN/H₂O (2:2:1, 4 mL) and purified by RP HPLC (YMC column, 20-80% acetonitrile in water) afford white solid TFA salt of [cis-4-({2-[5-(5-fluoro-1H-benzimidazol-2-yl)pyridin-2-yl]-2-azabicyclo[2.2.1]hept-5-yl}oxy)cyclohexyl]acetic acid enantiomer 1. LC-MS (ES, m/z) C₂₆H₂₉N₄O₃: 464; Found: 465 [M+H]⁺.

### Example 29

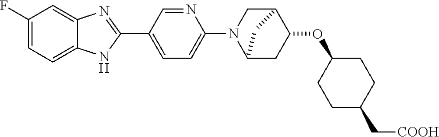

[cis-4-({2-[5-(5-fluoro-1H-benzimidazol-2-yl)pyridin-2-yl]-2-azabicyclo[2.2.1]hept-5-yl}oxy)cyclohexyl]acetic acid enantiomer 2

Prepared the same as the preceding example using {cis-4-[2-azabicyclo[2.2.1]hept-5-yloxy]cyclohexyl}acetic acid enantiomer 2 as the starting material to afford TFA salt of [cis-4-({2-[5-(5-fluoro-1H-benzimidazol-2-yl)pyridin-2-yl]-2-azabicyclo[2.2.1]hept-5-yl}oxy)cyclohexyl]acetic acid enantiomer 2. LC-MS (ES, m/z) C₂₆H₂₉N₄O₃: 464; Found: 465 [M+H]⁺.

### Example 30

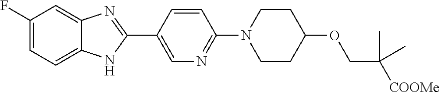 [SMILES: COC(=O)C(C)(C)COC1CCN(c2ccc(-c3nc4cc(F)ccc4[nH]3)cn2)CC1]

### methyl 3-({1-[5-(5-fluoro-1H-benzimidazol-2-yl)pyridin-2-yl]piperidin-4-yl}oxy)-2,2-dimethylpropanoate

To a solution of methyl 2,2-dimethyl-3-(piperidin-4-yloxy)propanoate (0.258 g, 1.2 mmol) in NMP (4 ml) was added 6-fluoro-2-(6-fluoropyridin-3-yl)-1H-benzimidazole (0.333 g, 1.440 mmol) was treated with sodium bicarbonate (2.02 g, 24.00 mmol) and heated at 110° C. overnight. The reaction mixture was added water, extracted with EtOAc, dried over Na₂SO₄, filtered and concentrated, separated by MPLC (10-100% EtOAc in hexane) to give methyl 3-({1-[5-(5-fluoro-1H-benzimidazol-2-yl)pyridin-2-yl]piperidin-4-yl}oxy)-2,2-dimethylpropanoate (0.17 g). LC-MS (ES, m/z) C₂₃H₂₇FN₄O₃: 426; Found: 427 [M+H]⁺.

### Example 31

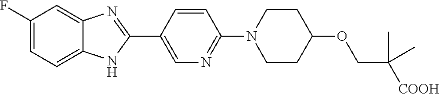 [SMILES: CC(C)(COC1CCN(c2ccc(-c3nc4cc(F)ccc4[nH]3)cn2)CC1)C(=O)O]

### 3-({1-[5-(5-fluoro-1H-benzimidazol-2-yl)pyridin-2-yl],piperidin-4-yl}oxy)-2,2-dimethylpropanoic acid

To a solution of methyl 3-({1-[5-(5-fluoro-1H-benzimidazol-2-yl)pyridin-2-yl]piperidin-4-yl}oxy)-2,2-dimethylpropanoate (0.170 g, 0.399 mmol) in THF/H₂O (4:1, 2.5 ml) was added Lithium hydroxide monohydrate (84 mg, 1.99 mmol). After stirred at 40° C. for over weekend, the organic solvent was removed. The aqueous was acidified with TFA, and concentrated. The residue was dissolved in DMSO (4 ml) filtered and diluted with DMSO/AcCN/H₂O (2:1:1, 4 ml), then purified by Gilson (25-100% AcCN in H₂O containing 0.05% TFA in 18 min linear, flow rate 30 ml/min, injection 1 ml). Desired fraction was collected and lyophilized to give TFA salt of 3-({1-[5-(5-fluoro-1H-benzimidazol-2-yl)pyridin-2-yl]piperidin-4-yl}oxy)-2,2-dimethylpropanoic acid (0.170 g) as white solid. LC-MS (ES, m/z) C₂₂H₂₆FN₄O₃: 412; Found: 413 [M+H]

### Example 32

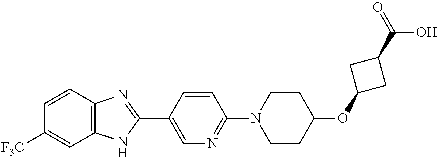 [SMILES: O=C(O)[C@H]1C[C@@H](OC2CCN(c3ccc(-c4nc5ccc(C(F)(F)F)cc5[nH]4)cn3)CC2)C1]

### cis-3-[(1-{5-[6-(trifluoromethyl)-1H-benzimidazol-2-yl]pyridin-2-yl}piperidin-4-yl)oxy]cyclobutanecarboxylic acid

A mixture of ethyl cis-3-[(1-{5-[6-(trifluoromethyl)-1H-benzimidazol-2-yl]pyridin-2-yl}piperidin-4-yl)oxy]cyclobutanecarboxylate (0.1 g, 0.205 mmol) and lithium hydroxide (0.034 g, 1.433 mmol) in THF (2 ml), methanol (2 ml) and water (1 ml). The reaction mixture stirred at room temperature over night then concentrated under vacuum. Then applied onto a YMC-pak ODS-AQ column (5u, 150×20 mm I.D). Mobile phase: A for water (0.005 mol/l ammonium acetate) and B for acetonitrile. Gradient: B 20%-50%. This resulted in 0.025 g (25.6%) of cis-3-[(1-{5-[6-(trifluoromethyl)-1H-benzimidazol-2-yl]pyridin-2-yl]piperidin-4-yl)oxy}cyclobutanecarboxylic acid as a white solid. LC-MS (ES, m/z) C₂₃H₂₃F₃N₄O₃: 460; Found: 461[M+H]⁺.

### Example 33

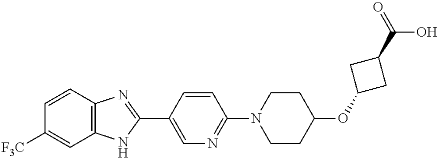 [SMILES: O=C(O)[C@H]1C[C@H](OC2CCN(c3ccc(-c4nc5ccc(C(F)(F)F)cc5[nH]4)cn3)CC2)C1]

### trans-3-[(1-{5-[6-(trifluoromethyl)-1H-benzimidazol-2-yl]pyridin-2-yl}piperidin-4-yl)oxy]cyclobutanecarboxylic acid

A mixture of ethyl trans-3-[(1-{5-[6-(trifluoromethyl)-1H-benzimidazol-2-yl]pyridin-2-yl}piperidin-4-yl)oxy]cyclobutanecarboxylate (0.1 g, 0.205 mmol) and lithium hydroxide (0.034 g, 1.433 mmol) in THF (2 ml), methanol (2 ml) and water (1 ml) was stirred at room temperature over night then concentrated under vacuum. The mixture was applied onto a YMC-pak ODS-AQ column (5u, 150×20 mm I.D). Mobile phase: A for water (0.005 mol/l ammonium acetate) and B for acetonitrile. Gradient: B 20%-50%. This resulted in 0.009 g (9.2%) of trans-3-[(1-{5-[6-(trifluoromethyl)-1H-benzimidazol-2-yl]pyridin-2-yl}piperidin-4-yl)oxy]cyclobutanecarboxylic acid as a white solid. LC-MS (ES, m/z) C₂₃H₂₃F₃N₄O₃: 460; Found: 461 [M+H]+.

### Examples 34-59

Synthesized following the procedure described above, starting with appropriate starting materials.

| | | [MH]⁺ m/z |
|---|---|---|
| Example | Structure | found |
| 34 | 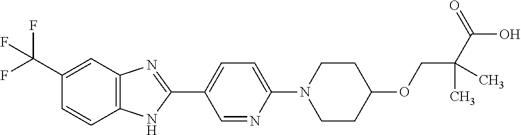 [SMILES: CC(C)(COC1CCN(c2ccc(-c3nc4cc(C(F)(F)F)ccc4[nH]3)cn2)CC1)C(=O)O] | Found: 463 [M + H]⁺ |
| 35 | 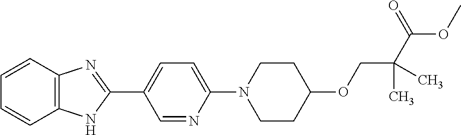 [SMILES: COC(=O)C(C)(C)COC1CCN(c2ccc(-c3nc4ccccc4[nH]3)cn2)CC1] | Found: 409 [M + H]⁺ |
| 36 | 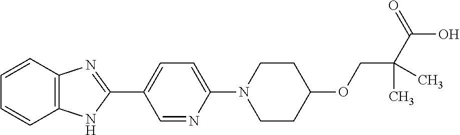 [SMILES: CC(C)(COC1CCN(c2ccc(-c3nc4ccccc4[nH]3)cn2)CC1)C(=O)O] | Found: 395 [M + H]⁺ |
| 37 | 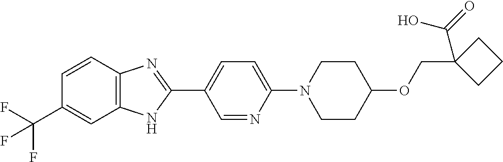 [SMILES: O=C(O)C1(COC2CCN(c3ccc(-c4nc5ccc(C(F)(F)F)cc5[nH]4)cn3)CC2)CCC1] | Found: 475 [M + H]⁺ |
| 38 | 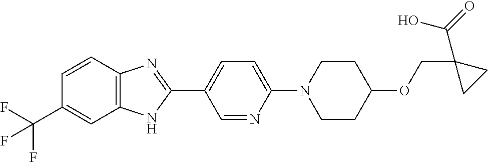 [SMILES: O=C(O)C1(COC2CCN(c3ccc(-c4nc5ccc(C(F)(F)F)cc5[nH]4)cn3)CC2)CC1] | Found: 461 [M + H]⁺ |
| 39 | 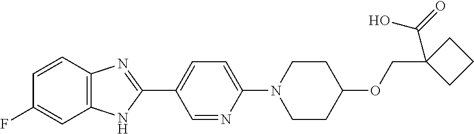 [SMILES: O=C(O)C1(COC2CCN(c3ccc(-c4nc5ccc(F)cc5[nH]4)cn3)CC2)CCC1] | Found: 425 [M + H]⁺ |
| 40 | 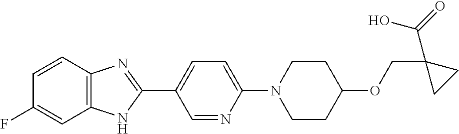 [SMILES: O=C(O)C1(COC2CCN(c3ccc(-c4nc5ccc(F)cc5[nH]4)cn3)CC2)CC1] | Found: 411 [M + H]⁺ |
| 41 | 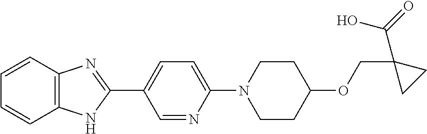 [SMILES: O=C(O)C1(COC2CCN(c3ccc(-c4nc5ccccc5[nH]4)cn3)CC2)CC1] | Found: 393 [M + H]⁺ |
| 42 | 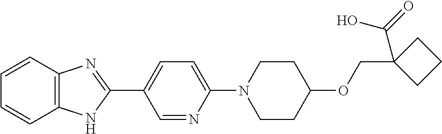 [SMILES: O=C(O)C1(COC2CCN(c3ccc(-c4nc5ccccc5[nH]4)cn3)CC2)CCC1] | Found: 407 [M + H]⁺ |
| 43 | 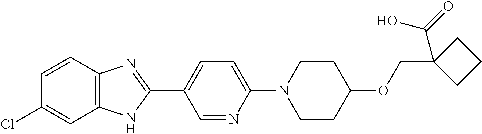 [SMILES: O=C(O)C1(COC2CCN(c3ccc(-c4nc5ccc(Cl)cc5[nH]4)cn3)CC2)CCC1] | Found: 441 [M + H]⁺ |
| 44 | 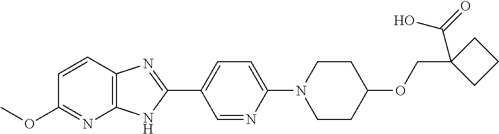 [SMILES: COc1ccc2nc(-c3ccc(N4CCC(OCC5(C(=O)O)CCC5)CC4)nc3)[nH]c2n1] | Found: 438 [M + H]⁺ |
| 45 | 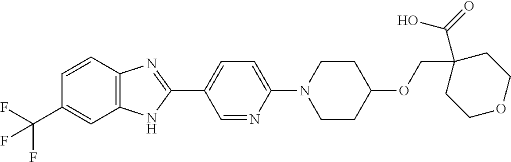 [SMILES: O=C(O)C1(COC2CCN(c3ccc(-c4nc5ccc(C(F)(F)F)cc5[nH]4)cn3)CC2)CCOCC1] | Found: 505 [M + H]⁺ |
| 46 | 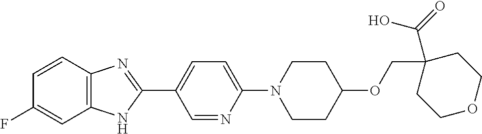 [SMILES: O=C(O)C1(COC2CCN(c3ccc(-c4nc5ccc(F)cc5[nH]4)cn3)CC2)CCOCC1] | Found: 455 [M + H]⁺ |
| 47 | 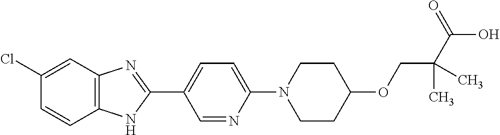 [SMILES: CC(C)(COC1CCN(c2ccc(-c3nc4cc(Cl)ccc4[nH]3)cn2)CC1)C(=O)O] | Found: 429 [M + H]⁺ |
| 48 | 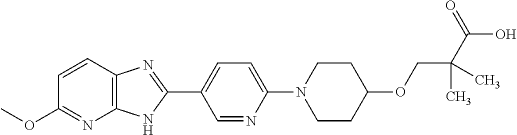 [SMILES: COc1ccc2nc(-c3ccc(N4CCC(OCC(C)(C)C(=O)O)CC4)nc3)[nH]c2n1] | Found: 426 [M + H]⁺ |
| 49 | 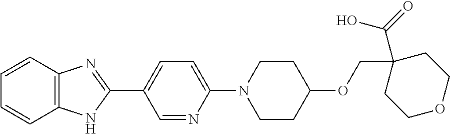 [SMILES: O=C(O)C1(COC2CCN(c3ccc(-c4nc5ccccc5[nH]4)cn3)CC2)CCOCC1] | Found: 437 [M + H]⁺ |
| 50 | 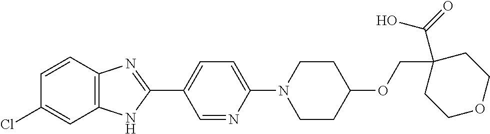 [SMILES: O=C(O)C1(COC2CCN(c3ccc(-c4nc5ccc(Cl)cc5[nH]4)cn3)CC2)CCOCC1] | Found: 471 [M + H]⁺ |
| 51 | 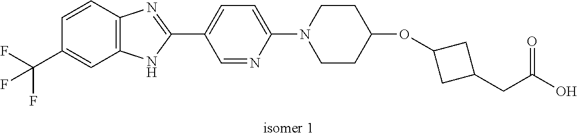 [SMILES: O=C(O)CC1CC(OC2CCN(c3ccc(-c4nc5ccc(C(F)(F)F)cc5[nH]4)cn3)CC2)C1] | Found: 475 [M + H]⁺ |
| 52 | 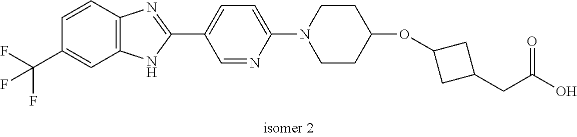 [SMILES: O=C(O)CC1CC(OC2CCN(c3ccc(-c4nc5ccc(C(F)(F)F)cc5[nH]4)cn3)CC2)C1] | Found: 475 [M + H]⁺ |
| 53 | 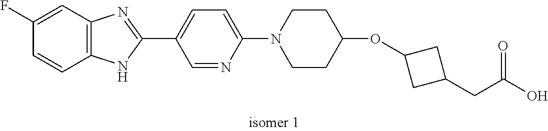 [SMILES: O=C(O)CC1CC(OC2CCN(c3ccc(-c4nc5cc(F)ccc5[nH]4)cn3)CC2)C1] | Found: 425 [M + H]⁺ |
| 54 | 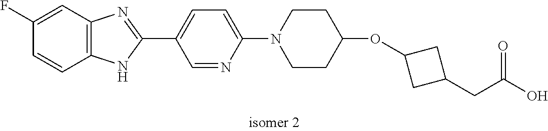 [SMILES: O=C(O)CC1CC(OC2CCN(c3ccc(-c4nc5cc(F)ccc5[nH]4)cn3)CC2)C1] | Found: 425 [M + H]⁺ |
| 55 | 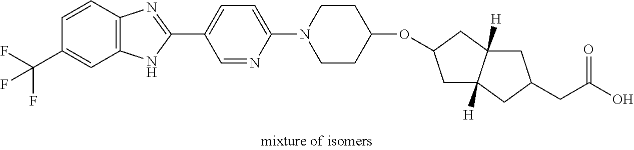 [SMILES: O=C(O)CC1C[C@@H]2CC(OC3CCN(c4ccc(-c5nc6ccc(C(F)(F)F)cc6[nH]5)cn4)CC3)C[C@@H]2C1] | Found: 529 [M + H]⁺ |
| 56 | 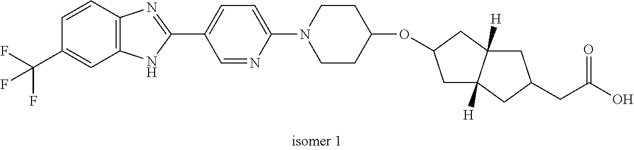 [SMILES: O=C(O)CC1C[C@@H]2CC(OC3CCN(c4ccc(-c5nc6ccc(C(F)(F)F)cc6[nH]5)cn4)CC3)C[C@@H]2C1] | Found: 529 [M + H]⁺ |
| 57 | 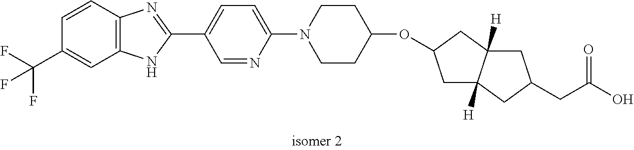 [SMILES: O=C(O)CC1C[C@@H]2CC(OC3CCN(c4ccc(-c5nc6ccc(C(F)(F)F)cc6[nH]5)cn4)CC3)C[C@@H]2C1] | Found: 529 [M + H]⁺ |
| 58 | 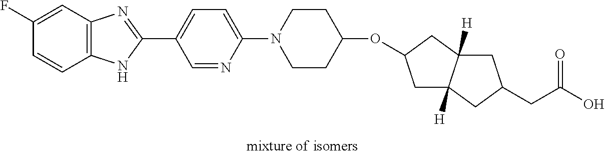 [SMILES: O=C(O)CC1C[C@@H]2CC(OC3CCN(c4ccc(-c5nc6cc(F)ccc6[nH]5)cn4)CC3)C[C@@H]2C1] | Found: 479 [M + H]⁺ |
| 59 | 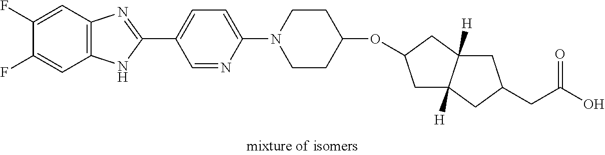 [SMILES: O=C(O)CC1C[C@@H]2CC(OC3CCN(c4ccc(-c5nc6cc(F)c(F)cc6[nH]5)cn4)CC3)C[C@@H]2C1] | Found: 497 [M + H]⁺ |

### Examples 60-67

Synthesized following the procedure described for 3-({1-[5-(5-fluoro-1H-benzimidazol-2-yl)pyridin-2-yl]piperidin-4-yl}oxy)-2,2-dimethylpropanoic acid, using the appropriate diamines and methyl 3-(1-(5-formylpyridin-2-yl)piperidin-4-yloxy)-2,2-dimethylpropanoate.

| | | [MH]⁺ m/z |
|---|---|---|
| Example | Structure | found |
| 60 | 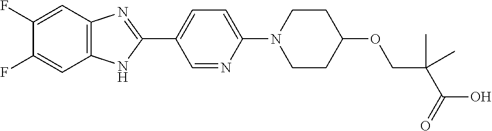 [SMILES: CC(C)(COC1CCN(c2ccc(-c3nc4cc(F)c(F)cc4[nH]3)cn2)CC1)C(=O)O] | Found: 431 [M + H]⁺ |
| 61 | 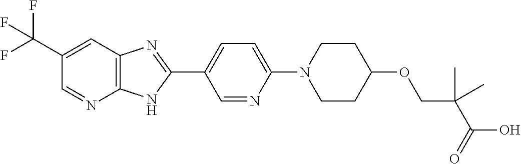 [SMILES: CC(C)(COC1CCN(c2ccc(-c3nc4cc(C(F)(F)F)cnc4[nH]3)cn2)CC1)C(=O)O] | Found: 464 [M + H]⁺ |
| 62 | 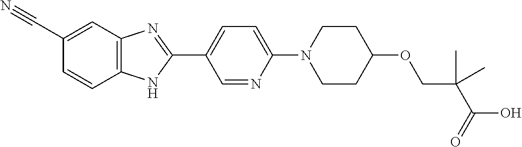 [SMILES: CC(C)(COC1CCN(c2ccc(-c3nc4cc(C#N)ccc4[nH]3)cn2)CC1)C(=O)O] | Found: 420 [M + H]⁺ |
| 63 | 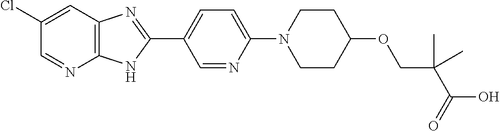 [SMILES: CC(C)(COC1CCN(c2ccc(-c3nc4cc(Cl)cnc4[nH]3)cn2)CC1)C(=O)O] | Found: 430 [M + H]⁺ |
| 64 | 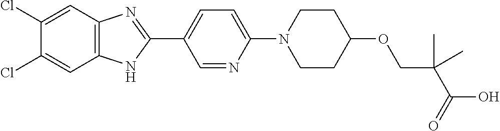 [SMILES: CC(C)(COC1CCN(c2ccc(-c3nc4cc(Cl)c(Cl)cc4[nH]3)cn2)CC1)C(=O)O] | Found: 463 [M + H]⁺ |
| 65 | 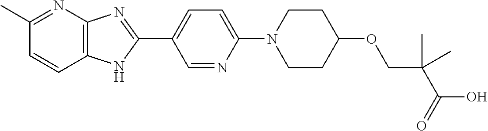 [SMILES: Cc1ccc2[nH]c(-c3ccc(N4CCC(OCC(C)(C)C(=O)O)CC4)nc3)nc2n1] | Found: 410 [M + H]⁺ |
| 66 | 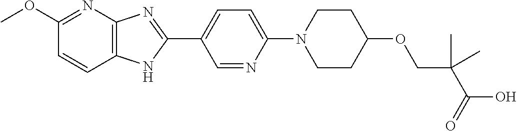 [SMILES: COc1ccc2[nH]c(-c3ccc(N4CCC(OCC(C)(C)C(=O)O)CC4)nc3)nc2n1] | Found: 426 [M + H]⁺ |
| 67 | 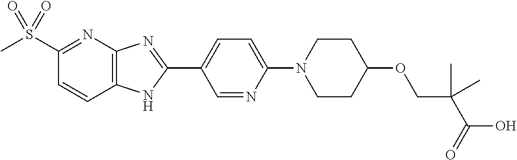 [SMILES: CC(C)(COC1CCN(c2ccc(-c3nc4nc(S(C)(=O)=O)ccc4[nH]3)cn2)CC1)C(=O)O] | Found: 473 [M + H]⁺ |

### Example 68

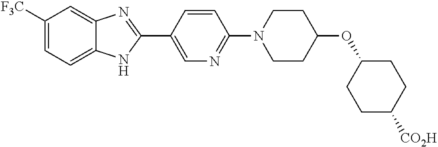 [SMILES: O=C(O)[C@H]1CC[C@@H](OC2CCN(c3ccc(-c4nc5cc(C(F)(F)F)ccc5[nH]4)cn3)CC2)CC1]

### cis-4-[(1-{5-[5-(trifluoromethyl)-1H-benzimidazol-2-yl]pyridin-2-yl}piperidin-4-yl)oxy]cyclohexanecarboxylic acid

### Step 1

To the solution of cis-ethyl 4-(piperidin-4-yloxy)cyclohexanecarboxylate (0.19 g, 0.744 mmol) in DMF (2.48 ml) was added 2-(6-fluoropyridin-3-yl)-5-(trifluoromethyl)-1H-benzimidazole (209 mg, 0.744 mmol) was treated with sodium bicarbonate (0.313 mg, 3.72 mmol) and heated at 110° C. overnight. The reaction mixture was added to water and lyophilized, separated by MPLC (80 g column, 0-20% Acetone in DCM to give ethyl cis-4-[(1-{5-[5-(trifluoromethyl)-1H-benzimidazol-2-yl]pyridin-2-yl}piperidin-4-yl)oxy]cyclohexanecarboxylate (0.070 g). LC-MS (ES, m/z) C₂₇H₃₁F₃N₄O₃: 516; Found: 517 [M+H]⁺.

### Step 2

To a solution of ethyl cis-4-[(1-{5-[5-(trifluoromethyl)-1H-benzimidazol-2-yl]pyridin-2-yl}piperidin-4-yl)oxy]cyclohexanecarboxylate (70 mg, 0.136 mmol) in THF/H₂O (4:1, 2.7 ml) was added Lithium hydroxide monohydrate (17.1 mg, 0.407 mmol). After stirring at 40° C. overnight, the organic solvent was removed. The aqueous was acidified with TFA, and concentrated. The residue was dissolved in DMSO/AcCN/H₂O (2:1:1, 4 ml), filtered with syringe-driven filter, and purified by Gilson (20-100% AcCN in H₂O containing 0.05% TFA in 18 min linear, flow rate 30 ml/min, injection 2 ml). The desired fraction was collected and lyophilized to give TFA salt of cis-4-[(1-{5-[5-(trifluoromethyl)-1H-benzimidazol-2-yl]pyridin-2-yl}piperidin-4-yl)oxy]cyclohexanecarboxylic acid (0.06 g) as white solid. LC-MS (ES, m/z) C₂₅H₂₇F₃N₄O₃: 488; Found: 489 [M+H]⁺.

### Example 69

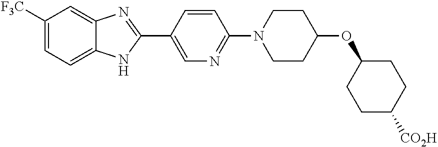 [SMILES: O=C(O)[C@H]1CC[C@H](OC2CCN(c3ccc(-c4nc5cc(C(F)(F)F)ccc5[nH]4)cn3)CC2)CC1]

### trans-4-[(1-{5-[5-(trifluoromethyl)-1H-benzimidazol-2-yl]pyridin-2-yl}piperidin-4-yl)oxy]cyclohexanecarboxylic acid

### Step 1

To the solution of ethyl trans-4-(piperidin-4-yloxy)cyclohexanecarboxylate (2.6 g, 10.2 mmol) in NMP (20 ml) was added 2-(6-fluoropyridin-3-yl)-5-(trifluoromethyl)-1H-benzimidazole (3.16 g, 11.22 mmol), sodium bicarbonate (17.14 g, 204 mmol) and heated at 110° C. overnight. The reaction mixture was added to water, extracted with EtOAc, washed with water and brine, dried over Na₂SO₄, filtered and concentrated, separated by MPLC (10-75% EtOAc in hexane) to give ethyl trans-4-[(1-{5-[5-(trifluoromethyl)-1H-benzimidazol-2-yl]pyridin-2-yl}piperidin-4-yl)oxy]cyclohexanecarboxylate (2.1 g). LC-MS (ES, m/z) C₂₇H₃₁F₃N₄O₃: 516; Found: 517 [M+H]⁺.

Alternatively, ethyl trans-4-[(1-{5-[5-(trifluoromethyl)-1H-benzimidazol-2-yl]pyridin-2-yl}piperidin-4-yl)oxy]cyclohexanecarboxylate was prepared from tert-butyl 4-(trans-4-(ethoxycarbonyl)cyclohexyloxy)piperidine-1-carboxylate:

To a solution of tert-butyl 4-(trans-4-(ethoxycarbonyl)cyclohexyloxy) piperidine-1-carboxylate (0.233 mg, 0.655 mmol) in DCM (5 ml) was added HCl (4 M in dioxane, 5 ml) and stirred at room temperature for 1 h. The reaction mixture was concentrated and the residue was dissolved in NMP (5 ml). To the mixture was added 2-(6-fluoropyridin-3-yl)-5-(trifluoromethyl)-1H-benzimidazole (0.203 g, 0.721 mmol) followed by sodium bicarbonate (1.10 g, 13.1 mmol). The mixture was heated at 110° C. over night. The reaction mixture was added to water, extracted with EtOAc, washed with water and brine, dried over Na₂SO₄, filtered and concentrated to afford a crude product which was separated by MPLC (10-75% EtOAc in hexane) to give ethyl trans-4-[(1-{5-[5-(trifluoromethyl)-1H-benzimidazol-2-yl]pyridin-2-yl}piperidin-4-yl)oxy]cyclohexanecarboxylate (0.170 g). LC-MS (ES, m/z) C₂₇H₃₁F₃N₄O₃: 516; Found: 517 [M+H]⁺.

### Step 2

To a solution of ethyl trans-4-[(1-{5-[5-(trifluoromethyl)-1H-benzimidazol-2-yl]pyridin-2-yl}piperidin-4-yl)oxy]cyclohexanecarboxylate (0.60 g, 1.162 mmol) in THF/H₂O (4:1, 12.5 ml) was added Lithium hydroxide monohydrate (146 mg, 3.48 mmol). After stirring at 40° C. over night, the organic solvent was removed. The aqueous was acidified with TFA, and concentrated. The residue was dissolved in DMSO (10 ml) filtered with syringe-driven filter and diluted with DMSO/AcCN/H₂O (2:1:1, 14 ml), then purified by Gilson (20-100% AcCN in H₂O containing 0.05% TFA in 18 min linear, flow rate 30 ml/min, injection 1.5 ml). The desired fraction was collected and lyophilized to give trans-4-[(1-{5-[5-(trifluoromethyl)-1H-benzimidazol-2-yl]pyridin-2-yl}piperidin-4-yl)oxy]cyclohexanecarboxylic acid as a white solid. LC-MS (ES, m/z) C₂₅H₂₇F₃N₄O₃: 488; Found: 489 [M+H]⁺.

### Example 70

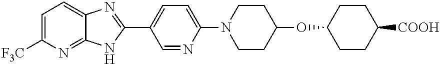 [SMILES: O=C(O)[C@H]1CC[C@H](OC2CCN(c3ccc(-c4nc5ccc(C(F)(F)F)nc5[nH]4)cn3)CC2)CC1]

### trans-4-[(1-{5-[5-(trifluoromethyl)-3H-imidazo[4,5-b]pyridin-2-yl]pyridin-2-yl}piperidin-4-yl)oxy]cyclohexanecarboxylic acid

### Step 1

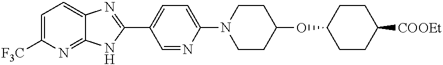 [SMILES: CCOC(=O)[C@H]1CC[C@H](OC2CCN(c3ccc(-c4nc5ccc(C(F)(F)F)nc5[nH]4)cn3)CC2)CC1]

6-Trifluoromethyl-pyridine-2,3-diamine-2HCl (72.8 mg, 0.29 mmol) in DMF/water (0.9 ml/0.03 ml) was added ethyl trans-4-{[1-(5-formylpyridin-2-yl)piperidin-4-yl]oxy}cyclohexanecarboxylate (100 mg, 0.277 mmol) and Oxone (111 mg, 0.18 mmol). The mixture was stirred at 50° C. for 16 hours. The mixture was poured into a 1 M K₂CO₃ (1.5 ml) in 10 ml water, and stirred for 10 minutes, then extracted with EtOAc (2×30 ml). The organic was dried over MgSO₄, filtered and concentrated. The residue was purified by preparative TLC (60% EtOAc/Hexane) to give ethyl trans-4-[(1-{5-[5-(trifluoromethyl)-3H-imidazo[4,5-b]pyridin-2-yl]pyridin-2-yl}piperidin-4-yl)oxy]cyclohexanecarboxylate as a solid. LC-MS (ES, m/z): C₂₆H₃₀F₃N₅O₃: 517; Found: 518 [M+H]⁺.

### Step 2

Ethyl trans-4-[(1-{5-[5-(trifluoromethyl)-3H-imidazo[4,5-b]pyridin-2-yl]pyridin-2-yl}piperidin-4-yl)oxy]cyclohexanecarboxylate (55 mg, 0.11 mmol) in THF/water (0.8 ml/0.2 ml) was added LiOH (15.2 mg, 0.63 mmol). The mixture was stirred at 40° C. for 12 hours. Concentrated in vacuum. The residue was purified by reverse HPLC to afford TFA salt of trans-4-[(1-{5-[5-(trifluoromethyl)-3H-imidazo[4,5-b]pyridin-2-yl]pyridin-2-yl}piperidin-4-yl)oxy]cyclohexanecarboxylic acid. LC-MS (ES, m/z): C₂₄H₂₆F₃N₅O₃: 489; Found: 490 [M+H]⁺.

### Examples 71-79

Synthesized following the procedures described above using appropriate starting materials.

| | | [MH]⁺ m/z |
|---|---|---|
| Example | Structure | found |
| 71 | 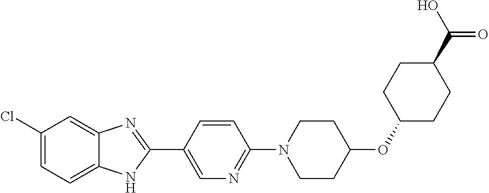 [SMILES: O=C(O)[C@H]1CC[C@H](OC2CCN(c3ccc(-c4nc5cc(Cl)ccc5[nH]4)cn3)CC2)CC1] | Found: 455 [M + H]⁺ |
| 72 | 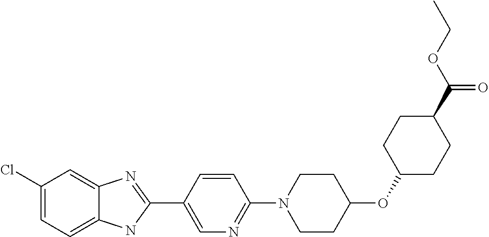 | Found: 483 [M + H]⁺ |
| 73 | 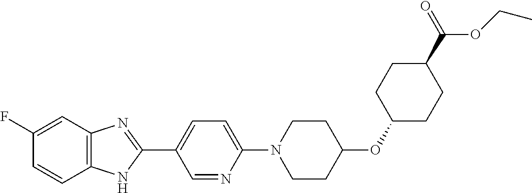 [SMILES: CCOC(=O)[C@H]1CC[C@H](OC2CCN(c3ccc(-c4nc5cc(F)ccc5[nH]4)cn3)CC2)CC1] | Found: 467 [M + H]⁺ |
| 74 | 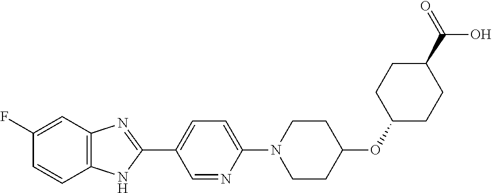 [SMILES: O=C(O)[C@H]1CC[C@H](OC2CCN(c3ccc(-c4nc5cc(F)ccc5[nH]4)cn3)CC2)CC1] | Found: 439 [M + H]⁺ |
| 75 | 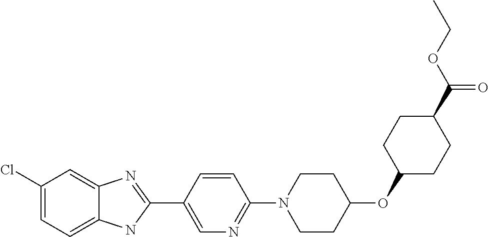 | Found: 483 [M + H]⁺ |
| 76 | 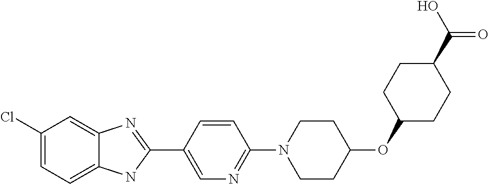 | Found: 455 [M + H]⁺ |
| 77 | 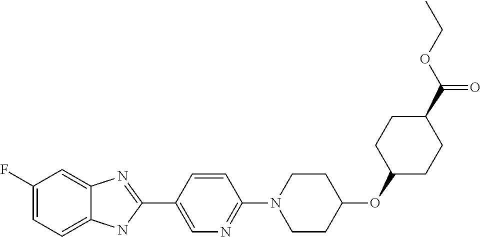 | Found: 467 [M + H]⁺ |
| 78 | 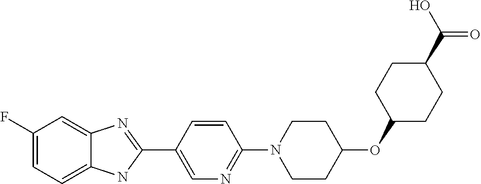 | Found: 439 [M + H]⁺ |
| 79 | 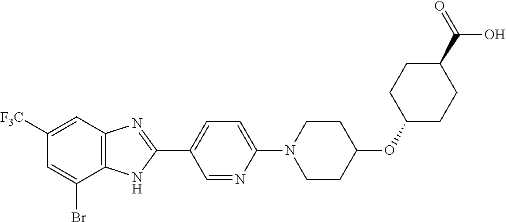 [SMILES: O=C(O)[C@H]1CC[C@H](OC2CCN(c3ccc(-c4nc5cc(C(F)(F)F)cc(Br)c5[nH]4)cn3)CC2)CC1] | Found: 569 [M + H]⁺ |

### Example 80

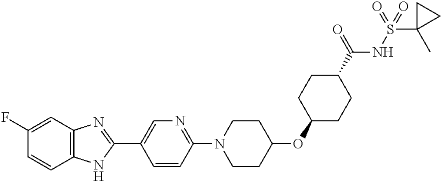 [SMILES: CC1(S(=O)(=O)NC(=O)[C@H]2CC[C@H](OC3CCN(c4ccc(-c5nc6cc(F)ccc6[nH]5)cn4)CC3)CC2)CC1]

### trans-4-({1-[5-(5-fluoro-1H-benzimidazol-2-yl)pyridin-2-yl]piperidin-4-yl}oxy)-N-[(1-methylcyclopropyl)sulfonyl]cyclohexanecarboxamide

To a mixture of trans-4-({1-[5-(5-fluoro-1H-benzimidazol-2-yl)pyridin-2-yl]piperidin-4-yl}oxy)cyclohexanecarboxylic acid (30 mg, 0.068 mmol), 1-methylcyclopropane-1-sulfonamide (18.5 mg, 0.137 mmol) and HATU (78 mg, 0.205 mmol) was added DCM (anhydrous, 2 mL) and DIEA (63 μL, 0.342 mmol). The reaction was stirred at ambient temperature for 16 hours. The solvent was evaporated and HOAc (60 μL) was added. The resulting mixture was extracted between EtOAc (4 mL×2) and water (1 mL). The organic phase was combined and concentrated. The crude product was purified by using reversed-phase HPLC to give the product as a TFA salt. LC-MS (ES, m/z) C₂₈H₃₄FN₅O₄S: 555; Found: 556 [M+H]⁺.

### Examples 81-91

Synthesized following the procedure described above using the appropriate sulfonamide.

| Example | Structure | [MH]⁺ m/z found |
|---|---|---|
| 81 | 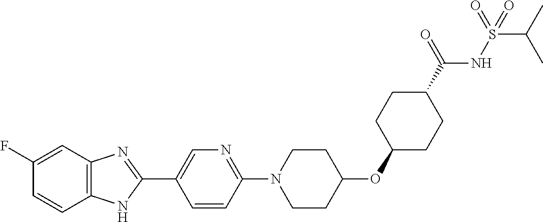 [SMILES: CC(C)S(=O)(=O)NC(=O)[C@H]1CC[C@H](OC2CCN(c3ccc(-c4nc5cc(F)ccc5[nH]4)cn3)CC2)CC1] | Found: 544 [M + H]⁺ |
| 82 | 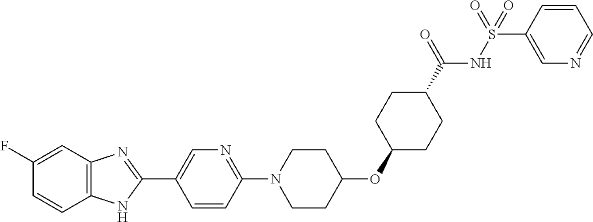 [SMILES: O=C(NS(=O)(=O)c1cccnc1)[C@H]1CC[C@H](OC2CCN(c3ccc(-c4nc5cc(F)ccc5[nH]4)cn3)CC2)CC1] | Found: 579 [M + H]⁺ |
| 83 | 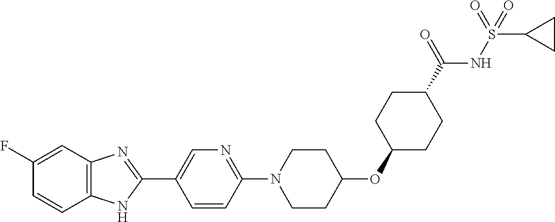 [SMILES: O=C(NS(=O)(=O)C1CC1)[C@H]1CC[C@H](OC2CCN(c3ccc(-c4nc5cc(F)ccc5[nH]4)cn3)CC2)CC1] | Found: 542 [M + H]⁺ |
| 84 | 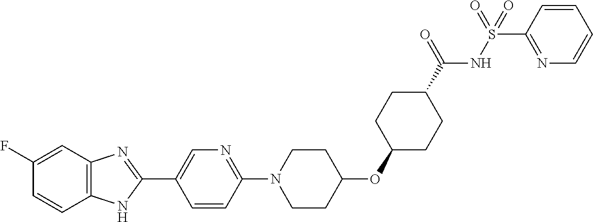 [SMILES: O=C(NS(=O)(=O)c1ccccn1)[C@H]1CC[C@H](OC2CCN(c3ccc(-c4nc5cc(F)ccc5[nH]4)cn3)CC2)CC1] | Found: 579 [M + H]⁺ |
| 85 | 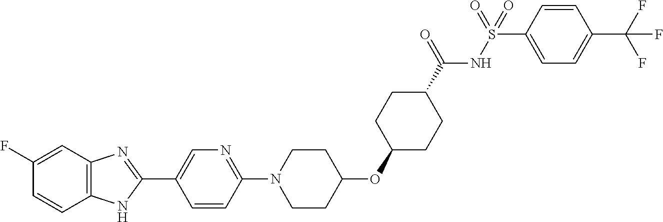 [SMILES: O=C(NS(=O)(=O)c1ccc(C(F)(F)F)cc1)[C@H]1CC[C@H](OC2CCN(c3ccc(-c4nc5cc(F)ccc5[nH]4)cn3)CC2)CC1] | Found: 646 [M + H]⁺ |
| 86 | 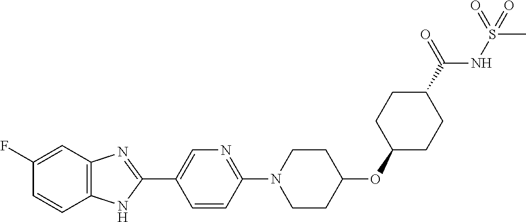 [SMILES: CS(=O)(=O)NC(=O)[C@H]1CC[C@H](OC2CCN(c3ccc(-c4nc5cc(F)ccc5[nH]4)cn3)CC2)CC1] | Found: 516 [M + H]⁺ |
| 87 | 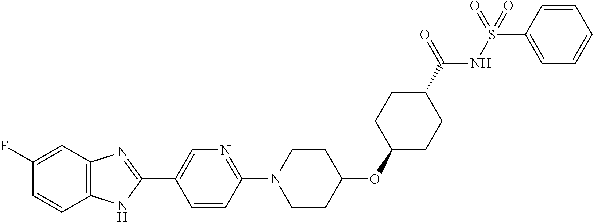 [SMILES: O=C(NS(=O)(=O)c1ccccc1)[C@H]1CC[C@H](OC2CCN(c3ccc(-c4nc5cc(F)ccc5[nH]4)cn3)CC2)CC1] | Found: 578 [M + H]⁺ |
| 88 | 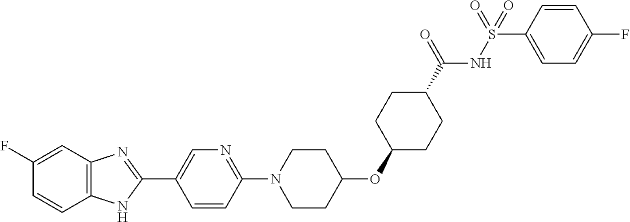 [SMILES: O=C(NS(=O)(=O)c1ccc(F)cc1)[C@H]1CC[C@H](OC2CCN(c3ccc(-c4nc5cc(F)ccc5[nH]4)cn3)CC2)CC1] | Found: 596 [M + H]⁺ |
| 89 | 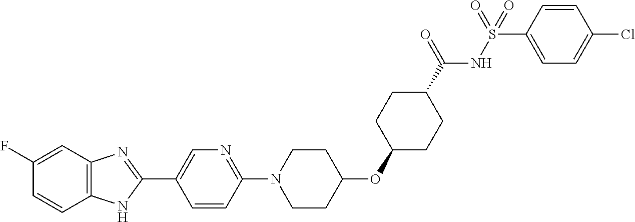 [SMILES: O=C(NS(=O)(=O)c1ccc(Cl)cc1)[C@H]1CC[C@H](OC2CCN(c3ccc(-c4nc5cc(F)ccc5[nH]4)cn3)CC2)CC1] | Found: 612 [M + H]⁺ |
| 90 | 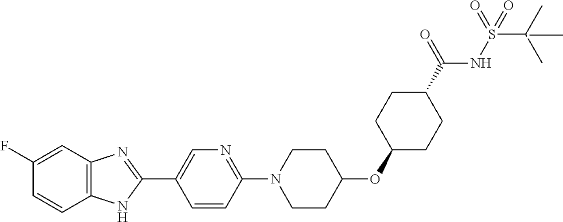 | Found: 558 [M + H]⁺ |
| 91 | 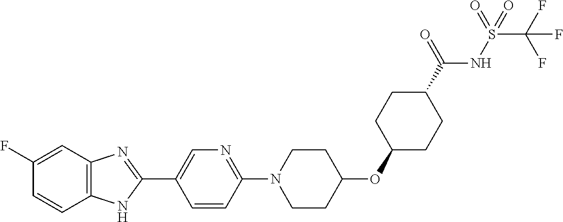 [SMILES: O=C(NS(=O)(=O)C(F)(F)F)[C@H]1CC[C@H](OC2CCN(c3ccc(-c4nc5cc(F)ccc5[nH]4)cn3)CC2)CC1] | Found: 570 [M + H]⁺ |

### Examples 92-97

Synthesized following the Method A described for [trans-4-({1-[5-(5,6-difluoro-1H-benzimidazol-2-yl)pyridin-2-yl]piperidin-4-yl}oxy)cyclohexyl]acetic acid, using the appropriate diamines and diethyl 3-(1-(5-formylpyridin-2-yl)piperidin-4-yloxy)cyclobutane-1,1-dicarboxylate.

| Example | Structure | [MH]⁺ m/z found |
|---|---|---|
| 92 | 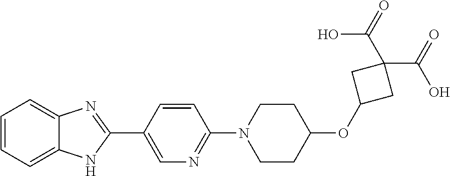 | Found: 437 [M + H]⁺ |
| 93 | 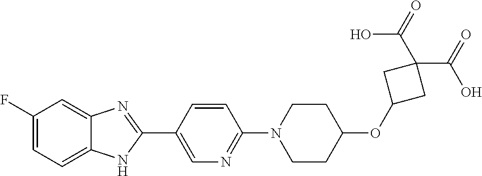 | Found: 455 [M + H]⁺ |
| 94 | 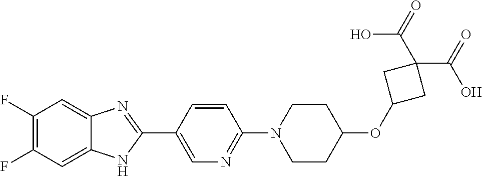 | Found: 473 [M + H]⁺ |
| 95 | 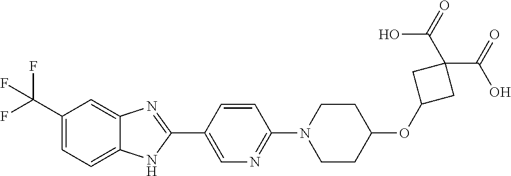 | Found: 505 [M + H]⁺ |
| 96 | 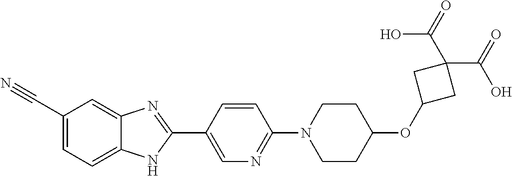 | Found: 462 [M + H]⁺ |
| 97 | 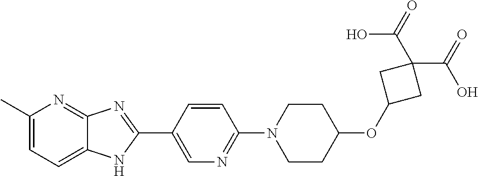 | Found: 452 [M + H]⁺ |

### Example 98

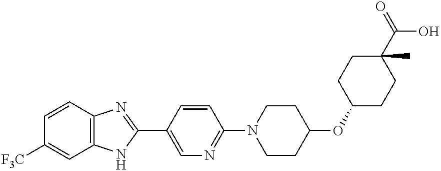 [SMILES: C[C@]1(C(=O)O)CC[C@@H](OC2CCN(c3ccc(-c4nc5ccc(C(F)(F)F)cc5[nH]4)cn3)CC2)CC1]

### cis-1-methyl-4-[(1-{5-[6-(trifluoromethyl)-1H-benzimidazol-2-yl]pyridin-2-yl}piperidin-4-yl)oxy]cyclohexanecarboxylic acid

### Step 1

A mixture of ethyl cis-1-methyl-4-(piperidin-4-yloxy)cyclohexanecarboxylate (0.2 g, 0.742 mmol), 2-(6-fluoropyridin-3-yl)-5-(trifluoromethyl)-1H-benzimidazole (0.209 g, 0.742 mmol) and sodium bicarbonate (0.624 g, 7.42 mmol) in NMP (4 ml) was heated at 110° C. in an oil bath for 4 hours under N₂. The reaction mixture was cooled to room temperature, water (10 ml) added, extracted with 3×15 mL ethyl acetate. The organic layers were combined, washed with 2×10 mL of saturated brine, dried over anhydrous sodium sulfate and concentrated under vacuum. The residue was applied onto a silica gel column and eluted with ethyl acetate/hexane 0-90%. This resulted in 0.32 g (81%) of ethyl cis-1-methyl-4-[(1-{5-[6-(trifluoromethyl)-1H-benzimidazol-2-yl]pyridin-2-yl}piperidin-4-yl)oxy]cyclohexanecarboxylate as a white solid. LC-MS (ES, m/z) C₂₈H₃₃F₃N₄O₃: 530; Found: 531 [M+H]⁺.

### Step 2

Ethyl cis-1-methyl-4-[(1-{5-[6-(trifluoromethyl)-1H-benzimidazol-2-yl]pyridin-2-yl}piperidin-4-yl)oxy]cyclohexanecarboxylate (0.15 g, 0.283 mmol) in 18.5% hydrochloride acid (3.5 ml, 21.5 mmol). The reaction mixture heat at 90° C. in an oil bath for 30 min, and then concentrated under vacuum, then purified by Gilson, acetonitrile (0.05% TFA)/water (0.05% TFA) 20-100%. This resulted in 0.048 g (27.5%) of cis-1-methyl-4-[(1-{5-[6-(trifluoromethyl)-1H-benzimidazol-2-yl]pyridin-2-yl}piperidin-4-yl)oxy]cyclohexanecarboxylic acid as a white solid. LC-MS (ES, m/z) C₂₆H₂₉F₃N₄O₃: 502; Found: 503 [M+H]⁺.

### Examples 99-104

Synthesized following the procedure described above using the appropriate F-pyridine benzimidazole pieces and piperidine pieces.

| Example | Structure | [MH]⁺ m/z found |
|---|---|---|
| 99 | 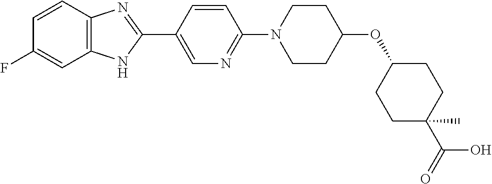 [SMILES: C[C@]1(C(=O)O)CC[C@H](OC2CCN(c3ccc(-c4nc5ccc(F)cc5[nH]4)cn3)CC2)CC1] | Found: 453 [M + H]⁺ |
| 100 | 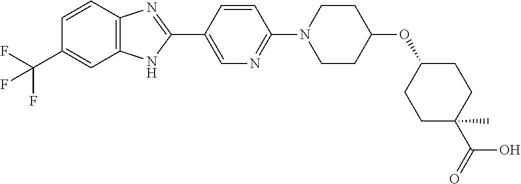 [SMILES: C[C@]1(C(=O)O)CC[C@H](OC2CCN(c3ccc(-c4nc5ccc(C(F)(F)F)cc5[nH]4)cn3)CC2)CC1] | Found: 503 [M + H]⁺ |
| 101 | 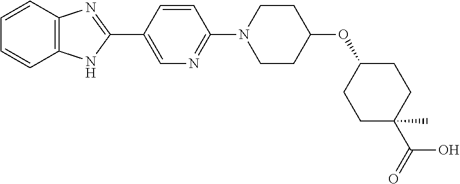 [SMILES: C[C@]1(C(=O)O)CC[C@H](OC2CCN(c3ccc(-c4nc5ccccc5[nH]4)cn3)CC2)CC1] | Found: 435 [M + H]⁺ |
| 102 | 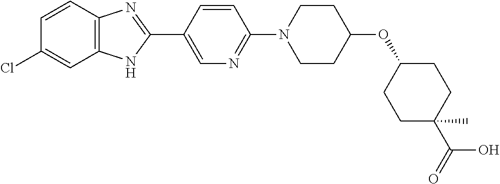 [SMILES: C[C@]1(C(=O)O)CC[C@H](OC2CCN(c3ccc(-c4nc5ccc(Cl)cc5[nH]4)cn3)CC2)CC1] | Found: 469 [M + H]⁺ |
| 103 | 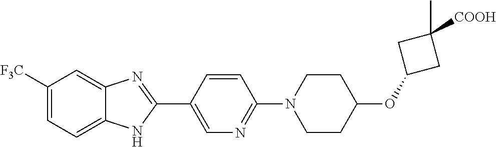 | Found: 475 [M + H]⁺ |
| 104 | 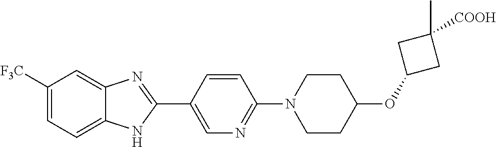 | Found: 475 [M + H]⁺ |

### Example 105

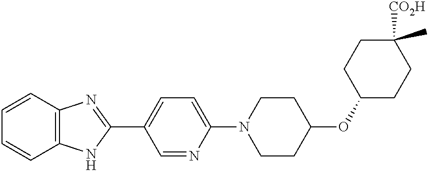 [SMILES: C[C@]1(C(=O)O)CC[C@@H](OC2CCN(c3ccc(-c4nc5ccccc5[nH]4)cn3)CC2)CC1]

### cis-4-({1-[5-(1H-benzimidazol-2-yl)pyridin-2-yl}piperidin-4-yl]oxy)-1-methylcyclohexanecarboxylic acid

### Step 1

Following the procedure described for Step 1 of Method A for the preparation of [trans-4-({1-[5-(5,6-difluoro-1H-benzimidazol-2-yl)pyridin-2-yl]piperidin-4-yl}oxy)cyclohexyl]acetic acid, starting from ethyl cis-4-{[1-(5-formylpyridin-2-yl)piperidin-4-yl]oxy}-1-methylcyclohexanecarboxylate (45 mg, 0.120 mmol), ethyl cis-4-({1-[5-(1H-benzimidazol-2-yl)pyridin-2-yl]piperidin-4-yl}oxy)-1-methylcyclohexanecarboxylate was prepared.

### Step 2

To the residue obtained from Step 1 was added HBr/H₂O (5 M, 1 mL) and the reaction was stirred at 65° C. for 1 hour. LC-MS showed that the hydrolysis was completed. The solvent was evaporated and H₂O (1 mL) was added. The solution was neutralized to pH ˜5 by adding solid K₂CO₃. The resulting mixture was extracted with EtOAc (4 mL×2) and the organic phase was concentrated. The crude product was purified by using reversed-phase HPLC to give cis-4-({1-[5-(1H-benzimidazol-2-yl)pyridin-2-yl]piperidin-4-yl}oxy)-1-methylcyclohexanecarboxylic acid as a TFA salt. LC-MS (ES, m/z) C₂₅H₃₀N₄O₃: 434; Found: 435 [M+H]⁺.

### Examples 106-112

Synthesized following the procedure described above using the appropriate diamines.

| Example | Structure | [MH]⁺ m/z found |
|---|---|---|
| 106 | 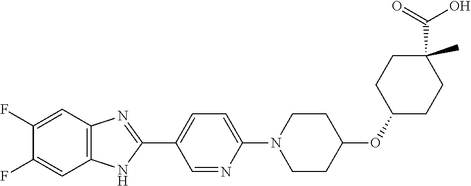 [SMILES: C[C@]1(C(=O)O)CC[C@@H](OC2CCN(c3ccc(-c4nc5cc(F)c(F)cc5[nH]4)cn3)CC2)CC1] | Found: 471 [M + H]⁺ |
| 107 | 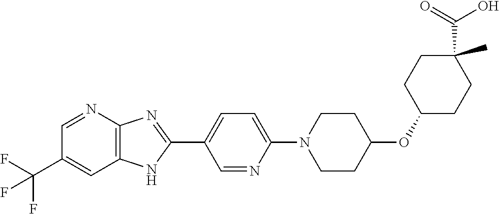 [SMILES: C[C@]1(C(=O)O)CC[C@@H](OC2CCN(c3ccc(-c4nc5ncc(C(F)(F)F)cc5[nH]4)cn3)CC2)CC1] | Found: 504 [M + H]⁺ |
| 108 | 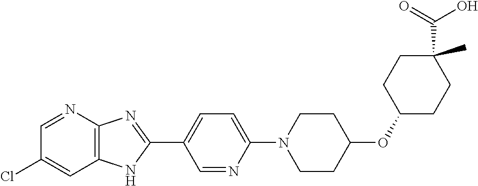 [SMILES: C[C@]1(C(=O)O)CC[C@@H](OC2CCN(c3ccc(-c4nc5ncc(Cl)cc5[nH]4)cn3)CC2)CC1] | Found: 470 [M + H]⁺ |
| 109 | 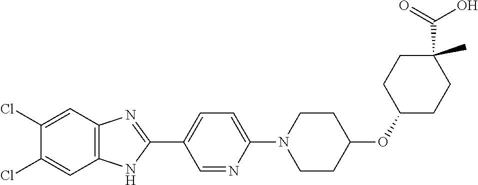 [SMILES: C[C@]1(C(=O)O)CC[C@@H](OC2CCN(c3ccc(-c4nc5cc(Cl)c(Cl)cc5[nH]4)cn3)CC2)CC1] | Found: 503 [M + H]⁺ |
| 110 | 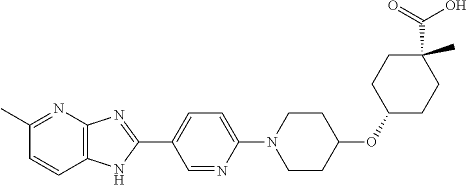 | Found: 450 [M + H]⁺ |
| 111 | 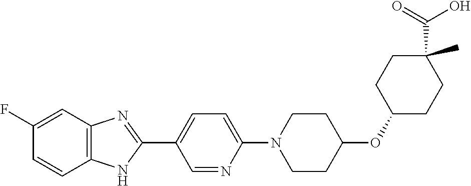 [SMILES: C[C@]1(C(=O)O)CC[C@@H](OC2CCN(c3ccc(-c4nc5cc(F)ccc5[nH]4)cn3)CC2)CC1] | Found: 453 [M + H]⁺ |
| 112 | 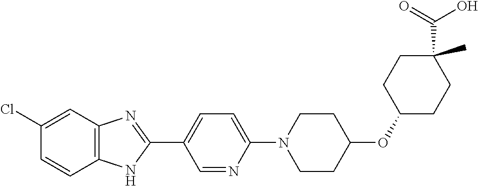 [SMILES: C[C@]1(C(=O)O)CC[C@@H](OC2CCN(c3ccc(-c4nc5cc(Cl)ccc5[nH]4)cn3)CC2)CC1] | Found: 469 [M + H]⁺ |

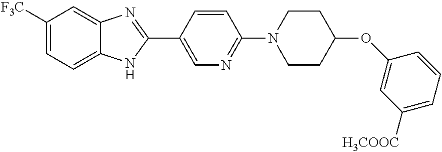

### Methyl 3-[(1-{5-[5-(trifluoromethyl)-1H-benzimidazol-2-yl]pyridin-2-yl}piperidin-4-yl)oxy]benzoate

To methyl-3-(4-piperidinyloxy)benzoate hydrochloride (0.202 g, 0.743 mmol) was added CH₂Cl₂ (20 ml) and washed with NaHCO₃ (sat.), dried over Na₂SO₄ and concentrated and transfer to pyrex microwave reaction vial (10 ml) with DMF (2.5 ml), then added 2-(6-fluoropyridin-3-yl)-5-(trifluoromethyl)-1H-benzo[d]imidazole (0.190 g, 0.676 mmol) and cesium carbonate (0.330 g, 1.013 mmol). The mixture was then exposed to microwave at 160° C. for 1 hr and then cooled to room temperature. The mixture was partitioned between ethyl acetate and water. The organic layer was separated and the aqueous layer was extracted with EtOAc. The combined organic phases were washed with brine, dried over MgSO₄, filtered and concentrated. The residue was purified by MPLC (50 g silica gel, 0 to 100% ethyl acetate in hexanes) to afford methyl 3-[(1-{5-[5-(trifluoromethyl)-1H-benzimidazol-2-yl]pyridin-2-yl}piperidin-4-yl)oxy]benzoate (0.11 g) as brown solid. LC-MS (ES, m/z) C₂₆H₂₃F₃N₄O₃: 496; Found: 497 [M+H]⁺.

### Example 114

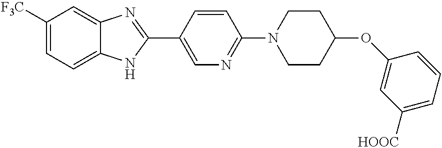 [SMILES: O=C(O)c1cccc(OC2CCN(c3ccc(-c4nc5cc(C(F)(F)F)ccc5[nH]4)cn3)CC2)c1]

### 3-[(1-{5-[5-(trifluoromethyl)-1H-benzimidazol-2-yl]pyridin-2-yl}piperidin-4-yl)oxy]benzoic acid

To a solution of methyl 3-[(1-{5-[5-(trifluoromethyl)-1H-benzimidazol-2-yl]pyridin-2-yl}piperidin-4-yl)oxy]benzoate in THF/H₂O (4:1, 7.5 ml) was added Lithium hydroxide monohydrate (17.75 mg, 0.423 mmol). After stirring at 40° C. for overnight, the organic solvent was removed. The aqueous was acidified with TFA, and concentrated. The residue was dissolved in DMSO/AcCN/H₂O (2:1:1, 8 ml), filtered through syringe-driven filter, and then purified by Gilson (30-80% AcCN in H₂O containing 0.05% TFA in 18 min linear, flow rate 30 ml/min, injection 1 ml). The desired fraction was collected and lyophilized to give 3-[(1-{5-[5-(trifluoromethyl)-1H-benzimidazol-2-yl]pyridin-2-yl}piperidin-4-yl)oxy]benzoic acid (13 mg) as yellow solid LC-MS (ES, m/z) C₂₅H₂₁F₃N₄O₃: 482; Found: 483 [M+H]⁺.

### Example 115

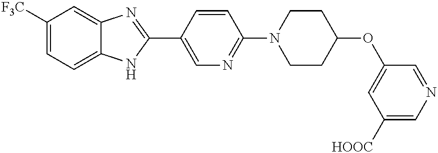 [SMILES: O=C(O)c1cncc(OC2CCN(c3ccc(-c4nc5cc(C(F)(F)F)ccc5[nH]4)cn3)CC2)c1]

### 5-[(1-{5-[5-(trifluoromethyl)-1H-benzimidazol-2-yl]pyridin-2-yl}piperidin-4-yl)oxy]pyridine-3-carboxylic acid

### Step 1

To a solution of methyl 5-(1-(tert-butoxycarbonyl)piperidin-4-yloxy)nicotinate (0.20 g, 0.595 mmol) in DCM (1 ml) was added HCl (4.0 M in dioxane, 1 ml) and stirred at room temperature for 0.5 h. The reaction mixture was concentrated and the residue was dissolved in NMP (2.0 ml), added 2-(6-fluoropyridin-3-yl)-5-(trifluoromethyl)-1H-benzo[d]imidazole (184 mg, 0.654 mmol)ts] and sodium bicarbonate (0.250 g, 2.98 mmol), and heated at 110° C. overnight. The reaction mixture was added water, extracted with EtOAc, dried over Na₂SO₄, filtered and concentrated, separated by Thar 80 preparative SFC (column: ChiralPak OD-H-10 μm 300×50 mmI.D.; Mobile phase: A for CO₂ and B for ethanol; Gradient: B 45%; Flow rate: 80 ml/min; Sample preparation: dissolved in ethanol, 70 mg/ml; Injection: 1 ml per injection) After separation, the desired fractions were dried off via rotary evaporator at bath temperature 40° C. to give methyl 5-[(1-{5-[5-(trifluoromethyl)-1H-benzimidazol-2-yl]pyridin-2-yl}piperidin-4-yl)oxy]pyridine-3-carboxylate. LC-MS (ES, m/z) C₂₅H₂₂F₃N₅O₃: 497; Found: 498 [M+H]⁺.

### Step 2

To a solution of methyl 5-[(1-{5-[5-(trifluoromethyl)-1H-benzimidazol-2-yl]pyridin-2-yl}piperidin-4-yl)oxy]pyridine-3-carboxylate (40 mg, 0.080 mmol) in THF/H₂O (4:1, 2.5 ml) was added Lithium hydroxide monohydrate (9.63 mg, 0.402 mmol). After stirred at 40° C. for overnight, reaction mixture was concentrated. The residue was dissolved in DMSO (2 ml) and DMSO/H₂O/AcCN (1:1:2, 3 ml), filtered with syringe-driven filter, and purified by Gilson (20-100% AcCN in H₂O containing 0.05% TFA in 18 min linear, flow rate 30 ml/min, injection 2.5 ml). The desired fraction was collected and lyophilized to give 5-[(1-{5-[5-(trifluoromethyl)-1H-benzimidazol-2-yl]pyridin-2-yl}piperidin-4-yl)oxy]pyridine-3-carboxylic acid (25 mg) as white solid. LC-MS (ES, m/z) C₂₄H₂₁F₃N₅O₃: 483; Found: 484 [M+H]⁺.

### Examples 116-126

Synthesized following the procedure described above, using appropriate F-pyridine benzimidazole pieces and piperidine pieces.

| | | [MH]⁺ m/z |
|---|---|---|
| Example | Structure | found |
| 116 | 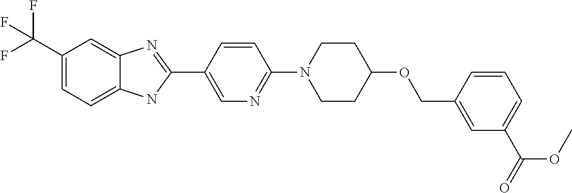 [SMILES: COC(=O)c1cccc(COC2CCN(c3ccc(-c4nc5cc(C(F)(F)F)ccc5[nH]4)cn3)CC2)c1] | Found: 511 [M + H]⁺ |
| 117 | 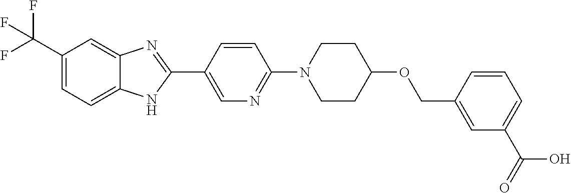 [SMILES: O=C(O)c1cccc(COC2CCN(c3ccc(-c4nc5cc(C(F)(F)F)ccc5[nH]4)cn3)CC2)c1] | Found: 497 [M + H]⁺ |
| 118 | 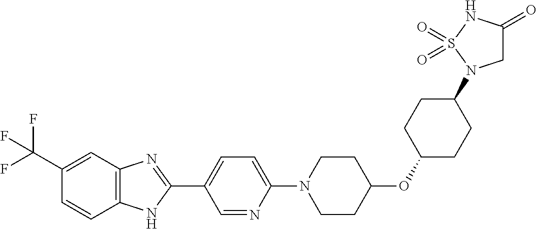 [SMILES: O=C1CN([C@H]2CC[C@H](OC3CCN(c4ccc(-c5nc6cc(C(F)(F)F)ccc6[nH]5)cn4)CC3)CC2)S(=O)(=O)N1] | Found: 580 [M + 2H]⁺ |
| 119 | 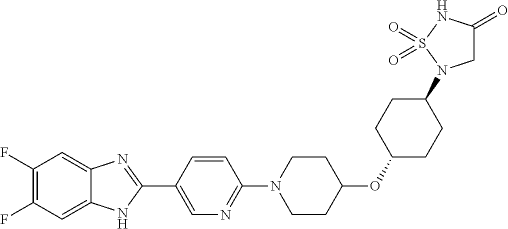 [SMILES: O=C1CN([C@H]2CC[C@H](OC3CCN(c4ccc(-c5nc6cc(F)c(F)cc6[nH]5)cn4)CC3)CC2)S(=O)(=O)N1] | Found: 546 [M]⁺ |
| 120 | 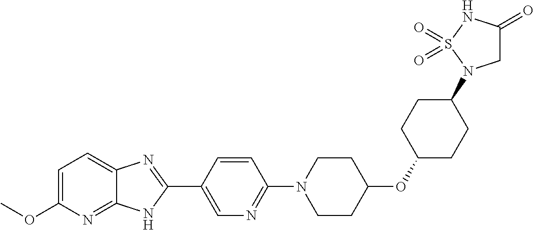 [SMILES: COc1ccc2nc(-c3ccc(N4CCC(O[C@H]5CC[C@H](N6CC(=O)NS6(=O)=O)CC5)CC4)nc3)[nH]c2n1] | Found: 542 [M + H]⁺ |
| 121 | 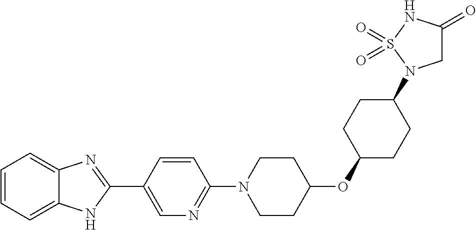 [SMILES: O=C1CN([C@H]2CC[C@@H](OC3CCN(c4ccc(-c5nc6ccccc6[nH]5)cn4)CC3)CC2)S(=O)(=O)N1] | Found: 511 [M + H]⁺ |
| 122 | 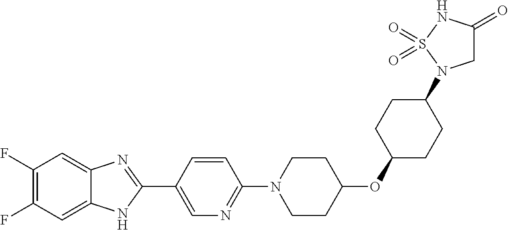 [SMILES: O=C1CN([C@H]2CC[C@@H](OC3CCN(c4ccc(-c5nc6cc(F)c(F)cc6[nH]5)cn4)CC3)CC2)S(=O)(=O)N1] | Found: 547 [M + H]⁺ |
| 123 | 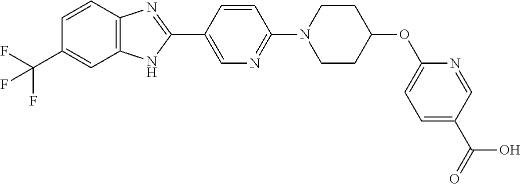 [SMILES: O=C(O)c1ccc(OC2CCN(c3ccc(-c4nc5ccc(C(F)(F)F)cc5[nH]4)cn3)CC2)nc1] | Found: 484 [M + H]⁺ |
| 124 | 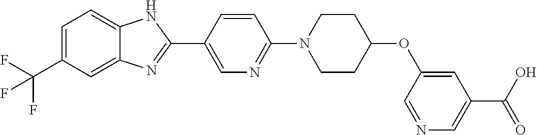 [SMILES: O=C(O)c1cncc(OC2CCN(c3ccc(-c4nc5cc(C(F)(F)F)ccc5[nH]4)cn3)CC2)c1] | Found: 484 [M + H]⁺ |
| 125 | 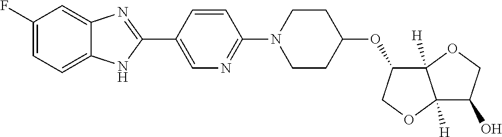 [SMILES: O[C@@H]1CO[C@H]2[C@@H]1OC[C@@H]2OC1CCN(c2ccc(-c3nc4cc(F)ccc4[nH]3)cn2)CC1] | Found: 441 [M + H]⁺ |
| 126 | 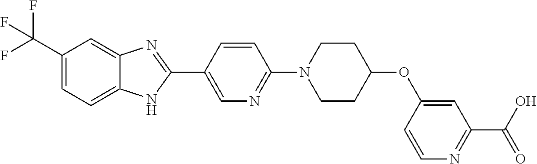 [SMILES: O=C(O)c1cc(OC2CCN(c3ccc(-c4nc5cc(C(F)(F)F)ccc5[nH]4)cn3)CC2)ccn1] | Found: 484 [M + H]⁺ |

### Example 127

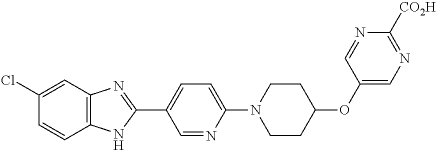 [SMILES: O=C(O)c1ncc(OC2CCN(c3ccc(-c4nc5cc(Cl)ccc5[nH]4)cn3)CC2)cn1]

### 5-(1-(5-(5-chloro-1H-benzo[d]imidazol-2-yl)pyridin-2-yl)piperidin-4-yloxy)pyrimidine-2-carboxylic acid

### Step 1

To a solution of 1-[5-(5-chloro-1H-benzimidazol-2-yl)pyridin-2-yl]piperidin-4-ol (30 mg, 0.091 mmol), methyl 5-hydroxypyrimidine-2-carboxylate (28 mg, 0.182 mmol) and triphenyl phosphine (72 mg, 0.275 mmol) in DCM (anhydrous, 1 mL) was added diisopropyl diazene-1,2-dicarboxylate (DIED, 90 μL, 0.46 mmol) drop wise. The reaction was stirred at ambient temperature for 16 hours. LC-MS showed that the product was formed. The solvent was evaporated and the residue was used in Step 2 without purification.

### Step 2

The procedure described for the preparation of [trans-4-({1-[5-(5,6-difluoro-1H-benzimidazol-2-yl)pyridin-2-yl]piperidin-4-yl}oxy)cyclohexyl]acetic acid (Method A, Step 2) was used, starting from the crude product obtained from Step 1 above. 5-(1-(5-(5-Chloro-1H-benzo[d]imidazol-2-yl)pyridin-2-yl)piperidin-4-yloxy)pyrimidine-2-carboxylic acid was afforded as the TFA salt after RP HPLC purification. LC-MS (ES, m/z) C₂₂H₁₉ClN₆O₃: 450; Found: 451 [M+H]⁺.

### Examples 128-132

Synthesized following the method described above.

| Example | Structure | [MH]⁺ m/z found |
|---|---|---|
| 128 | 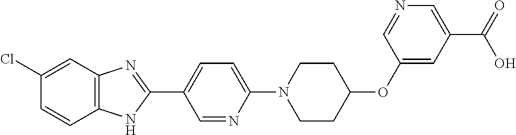 [SMILES: O=C(O)c1cncc(OC2CCN(c3ccc(-c4nc5cc(Cl)ccc5[nH]4)cn3)CC2)c1] | Found: 450 [M + H]⁺ |
| 129 | 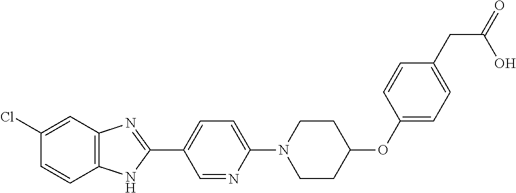 [SMILES: O=C(O)Cc1ccc(OC2CCN(c3ccc(-c4nc5cc(Cl)ccc5[nH]4)cn3)CC2)cc1] | Found: 463 [M + H]⁺ |
| 130 | 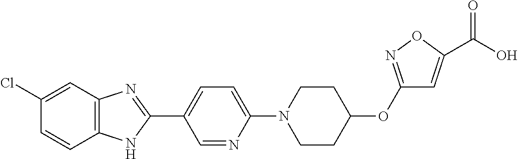 [SMILES: O=C(O)c1cc(OC2CCN(c3ccc(-c4nc5cc(Cl)ccc5[nH]4)cn3)CC2)no1] | Found: 440 [M + H]⁺ |
| 131 | 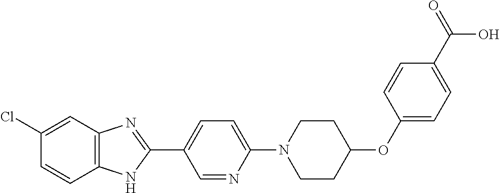 [SMILES: O=C(O)c1ccc(OC2CCN(c3ccc(-c4nc5cc(Cl)ccc5[nH]4)cn3)CC2)cc1] | Found: 449 [M + H]⁺ |
| 132 | 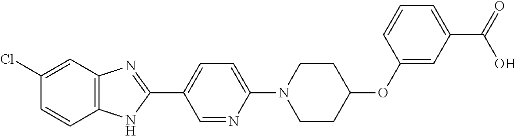 [SMILES: O=C(O)c1cccc(OC2CCN(c3ccc(-c4nc5cc(Cl)ccc5[nH]4)cn3)CC2)c1] | Found: 449 [M + H]⁺ |

### Example 133

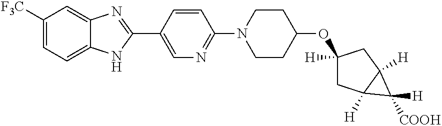 [SMILES: O=C(O)[C@H]1[C@@H]2C[C@H](OC3CCN(c4ccc(-c5nc6cc(C(F)(F)F)ccc6[nH]5)cn4)CC3)C[C@@H]21]

### (1R,3R,5S,6r)-3-[(1-{5-[5-(trifluoromethyl)-1H-benzimidazol-2-yl]pyridin-2-yl}piperidin-4-yl)oxy]bicyclo[3.1.0]hexane-6-carboxylic acid

### Step 1

Benzyl 4-{[(1R,3R,5S,6r)-6-(ethoxycarbonyl)bicyclo[3.1.0]hex-3-yl]oxy}piperidine-1-carboxylate (0.5 g, 1.29 mmol) was dissolved in ethanol (10 ml), 10% palladium on carbon (0.0069 g, 0.085 mmol) was added. The reaction mixture was stirred at 1 atm H₂ for 2 days. The reaction mixture was filtered and the filtrate was concentrated under vacuum to result in ethyl (1R,3R,5S,6r)-3-(piperidin-4-yloxy)bicyclo[3.1.0]hexane-6-carboxylate as a colorless oil. LC-MS (ES, m/z) C₁₄H₂₃NO₃: 253; Found: 254 [M+H]⁺.

### Step 2

A mixture of ethyl (1R,3R,5S,6r)-3-(piperidin-4-yloxy)bicyclo[3.1.0]hexane-6-carboxylate (0.327 g, 1.291 mmol), 2-(6-fluoropyridin-3-yl)-5-(trifluoromethyl)-1H-benzimidazole (0.363 g, 1.291 mmol) and sodium bicarbonate (1.08 g, 12.91 mmol) in NMP (4 ml) was heated at 110° C. in an oil bath over night under N₂. The reaction mixture was cooled to room temperature, water (10 ml) added, extracted with 3×15 ml ethyl acetate. The organic layers were combined, washed with 2×10 ml of saturated brine, dried over anhydrous sodium sulfate and concentrated under vacuum. The residue was purified by silica gel column eluted with ethyl acetate/hexane 20-100%. This resulted in ethyl (1R,3R,5S,6r)-3-[(1-{5-[5-(trifluoromethyl)-1H-benzimidazol-2-yl]pyridin-2-yl}piperidin-4-yl)oxy]bicyclo[3.1.0]hexane-6-carboxylate as a white solid. LC-MS (ES, m/z) C₂₇H₂₉F₃N₄O₃: 514; Found: 515 [M+H]⁺.

### Step 3

A mixture of ethyl (1R,3R,5S,6r)-3-[(1-{5-[5-(trifluoromethyl)-1H-benzimidazol-2-yl]pyridin-2-yl}piperidin-4-yl)oxy]bicyclo[3.1.0]hexane-6-carboxylate and lithium hydroxide (0.117 g, 4.9 mmol) in THF (2 ml) and water (1 ml) was stirred at room temperature over night then concentrated under vacuum. The residue was purified by Gilson reverse HPLC (acetonitrile (0.05% TFA)/water (0.05% TFA) 20-100%). This resulted in TFA salt of (1R,3R,5S,6r)-3-[(1-{5-[5-(trifluoromethyl)-1H-benzimidazol-2-yl]pyridin-2-yl}piperidin-4-yl)oxy]bicyclo[3.1.0]hexane-6-carboxylic acid as a white solid. LC-MS (ES, m/z) C₂₅H₂₅F₃N₄O₃: 486; Found: 487 [M+H]⁺.

### Examples 134-136

Using the appropriate Cbz protected piperidine pieces, following the same procedure as Example 132, Examples 133-136 were prepared

| Example | Structure | [MH]⁺ m/z found |
|---|---|---|
| 134 | 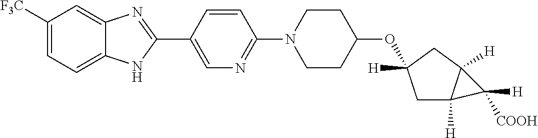 [SMILES: O=C(O)[C@H]1[C@@H]2C[C@@H](OC3CCN(c4ccc(-c5nc6cc(C(F)(F)F)ccc6[nH]5)cn4)CC3)C[C@@H]21] | Found: 487 [M + H]⁺ |
| 135 | 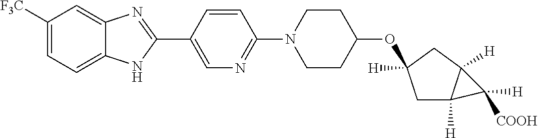 [SMILES: O=C(O)[C@@H]1[C@@H]2C[C@H](OC3CCN(c4ccc(-c5nc6cc(C(F)(F)F)ccc6[nH]5)cn4)CC3)C[C@@H]21] | Found: 487 [M + H]⁺ |
| 136 | 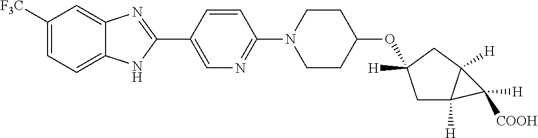 [SMILES: O=C(O)[C@@H]1[C@@H]2C[C@@H](OC3CCN(c4ccc(-c5nc6cc(C(F)(F)F)ccc6[nH]5)cn4)CC3)C[C@@H]21] | Found: 487 [M + H]⁺ |

### Example 137

### [trans-4-({(3R)-1-[5-(5-fluoro-1H-benzimidazol-2-yl)pyridin-2-yl]pyrrolidin-3-yl}oxy)cyclohexyl]acetic acid

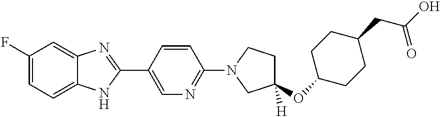 [SMILES: O=C(O)C[C@H]1CC[C@H](O[C@@H]2CCN(c3ccc(-c4nc5cc(F)ccc5[nH]4)cn3)C2)CC1]

### [trans-4-({(3R)-1-[5-(5-fluoro-1H-benzimidazol-2-yl)pyridin-2-yl]pyrrolidin-3-yl}oxy)cyclohexyl]acetic acid

A mixture of methyl [trans-4-({(3R)-1-[5-(5-fluoro-1H-benzimidazol-2-yl)pyridin-2-yl]pyrrolidin-3-yl}oxy)cyclohexyl]acetate (0.188 g, 0.415 mmol) and lithium hydroxide (0.07 g, 2.91 mmol) in THF (4 ml) and water (1 ml). The reaction mixture was stirred at room temperature over night then concentrated under vacuum. The mixture was applied onto a synergi C18 column (10u, 250×50 mm I.D). Mobile phase: A for water (0.1% TFA) and B for acetonitrile (0.1% TFA). Gradient: B 20%-50%. This resulted in [trans-4-({(3R)-1-[5-(5-fluoro-1H-benzimidazol-2-yl)pyridin-2-yl]pyrrolidin-3-yl}oxy)cyclohexyl]acetic acid as a white solid. LC-MS (ES, m/z) C₂₄H₂₇FN₄O₃: 438; Found: 439 [M+H]⁺.

### Examples 138-140

Synthesized using the appropriate Cbz protected piperidine pieces, following the same procedure described for [trans-4-({(3R)-1-[5-(5-fluoro-1H-benzimidazol-2-yl)pyridin-2-yl]pyrrolidin-3-yl}oxy)cyclohexyl]acetic acid.

| Example | Structure | [MH]⁺ m/z found |
|---|---|---|
| 138 | 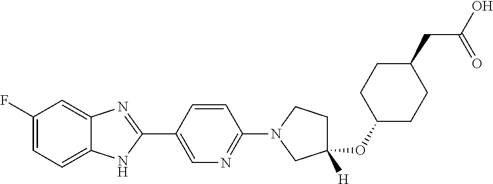 [SMILES: O=C(O)C[C@H]1CC[C@H](O[C@H]2CCN(c3ccc(-c4nc5cc(F)ccc5[nH]4)cn3)C2)CC1] | Found: 439 [M + H]⁺ |
| 139 | 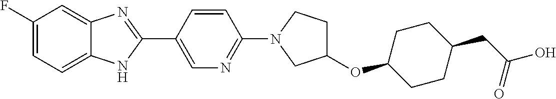 [SMILES: O=C(O)C[C@H]1CC[C@@H](OC2CCN(c3ccc(-c4nc5cc(F)ccc5[nH]4)cn3)C2)CC1] | Found: 439 [M + H]⁺ |
| 140 | 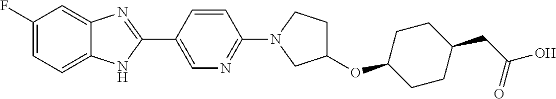 [SMILES: O=C(O)C[C@H]1CC[C@@H](OC2CCN(c3ccc(-c4nc5cc(F)ccc5[nH]4)cn3)C2)CC1] | Found: 439 [M + H]⁺ |

### Example 141

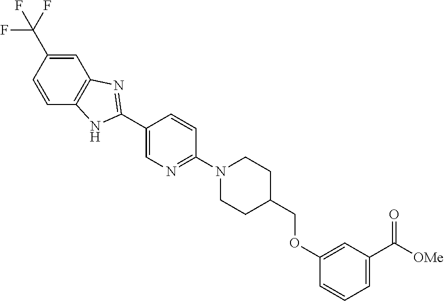 [SMILES: COC(=O)c1cccc(OCC2CCN(c3ccc(-c4nc5cc(C(F)(F)F)ccc5[nH]4)cn3)CC2)c1]

### methyl 3-[[1-[5-[5-(trifluoromethyl)-1H-benzol[d]imidazol-2-yl]pyridine-2-yl]piperidin-4-yl]methoxy]benzoate

A microwave vial was charged with Intermediate 1 (100 mg, 0.23 mmol), 2-chloro-5-(trifluoromethyl-1H-benzo[d]imidazole (40 mg, 0.18 mmol), bis(triphenylphosphine)palladium-(II)dichloride (35 mg, 0.05 mmol), potassium carbonate (75 mg, 0.54 mmol) and acetonitrile/water (4:1, 2.5 mL) then heated at 150° C. for 30 mins in a microwave reactor. The mixture was filtered through celite, concentrated and purified by a Gilson HPLC to yield the title compound as a pale yellow solid. LC/MS=511.3 [M+1].

### Example 142

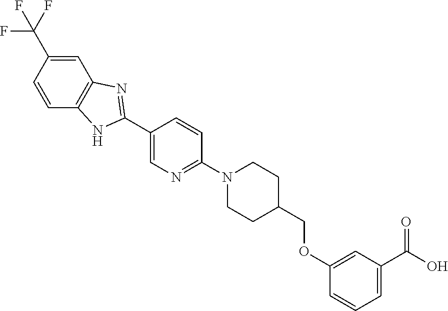 [SMILES: O=C(O)c1cccc(OCC2CCN(c3ccc(-c4nc5cc(C(F)(F)F)ccc5[nH]4)cn3)CC2)c1]

### 3-[[1-[5-[5-(trifluoromethyl)-1H-benzol[d]imidazol-2-yl]pyridine-2-yl]piperidin-4-yl]methoxy]benzoic acid

To a stirred solution of the product obtained in Example 1 (38 mg, 0.075 mmol) in MeOH (1 mL), THF (1 mL), and water (0.5 mL) was added lithium hydroxide (16 mg, 0.370 mmol). The reaction mixture was stirred at RT for 5 h then acidified with 1 N HCl (0.5 mL). The solution was concentrated and purified by a Gilson HPLC to yield the title compound as a white solid. LC/MS=497.3 [M+1].

The following compounds were prepared by using methods described in Examples 1-2.

| Example | Structure | LC-MS |
|---|---|---|
| 143 | 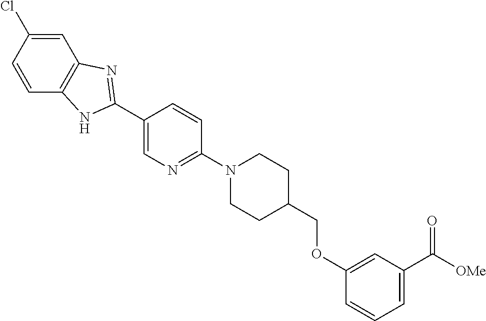 [SMILES: COC(=O)c1cccc(OCC2CCN(c3ccc(-c4nc5cc(Cl)ccc5[nH]4)cn3)CC2)c1] | 477.3 [M + 1]. |
| 144 | 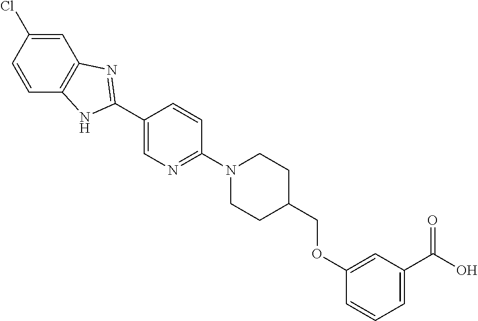 [SMILES: O=C(O)c1cccc(OCC2CCN(c3ccc(-c4nc5cc(Cl)ccc5[nH]4)cn3)CC2)c1] | 463.3 [M + 1]. |

### Example 145

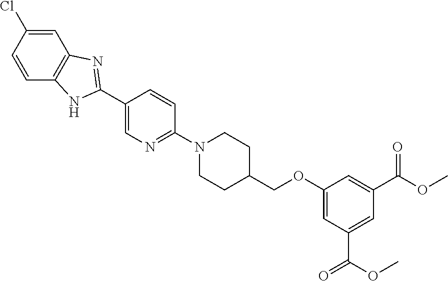 [SMILES: COC(=O)c1cc(OCC2CCN(c3ccc(-c4nc5cc(Cl)ccc5[nH]4)cn3)CC2)cc(C(=O)OC)c1]

### dimethyl 5-((1-(5-(5-chloro-1H-benzo[d]imidazol-2-yl)pyridin-2-yl)piperidin-4-yl)methoxy)isophthalate

To (1-(5-(5-chloro-1H-benzo[d]imidazol-2-yl)pyridin-2-yl)piperidin-4-yl)methanol (100 mg, 0.30 mmol) and dimethyl 5-hydroxyisophthalate (60 mg, 0.30 mmol) in THF (10 mL) was added diethyl azodicarboxylate (0.09 mL, 0.60 mmol) and triphenylphosphine (157 mg, 0.60 mmol). The reaction mixture was stirred at RT overnight and concentrated. Purification by silica gel chromatography (eluant: 1:1 EtOAc:hexanes) to obtain dimethyl 5-((1-(5-(5-chloro-1H-benzo[d]imidazol-2-yl)pyridin-2-yl)piperidin-4-yl)methoxy)isophthalate as a white solid. LC/MS=535 [M+1].

### Example 146

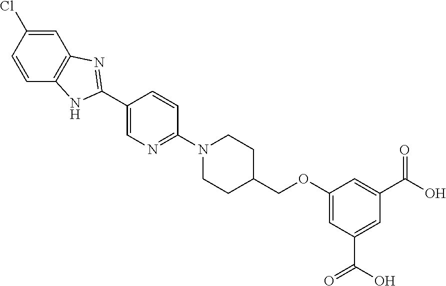 [SMILES: O=C(O)c1cc(OCC2CCN(c3ccc(-c4nc5cc(Cl)ccc5[nH]4)cn3)CC2)cc(C(=O)O)c1]

### 5-((1-(5-(5-chloro-1H-benzo[d]imidazol-2-yl)pyridin-2-yl)piperidin-4-yl)methoxy)isophthalic acid

To a stirred solution of 5-((1-(5-(5-chloro-1H-benzo[d]imidazol-2-yl)pyridin-2-yl)piperidin-4-yl)methoxy)isophthalate (96.3 mg, 0.18 mmol) in MeOH (3.0 mL), THF (3.0 mL), and water (2.0 mL) was added 1 N aqueous sodium hydroxide (2.0 mL). The reaction mixture was stirred at RT for 5 h. 1 N HCl (2.5 mL) was added, and the solution concentrated. The title compound 5-((1-(5-(5-chloro-1H-benzo[d]imidazol-2-yl)pyridin-2-yl)piperidin-4-yl)methoxy)isophthalic acid was obtained after purification with a Gilson HPLC (eluant: H₂O:CH₃CN) as a beige solid. LC/MS=507 [M+1].

### Example 147

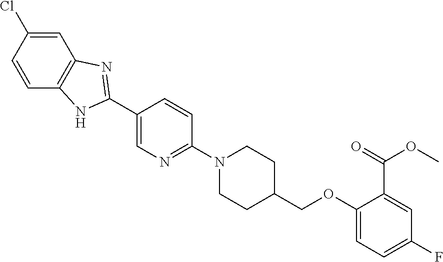 [SMILES: COC(=O)c1cc(F)ccc1OCC1CCN(c2ccc(-c3nc4cc(Cl)ccc4[nH]3)cn2)CC1]

### Methyl 2-[[1-[5-(6-chloro-1H-benzimidazol-2-yl)-2-pyridinyl]-4-piperidinyl]methoxyl]-5-fluorobenzoate

4-[[4-Fluoro-2-(methoxycarbonyl)phenoxy]methyl]-piperidine (HCl salt, 61 mg. 0.2 mmol) was mixed with 5-chloro-2-[6-fluoro-pyridine-3-yl]-1H-benzon[d]imidazole (50 mg, 0.2 mmol) and diisopropylethylamine (0.11 mL, 0.6 mmol) in 3 mL of DMF. The mixture was heated to 190° C. for 50 mins by a microwave reactor. After cooling to RT, the mixture was purified by Gilson prep HPLC to give methyl 2-[[1-[5-(6-chloro-1H-benzimidazol-2-yl)-2-pyridinyl]-4-piperidinyl]methoxy]-5-fluorobenzoate. LC/MS=495.2 [M+1].

### Example 148

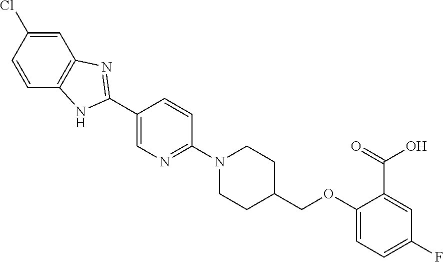 [SMILES: O=C(O)c1cc(F)ccc1OCC1CCN(c2ccc(-c3nc4cc(Cl)ccc4[nH]3)cn2)CC1]

### 2-[[1-[5-(6-chloro-1H-benzimidazol-2-yl)-2-pyridinyl]-4-piperidinyl]methoxy]-5-fluorobenzoic acid

Methyl 2-[[1-[5-(6-chloro-1H-benzimidazol-2-yl)-2-pyridinyl]-4-piperidinyl]methoxy]-5-fluorobenzoate (30 mg) was mixed with lithium hydroxide (50 mg) in a mixed solvent of THF (2 mL), MeOH (0.5 mL) and water (0.5 mL). The mixture was stirred at RT overnight, then purified with Gilson prep HPLC to give 2-[[1-[5-(6-chloro-1H-benzimidazol-2-yl)-2-pyridinyl]-4-piperidinyl]methoxy]-5-fluorobenzoic acid (24 mg). LC/MS=481.2 [M+1].

### Example 149

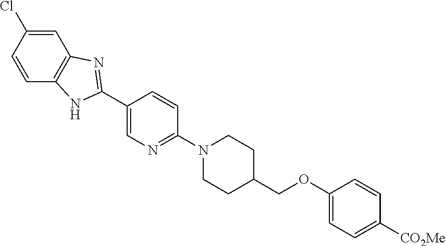 [SMILES: COC(=O)c1ccc(OCC2CCN(c3ccc(-c4nc5cc(Cl)ccc5[nH]4)cn3)CC2)cc1]

### Methyl 4-[[1-[5-(6-chloro-1H-benzimidazol-2-yl)-2-pyridinyl]-4-piperidinyl]methoxy]benzoate

4-Chloro-1,2-benzenediamine (72 mg, 0.5 mmol) was mixed with Oxone (0.15 g, 0.25 mmol) in DMF (2 mL) and water (0.1 mL). Methyl 4-[[1-[5-formyl-2-pyridinyl]-piperidin-4-yl]methoxy]-benzoate (0.4 mmol, reaction mixture from Step C) was then added dropwise at RT. The resulting mixture was stirred at RT overnight then poured into 100 mL of water, and the pH was adjusted to 7-8 with solid sodium carbonate. The precipitate was collected by filtration, washed with water, and dried to give methyl 4-[[1-[5-(6-chloro-1H-benzimidazol-2-yl)-2-pyridinyl]-4-piperidinyl]methoxy]benzoate as a light-brown product. LC/MS=477.2 [M+1].

### Example 150

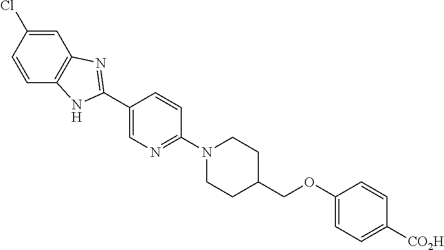 [SMILES: O=C(O)c1ccc(OCC2CCN(c3ccc(-c4nc5cc(Cl)ccc5[nH]4)cn3)CC2)cc1]

### 4-[[1-[5-(6-Chloro-1H-benzimidazol-2-yl)-2-pyridinyl]-4-piperidinyl]methoxy]benzoic acid

Methyl 4-[[1-[5-(6-chloro-1H-benzimidazol-2-yl)-2-pyridinyl]-4-piperidinyl]methoxy]benzoate (150 mg) was mixed with lithium hydroxide (200 mg) in a mixed solvent of THF (4 mL), MeOH (1 mL) and water (1 mL). The mixture was stirred at RT overnight, then purified with Gilson prep HPLC to give 4-[[1-[5-(6-chloro-1H-benzimidazol-2-yl)-2-pyridinyl]-4-piperidinyl]methoxy]benzoic acid as white solid. LC/MS=463.1 [M+1].

The following compounds were prepared by using methods described in Examples 52-53.

| Example | Structure | LC-MS |
|---|---|---|
| 151 | 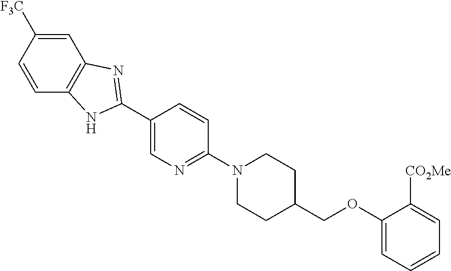 [SMILES: COC(=O)c1ccccc1OCC1CCN(c2ccc(-c3nc4cc(C(F)(F)F)ccc4[nH]3)cn2)CC1] | 511.2 [M + 1]. |
| 152 | 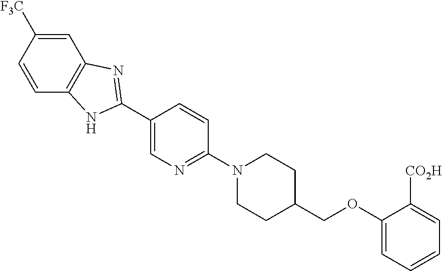 [SMILES: O=C(O)c1ccccc1OCC1CCN(c2ccc(-c3nc4cc(C(F)(F)F)ccc4[nH]3)cn2)CC1] | 497.2 [M + 1]. |
| 153 | 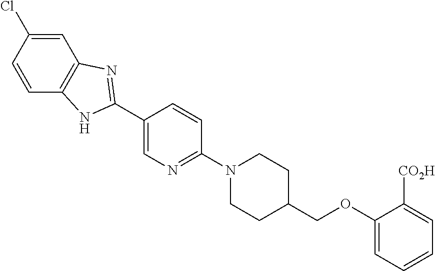 [SMILES: O=C(O)c1ccccc1OCC1CCN(c2ccc(-c3nc4cc(Cl)ccc4[nH]3)cn2)CC1] | 463.1 [M + 1]. |
| 154 | 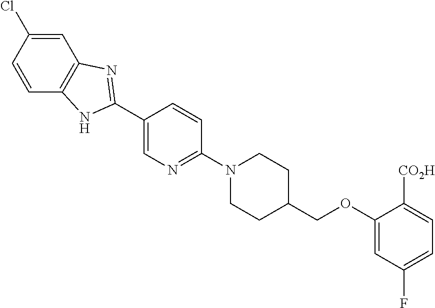 [SMILES: O=C(O)c1ccc(F)cc1OCC1CCN(c2ccc(-c3nc4cc(Cl)ccc4[nH]3)cn2)CC1] | 481.2 [M + 1]. |
| 155 | 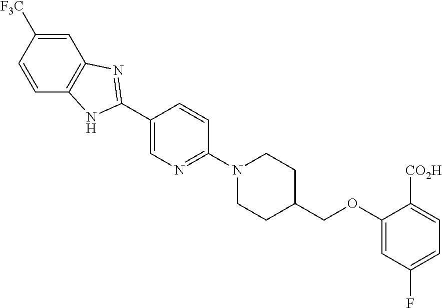 [SMILES: O=C(O)c1ccc(F)cc1OCC1CCN(c2ccc(-c3nc4cc(C(F)(F)F)ccc4[nH]3)cn2)CC1] | 515.2 [M + 1]. |
| 156 | 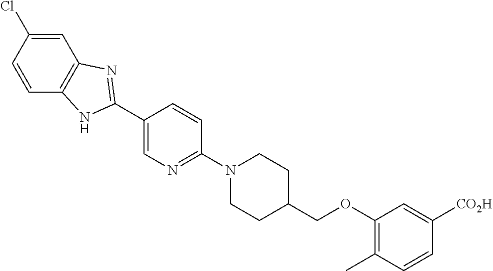 [SMILES: Cc1ccc(C(=O)O)cc1OCC1CCN(c2ccc(-c3nc4cc(Cl)ccc4[nH]3)cn2)CC1] | 477.2 [M + 1]. |
| 157 | 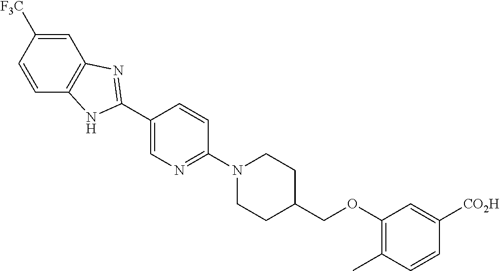 [SMILES: Cc1ccc(C(=O)O)cc1OCC1CCN(c2ccc(-c3nc4cc(C(F)(F)F)ccc4[nH]3)cn2)CC1] | 511.1 [M + 1]. |
| 158 | 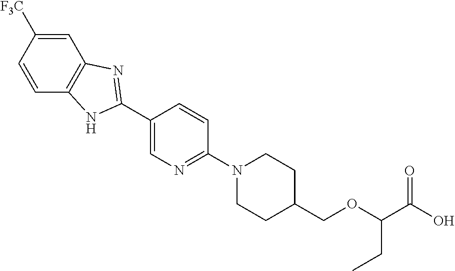 [SMILES: CCC(OCC1CCN(c2ccc(-c3nc4cc(C(F)(F)F)ccc4[nH]3)cn2)CC1)C(=O)O] | 463.2 [M + 1]. |
| 159 | 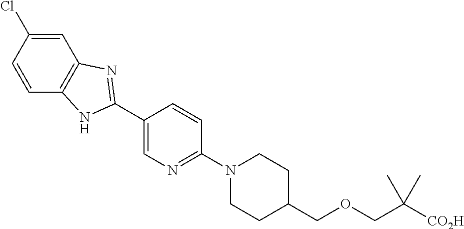 [SMILES: CC(C)(COCC1CCN(c2ccc(-c3nc4cc(Cl)ccc4[nH]3)cn2)CC1)C(=O)O] | 443.2 [M + 1]. |
| 160 | 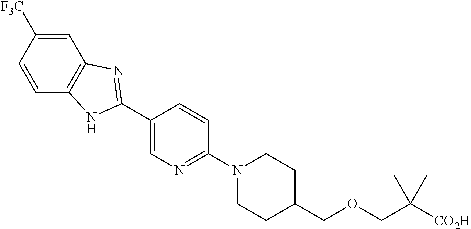 [SMILES: CC(C)(COCC1CCN(c2ccc(-c3nc4cc(C(F)(F)F)ccc4[nH]3)cn2)CC1)C(=O)O] | 477.2 [M + 1]. |
| 161 | 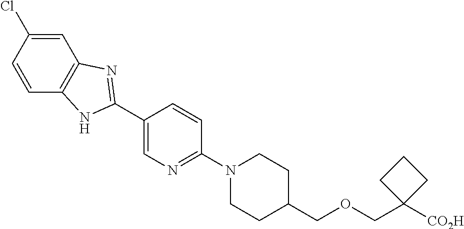 [SMILES: O=C(O)C1(COCC2CCN(c3ccc(-c4nc5cc(Cl)ccc5[nH]4)cn3)CC2)CCC1] | 455.2 [M + 1]. |
| 162 | 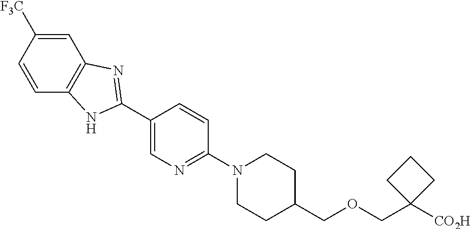 [SMILES: O=C(O)C1(COCC2CCN(c3ccc(-c4nc5cc(C(F)(F)F)ccc5[nH]4)cn3)CC2)CCC1] | 489.2 [M + 1]. |

The compounds of the formulas described herein, particularly the Examples listed in the table below, had activity inhibiting DGAT-1 enzyme with an IC₅₀ value of less than 10 μM and more typically of less than 1 μM or less than 0.1 μM. Such results are indicative of the activity of the compounds described herein for use as DGAT-1 inhibitors.

### DGAT1 CPM Assay

If Examples 1-140 were assayed, they were assayed as follows: 20 uL substrate mixture of 300 uM diolein, 40 uM oleoyl-CoA, 10% ethanol and 1 uL of the compound with different concentrations were delivered to a 384 well assay plate (Corning 3573) using a Tecan with TeMO module. Later 19 uL of enzyme mixture of 1.05 ug/ml human DGAT1 in buffer (200 mM Tris, pH7, 200 mM sucrose, 200 mM MgCl2+20 ug/ml NEM-treated BSA) was added via a Multidrop Combi using a microcassette. 20 uL of 90 uM CPM reagent in 90% ethanol was added after 1 hour incubation at room temperature. After 30 minutes at room temperature in dark, fluorescence measurement on Envision was carried out and IC₅₀s were calculated.

| Example | IC50 |
|---|---|
| 1 | 6 |
| 2 | 8 |
| 4 | 27 |
| 5 | 5 |
| 7 | 19 |
| 8 | 15 |
| 9 | 20 |
| 10 | 7900 |
| 11 | 6600 |
| 12 | 349 |
| 13 | 147 |
| 14 | 7 |
| 15 | 2 |
| 16 | 4 |
| 17 | 24 |
| 18 | 26 |
| 19 | 3 |
| 20 | 61 |
| 21 | 440 |
| 22 | 51 |
| 23 | 16 |
| 24 | 4 |
| 25 | 3 |
| 26 | 3 |
| 27 | 13 |
| 28 | 23 |
| 29 | 5000 |
| 32 | 9 |
| 33 | 7 |
| 34 | 4 |
| 35 | 29 |
| 36 | 67 |
| 37 | 2 |
| 38 | 8 |
| 39 | 15 |
| 41 | 1200 |
| 42 | 50 |
| 43 | 6 |
| 44 | 89 |
| 45 | 7 |
| 46 | 97 |
| 47 | 5 |
| 48 | 60 |
| 49 | 220 |
| 50 | 17 |
| 51 | 6 |
| 52 | 10 |
| 53 | 56 |
| 54 | 51 |
| 56 | 2 |
| 57 | 2 |
| 60 | 16 |
| 61 | 46 |
| 62 | 90 |
| 63 | 118 |
| 64 | 2 |
| 65 | 790 |
| 66 | 140 |
| 67 | 1400 |
| 68 | 2 |
| 69 | 2 |
| 70 | 11 |
| 71 | 3 |
| 72 | 35 |
| 73 | 71 |
| 74 | 15 |
| 75 | 14 |
| 76 | 2 |
| 77 | 17 |
| 78 | 6 |
| 80 | 23 |
| 81 | 17 |
| 82 | 19 |
| 83 | 17 |
| 84 | 25 |
| 85 | 9 |
| 86 | 19 |
| 87 | 14 |
| 88 | 9 |
| 89 | 4 |
| 90 | 15 |
| 91 | 7 |
| 92 | 147 |
| 93 | 71 |
| 94 | 122 |
| 95 | 85 |
| 96 | 277 |
| 97 | 1000 |
| 98 | 4 |
| 99 | 9 |
| 100 | 2 |
| 101 | 27 |
| 102 | 4 |
| 103 | 4 |
| 104 | 6 |
| 105 | 44 |
| 106 | 15 |
| 107 | 48 |
| 108 | 125 |
| 109 | 2 |
| 110 | 425 |
| 111 | 21 |
| 113 | 520 |
| 114 | 9 |
| 115 | 28 |
| 116 | 99 |
| 117 | 4 |
| 118 | 5 |
| 119 | 4 |
| 120 | 29 |
| 121 | 45 |
| 122 | 10 |
| 123 | 2 |
| 125 | 64 |
| 127 | 100 |
| 128 | 324 |
| 129 | 1 |
| 130 | 15 |
| 131 | 2 |
| 132 | 2 |
| 133 | 2 |
| 134 | 4 |
| 135 | 11 |
| 136 | 6 |
| 138 | 45 |
| 139 | 12 |
| 140 | 89 |

### Assay

If compounds 141-162 were assayed they were assayed as follows: the in vitro assay to identify DGAT1 inhibitors uses human DGAT1 enzyme expressed in Sf9 insect cells prepared as microsomes. The reaction is initiated by the addition of the combined substrates 1,2-dioleoyl-sn-glycerol and [¹⁴C]-palmitoyl-Co A and incubated with test compounds and microsomal membranes for 2 hours at room temperature. The assay is stopped by adding 0.5 mg wheat germ agglutinin beads in assay buffer with 1% Brij-35 and 1% 3-cholamidopropyldimethyl-ammonio-1-propane sulfonate. Plates are sealed with TopSeal and incubated for 18 hours to allow the radioactive triglyceride product to come into proximity with the bead. Plates are read on a TopCount instrument.

Percent inhibition was calculated as the percent of (test compound inhibition minus non-specific binding) relative to (total binding minus non-specific binding). IC₅₀ values were determined by curve fitting the data to a Sigmoidal dose-response in GraphPad Prism utilizing the following equation:

Y=A+(B−A)/(1+10̂((Log IC₅₀−X))),

where A and B are the bottom and top of the curve (highest and lowest inhibition), respectively, and X is the logarithm of concentration.

### Potency of DGAT-1 Inhibitors

| Example | IC₅₀ (nM) |
|---|---|
| 141 | 2500 |
| 142 | 13 |
| 143 | 1830 |
| 144 | 8 |
| 146 | 18 |
| 148 | 1070 |
| 150 | 67 |
| 151 | 2700 |
| 152 | 14 |
| 153 | 85 |
| 154 | 58 |
| 155 | 31 |
| 156 | 12 |
| 157 | 15 |
| 159 | 108 |
| 160 | 84 |
| 161 | 95 |
| 162 | 62 |

What is claimed is:

1. A compound of formula (I): 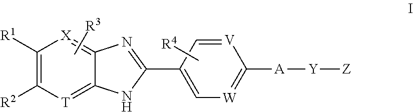
or pharmaceutically acceptable salts thereof, wherein A is a non-aromatic, nitrogen-containing ring selected from the group consisting of: 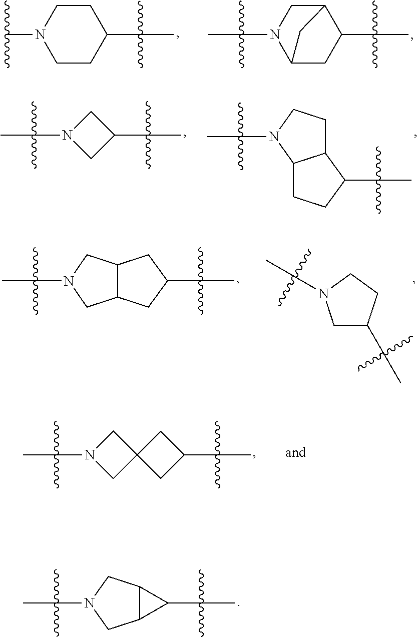
wherein A is unsubstituted or substituted with one or more substituents selected from R⁵;
wherein each occurrence of T, X, V and W are independently selected from the group consisting of —CH— and —N—;
wherein Y is —(CH₂)m-O—(CH₂)n-;
Z is selected from the group consisting of C₁-C₆alkyl, aryl, C₃-C₈cycloalkyl and heterocycle, wherein the C₁-C₆alkyl, aryl, cycloalkyl and heterocycle can be unsubstituted or substituted with 1-3 substituents selected from R⁶;
R¹, R², R³, R⁴ and R⁵ are independently selected from the group consisting of hydrogen, halogen, C₁-C₆alkyl, halogen-substitutedC₁-C₆alkyl, —OH, C₁-C₆alkylOH, —OC₁-C₆alkyl, —Ohalogen-substitutedC₁-C₆alkyl, —SO²C₁-C₆alkyl and —CN or when taken together R¹ and R² form pyrazol;
R⁶ is selected from the group consisting of halogen, C₁-C₆alkyl, halogen-substitutedC₁-C₆alkyl, COC₁-C₆alkyl, COhalogen-substitutedC₁-C₆alkyl, —OH, C₁-C₆alkylOH, —COOH, —COCOOH, —COOC₁-C₆alkyl, —C₁-C₆alkylCOOC₁-C₆alkyl, —C₁-C₆alkylCOOH, —OC₁-C₆alkylCOOH, —CN, C₁-C₆alkylCN, heterocycle, CONHSO₂C₁-C₆alkyl, CONHSO₂halogen-substitutedC₁-C₆alkyl, CONHSO₂C₃-C₆cycloalkyl, CONHSO₂C₃-C₆cycloalkylC₁-C₆alkyl, CONHSO₂heteroaryl, CONHSO₂aryl, CONHSO₂halogen-substitutedaryl and CONHSO₂arylhalogen-substitutedC₁-C₆alkyl; and
m and n are independently selected from the list consisting of 0, 1 or 2.

2. A compound of claim 1 or pharmaceutically acceptable salt thereof having formula Ia, formula Ib, formula Ic or formula Id, wherein X, T, R¹, R², R³, R⁴, R⁵ and R⁶ are defined as in claim 1:

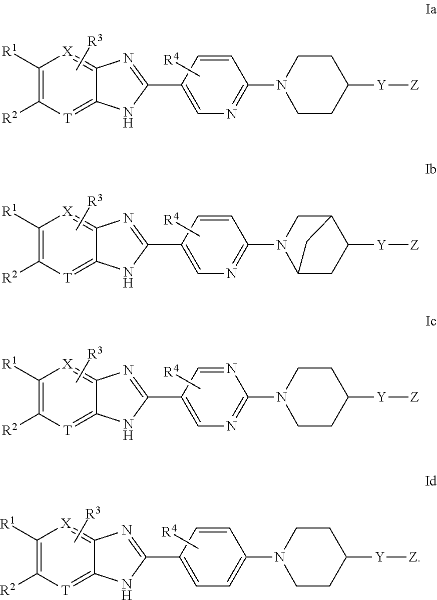

3. A compound of claim 1 or pharmaceutically acceptable salt thereof having formula Ie, formula If, formula Ig, formula Ih, formula Ii or formula Ij wherein X, T, R¹, R², R³, R⁴, R⁵ and R⁶ are defined as in claim 1:

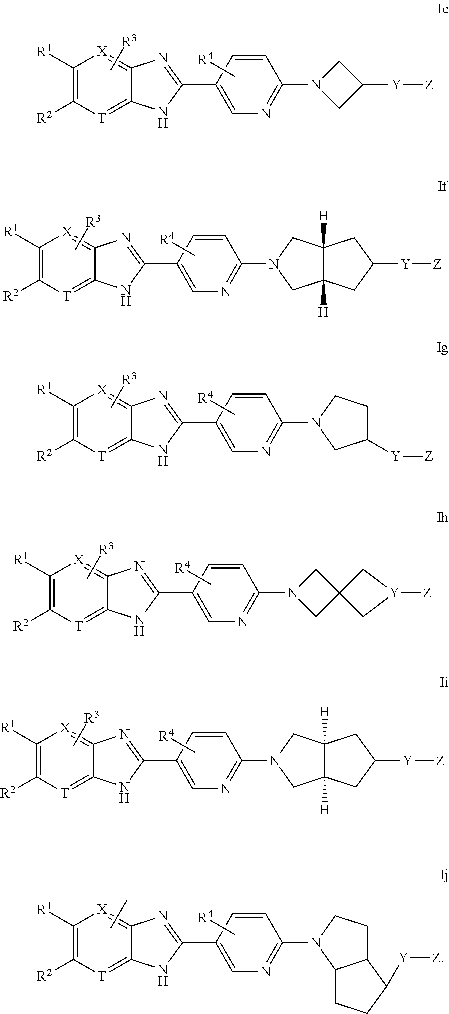

4. A compound of claim 1 or pharmaceutically acceptable salt thereof having formula Ik or formula Il wherein T, X, R¹, R², R³, R⁴, R⁵ and R⁶ are defined as in claim 1:

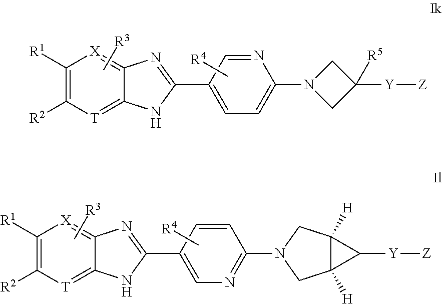

5. A compound of claim 1 or pharmaceutically acceptable salt thereof wherein T and X are both —CH—.

6. A compound of claim 1 or pharmaceutically acceptable salt thereof wherein V is —N— and W is —CH—.

7. A compound of claim 1 or pharmaceutically acceptable salt thereof wherein T is —N— and X is —CH—.

8. A compound of claim 1, or pharmaceutically acceptable salt thereof, wherein A is

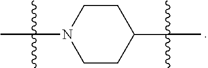 [SMILES: *C1CCN(*)CC1]

9. A compound of claim 1 or pharmaceutically acceptable salt thereof wherein R¹ is hydrogen or halogen.

10. A compound of claim 1 or pharmaceutically acceptable salt thereof wherein R² is hydrogen or halogen.

11. A compound of claim 1 or pharmaceutically acceptable salt thereof wherein R³ is hydrogen, methyl or halogen.

12. A compound of claim 1 or pharmaceutically acceptable salt thereof wherein R⁴ is hydrogen or halogen.

13. A compound of claim 1 or pharmaceutically acceptable salt thereof wherein Z is selected from the group consisting of: C₁-C₆alkyl, phenyl, cyclohexyl, cyclobutyl, cyclopropyl, tetrahydropyran, pyridyl, pyrimidinyl, oxazole.

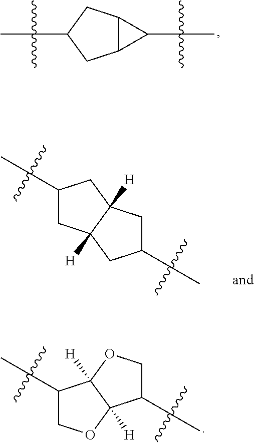 [SMILES: *C1CC2C(*)C2C1 ; *C1C[C@H]2CC(*)C[C@H]2C1 ; *C1CO[C@@H]2C(*)CO[C@H]12]

14. A compound of claim 1 or pharmaceutically acceptable salt thereof wherein m and n are independently selected from 0 or 1.

15. A compound of claim 1 or pharmaceutically acceptable salt thereof wherein R⁶ is selected from the group consisting of —OH, —COOH, —COOC₁-C₆alkyl, —C₁-C₆alkylCOOC₁-C₆alkyl, C₁-C₆alkyl or —C₁-C₆alkylCOOH.

16. A compound of claim 1 or pharmaceutically acceptable salt thereof wherein R⁶ is CONHSO₂C₁-C₆alkyl, CONHSO₂halogen-substitutedC₁-C₆alkyl, CONHSO₂C₃-C₆cycloalkyl, CONHSO₂C₃-C₆cycloalkylC₁-C₆alkyl, CONHSO₂heteroaryl, CONHSO₂aryl, CONHSO₂halogen-substitutedaryl and CONHSO₂arylhalogen-substitutedC₁-C₆alkyl.

17. A compound or pharmaceutically acceptable salt thereof selected from the group consisting of:

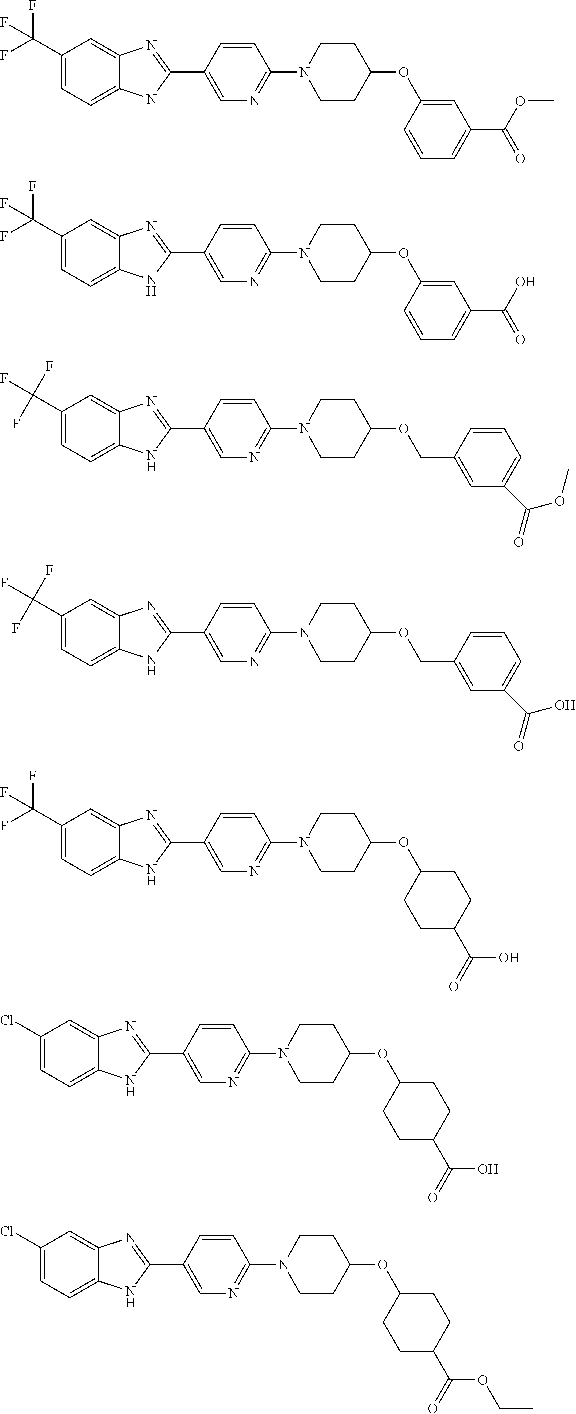

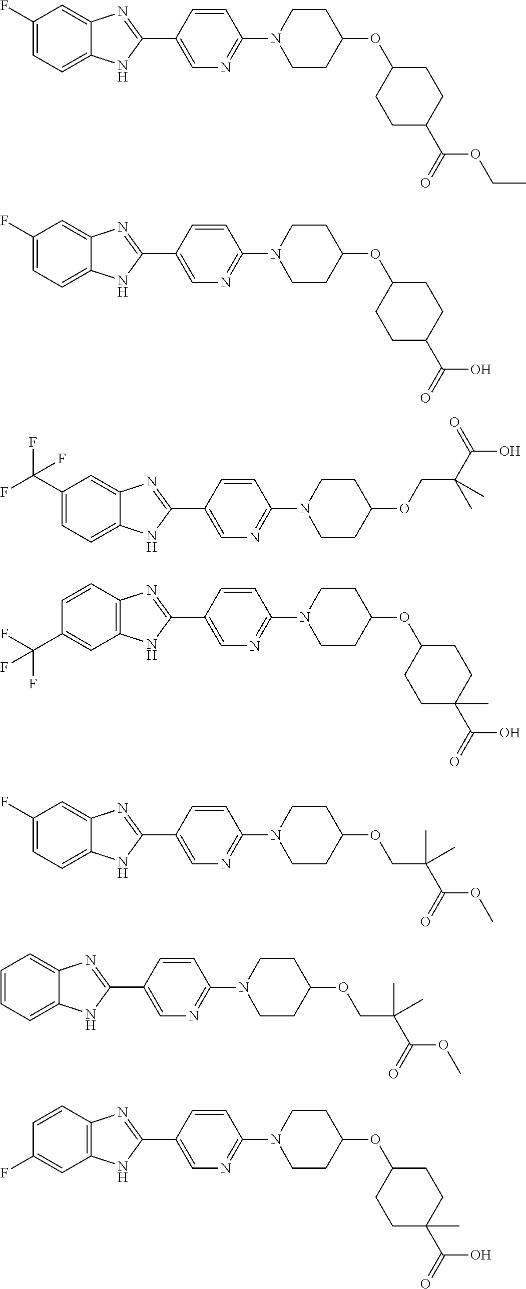

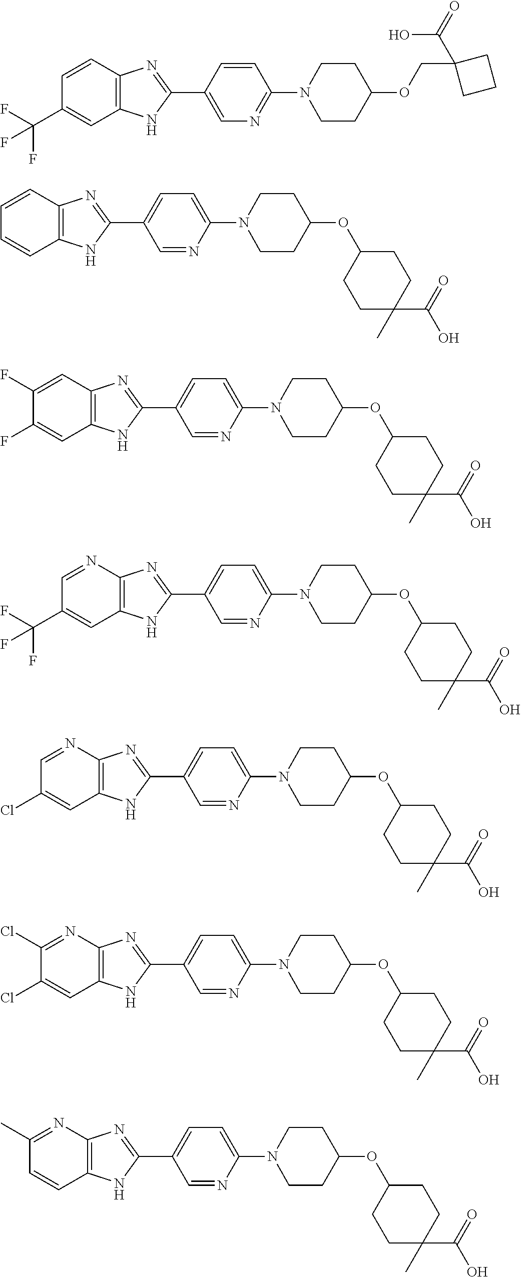

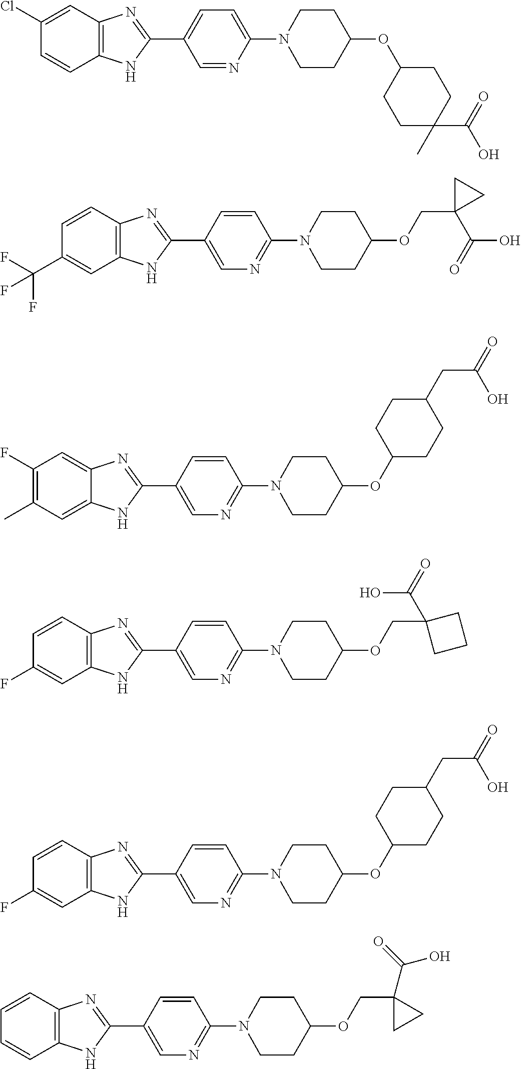

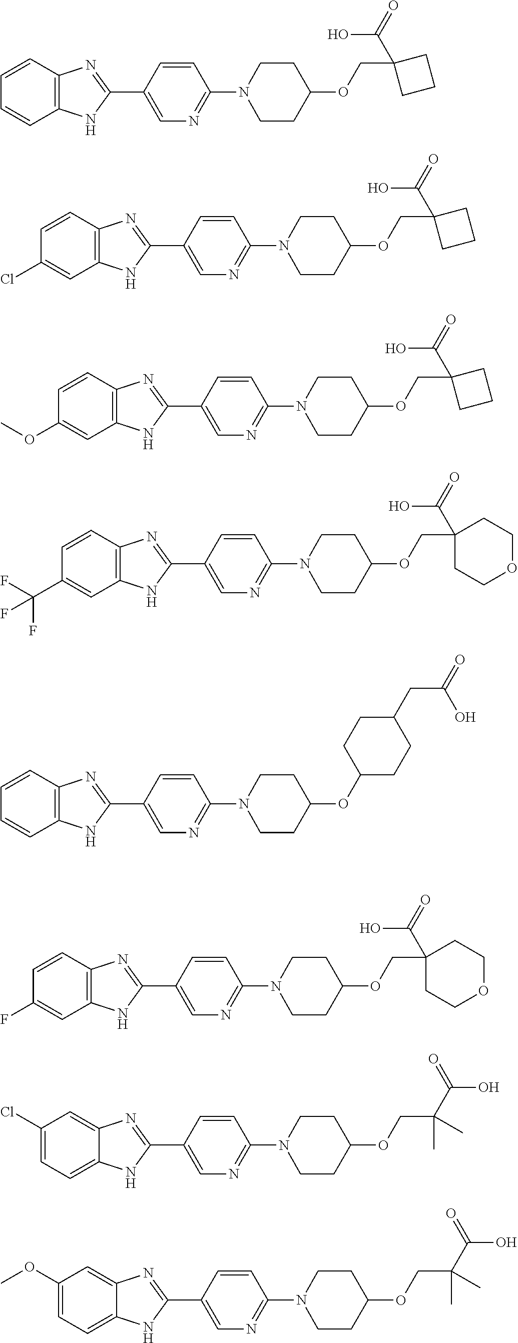

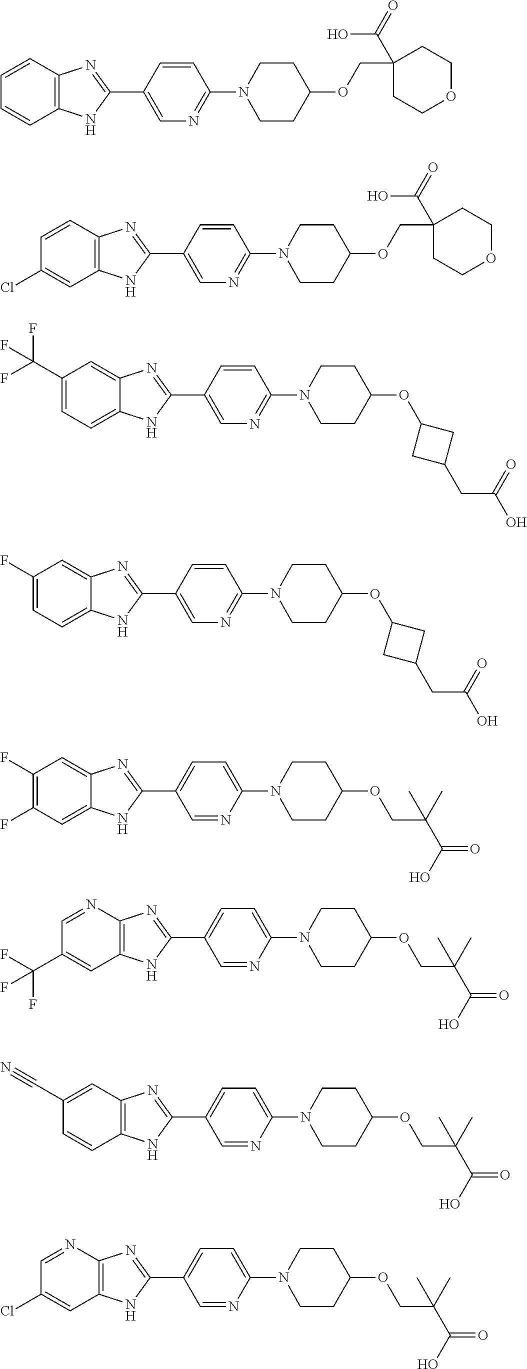

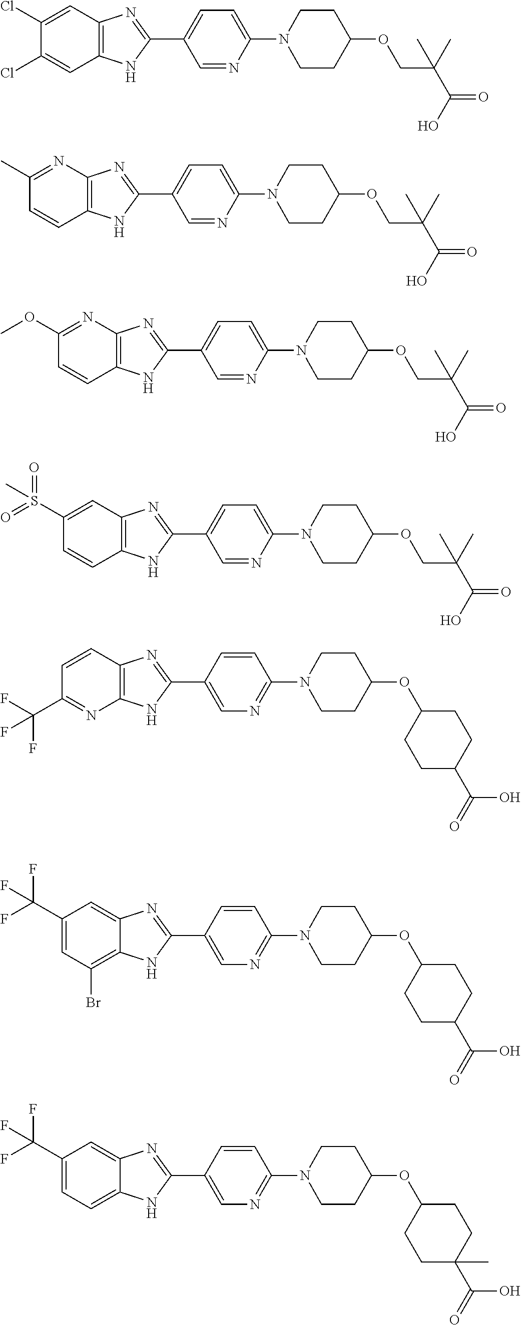

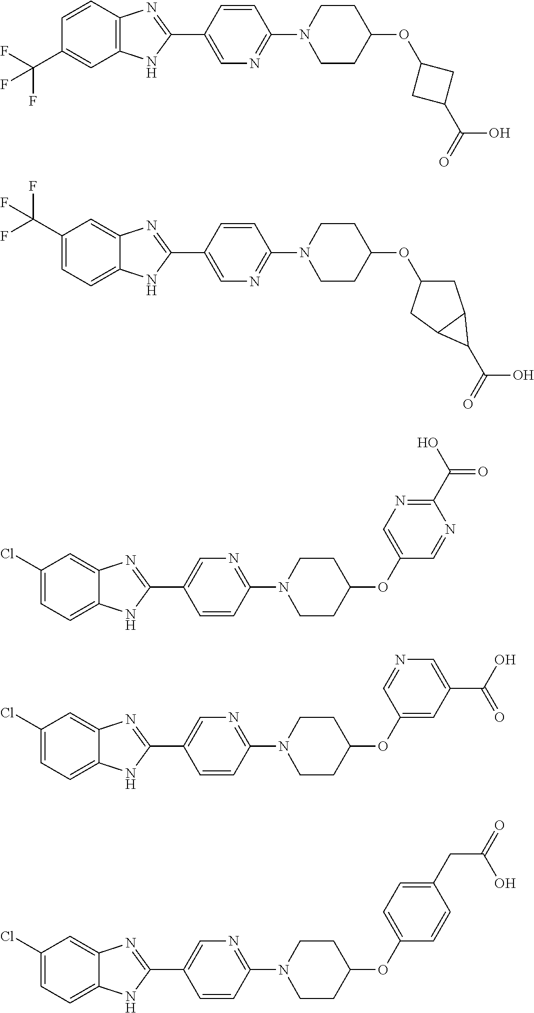 [SMILES: O=C(O)C1CC(OC2CCN(c3ccc(-c4nc5ccc(C(F)(F)F)cc5[nH]4)cn3)CC2)C1 ; O=C(O)C1C2CC(OC3CCN(c4ccc(-c5nc6cc(C(F)(F)F)ccc6[nH]5)cn4)CC3)CC21 ; O=C(O)c1ncc(OC2CCN(c3ccc(-c4nc5cc(Cl)ccc5[nH]4)cn3)CC2)cn1 ; O=C(O)c1cncc(OC2CCN(c3ccc(-c4nc5cc(Cl)ccc5[nH]4)cn3)CC2)c1 ; O=C(O)Cc1ccc(OC2CCN(c3ccc(-c4nc5cc(Cl)ccc5[nH]4)cn3)CC2)cc1]

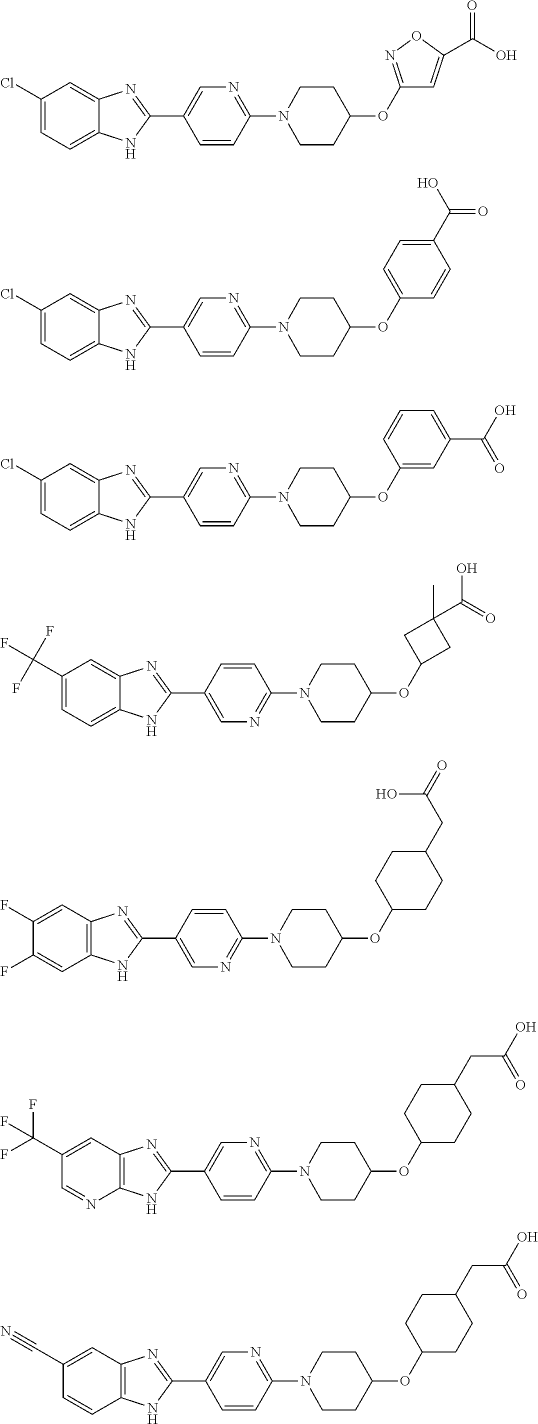

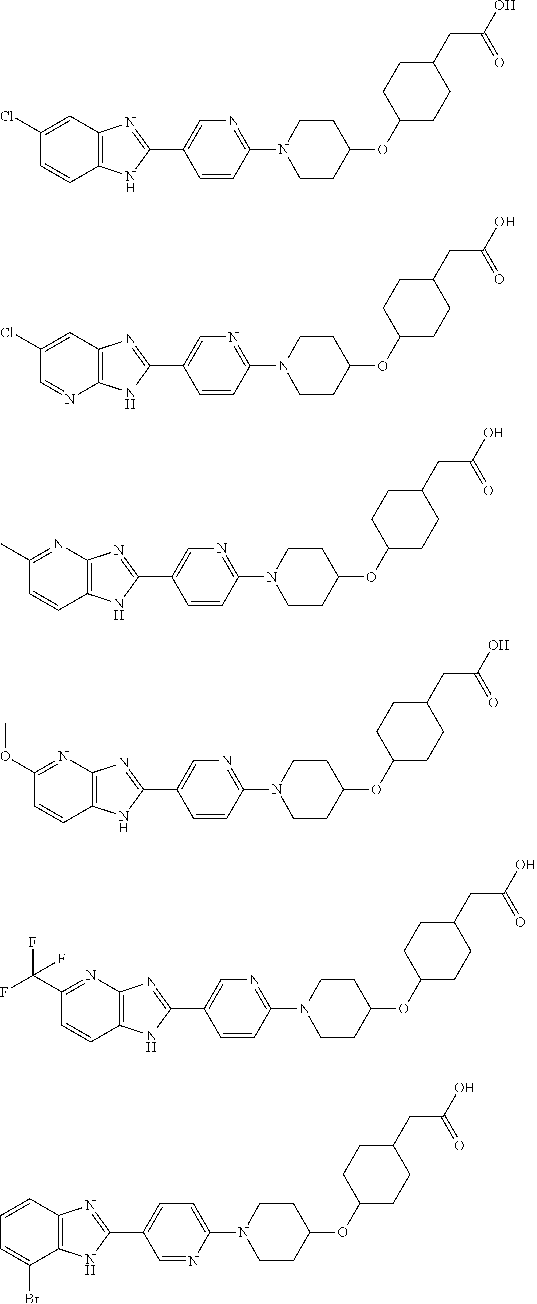 [SMILES: O=C(O)CC1CCC(OC2CCN(c3ccc(-c4nc5cc(Cl)ccc5[nH]4)cn3)CC2)CC1 ; O=C(O)CC1CCC(OC2CCN(c3ccc(-c4nc5cc(Cl)cnc5[nH]4)cn3)CC2)CC1 ; Cc1ccc2[nH]c(-c3ccc(N4CCC(OC5CCC(CC(=O)O)CC5)CC4)nc3)nc2n1 ; COc1ccc2[nH]c(-c3ccc(N4CCC(OC5CCC(CC(=O)O)CC5)CC4)nc3)nc2n1 ; O=C(O)CC1CCC(OC2CCN(c3ccc(-c4nc5nc(C(F)(F)F)ccc5[nH]4)cn3)CC2)CC1 ; O=C(O)CC1CCC(OC2CCN(c3ccc(-c4nc5cccc(Br)c5[nH]4)cn3)CC2)CC1]

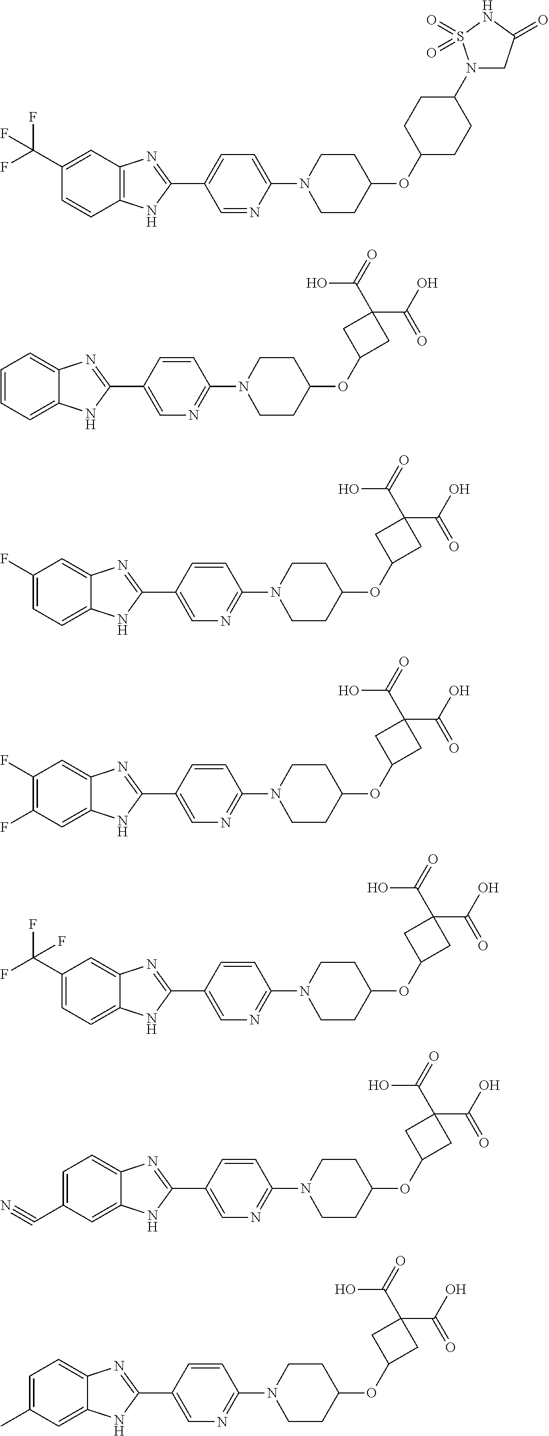

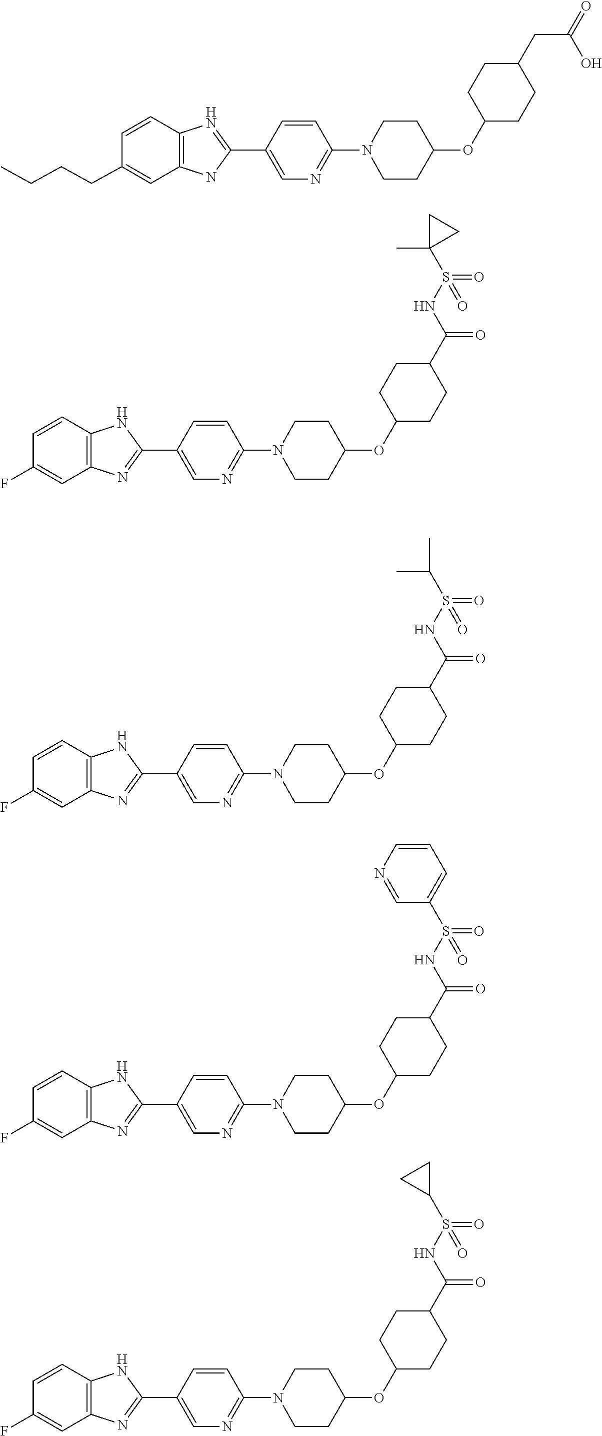

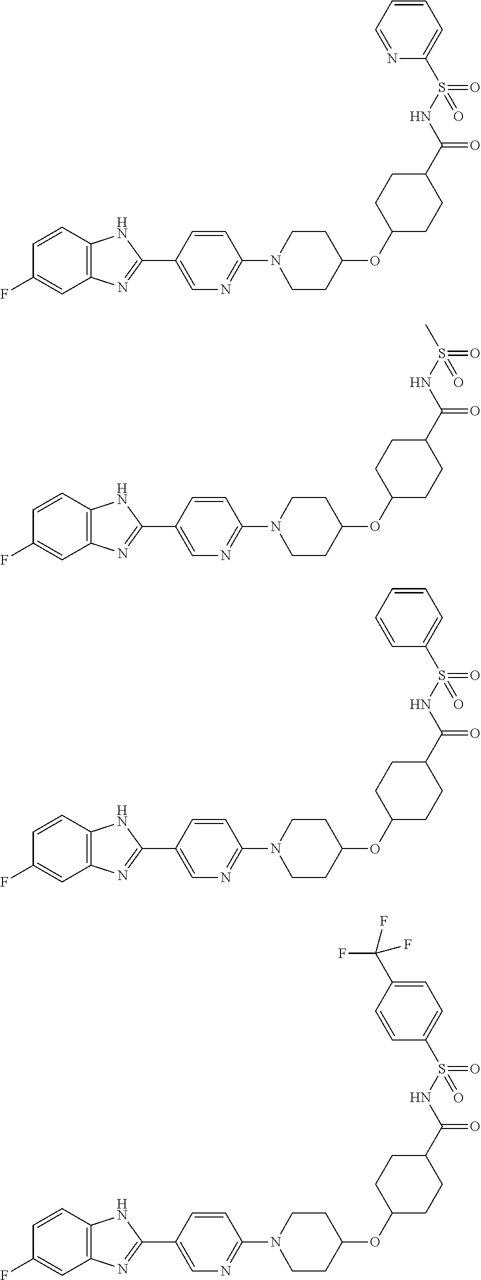 [SMILES: O=C(NS(=O)(=O)c1ccccn1)C1CCC(OC2CCN(c3ccc(-c4nc5cc(F)ccc5[nH]4)cn3)CC2)CC1 ; CS(=O)(=O)NC(=O)C1CCC(OC2CCN(c3ccc(-c4nc5cc(F)ccc5[nH]4)cn3)CC2)CC1 ; O=C(NS(=O)(=O)c1ccccc1)C1CCC(OC2CCN(c3ccc(-c4nc5cc(F)ccc5[nH]4)cn3)CC2)CC1 ; O=C(NS(=O)(=O)c1ccc(C(F)(F)F)cc1)C1CCC(OC2CCN(c3ccc(-c4nc5cc(F)ccc5[nH]4)cn3)CC2)CC1]

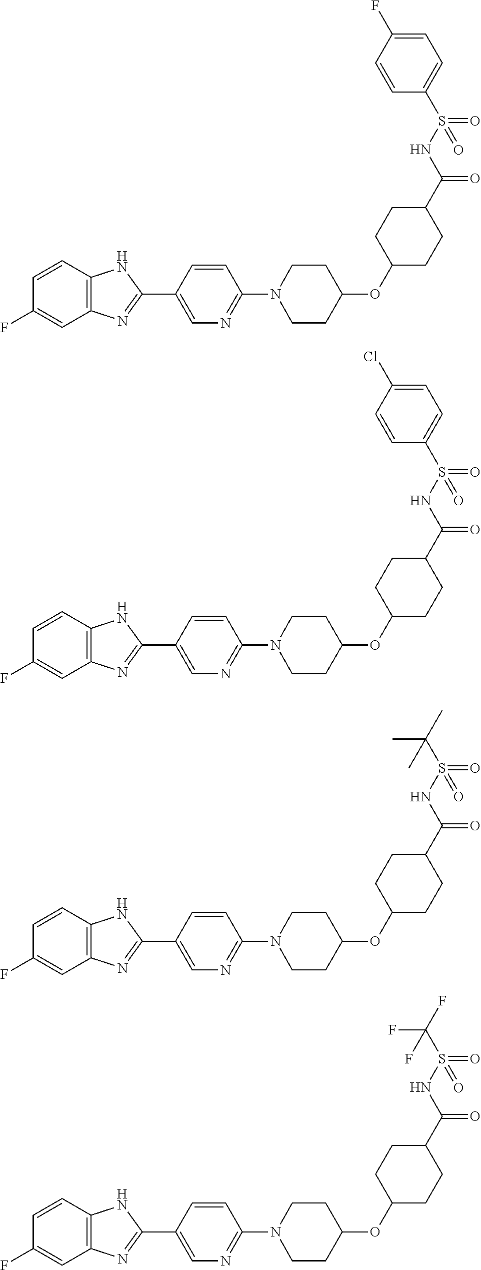 [SMILES: O=C(NS(=O)(=O)c1ccc(F)cc1)C1CCC(OC2CCN(c3ccc(-c4nc5cc(F)ccc5[nH]4)cn3)CC2)CC1 ; O=C(NS(=O)(=O)c1ccc(Cl)cc1)C1CCC(OC2CCN(c3ccc(-c4nc5cc(F)ccc5[nH]4)cn3)CC2)CC1 ; CC(C)(C)S(=O)(=O)NC(=O)C1CCC(OC2CCN(c3ccc(-c4nc5cc(F)ccc5[nH]4)cn3)CC2)CC1 ; O=C(NS(=O)(=O)C(F)(F)F)C1CCC(OC2CCN(c3ccc(-c4nc5cc(F)ccc5[nH]4)cn3)CC2)CC1]

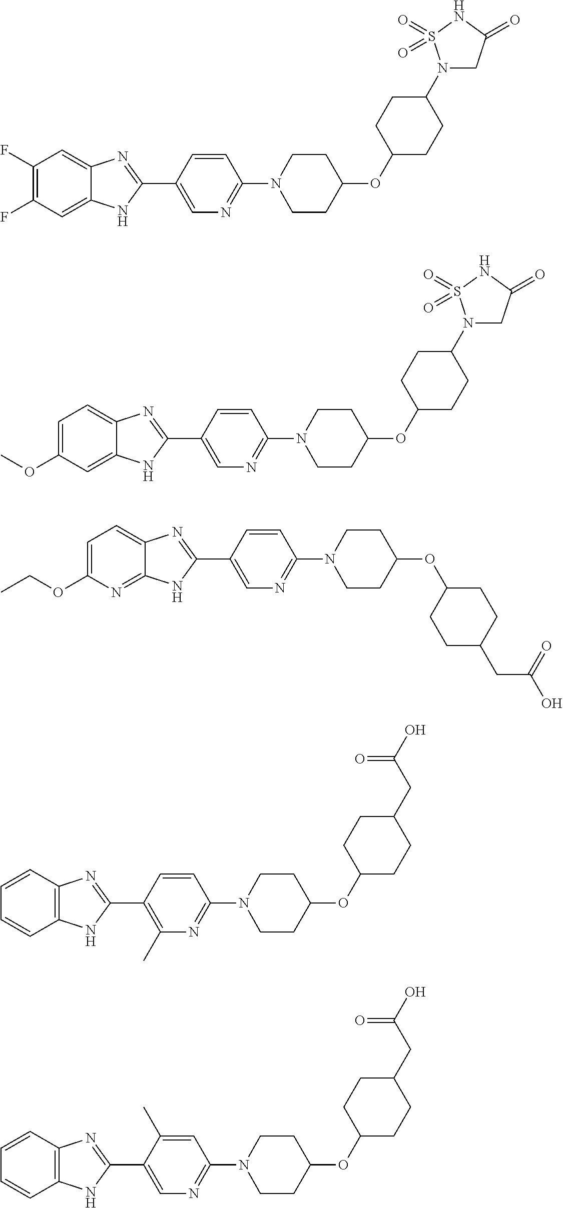 [SMILES: O=C1CN(C2CCC(OC3CCN(c4ccc(-c5nc6cc(F)c(F)cc6[nH]5)cn4)CC3)CC2)S(=O)(=O)N1 ; COc1ccc2nc(-c3ccc(N4CCC(OC5CCC(N6CC(=O)NS6(=O)=O)CC5)CC4)nc3)[nH]c2c1 ; CCOc1ccc2nc(-c3ccc(N4CCC(OC5CCC(CC(=O)O)CC5)CC4)nc3)[nH]c2n1 ; Cc1nc(N2CCC(OC3CCC(CC(=O)O)CC3)CC2)ccc1-c1nc2ccccc2[nH]1 ; Cc1cc(N2CCC(OC3CCC(CC(=O)O)CC3)CC2)ncc1-c1nc2ccccc2[nH]1]

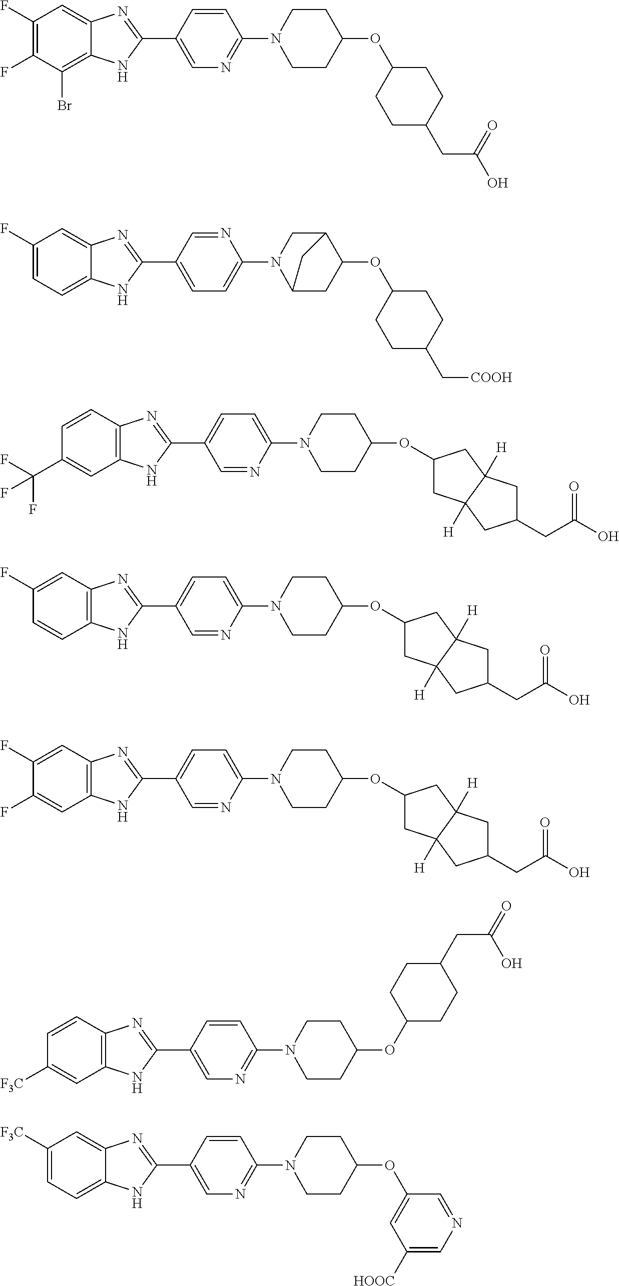

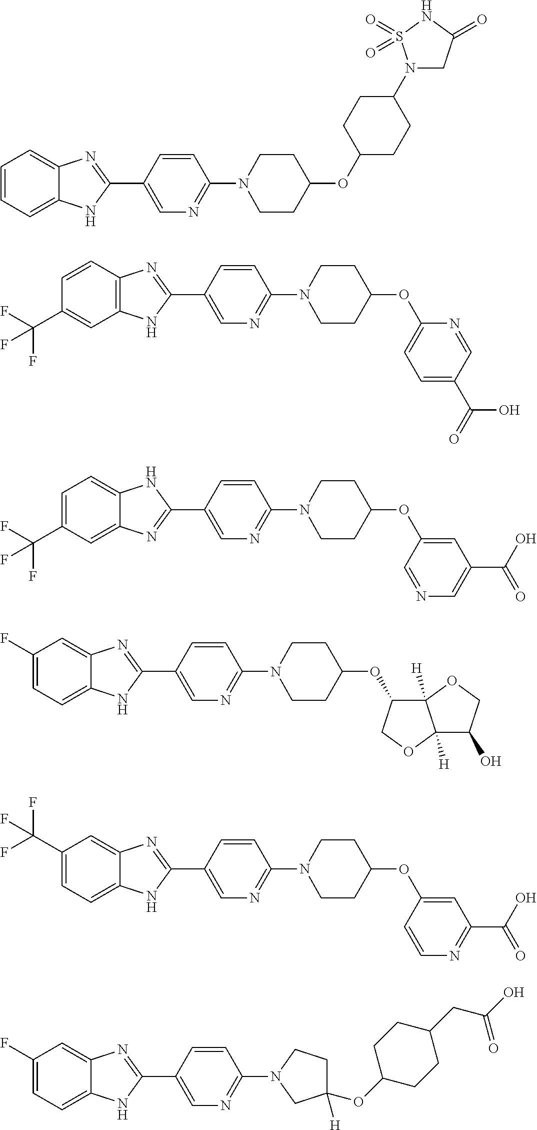

18. A pharmaceutical composition comprising a compound of claim 1, or a pharmaceutically acceptable salt thereof, and a pharmaceutically acceptable carrier.

19. (canceled)

20. A method for the treatment of a condition selected from the group consisting of obesity and diabetes comprising administering to an individual a pharmaceutical composition comprising the compound of claim 1.
